(19) United States
(12) Patent Application Publication
(10) Pub. No.: US 2023/0416308 A1
(43) Pub. Date: Dec. 28, 2023

(54) ENCAPSULATED RNA REPLICONS AND METHODS OF USE

(71) Applicant: Oncorus, Inc., Andover, MA (US)

(72) Inventors: Lorena LERNER, Cambridge, MA (US); Edward M. KENNEDY, Cambridge, MA (US); Jennifer S. LEE, Cambridge, MA (US)

(21) Appl. No.: 17/999,582

(22) PCT Filed: May 28, 2021
(86) PCT No.: PCT/US2021/034787

§ 371 Date: Nov. 22, 2022

Related U.S. Application Data
(60) Provisional application No. 63/032,000, filed on May 29, 2020.

(51) Int. Cl. C07K 14/005; C12N 15/86; C07K 14/55; C07K 14/54; C07K 16/28; C07K 14/52; A61K 9/127; A61P 35/00; A61K 39/125

(52) U.S. Cl. CPC C07K 14/005; A61K 2039/86; C12Y 304/23016; C07K 14/55; C07K 14/54; C07K 16/28; C07K 16/2809; C07K 14/522; A61K 9/127; A61P 35/00; C07K 14/5434; C07K 14/523; A61K 39/125; C12N 2770/32052; C12N 2770/32043; C12N 2770/32071; C07K 2319/50; C07K 2319/21; C07K 2317/622; C07K 2317/31; C07K 2317/569; C12N 15/86

(57) ABSTRACT

The disclosure relates to oncolytic virus derived replicons and capsidation of the same. The disclosure also relates to the incorporation of one or more transgenes encoding payload molecules into the replicon. The disclosure further relates to the encapsulation of the replicon and/or recombinant RNA molecules encoding oncolytic viruses into particles and the use of the replicon and/or particles for the treatment and prevention of cancer.

### CROSS-REFERENCE TO RELATED APPLICATIONS

This application is a U.S. National Phase Application, filed under 35 U.S.C. § 371, of International Application No. PCT/US2021/034787, filed May 28, 2021, which claims priority to U.S. Provisional Application No. 63/032,000, filed May 29, 2020, the contents of each of which are incorporated by reference in their entirety.

### INCORPORATION BY REFERENCE OF SEQUENCE LISTING

The contents of the text file submitted electronically herewith are incorporated herein by reference in their entirety: A computer readable format copy of the Sequence Listing (filename: ONCR_019_01US_SeqList_ST25.txt, date created: Nov. 21, 2022, file size: 790,366 bytes).

### FIELD

The present disclosure generally relates to the fields of immunology, inflammation, and cancer therapeutics. More specifically, the present disclosure relates to viral replicons with improved loading capacity and heterologous polynucleotide encoding payload molecules, as well as particle-encapsulated viral replicons. The disclosure further relates to the treatment and prevention of proliferative disorders such as cancer.

### BACKGROUND

There remains a long-felt and unmet need in the art for compositions and methods related to therapeutic use of virus and/or viral replicons comprising improved loading capacity and/or functionality for one or more therapeutic molecules. The present disclosure provides such compositions and methods, and more.

### SUMMARY

The disclosure provides recombinant RNA replicons comprising: a) a picornavirus genome, wherein the picornavirus genome comprises a deletion or a truncation in one or more protein coding regions; and b) a heterologous polynucleotide. In some embodiments, the picornavirus genome comprises the deletion or the truncation in one or more VP coding regions. In some embodiments, the picornavirus genome comprises the deletion or the truncation in each of the VP1, VP3 and VP2 coding regions. In some embodiments, the picornavirus genome comprises the deletion of the VP1 and VP3 coding regions and the truncation of the VP2 coding region. In some embodiments, the picornavirus is selected from a senecavirus, a cardiovirus, and an enterovirus. In some embodiments, the deletion or the truncation comprises at least 500 bp, at least 1000 bp, at least 1500 bp, at least 2000 bp, at least 2500 bp, or at least 3000 bp. In some embodiments, the deletion or the truncation comprises at least 2000 bp. In some embodiments, a site of the deletion or a site of the truncation comprises the heterologous polynucleotide. In some embodiments, the heterologous polynucleotide is inserted between a 2A coding region and a 2B coding region. In some embodiments, the heterologous polynucleotide is inserted between a 3D coding region and a 3′ untranslated region (UTR). In some embodiments, the heterologous polynucleotide comprises at least 1000 bp, at least 2000 bp, or at least 3000 bp.

The disclosure provides recombinant RNA replicons comprising: a) a Seneca Valley Virus (SVV) genome, wherein the SVV genome comprises a deletion or a truncation in one or more protein coding regions; and b) a heterologous polynucleotide (i.e., the replicon is a SVV derived replicon). In some embodiments, the deletion or the truncation comprises one or more nucleotides between nucleotide 1261 and 3477, inclusive of the endpoints, according to the numbering of SEQ ID NO: 1. In some embodiments, the deletion or the truncation comprises nucleotide 1261 to 3477, inclusive of the endpoints, according to the numbering of SEQ ID NO: 1. In some embodiments, the deletion or the truncation comprises at least 500 bp, at least 1000 bp, at least 1500 bp, or at least 2000 bp. In some embodiments, the deletion or the truncation comprises at least 2000 bp. In some embodiments, the SVV genome comprises a 5′ leader protein coding sequence. In some embodiments, the SVV genome comprises a VP4 coding region. In some embodiments, the SVV genome comprises a VP2 coding region or a truncation thereof. In some embodiments, the SVV genome comprises, from 5′ to 3′ direction, the 5′ leader protein coding sequence, the VP4 coding region, and the VP2 coding region or a truncation thereof. In some embodiments, a portion of the SVV genome comprising the 5′ leader protein coding sequence, the VP4 coding region, and the VP2 coding region or a truncation thereof has at least 90% sequence identity to nucleotide 1 to 1260 of SEQ ID NO: 1. In some embodiments, the SVV genome comprises, from 5′ to 3′ direction, the 5′ leader protein coding sequence, the VP4 coding region, the VP2 coding region or a truncation thereof, and the heterologous polynucleotide. In some embodiments, the SVV genome comprises a cis-acting replication element (CRE). In some embodiments, the CRE comprises between 10-200 bp. In some embodiments, the CRE comprises one or more nucleotides within the region corresponding to nucleotide 1000 to nucleotide 1260 according to SEQ ID NO: 1. In some embodiments, the CRE comprises one or more nucleotides within the region corresponding to nucleotide 1117 to nucleotide 1260 according to SEQ ID NO: 1. In some embodiments, the CRE comprises a polynucleotide sequence having at least 70%, at least 75%, at least 80%, at least 85%, at least 90%, at least 95%, at least 96%, at least 97%, at least 98%, at least 99%, or 100% identity to SEQ ID NO: 149. In some embodiments, the SVV genome further comprises a 2A coding region. In some embodiments, the 2A coding region is located between the VP2 coding region or a truncation thereof and the heterologous polynucleotide. In some embodiments, the SVV genome comprises one or more of a 2B coding region, a 2C coding region, a 3A coding region, a 3B coding region, a 3Cpro coding region, and a 3D(RdRp) coding region. In some embodiments, the SVV genome comprises a 2B coding region, a 2C coding region, a 3A coding region, a 3B coding region, a 3Cpro coding region, and a 3D(RdRp) coding region. In some embodiments, the SVV genome comprises, from 5′ to 3′, the 2B coding region, the 2C coding region, the 3A coding region, the 3B coding region, the 3Cpro coding region, and the 3D(RdRp) coding region. In some embodiments, a portion of the SVV genome comprising the 2B coding region, the 2C coding region, the 3A coding region, the 3B coding region, the 3Cpro coding region, and the 3D(RdRp) coding region has at least 90% sequence identity to nucleotide 3505 to 7310 according to SEQ ID NO: 1. In some embodiments, the SVV genome comprises, from 5′ to 3′, the heterologous polynucleotide and the 2B coding region.

The disclosure provides recombinant RNA replicons comprising: a) a coxsackievirus genome, wherein the coxsackievirus genome comprises a deletion or a truncation in one or more protein coding regions; and b) a heterologous polynucleotide (i.e., the replicon is a coxsackievirus derived replicon). In some embodiments, the deletion or the truncation comprises one or more nucleotides between nucleotide 717 to 3332, inclusive of the endpoints, according to the numbering of SEQ ID NO: 3. In some embodiments, the deletion or the truncation comprises nucleotide 717 to 3332, inclusive of the endpoints, according to the numbering of SEQ ID NO: 3. In some embodiments, the deletion or the truncation comprises at least 500 bp, at least 1000 bp, at least 1500 bp, at least 2000 bp, or at least 2600 bp. In some embodiments, the coxsackievirus genome comprises a 5′ UTR. In some embodiments, a portion of the coxsackievirus genome comprising the 5′ UTR has at least 90% sequence identity to SEQ ID NO: 4. In some embodiments, the coxsackievirus genome comprises one or more of a 2A coding region, a 2B coding region, a 2C coding region, a 3A coding region, a 3B coding region, a VPg coding region, a 3C coding region, a 3D pol coding region, and a 3′ UTR. In some embodiments, the coxsackievirus genome comprises a 2A coding region, a 2B coding region, a 2C coding region, a 3A coding region, a 3B coding region, a VPg coding region, a 3C coding region, a 3D pol coding region, and a 3′ UTR. In some embodiments, the coxsackievirus genome comprises, from 5′ to 3′ direction, the 2A coding region, the 2B coding region, the 2C coding region, the 3A coding region, the 3B coding region, the VPg coding region, the 3C coding region, the 3D pol coding region, and the 3′ UTR. In some embodiments, a portion of the coxsackievirus genome comprising the 2A coding region, the 2B coding region, the 2C coding region, the 3A coding region, the 3B coding region, the VPg coding region, the 3C coding region, the 3D pol coding region, and the 3′ UTR has at least 90% sequence identity to nucleotide 3492 to 7435 in SEQ ID NO: 3. In some embodiments, the coxsackievirus genome comprises, from 5′ to 3′, the 5′ UTR, the heterologous polynucleotide, and the 2A coding region.

The disclosure provides recombinant RNA replicons comprising: a) a encephalomyocarditis virus (EMCV) genome, wherein the EMCV genome comprises a deletion or a truncation in one or more protein coding regions; and b) a heterologous polynucleotide (i.e., the replicon is a EMCV derived replicon).

In some embodiments, the recombinant RNA replicon comprises an internal ribosome entry site (IRES) inserted between the heterologous polynucleotide and the 2B coding region.

In some embodiments, the heterologous polynucleotide of the recombinant RNA replicon encodes one or more payload molecules. In some embodiments, the heterologous polynucleotide of the recombinant RNA replicon encodes two or more payload molecules. In some embodiments, the two or more payload molecules are operably linked by one or more cleavage polypeptides. In some embodiments, the cleavage polypeptide comprises a 2A family self-cleaving peptide, a 3C cleavage site, a furin site, an IGSF1 polypeptide, or a HIV protease site. In some embodiments, the cleavage polypeptide comprises an IGSF1 polypeptide, and wherein the IGSF1 polypeptide comprises an amino acid sequence having at least 90% identity to SEQ ID NO: 75. In some embodiments, the cleavage polypeptide comprises an HIV protease site. In some embodiments, the cleavage polypeptide comprises a 2A family self-cleaving peptide. In some embodiments, the cleavage polypeptide comprises a furin site. In some embodiments, the heterologous polynucleotide encodes a polypeptide comprising the two or more payload molecules and the cleavage polypeptide comprising, from N-terminus to C-terminus: N′-payload molecule 1-cleavage polypeptide-payload molecule 2-C′. In some embodiments, the heterologous polynucleotide further comprises a coding region that encodes an HIV protease, and wherein the heterologous polynucleotide comprises a coding region that encodes a polypeptide comprising, from N-terminus to C-terminus: N′-Payload molecule 1-HIV protease site-HIV protease-HIV protease site-Payload molecule 2-C′. In some embodiments, the heterologous polynucleotide further comprises a coding region that encodes a third payload molecule, and wherein the heterologous polynucleotide comprises a coding region that encodes a polypeptide comprising, from N-terminus to C-terminus: N′-Payload molecule 1-HIV protease site-HIV protease-HIV protease site-Payload molecule 2-HIV protease site-Payload molecule 3-C′. In some embodiments, the recombinant RNA replicon of the disclosure further comprises a cleavage polypeptide at the C-terminus of the encoded polypeptide.

In some embodiments, the payload molecules are selected from a fluorescent protein, an enzyme, a cytokine, a chemokine, an antigen, an antigen-binding molecule capable of binding to a cell surface receptor, and a ligand for a cell-surface receptor. In some embodiments, the payload molecules are selected from:
a) one or more cytokines comprising IFNγ, GM-CSF, IL-2, IL-12, IL-15, IL-18, IL-23, and IL-36γ;
b) one or more chemokines comprising CXCL10, CCL4, CCL5, and CCL21;
c) one or more antibodies comprising an anti-PD1-VHH-Fc antibody, an anti-CD47-VHH-Fc antibody, and an anti-TGFβ-VHH(or scFv)-Fc antibody;
d) one or more bipartite polypeptides comprising a bipartite polypeptide binding to DLL3 and an effector cell target antigen, a bipartite polypeptide binding to FAP and an effector cell target antigen, and a bipartite polypeptide binding to EpCAM and an effector cell target antigen;
e) one or more tumor-associated antigens comprising survivin, MAGE family proteins, and all antigens according to Table 6;
f) one or more tumor neoantigens;
g) one or more bipartite polypeptides binding to MHC-peptide antigen complex;
h) one or more fusogenic proteins comprising herpes simplex virus (HSV) UL27/glycoprotein B/gB, HSV UL53/glycoprotein K/gK, Respiratory syncytial virus (RSV) F protein, FASTp15, VSV-G, syncitin-1 (from human endogenous retrovirus-W (HERV-W)) or syncitin-2 (from HERVFRDE1), paramyxovirus SV5-F, measles virus-H, measles virus-F, the glycoprotein from a retrovirus or lentivirus, such as gibbon ape leukemia virus (GALV), murine leukemia virus (MLV), Mason-Pfizer monkey virus (MPMV) and equine infectious anemia virus (EIAV), optionally with the R transmembrane peptide removed (R-versions);
i) one or more other payload molecules comprising IL15R, PGDH, ADA, ADA2, HYAL1, HYAL2, CHIPS, MLKL (or its 4HB domain only), GSDMD (or its L192A mutant, or its amino acids 1-233 fragment, or its amino acids 1-233 fragment with L192A mutation), GSDME (or its amino acid 1-237 fragment), HMGB1 (or its Box B domain only), Melittin (e.g., alpha-Melittin), SMAC/Diablo (or its amino acid 56-239 fragment), Snake LAAO, Snake disintegrin, Leptin, FLT3L, TRAIL, Gasdermin D or a truncation thereof, Gasdermin E or a truncation thereof;
j) one or more antigens from pathogens comprising Dengue virus, Chikungunya virus, Mycobacterium tuberculosis, Human immunodeficiency viruses, SARS-CoV-2, Coronavirus, Hepatitis B Virus, Togaviridae family virus, Flaviviridae family virus, Influenza A virus, Influenza B virus, and a veterinary virus; or
k) any combination thereof.

In some embodiments, the two or more payload molecules are selected from the group consisting of a fluorescent protein, an enzyme, a cytokine, a chemokine, an antigen-binding molecule capable of binding to a cell surface receptor, and a ligand for a cell-surface receptor. In some embodiments, the heterologous polynucleotide encodes two or more payload molecules comprising:
IL-2 and IL-36γ;
CXCL10 and an antigen binding molecule binding to FAP and CD3;
IL-2 and an antigen binding molecule binding to DLL3 and CD3;
IL-36γ and an antigen binding molecule binding to DLL3 and CD3; or
IL-2, IL-36γ and an antigen binding molecule binding to DLL3 and CD3.

In some embodiments, the recombinant RNA replicon of the disclosure further comprises a microRNA (miRNA) target sequence (miR-TS) cassette comprising one or more miRNA target sequences. In some embodiments, the one or more miRNAs comprise miR-124, miR-1, miR-143, miR-128, miR-219, miR-219a, miR-122, miR-204, miR-217, miR-137, and miR-126.

The disclosure provides recombinant DNA molecules comprising, from 5′ to 3′, a promoter sequence, a 5′ junctional cleavage sequence, a polynucleotide sequence encoding the recombinant RNA replicon of the disclosure, and a 3′ junctional cleavage sequence. In some embodiments, the promoter sequence is a T7 promoter sequence. In some embodiments, the 5′ junctional cleavage sequence is a ribozyme sequence and the 3′ junctional cleavage sequence is a ribozyme sequence. In some embodiments, the 5′ ribozyme sequence is a hammerhead ribozyme sequence and wherein the 3′ ribozyme sequence is a hepatitis delta virus ribozyme sequence. In some embodiments, the 5′ junctional cleavage sequence is a ribozyme sequence and the 3′ junctional cleavage sequence is a restriction enzyme recognition sequence. In some embodiments, the 5′ ribozyme sequence is a hammerhead ribozyme sequence, a Pistol ribozyme sequence, or a modified Pistol ribozyme sequence. In some embodiments, 3′ restriction enzyme recognition sequence is a Type IIS restriction enzyme recognition sequence. In some embodiments, the Type IIS recognition sequence is a SapI recognition sequence. In some embodiments, the 5′ junctional cleavage sequence is an RNAseH primer binding sequence and the 3′ junctional cleavage sequence is a restriction enzyme recognition sequence.

The disclosure provides methods of producing the recombinant RNA replicon comprising in vitro transcription of the DNA molecule of the disclosure and purification of the resulting recombinant RNA replicon.

The disclosure provides compositions comprising an effective amount of the recombinant RNA replicon of the disclosure and a carrier suitable for administration to a mammalian subject.

The disclosure provides vectors comprising the recombinant RNA replicon of the disclosure. In some embodiments, the vector is a viral vector. In some embodiments, the vector is a non-viral vector.

The disclosure provides particles comprising the recombinant RNA replicon of the disclosure. In some embodiments, the particle is selected from the group consisting of a nanoparticle, an exosome, a liposome, and a lipoplex. In some embodiments, the nanoparticle is a lipid nanoparticle (LNP) comprising a cationic lipid, one or more helper lipids, and a phospholipid-polymer conjugate. In some embodiments, the cationic lipid is selected from DLinDMA, DLin-KC2-DMA, DLin-MC3-DMA (MC3), COATSOME® SS-LC (former name: SS-18/4PE-13), COATSOME® SS-EC (former name: SS-33/4PE-15), COATSOME® SS-OC, COATSOME® SS-OP, Di((Z)-non-2-en-1-yl)9-((4-dimethylamino)butanoyl)oxy)heptadecanedioate (L-319), or N-(2,3-dioleoyloxy)propyl)-N,N,N-trimethylammonium chloride (DOTAP). In some embodiments, the helper lipid is selected from 1,2-distearoyl-sn-glycero-3-phosphocholine (DSPC); 1,2-dilauroyl-sn-glycero-3-phosphoethanolamine (DLPE); 1,2-dioleoyl-sn-glycero-3-phosphocholine (DOPC); 1,2-dioleoyl-sn-glycero-3-phosphoethanolamine (DOPE); and cholesterol. In some embodiments, the cationic lipid is 1,2-dioleoyl-3-trimethylammonium-propane (DOTAP), and wherein the neutral lipid is 1,2-Dilauroyl-sn-glycero-3-phosphoethanolamine (DLPE) or 1,2-Dioleoyl-sn-glycero-3-phosphoethanolamine (DOPE). In some embodiments, the phospholipid-polymer conjugate is selected from 1,2-distearoyl-sn-glycero-3-phosphoethanolamine-N-[amino(polyethyleneglycol)] (DSPE-PEG); 1,2-dipalmitoyl-rac-glycerol methoxypolyethylene glycol (DPG-PEG); 1,2-distearoyl-rac-glycero-3-methylpolyoxyethylene (DSG-PEG); 1,2-distearoyl-rac-glycero-3-methylpolyoxyethylene (DSG-PEG); 1,2-dimyristoyl-rac-glycero-3-methylpolyoxyethylene (DMG-PEG); and 1,2-dimyristoyl-rac-glycero-3-methylpolyoxyethylene (DMG-PEG), or 1,2-distearoyl-sn-glycero-3-phosphoethanolamine-N-[amino(polyethylene glycol)] (DSPE-PEG-amine). In some embodiments, the phospholipid-polymer conjugate is selected from 1,2-distearoyl-sn-glycero-3-phosphoethanolamine-N-[amino(polyethyleneglycol)-5000] (DSPE-PEG5K); 1,2-dipalmitoyl-rac-glycerol methoxypolyethylene glycol-2000 (DPG-PEG2K); 1,2-distearoyl-rac-glycero-3-methylpolyoxyethylene-5000 (DSG-PEG5K); 1,2-distearoyl-rac-glycero-3-methylpolyoxyethylene-2000 (DSG-PEG2K); 1,2-dimyristoyl-rac-glycero-3-methylpolyoxyethylene-5000 (DMG-PEG5K); and 1,2-dimyristoyl-rac-glycero-3-methylpolyoxyethylene-2000 (DMG-PEG2K). In some embodiments, the cationic lipid comprises COATSOME® SS-OC, wherein the one or more helper lipids comprise cholesterol (Chol) and DSPC, and wherein the phospholipid-polymer conjugate comprises DPG-PEG2000. In some embodiments, the ratio of SS-OC:DSPC:Chol:DPG-PEG2K (as a percentage of total lipid content) is A:B:C:D, wherein:
(a) A=40%-60%, B=10%-25%, C=20%-30%, and D=0%-3% and wherein A+B+C+D=100%;
(b) A=45%-50%, B=20%-25%, C=25%-30%, and D=0%-1% and wherein A+B+C+D=100%
(c) A=40%-60%, B=10%-30%, C=20%-45%, and D=0%-3% and wherein A+B+C+D=100%;
(d) A=40%-60%, B=10%-30%, C=25%-45%, and D=0%-3% and wherein A+B+C+D=100%;
(e) A=45%-55%, B=10%-20%, C=30%-40%, and D=1%-2% and wherein A+B+C+D=100%;
(f) A=45%-50%, B=10%-15%, C=35%-40%, and D=1%-2% and wherein A+B+C+D=100%;
(g) A=45%-65%, B=5%-20%, C=20%-45%, and D=0%-3% and wherein A+B+C+D=100%;
(h) A=50%-60%, B=5%-15%, C=30%-45%, and D=0%-3% and wherein A+B+C+D=100%;
(i) A=55%-60%, B=5%-15%, C=30%-40%, and D=1%-2% and wherein A+B+C+D=100%; or
(j) A=55%-60%, B=5%-10%, C=30%-35%, and D=1%-2% and wherein A+B+C+D=100%.

In some embodiments, the ratio of SS-OC:DSPC:Chol:DPG-PEG2K (as a percentage of total lipid content) is: about 49:22:28.5:0.5; about 49:11:38.5:1.5; or about 58:7:33.5:1.5. In some embodiments, the ratio of SS-OC:DSPC:Chol:DPG-PEG2K (as a percentage of total lipid content) is about 49:22:28.5:0.5. In some embodiments, the cationic lipid is 1,2-dioleoyl-3-trimethylammonium-propane (DOTAP), and wherein the neutral lipid is 1,2-Dilauroyl-sn-glycero-3-phosphoethanolamine (DLPE) or 1,2-Dioleoyl-sn-glycero-3-phosphoethanolamine (DOPE).

In some embodiments, the particle of the disclosure further comprises a phospholipid-polymer conjugate, wherein the phospholipid-polymer conjugate is 1, 2-Distearoyl-sn-glycero-3-phosphoethanolamine-Poly(ethylene glycol) (DSPE-PEG) or 1,2-distearoyl-sn-glycero-3-phosphoethanolamine-N-[amino(polyethylene glycol)] (DSPE-PEG-amine).

In some embodiments, the particle of the disclosure further comprises a second recombinant RNA molecule encoding an oncolytic virus. In some embodiments, the oncolytic virus is a picornavirus. In some embodiments, the picornavirus is selected from a senecavirus, a cardiovirus, and an enterovirus. In some embodiments, the picornavirus is a Seneca Valley Virus (SVV). In some embodiments, the picornavirus is a Coxsackievirus. In some embodiments, the picornavirus is an encephalomyocarditis virus (EMCV).

The disclosure provides therapeutic compositions comprising a plurality of lipid nanoparticles of the disclosure. In some embodiments, the plurality of LNPs have an average size of about 50 nm to about 120 nm. In some embodiments, the plurality of LNPs have an average size of about 100 nm. In some embodiments, the plurality of LNPs have an average zeta-potential of between about 20 mV to about −20 mV, about 10 mV to about −10 mV, about 5 mV to about −5 mV, or about 20 mV to about −40 mV, −50 mV to about −20 mV, about −40 mV to about −20 mV, or about −30 mV to about −20 mV. In some embodiments, the plurality of LNPs have an average zeta-potential of about −30 mV, about −31 mV, about −32 mV, about −33 mV, about −34 mV, about −35 mV, about −36 mV, about −37 mV, about −38 mV, about −39 mV, or about −40 mV.

The disclosure provides methods of killing a cancerous cell comprising exposing the cancerous cell to the particle, the vector, the recombinant RNA replicon, or compositions of the disclosure. In some embodiments, the method is performed in vivo, in vitro, or ex vivo.

The disclosure provides methods of treating a cancer in a subject comprising administering to the subject suffering from the cancer an effective amount of the particle, the vector, the recombinant RNA replicon, or compositions of the disclosure. In some embodiments, the recombinant RNA replicon, or composition thereof is administered intravenously, intranasally, as an inhalant, or is injected directly into a tumor. In some embodiments, the particle, the recombinant RNA replicon, or composition thereof is administered to the subject repeatedly. In some embodiments, the subject is a mouse, a rat, a rabbit, a cat, a dog, a horse, a non-human primate, or a human.

In some embodiments, the cancer is selected from lung cancer, breast cancer, ovarian cancer, cervical cancer, prostate cancer (e.g., Castration resistant neuroendocrine prostate cancer), testicular cancer, colorectal cancer, colon cancer, pancreatic cancer, liver cancer, gastric cancer, head and neck cancer, thyroid cancer, malignant glioma, glioblastoma, melanoma, B-cell chronic lymphocytic leukemia, diffuse large B-cell lymphoma (DLBCL), sarcoma, a neuroblastoma, a neuroendocrine cancer, a rhabdomyosarcoma, a medulloblastoma, a bladder cancer, marginal zone lymphoma (MZL), Merkel cell carcinoma, and renal cell carcinoma. In some embodiments, the lung cancer is small cell lung cancer or non-small cell lung cancer; the liver cancer is hepatocellular carcinoma (HCC); and/or the prostate cancer is treatment-emergent neuroendocrine prostate cancer. In some embodiments, the cancer is a neuroendocrine cancer.

The disclosure provides methods of immunizing a subject against a disease, comprising administering to the subject an effective amount of the particle, the vector, the recombinant RNA replicon, or compositions of the disclosure. In some embodiments, the particle, the recombinant RNA replicon, or composition thereof is administered intravenously, intramuscularly, intradermally, intranasally, or as an inhalant. In some embodiments, the particle, the recombinant RNA replicon, or composition thereof is administered to the subject repeatedly. In some embodiments, the disease is an infectious disease. In some embodiments, the infectious disease is caused by one of the pathogens comprising Dengue virus, Chikungunya virus, Mycobacterium tuberculosis, Human immunodeficiency virus, SARS-CoV-2, Coronavirus, Hepatitis B virus, Togaviridae family virus, Flaviviridae family virus, Influenza A virus, Influenza B virus and a veterinary virus.

The disclosure provides recombinant RNA replicons comprising a picornavirus genome and a heterologous polynucleotide. In some embodiments, the heterologous polynucleotide is inserted between a 2A coding region and a 2B coding region. In some embodiments, the heterologous polynucleotide is inserted between a 5′ UTR and a 2A coding region. In some embodiments, the heterologous polynucleotide is inserted between a 3D coding region and a 3′ UTR. In some embodiments, the picornavirus is selected from a senecavirus, a cardiovirus, and an enterovirus.

### BRIEF DESCRIPTION OF THE DRAWINGS

FIG. 1 is a schematic depicting a wildtype SVV viral genome and an exemplary SVV derived recombinant RNA replicon.

FIG. 2 is a series of charts showing viral replication rate of SVV comprising heterologous polynucleotide of various lengths.

FIG. 3A is a schematic depicting various SVV derived recombinant RNA replicon constructs with mCherry reporter gene. FIG. 3B is a series of imaging figures showing the expression of mCherry in H1299 cells transected with the replicons.

FIG. 4A is a series of imaging figures showing the expression of mCherry in H1299 cells transected with the replicon Trunc5 and/or wildtype SVV viral genome. FIG. 4B contains charts showing the result of an IC50 assay in H446 cells for evaluation of viral titer.

FIG. 5A is a schematic depicting various SVV derived recombinant RNA replicon constructs with mCherry reporter gene. FIG. 5B is a gel image showing the result of in vitro T7 RNA synthesis. FIG. 5C is a series of imaging figures showing mCherry signal of cells transfected with each replicon.

FIG. 6A is a schematic depicting a wildtype SVV viral genome and an SVV derived recombinant RNA replicon carrying a mCherry reporter gene. FIG. 6B is a schematic depicting SVV derived recombinant RNA replicon Trunc10 carrying different reporter genes.

FIG. 7A is a schematic depicting an SVV-replicon Trunc10 carrying a transgene encoding murine IL-2 payload. FIG. 7B contains two charts showing the result of mIL-2 expression. FIG. 7C is a chart showing the result of RNA copy numbers analyzed by taqman assay.

FIG. 8A is a schematic depicting an SVV-replicon Trunc10 carrying a transgene encoding single chain mIL-12 (scmIL-12), with and without a signal sequence. FIG. 8B is a chart showing the result of RNA copy numbers analyzed by taqman assay. FIG. 8C contains two charts showing the expression of murine IL-12.

FIG. 9A is a schematic depicting SVV-replicons Trunc10 carrying a transgene encoding human IL-36γ, with the native signal sequence or with the IL2 signal sequence. FIG. 9B contains two charts showing the secretion of hIL-36γ after transfection or trans-encapsidation. FIG. 9C contains two charts showing the result of RNA copy numbers analyzed by taqman assay.

FIG. 10A is a schematic depicting bicistronic replicons incorporated with an encephalomyocarditis virus (EMCV) IRES downstream of a single payload. FIG. 10B is a chart showing the result of RNA copy numbers analyzed by taqman assay.

FIG. 11A is a schematic depicting dicistronic dual payload replicons incorporated with an encephalomyocarditis virus (EMCV) IRES downstream of multiple payloads separated by a furin-T2A site between the first payload and the second payload (eGFP). FIG. 11B is a series of images showing the expression of mCherry and GFP 24 hours post infection.

FIG. 12A is a schematic depicting a dual payload replicon incorporated with a second payload at the 3′ end of the replicon between the RdRp and the 3′UTR. FIG. 12B is a chart showing the secretion of hIL-36γ after transfection. FIG. 12C is a chart showing the result of RNA copy numbers analyzed by taqman assay.

FIG. 13A is an anti-His western blot that analyzes the expression of his-tagged 1DLT176-MTT10-DLL3-VHH-CD3 LiTE. FIG. 13B is a chart showing the result of RNA copy numbers analyzed by taqman assay.

FIG. 14A is an anti-His western blot that analyzes the expression of his-tagged rDLL3-αCD3-BiTE. FIG. 14B is a chart showing the result of RNA copy numbers analyzed by taqman assay.

FIG. 15A is a schematic depicting Trunc10 replicon comprising alternate cleavage peptides (3C, or furin-3C, or furinT2A) between his-tagged anti mFAP BiTE and CXCL10. FIG. 15B is a chart showing the result of expression of CXCL10. FIG. 15C is a chart showing the result of RNA copy numbers analyzed by taqman assay.

FIG. 16A is a schematic depicting Trunc10 replicon comprising alternate cleavage peptides (T2A, P2A, F2A, or E2A) between his-tagged anti-mFAP BiTE and CXCL10. FIG. 16B is a chart showing the result of expression of CXCL10. FIG. 16C is a chart showing the result of RNA copy numbers analyzed by taqman assay.

FIG. 17A is a schematic depicting a configuration of replicon polynucleotide encoding dual payload molecules operably linked by an IGSF1 polypeptide (SEQ ID NOs: 75 and 76). FIG. 17B is a chart showing the result of expression of IL-36γ. FIG. 17C is a chart showing the result of expression of IL-2. FIG. 17D is a chart showing the result of RNA copy numbers analyzed by taqman assay.

FIG. 18A is a schematic depicting schematic for HIV-1 protease mediated processing of two secreted payloads in the same open reading frame. FIG. 18B is a chart showing the result of RNA copy numbers analyzed by taqman assay. FIG. 18C contains two charts showing the result of expression of both payloads.

FIG. 19A is a schematic depicting a dual payload replicon comprising a BiTE and hIL-36γ. FIG. 19B is a chart showing expression of hIL-36γ. FIG. 19C is a chart showing the result of RNA copy numbers analyzed by taqman assay.

FIG. 20A is a schematic depicting a triple payload replicon T10-BiTE-IL3g6-IL2. FIG. 20B contains two charts showing the expression of hIL-36 and mIL-2. FIG. 20C is a chart showing the result of RNA copy numbers analyzed by taqman assay.

FIG. 21A is a schematic depicting an alternative design of triple payload replicon T10-mIL2-BiTE-hIL-36γ. FIG. 21B contains two charts showing the expression of hIL-36 and mIL-2. FIG. 21C is a chart showing the result of RNA copy numbers analyzed by taqman assay.

FIG. 22A is a schematic depicting another design of triple payload replicon T10-mIL2-hIL-36γ-BiTE. FIG. 22B is a chart showing the result of RNA copy numbers analyzed by taqman assay. FIG. 22C is a series of charts showing the expression of hIL-36 and mIL-2 in supernatant and lysate.

FIG. 23A is a chart showing the result of in vivo hIL-36γ expression in a NCI-H69 cells based mouse model. FIG. 23B is a chart showing the result of in vivo hIL-36γ expression in a NCI-H446 cells based mouse model.

FIG. 24 is a schematic depicting a wildtype coxsackievirus viral genome and an exemplary coxsackievirus derived recombinant RNA replicon carrying an mCherry reporter gene.

FIG. 25A is a series of images showing mCherry and GFP expression in cells transfected with the replicon and/or control vectors. FIG. 25B contains two images showing the expression of mCherry which demonstrates trans-encapsidation of the replicon in co-transfection with wildtype viral genome.

FIG. 26 is a diagram depicting the in vitro transcription process for an SVV derived replicon and a Neg-RNA. Autocatalytic cleavage of SVV derived replicon by 5′ and 3′ ribozyme (Rib) generate SVV derived replicon with discrete 5′ and 3′ ends required for replication. By contrast, Neg-RNA construct lacks ribozyme sequence and is not able of replication and virion production.

FIG. 27 is a diagram depicting the use of junctional cleavage sequences to remove non-viral RNA polynucleotides from the genome transcripts in order to maintain the native 5′ and 3′ discrete ends of the replicon.

FIGS. 28A-28B are schematics showing hammerhead ribozymes for generation of discrete 5′ termini. FIG. 28A is a schematic showing a structural model of a minimal hammerhead ribozyme (HHR) (SEQ ID NO: 108) that anneals and cleaves at the 5′ terminus at the arrow. FIG. 28B is a schematic showing a structural model of a ribozyme with a stabilized stem I (STBL) (SEQ ID NO: 109) for cleavage of 5′ terminus at the arrow.

FIGS. 29A-29B are schematics showing pistol ribozymes for generation of discrete 5′ termini. FIG. 29A is a schematic showing wild type Pistol ribozyme (SEQ ID NOs: 110, 111) characteristics. FIG. 29B is a schematic showing Pistol ribozyme from P. Polymyxa (SEQ ID NO: 112) with a tetraloop added to fuse the P3 strands modeled by mFOLD. The dashed box is the area mutagenized to retain the fold of the ribozyme in the context of the viral sequence. The “GUC” sequence shown in the dashed box was mutated to “UCA” to generate Pistol 1 and the “GUC” sequence was mutated to “TTA” to generate Pistol 2.

### DETAILED DESCRIPTION

Oncolytic viruses are replication-competent viruses with lytic life-cycle able to infect and lyse tumor cells. Direct tumor cell lysis results not only in cell death, but also the generation of an adaptive immune response against tumor antigens taken up and presented by local antigen presenting cells. Therefore, oncolytic viruses combat tumor cell growth through both direct cell lysis and by promoting antigen-specific adaptive responses capable of maintaining anti-tumor responses after viral clearance.

Oncolytic viruses can be genetically engineered to express payload molecules—e.g., by incorporating a heterologous polynucleotide that encodes a desirable payload protein into the viral genome. However, due to the packaging capability of the viral capsid proteins, only polynucleotides with a limited length can be incorporated into the full viral genome without compromising the replication rate, encapsidation, and/or function of the viruses. In addition, expression of multiple functional payload molecules from a single synthetic viral genome or viral replicon can be challenging. These limitations in the incorporation of payload molecules limit the use of viral therapeutics in the treatment of metastatic cancers.

There is a need in the art for oncolytic virus derived replicons comprising improved capacity for the incorporation of heterologous polynucleotides encoding payload molecules, which can be used in various therapeutics such as anti-cancer therapeutics. Heterologous sequences may encode one or more molecules that may be referred to herein as payload molecules. In some embodiments, payload sequences and payload molecules of the disclosure do not mediate a viral function. In some embodiments, payload sequences and payload molecules of the disclosure may be isolated from or derived from a species matching or homologous to the species of the subject or cell intended for administration of the viral replication for expression of the payload sequence or payload molecule. Heterologous sequences may encode one or more of a coding or a noncoding nucleic acid sequence, a DNA sequence, an RNA sequence, an amino acid sequence, a peptide, a polypeptide, a protein or any combination thereof.

The disclosure provides recombinant RNA replicons derived from picornaviral genomes that possess improved capability for the incorporation of heterologous polynucleotides encoding payload molecules. In some embodiments, the recombinant RNA replicons of the disclosure express two or more functional payload molecules from the same replicon. Exemplary configuration of replicons expressing two or more payload molecules are described. The present disclosure further provides particles comprising recombinant RNA replicons. In some embodiments, the particles further comprise full viral genome. In some embodiments, the recombinant RNA replicons can be trans-encapsidated by the capsid proteins expressed by the full viral genome. In some embodiments, contacting cells with said particles allows production of two groups of infectious viral particles, one comprising a recombinant RNA replicon, and the other comprising the full viral genome. In some embodiments, viral particles of both groups can infect cells together which allows continuous production of viral particles of both groups, either in vivo or in vitro. In some embodiments, the present disclosure provides recombinant RNA replicons and methods of use for the treatment and prevention of proliferative diseases and disorders (e.g., cancer). The present disclosure enables the systemic delivery of an efficacious recombinant RNA replicons suitable to treat a broad array of proliferative disorders (e.g., cancers).

The section headings used herein are for organizational purposes only and are not to be construed as limiting the subject matter described. All documents, or portions of documents, cited herein, including but not limited to patents, patent applications, articles, books, and treatises, are hereby expressly incorporated by reference in their entirety for any purpose. In the event that one or more of the incorporated documents or portions of documents define a term that contradicts that term's definition in the application, the definition that appears in this application controls. However, mention of any reference, article, publication, patent, patent publication, and patent application cited herein is not, and should not be taken as an acknowledgment, or any form of suggestion, that they constitute valid prior art or form part of the common general knowledge in any country in the world.

### Recombinant RNA Replicon

Picornavirus genomes follow a conserved 4-3-4 format, where the single polyprotein is cleaved by virally encoded proteases into the 5′ leader protein (present only in some species), four structural and seven (3+4) nonstructural proteins. Picornaviral genomes start with a 5′ untranslated region (UTR) and include the internal ribosome entry site (IRES). Adjacent to the IRES, the 5′ leader protein is a protease that sits at the 5′ extreme of the translated picornaviral polyprotein, though it is not present in all members of the Picornaviridae family. This is followed by the P1 region of the polyprotein, encoding in order the capsid proteins VP4, VP2, VP3 and VP1 respectively. These proteins are encoded by the VP4 coding region, the VP2 coding region, the VP3 coding region and the VP1 coding region, respectively (together, these four coding regions are called the “VP coding regions”). The P2 region of the translated polyprotein consists of 2A, 2B and 2C. The picornaviral 2A is a protein which can be absent, or in some cases present in more than one copy in the picornaviral genome. The final segment of the picornaviral polyprotein is P3, comprising 3A, 3B, 3C and 3D. The 3B, also known as VPg, is a small protein which associates with the 5′ terminus of the genome and plays an essential role in genome replication. The protease encoded by 3C performs most of the cleavages of the picornaviral polyprotein as well as inhibiting host transcription. Last among the picornaviral proteins is 3D, the RNA-dependent RNA polymerase (RdRp). The 3′ UTR of picornaviruses typically have a poly-A tail.

The present disclosure provides recombinant RNA replicons comprising a picornavirus genome, wherein the picornavirus genome comprises a deletion and/or a truncation in one or more coding regions. In some embodiments, the coding regions encodes structural proteins (VP4, VP2, VP3 and VP1). In some embodiments, the picornavirus genome of the replicon comprises a deletion of all of the VP coding regions. In some embodiments, the picornavirus genome of the replicon comprises deletions and/or truncations in each of the VP1, VP3 and VP2 coding regions. In some embodiments, the picornavirus genome of the replicon comprises deletions of the VP1 and VP3 coding regions and truncation of the VP2 coding region. In some embodiments, the deletions and truncations within the VP coding regions of the picornavirus genome comprise at least 500 bp, at least 1000 bp, at least 1500 bp, at least 2000 bp, at least 2500 bp, or at least 3000 bp. In some embodiments, the total deletions and truncations within the VP coding regions of the picornavirus genome is at least 2000 bp.

In some embodiments, the recombinant RNA replicons comprise one or more heterologous polynucleotide. In some embodiments, the heterologous polynucleotide is inserted into a site of the deletion or truncation. In some embodiments, the heterologous polynucleotide is inserted between a 2A coding region and a 2B coding region. In some embodiments, the heterologous polynucleotide is inserted between a 3D(RdRp) coding region and a 3′ untranslated region (UTR). In some embodiments, the one or more heterologous polynucleotides comprise at least 1000 bp, at least 2000 bp, or at least 3000 bp.

In some embodiments, the picornavirus genome is selected from a senecavirus genome, a cardiovirus genome, an enterovirus genome, and an aphthovirus genome. In some embodiments, the viral genome is derived from a picornavirus selected from a Cardiovirus, a Cosavirus, an Enterovirus, a Hepatovirus, a Kobuvirus, a Parechovirus, a Rosavirus, a Salivirus, a Pasivirus, a Senecavirus, and a chimeric viral genome thereof. In some embodiments, the viral genome is derived from a picornavirus selected from Human Rhinovirus, HRV (SEQ ID NO: 5; GenBank accession No. K02121.1), Poliovirus, PV (SEQ ID NO: 6; GenBank accession No. AF111984.2), Coxsackievirus A, CVA (SEQ ID NO: 7; GenBank accession No. AF546702.1), Bovine Enterovirus, BEV (SEQ ID NO: 8; GenBank accession No. NC_001859.1), Enterovirus 71, EV71 (SEQ ID NO: 9; GenBank accession No. KJ686308.1), Echovirus, ECHO (SEQ ID NO: 10; GenBank accession No. AF029859.2), Foot-and-Mouth virus, FMDV (SEQ ID NO: 11; GenBank accession No. DQ989323.1), Seneca Valley virus, SVV (SEQ ID NO: 12; GenBank accession No. NC_011349.1), Theiler's Murine Encephalomyelitis virus, TMEV (SEQ ID NO: 13; GenBank accession No. M20301.1), Mengovirus, MEV (SEQ ID NO: 14; GenBank accession No. L22089.1), Encephalomyocarditis, EMCV (SEQ ID NO: 15; GenBank accession No. X74312.1)-the NCBI GenBank Accession No. of each virus is indicated in the parenthesis. In some embodiments, the picornavirus genome is a seneca valley virus genome. In some embodiments, the picornavirus genome is a coxsackievirus genome. In some embodiments, the picornavirus genome is an encephalomyocarditis virus genome. In some embodiments, the picornavirus genome is a poliovirus genome (including a chimeric polio virus such as PVS-RIPO).

In some embodiments, the recombinant RNA replicons described herein comprises a chimeric picornavirus genome (e.g., a viral genome comprising one portion, such as a capsid protein or an IRES, that is derived from a first picornavirus, and another portion, such as a non-structural protease or polymerase coding region derived from a second picornavirus).

In some embodiments, the recombinant RNA replicon retains competency for positive and/or negative strand RNA synthesis. In some embodiments, the rate of positive and/or negative strand RNA synthesis of the recombinant RNA replicon is at least 1%, at least 5%, at least 10%, at least 20%, at least 30%, at least 40%, at least 50%, at least 60%, at least 70%, at least 80%, at least 90%, or at least 100% of the rate of synthesis of the corresponding wild type viral genome.

In some embodiments, the recombinant RNA replicon retains a viral replication rate that is comparable to the wildtype viral genome. In some embodiments, the viral replication rate of the recombinant RNA replicon is at least 1%, at least 5%, at least 10%, at least 20%, at least 30%, at least 40%, at least 50%, at least 60%, at least 70%, at least 80%, at least 90%, or at least 100% of the viral replication rate of the corresponding wildtype viral genome.

In some embodiments, the recombinant RNA replicon is provided as a recombinant ribonucleic acid (RNA). In some embodiments, the recombinant RNA replicons comprise one or more nucleic acid analogues. Examples of nucleic acid analogues include 2′-O-methyl-substituted RNA, 2′-O-methoxy-ethyl bases, 2′ Fluoro bases, locked nucleic acids (LNAs), unlocked nucleic acids (UNA), bridged nucleic acids (BNA), morpholinos, and peptide nucleic acids (PNA). In some embodiments, the recombinant RNA replicon is a circular RNA molecule (circRNA) or a single stranded RNA (ssRNA). In some embodiments, the single-stranded RNA is a positive sense or negative sense strand.

In some embodiments, the recombinant RNA replicon is a circular RNA molecule (circRNA). CircRNA molecules lack the free ends necessary for exonuclease mediated degradation, thus extending the half-life of the RNA molecule and enabling more stable protein production over time. In order to produce a functional RNA replicon from a circRNA molecule, it is necessary to “break open” the circular construct once inside a cell so that the linear RNA replicon with the appropriate 3′ and 5′ native ends can be produced. Therefore, in some embodiments, the recombinant RNA replicon is provided as a circRNA molecule and further comprises one or more additional RNA sequences that facilitate the linearization of the circRNA molecule inside a cell. Examples of such additional RNA sequences include siRNA target sites, miRNA target sites, and guide RNA target sites. The corresponding siRNA, miRNA, or gRNA can be co-formulated with the circRNA molecule. Alternatively, the miRNA target site can be selected based on the expression of the cognate miRNA in a target cell, such that cleavage of the circRNA molecule and replication of the replicon is limited to target cells expressing a particular miRNA.

### Recombinant SVV Replicon

The disclosure provides recombinant RNA replicons comprising a Seneca Valley Virus (SVV) viral genome, wherein the SVV genome comprises a deletion or a truncation in one or more SVV protein coding regions. In some embodiments, the replicon comprises a heterologous polynucleotide.

In some embodiments, the SVV genome is selected from a wild-type SVV genome (such as SVV-A, SEQ ID NO: 1) or a mutant SVV genome (such as SVV-IR2, SEQ ID NO: 2). In some embodiments, the recombinant RNA replicon of the disclosure comprises a chimeric SVV genome.

For SVV viral genome, the VP4 coding region encompasses nucleotide 904 to nucleotide 1116 according to SEQ ID NO: 1. The VP2 coding region encompasses nucleotide 1117 to nucleotide 1968 according to SEQ ID NO: 1. The VP3 coding region encompasses nucleotide 1969 to nucleotide 2685 according to SEQ ID NO: 1. The VP1 coding region encompasses nucleotide 2686 to nucleotide 3477 according to SEQ ID NO: 1. The 2A coding region encompasses nucleotide 3478 to nucleotide 3504 according to SEQ ID NO: 1. The 2B coding region encompasses nucleotide 3505 to nucleotide 3888 according to SEQ ID NO: 1.

In some embodiments, the SVV genome of the replicon comprises deletions and/or truncations in one or more VP coding regions. In some embodiments, the replicons described herein are administered to subjects in combination with a synthetic viral genome. Without wishing to be bound by any particular theory, it is thought that deleting and/or truncating the VP coding regions in the replicon will: 1) facilitate accommodation of larger payload cassettes than the virus itself and/or 2) render the replicon by itself incapable of cell to cell spread.

In some embodiments, one, or at least one of the VP4, VP2, VP3 and VP1 coding regions are deleted and/or truncated. In some embodiments, two, or at least two, of the VP coding regions comprising VP4, VP2, VP3 and VP1 are deleted and/or truncated. In some embodiments, two, or at least two, of the VP coding regions comprising VP2, VP3 and VP1 are deleted and/or truncated. In some embodiments, three, or at least three, of the VP coding regions comprising VP4, VP2, VP3 and VP1 are deleted and/or truncated. In some embodiments, all of the VP4, VP2, VP3 and VP1 coding regions are deleted and/or truncated. In some embodiments, the VP2 coding region is truncated and one of the VP3 coding region and the VP1 coding region is deleted or truncated. In some embodiments, the VP2 coding region is truncated and both the VP3 and VP1 coding regions are deleted and/or truncated. In some embodiments, the SVV genome of the replicon comprises a deletion and/or truncation of each of the VP1, VP3 and VP2 coding regions. In some embodiments, the SVV genome of the replicon comprises a deletion of the VP1 and VP3 coding regions and a truncation of the VP2 coding region. In some embodiments, the SVV genome of the replicon comprises one or more deletions or truncations in the VP2-VP3-VP1 region following one of the patterns listed in Table 1 below.

TABLE 1
Pattern of Deletion or Truncation in the
VP2-VP3-VP1 Region of SVV Genome

| Pattern**\ | | | |
|---|---|---|---|
| coding region | VP2 | VP3 | VP1 |
| 1 | T | N | N |
| 2 | T | N | T |
| 3 | T | N | D |
| 4 | T | T | N |
| 5 | T | T | T |
| 6 | T | T | D |
| 7 | T | D | N |
| 8 | T | D | T |
| 9 | T | D | D |
| 10 | N | N | N |
| 11 | N | N | T |
| 12 | N | N | D |
| 13 | N | T | N |
| 14 | N | T | T |
| 15 | N | T | D |
| 16 | N | D | N |
| 17 | N | D | T |
| 18 | N | D | D |

**“D” indicates deletion; “T” indicates truncation; “N” indicates no truncation or deletion.

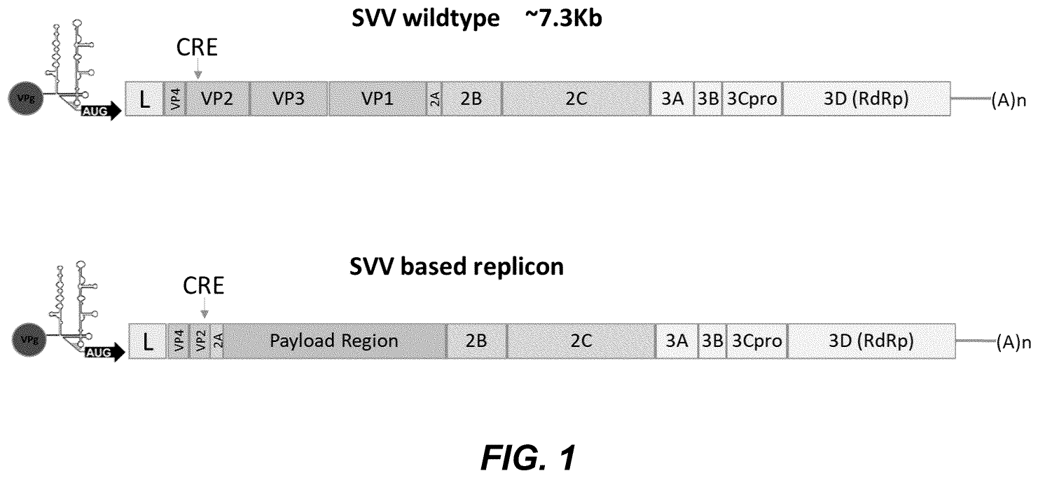

In some embodiments, the SVV genome of the replicon comprises, consists essentially of, or consists of, one or more deletions or truncations of the SVV genome within the region corresponding to nucleotide 1261 to nucleotide 3477, inclusive of the endpoints, according to SEQ ID NO: 1 and FIG. 1. In some embodiments, the SVV genome of the replicon comprises a deletion of the SVV genome region corresponding to nucleotide 1261 to nucleotide 3477, inclusive of the endpoints, according to SEQ ID NO: 1. In some embodiments, the replicon comprises one or more deletions or truncations within the region corresponding to nucleotide 1407 to nucleotide 3477 according to SEQ ID NO: 1. In some embodiments, the replicon comprises one or more deletions or truncations within the region corresponding to nucleotide 1599 to nucleotide 3477 according to SEQ ID NO: 1. In some embodiments, the replicon comprises one or more deletions or truncations within the region corresponding to nucleotide 1683 to nucleotide 3477 according to SEQ ID NO: 1. In some embodiments, the replicon comprises one or more deletions or truncations within the region corresponding to nucleotide 1924 to nucleotide 3477 according to SEQ ID NO: 1. In some embodiments, the replicon comprises one or more deletions or truncations within the region corresponding to nucleotide 2467 to nucleotide 3477 according to SEQ ID NO: 1. In some embodiments, the replicon comprises one or more deletions or truncations within the region corresponding to nucleotide 1261 to nucleotide 3300 according to SEQ ID NO: 1. In some embodiments, the replicon comprises one or more deletions or truncations within the region corresponding to nucleotide 1261 to nucleotide 3000 according to SEQ ID NO: 1. In some embodiments, the replicon comprises one or more deletions or truncations within the region corresponding to nucleotide 1261 to nucleotide 2700 according to SEQ ID NO: 1. In some embodiments, the replicon comprises one or more deletions or truncations within the region corresponding to nucleotide 1261 to nucleotide 2400 according to SEQ ID NO: 1. In some embodiments, the replicon comprises one or more deletions or truncations within the region corresponding to nucleotide 1261 to nucleotide 2100 according to SEQ ID NO: 1. All ranges are inclusive of the endpoints.

In some embodiments, each of the deletion or the truncation comprises 1 or more nucleotides. In some embodiments, each of the deletion or the truncation comprises 10 or more nucleotides. In some embodiments, each of the deletion or the truncation comprises 50 or more nucleotides. In some embodiments, each of the deletion or the truncation comprises 100 or more nucleotides. In some embodiments, each of the deletion or the truncation comprises 500 or more nucleotides. In some embodiments, each of the deletion or the truncation comprises 1000 or more nucleotides.

In some embodiments, the one or more deletions or truncations comprise at least 500 bp, at least 600 bp, at least 700 bp, at least 800 bp, at least 900 bp, at least 1000 bp, at least 1100 bp, at least 1200 bp, at least 1300 bp, at least 1400 bp, at least 1500 bp, at least 1600 bp, at least 1700 bp, at least 1800 bp, at least 1900 bp, at least 2000 bp, at least 2100 bp, or at least 2200 bp of nucleotides in total. In some embodiments, the one or more deletions or truncations consist of 500 bp, 600 bp, 700 bp, 800 bp, 900 bp, 1000 bp, 1100 bp, 1200 bp, 1300 bp, 1400 bp, 1500 bp, 1600 bp, 1700 bp, 1800 bp, 1900 bp, 2000 bp, 2100 bp, 2200 bp, 2300 bp, 2400 bp, or any values in between, of nucleotides in total. In some embodiments, the one or more deletions or truncations consist of between 500-2400 bp, between 500-2300 bp, between 500-2200 bp, between 500-2000 bp, between 500-1500 bp, between 500-1000 bp, between 1000-2300 bp, between 1000-2200 bp, between 1000-2000 bp, between 1000-1500 bp, between 1500-2300 bp, between 1500-2200 bp, between 1500-2000 bp, between 2000-2300 bp, or between 2000-2200 bp of nucleotides in total. All ranges are inclusive of the endpoints.

In some embodiments, the SVV genome of the replicon comprises a 5′ UTR. In some embodiments, the SVV genome of the replicon comprises a 5′ leader protein coding sequence. In some embodiments, the SVV genome of the replicon comprises a non-truncated VP4 coding region. In some embodiments, the SVV genome of the replicon comprises a VP2 coding region or a truncation thereof.

In some embodiments, the SVV genome of the replicon comprises, from 5′ to 3′, a 5′ leader protein coding sequence, a VP4 coding region, and a VP2 coding region or a truncation thereof. In some embodiments, a portion of the SVV genome of the replicon comprising the 5′ UTR, the 5′ leader protein coding sequence, the VP4 coding region and the VP2 coding region or a truncation thereof has at least 70%, at least 75%, at least 80%, at least 85%, at least 90%, at least 93%, at least 95%, at least 97%, at least 98%, at least 99%, at least 99.5%, or 100% sequence identity to nucleotide 1 to 1260 of SEQ ID NO: 1 or SEQ ID NO: 2. In some embodiments, a portion of the SVV genome of the replicon comprising the 5′ UTR, the 5′ leader protein coding sequence, the VP4 coding region and the VP2 coding region or a truncation thereof has about 70%, about 75%, about 80%, about 85%, about 90%, about 93%, about 95%, about 97%, about 98%, about 99%, about 99.5%, or 100% sequence identity to nucleotide 1 to 1260 of SEQ ID NO: 1 or SEQ ID NO: 2. In some embodiments, a portion of the SVV genome of the replicon comprising the 5′ UTR, the 5′ leader protein coding sequence, the VP4 coding region and the VP2 coding region or a truncation thereof has at most 1, at most 5, at most 10, or at most 20 nucleotide mutations according to nucleotide 1 to 1260 of SEQ ID NO: 1 or SEQ ID NO: 2.

In some embodiments, the SVV genome of the replicon comprises a 5′ portion of the VP2 coding region. In some embodiments, the 5′ portion of the endogenous VP2 coding region is at least 50 bp, at least 60 bp, at least 70 bp, at least 80 bp, at least 90 bp, at least 100 bp, at least 110 bp, at least 120 bp, at least 130 bp, at least 140 bp, or at least 145 bp in length. In some embodiments, the 5′ portion of the endogenous VP2 coding region comprises about 50 bp, about 60 bp, about 70 bp, about 80 bp, about 90 bp, about 100 bp, about 110 bp, about 120 bp, about 130 bp, about 140 bp, about 145 bp, or any value in between. In some embodiments, the 5′ portion of the endogenous VP2 coding region is less than 50 bp, less than 60 bp, less than 70 bp, less than 80 bp, less than 90 bp, less than 100 bp, less than 110 bp, less than 120 bp, less than 130 bp, less than 140 bp, or less than 145 bp in length. All ranges are inclusive of the endpoints.

In some embodiments, the SVV genome of the replicon comprises a cis-acting replication element (CRE). In some embodiments, the VP2 coding region or a truncation thereof of the SVV genome of the replicon comprises a CRE. In some embodiments, the region in the SVV genome of the replicon comprising a VP4 coding region and a VP2 coding region or a truncation thereof comprises a CRE.

In some embodiments, the CRE comprises about 10 bp, about 20 bp, about 30 bp, about 40 bp, about 50 bp, about 60 bp, about 70 bp, about 80 bp, about 90 bp, about 100 bp, about 110 bp, about 120 bp, about 130 bp, about 140 bp, about 150 bp, about 160 bp, about 170 bp, about 180 bp, about 190 bp, about 200 bp, or any value in between, of nucleotides. In some embodiments, the CRE comprises at least 10 bp, at least 20 bp, at least 30 bp, at least 40 bp, at least 50 bp, at least 60 bp, at least 70 bp, at least 80 bp, at least 90 bp, at least 100 bp, at least 110 bp, at least 120 bp, at least 130 bp, at least 140 bp, at least 150 bp, at least 160 bp, at least 170 bp, at least 180 bp, at least 190 bp, or at least 200 bp, of nucleotides. In some embodiments, the CRE comprises between 10-200 bp, between 10-150 bp, between 10-100 bp, between 10-75 bp, between 10-60 bp, between 10-50 bp, between 20-200 bp, between 20-150 bp, between 20-100 bp, between 20-75 bp, between 20-60 bp, between 20-50 bp, between 30-200 bp, between 30-150 bp, between 30-100 bp, between 30-75 bp, between 30-60 bp, between 30-50 bp, between 40-200 bp, between 40-150 bp, between 40-100 bp, between 40-75 bp, between 40-60 bp, between 40-50 bp, between 50-200 bp, between 50-150 bp, between 50-100 bp, between 50-75 bp, or between 50-60 bp, of nucleotides. All ranges are inclusive of the endpoints.

In some embodiments, the CRE comprises one or more nucleotides within the region corresponding to nucleotide 1000 to nucleotide 1260 according to SEQ ID NO: 1. In some embodiments, the CRE comprises one or more nucleotides within the region corresponding to nucleotide 1000 to nucleotide 1260, nucleotide 1050 to nucleotide 1260, nucleotide 1100 to nucleotide 1260, nucleotide 1150 to nucleotide 1260, nucleotide 1200 to nucleotide 1260, nucleotide 1000 to nucleotide 1250, nucleotide 1050 to nucleotide 1250, nucleotide 1100 to nucleotide 1250, nucleotide 1150 to nucleotide 1250, nucleotide 1200 to nucleotide 1250, nucleotide 1000 to nucleotide 1200, nucleotide 1050 to nucleotide 1200, nucleotide 1100 to nucleotide 1200, nucleotide 1150 to nucleotide 1200, nucleotide 1000 to nucleotide 1150, nucleotide 1050 to nucleotide 1150, nucleotide 1100 to nucleotide 1150, nucleotide 1000 to nucleotide 1100, or nucleotide 1050 to nucleotide 1100, according to SEQ ID NO: 1. In some embodiments, the CRE is located within the region corresponding to nucleotide 1000 to nucleotide 1260, nucleotide 1050 to nucleotide 1260, nucleotide 1100 to nucleotide 1260, nucleotide 1150 to nucleotide 1260, nucleotide 1200 to nucleotide 1260, nucleotide 1000 to nucleotide 1250, nucleotide 1050 to nucleotide 1250, nucleotide 1100 to nucleotide 1250, nucleotide 1150 to nucleotide 1250, nucleotide 1200 to nucleotide 1250, nucleotide 1000 to nucleotide 1200, nucleotide 1050 to nucleotide 1200, nucleotide 1100 to nucleotide 1200, nucleotide 1150 to nucleotide 1200, nucleotide 1000 to nucleotide 1150, nucleotide 1050 to nucleotide 1150, nucleotide 1100 to nucleotide 1150, nucleotide 1000 to nucleotide 1100, or nucleotide 1050 to nucleotide 1100, according to SEQ ID NO: 1. All ranges are inclusive of the endpoints.

In some embodiments, the CRE comprises one or more nucleotides within the region corresponding to nucleotide 1000 to nucleotide 1260 according to SEQ ID NO: 1. In some embodiments, the CRE comprises the polynucleotide sequence corresponding to nucleotide 1000 to nucleotide 1260, nucleotide 1050 to nucleotide 1260, nucleotide 1100 to nucleotide 1260, nucleotide 1150 to nucleotide 1260, nucleotide 1200 to nucleotide 1260, nucleotide 1000 to nucleotide 1250, nucleotide 1050 to nucleotide 1250, nucleotide 1100 to nucleotide 1250, nucleotide 1150 to nucleotide 1250, nucleotide 1200 to nucleotide 1250, nucleotide 1000 to nucleotide 1200, nucleotide 1050 to nucleotide 1200, nucleotide 1100 to nucleotide 1200, nucleotide 1150 to nucleotide 1200, nucleotide 1000 to nucleotide 1150, nucleotide 1050 to nucleotide 1150, nucleotide 1100 to nucleotide 1150, nucleotide 1000 to nucleotide 1100, or nucleotide 1050 to nucleotide 1100, of SEQ ID NO: 1. In some embodiments, the CRE comprises a polynucleotide sequence having at least 70%, at least 75%, at least 80%, at least 85%, at least 90%, at least 95%, at least 96%, at least 97%, at least 98%, at least 99%, or 100% identity to the polynucleotide sequence corresponding to nucleotide 1000 to nucleotide 1260, nucleotide 1050 to nucleotide 1260, nucleotide 1100 to nucleotide 1260, nucleotide 1150 to nucleotide 1260, nucleotide 1200 to nucleotide 1260, nucleotide 1000 to nucleotide 1250, nucleotide 1050 to nucleotide 1250, nucleotide 1100 to nucleotide 1250, nucleotide 1150 to nucleotide 1250, nucleotide 1200 to nucleotide 1250, nucleotide 1000 to nucleotide 1200, nucleotide 1050 to nucleotide 1200, nucleotide 1100 to nucleotide 1200, nucleotide 1150 to nucleotide 1200, nucleotide 1000 to nucleotide 1150, nucleotide 1050 to nucleotide 1150, nucleotide 1100 to nucleotide 1150, nucleotide 1000 to nucleotide 1100, or nucleotide 1050 to nucleotide 1100, of SEQ ID NO: 1. All ranges are inclusive of the endpoints.

In some embodiments, the CRE comprises one or more nucleotides within the region corresponding to nucleotide 1117 to nucleotide 1260 according to SEQ ID NO: 1. The polynucleotide sequence of this CRE region is represented by SEQ ID NO: 149. In some embodiments, the CRE comprises a polynucleotide sequence having at least 70%, at least 75%, at least 80%, at least 85%, at least 90%, at least 95%, at least 96%, at least 97%, at least 98%, at least 99%, or 100% identity to SEQ ID NO: 149. In some embodiments, the CRE comprises a polynucleotide sequence having at least 70%, at least 75%, at least 80%, at least 85%, at least 90%, at least 95%, at least 96%, at least 97%, at least 98%, at least 99%, or 100% identity to a 10 consecutive nucleotide segment of SEQ ID NO: 149. In some embodiments, the CRE comprises a polynucleotide sequence having at least 70%, at least 75%, at least 80%, at least 85%, at least 90%, at least 95%, at least 96%, at least 97%, at least 98%, at least 99%, or 100% identity to a 20 consecutive nucleotide segment of SEQ ID NO: 149. In some embodiments, the CRE comprises a polynucleotide sequence having at least 70%, at least 75%, at least 80%, at least 85%, at least 90%, at least 95%, at least 96%, at least 97%, at least 98%, at least 99%, or 100% identity to a 30 consecutive nucleotide segment of SEQ ID NO: 149. In some embodiments, the CRE comprises a polynucleotide sequence having at least 70%, at least 75%, at least 80%, at least 85%, at least 90%, at least 95%, at least 96%, at least 97%, at least 98%, at least 99%, or 100% identity to a 40 consecutive nucleotide segment of SEQ ID NO: 149. In some embodiments, the CRE comprises a polynucleotide sequence having at least 70%, at least 75%, at least 80%, at least 85%, at least 90%, at least 95%, at least 96%, at least 97%, at least 98%, at least 99%, or 100% identity to a 50 consecutive nucleotide segment of SEQ ID NO: 149. In some embodiments, the CRE comprises a polynucleotide sequence having at least 70%, at least 75%, at least 80%, at least 85%, at least 90%, at least 95%, at least 96%, at least 97%, at least 98%, at least 99%, or 100% identity to a 60 consecutive nucleotide segment of SEQ ID NO: 149. In some embodiments, the CRE comprises a polynucleotide sequence having at least 70%, at least 75%, at least 80%, at least 85%, at least 90%, at least 95%, at least 96%, at least 97%, at least 98%, at least 99%, or 100% identity to a 70 consecutive nucleotide segment of SEQ ID NO: 149. In some embodiments, the CRE comprises a polynucleotide sequence having at least 70%, at least 75%, at least 80%, at least 85%, at least 90%, at least 95%, at least 96%, at least 97%, at least 98%, at least 99%, or 100% identity to a 80 consecutive nucleotide segment of SEQ ID NO: 149. In some embodiments, the CRE comprises a polynucleotide sequence having at least 70%, at least 75%, at least 80%, at least 85%, at least 90%, at least 95%, at least 96%, at least 97%, at least 98%, at least 99%, or 100% identity to a 90 consecutive nucleotide segment of SEQ ID NO: 149. In some embodiments, the CRE comprises a polynucleotide sequence having at least 70%, at least 75%, at least 80%, at least 85%, at least 90%, at least 95%, at least 96%, at least 97%, at least 98%, at least 99%, or 100% identity to a 100 consecutive nucleotide segment of SEQ ID NO: 149. In some embodiments, the CRE comprises a polynucleotide sequence having at least 70%, at least 75%, at least 80%, at least 85%, at least 90%, at least 95%, at least 96%, at least 97%, at least 98%, at least 99%, or 100% identity to a 110 consecutive nucleotide segment of SEQ ID NO: 149. In some embodiments, the CRE comprises a polynucleotide sequence having at least 70%, at least 75%, at least 80%, at least 85%, at least 90%, at least 95%, at least 96%, at least 97%, at least 98%, at least 99%, or 100% identity to a 120 consecutive nucleotide segment of SEQ ID NO: 149. In some embodiments, the CRE comprises a polynucleotide sequence having at least 70%, at least 75%, at least 80%, at least 85%, at least 90%, at least 95%, at least 96%, at least 97%, at least 98%, at least 99%, or 100% identity to a 130 consecutive nucleotide segment of SEQ ID NO: 149. In some embodiments, the CRE comprises a polynucleotide sequence having at least 70%, at least 75%, at least 80%, at least 85%, at least 90%, at least 95%, at least 96%, at least 97%, at least 98%, at least 99%, or 100% identity to a 140 consecutive nucleotide segment of SEQ ID NO: 149.

In some embodiments, the SVV genome of the replicon comprises one or more deletions or truncations of the SVV genome within the region corresponding to nucleotide 1261 to nucleotide 3477, inclusive of the endpoints and according to the numbering of SEQ ID NO: 1, wherein the one or more deletions or truncations comprise at least 500 bp in total, and wherein the SVV genome of the replicon comprises a CRE polynucleotide sequence having at least 70%, at least 75%, at least 80%, at least 85%, at least 90%, at least 95%, at least 96%, at least 97%, at least 98%, at least 99%, or 100% identity to SEQ ID NO: 149. In some embodiments, the SVV genome of the replicon comprises one or more deletions or truncations of the SVV genome within the region corresponding to nucleotide 1261 to nucleotide 3477, inclusive of the endpoints and according to the numbering of SEQ ID NO: 1, wherein the one or more deletions or truncations comprise at least 600 bp in total, and wherein the SVV genome of the replicon comprises a CRE polynucleotide sequence having at least 70%, at least 75%, at least 80%, at least 85%, at least 90%, at least 95%, at least 96%, at least 97%, at least 98%, at least 99%, or 100% identity to SEQ ID NO: 149. In some embodiments, the SVV genome of the replicon comprises one or more deletions or truncations of the SVV genome within the region corresponding to nucleotide 1261 to nucleotide 3477, inclusive of the endpoints and according to the numbering of SEQ ID NO: 1, wherein the one or more deletions or truncations comprise at least 700 bp in total, and wherein the SVV genome of the replicon comprises a CRE polynucleotide sequence having at least 70%, at least 75%, at least 80%, at least 85%, at least 90%, at least 95%, at least 96%, at least 97%, at least 98%, at least 99%, or 100% identity to SEQ ID NO: 149. In some embodiments, the SVV genome of the replicon comprises one or more deletions or truncations of the SVV genome within the region corresponding to nucleotide 1261 to nucleotide 3477, inclusive of the endpoints and according to the numbering of SEQ ID NO: 1, wherein the one or more deletions or truncations comprise at least 800 bp in total, and wherein the SVV genome of the replicon comprises a CRE polynucleotide sequence having at least 70%, at least 75%, at least 80%, at least 85%, at least 90%, at least 95%, at least 96%, at least 97%, at least 98%, at least 99%, or 100% identity to SEQ ID NO: 149. In some embodiments, the SVV genome of the replicon comprises one or more deletions or truncations of the SVV genome within the region corresponding to nucleotide 1261 to nucleotide 3477, inclusive of the endpoints and according to the numbering of SEQ ID NO: 1, wherein the one or more deletions or truncations comprise at least 900 bp in total, and wherein the SVV genome of the replicon comprises a CRE polynucleotide sequence having at least 70%, at least 75%, at least 80%, at least 85%, at least 90%, at least 95%, at least 96%, at least 97%, at least 98%, at least 99%, or 100% identity to SEQ ID NO: 149. In some embodiments, the SVV genome of the replicon comprises one or more deletions or truncations of the SVV genome within the region corresponding to nucleotide 1261 to nucleotide 3477, inclusive of the endpoints and according to the numbering of SEQ ID NO: 1, wherein the one or more deletions or truncations comprise at least 1000 bp in total, and wherein the SVV genome of the replicon comprises a CRE polynucleotide sequence having at least 70%, at least 75%, at least 80%, at least 85%, at least 90%, at least 95%, at least 96%, at least 97%, at least 98%, at least 99%, or 100% identity to SEQ ID NO: 149. In some embodiments, the SVV genome of the replicon comprises one or more deletions or truncations of the SVV genome within the region corresponding to nucleotide 1261 to nucleotide 3477, inclusive of the endpoints and according to the numbering of SEQ ID NO: 1, wherein the one or more deletions or truncations comprise at least 1100 bp in total, and wherein the SVV genome of the replicon comprises a CRE polynucleotide sequence having at least 70%, at least 75%, at least 80%, at least 85%, at least 90%, at least 95%, at least 96%, at least 97%, at least 98%, at least 99%, or 100% identity to SEQ ID NO: 149. In some embodiments, the SVV genome of the replicon comprises one or more deletions or truncations of the SVV genome within the region corresponding to nucleotide 1261 to nucleotide 3477, inclusive of the endpoints, and according to the numbering of SEQ ID NO: 1, wherein the one or more deletions or truncations comprise at least 1200 bp in total, and wherein the SVV genome of the replicon comprises a CRE polynucleotide sequence having at least 70%, at least 75%, at least 80%, at least 85%, at least 90%, at least 95%, at least 96%, at least 97%, at least 98%, at least 99%, or 100% identity to SEQ ID NO: 149. In some embodiments, the SVV genome of the replicon comprises one or more deletions or truncations of the SVV genome within the region corresponding to nucleotide 1261 to nucleotide 3477, inclusive of the endpoints, and according to the numbering of SEQ ID NO: 1, wherein the one or more deletions or truncations comprise at least 1300 bp in total, and wherein the SVV genome of the replicon comprises a CRE polynucleotide sequence having at least 70%, at least 75%, at least 80%, at least 85%, at least 90%, at least 95%, at least 96%, at least 97%, at least 98%, at least 99%, or 100% identity to SEQ ID NO: 149. In some embodiments, the SVV genome of the replicon comprises one or more deletions or truncations of the SVV genome within the region corresponding to nucleotide 1261 to nucleotide 3477, inclusive of the endpoints, and according to the numbering of SEQ ID NO: 1, wherein the one or more deletions or truncations comprise at least 1400 bp in total, and wherein the SVV genome of the replicon comprises a CRE polynucleotide sequence having at least 70%, at least 75%, at least 80%, at least 85%, at least 90%, at least 95%, at least 96%, at least 97%, at least 98%, at least 99%, or 100% identity to SEQ ID NO: 149. In some embodiments, the SVV genome of the replicon comprises one or more deletions or truncations of the SVV genome within the region corresponding to nucleotide 1261 to nucleotide 3477, inclusive of the endpoints, and according to the numbering of SEQ ID NO: 1, wherein the one or more deletions or truncations comprise at least 1500 bp in total, and wherein the SVV genome of the replicon comprises a CRE polynucleotide sequence having at least 70%, at least 75%, at least 80%, at least 85%, at least 90%, at least 95%, at least 96%, at least 97%, at least 98%, at least 99%, or 100% identity to SEQ ID NO: 149. In some embodiments, the SVV genome of the replicon comprises one or more deletions or truncations of the SVV genome within the region corresponding to nucleotide 1261 to nucleotide 3477, inclusive of the endpoints, and according to the numbering of SEQ ID NO: 1, wherein the one or more deletions or truncations comprise at least 1600 bp in total, and wherein the SVV genome of the replicon comprises a CRE polynucleotide sequence having at least 70%, at least 75%, at least 80%, at least 85%, at least 90%, at least 95%, at least 96%, at least 97%, at least 98%, at least 99%, or 100% identity to SEQ ID NO: 149. In some embodiments, the SVV genome of the replicon comprises one or more deletions or truncations of the SVV genome within the region corresponding to nucleotide 1261 to nucleotide 3477, inclusive of the endpoints, and according to the numbering of SEQ ID NO: 1, wherein the one or more deletions or truncations comprise at least 1700 bp in total, and wherein the SVV genome of the replicon comprises a CRE polynucleotide sequence having at least 70%, at least 75%, at least 80%, at least 85%, at least 90%, at least 95%, at least 96%, at least 97%, at least 98%, at least 99%, or 100% identity to SEQ ID NO: 149. In some embodiments, the SVV genome of the replicon comprises one or more deletions or truncations of the SVV genome within the region corresponding to nucleotide 1261 to nucleotide 3477, inclusive of the endpoints, and according to the numbering of SEQ ID NO: 1, wherein the one or more deletions or truncations comprise at least 1800 bp in total, and wherein the SVV genome of the replicon comprises a CRE polynucleotide sequence having at least 70%, at least 75%, at least 80%, at least 85%, at least 90%, at least 95%, at least 96%, at least 97%, at least 98%, at least 99%, or 100% identity to SEQ ID NO: 149. In some embodiments, the SVV genome of the replicon comprises one or more deletions or truncations of the SVV genome within the region corresponding to nucleotide 1261 to nucleotide 3477, inclusive of the endpoints, and according to the numbering of SEQ ID NO: 1, wherein the one or more deletions or truncations comprise at least 1900 bp in total, and wherein the SVV genome of the replicon comprises a CRE polynucleotide sequence having at least 70%, at least 75%, at least 80%, at least 85%, at least 90%, at least 95%, at least 96%, at least 97%, at least 98%, at least 99%, or 100% identity to SEQ ID NO: 149. In some embodiments, the SVV genome of the replicon comprises one or more deletions or truncations of the SVV genome within the region corresponding to nucleotide 1261 to nucleotide 3477, inclusive of the endpoints, and according to the numbering of SEQ ID NO: 1, wherein the one or more deletions or truncations comprise at least 2000 bp in total, and wherein the SVV genome of the replicon comprises a CRE polynucleotide sequence having at least 70%, at least 75%, at least 80%, at least 85%, at least 90%, at least 95%, at least 96%, at least 97%, at least 98%, at least 99%, or 100% identity to SEQ ID NO: 149. In some embodiments, the SVV genome of the replicon comprises one or more deletions or truncations of the SVV genome within the region corresponding to nucleotide 1261 to nucleotide 3477, inclusive of the endpoints, and according to the numbering of SEQ ID NO: 1, wherein the one or more deletions or truncations comprise at least 2100 bp in total, and wherein the SVV genome of the replicon comprises a CRE polynucleotide sequence having at least 70%, at least 75%, at least 80%, at least 85%, at least 90%, at least 95%, at least 96%, at least 97%, at least 98%, at least 99%, or 100% identity to SEQ ID NO: 149. In some embodiments, the SVV genome of the replicon comprises one or more deletions or truncations of the SVV genome within the region corresponding to nucleotide 1261 to nucleotide 3477, inclusive of the endpoints, and according to the numbering of SEQ ID NO: 1, wherein the one or more deletions or truncations comprise at least 2200 bp in total, and wherein the SVV genome of the replicon comprises a CRE polynucleotide sequence having at least 70%, at least 75%, at least 80%, at least 85%, at least 90%, at least 95%, at least 96%, at least 97%, at least 98%, at least 99%, or 100% identity to SEQ ID NO: 149.

In some embodiments, the SVV genome of the replicon comprises one or more of a 2B coding region, a 2C coding region, a 3A coding region, a 3B coding region, a 3Cpro coding region, and a 3D(RdRp) coding region. In some embodiments, the SVV genome of the replicon comprises a 2B coding region, a 2C coding region, a 3A coding region, a 3B coding region, a 3Cpro coding region, and a 3D(RdRp) coding region. In some embodiments, the SVV genome of the replicon comprises a 2C coding region, a 3A coding region, a 3B coding region, a 3Cpro coding region, and a 3D(RdRp) coding region. In some embodiments, the SVV genome of the replicon comprises, from 5′ to 3′, the 2B coding region, the 2C coding region, the 3A coding region, the 3B coding region, the 3Cpro coding region, and the 3D(RdRp) coding region. In some embodiments, a portion of the SVV genome of the replicon comprising the 2B coding region, the 2C coding region, the 3A coding region, the 3B coding region, the 3Cpro coding region, and the 3D(RdRp) coding region has at least 70%, at least 75%, at least 80%, at least 85%, at least 90%, at least 93%, at least 95%, at least 97%, at least 98%, at least 99%, at least 99.5%, or 100% sequence identity to nucleotide 3505 to 7310 according to SEQ ID NO: 1.

In some embodiments, the recombinant RNA replicon comprises, from 5′ to 3′, the 5′ leader protein coding sequence, the VP4 coding region, the VP2 coding region or a truncation thereof, and the heterologous polynucleotide. In some embodiments, the replicon comprises, from 5′ to 3′, the heterologous polynucleotide and the 2B coding region. In some embodiments, the recombinant RNA replicon comprises, from 5′ to 3′, the heterologous polynucleotide, the 2B coding region, the 2C coding region, the 3A coding region, the 3B coding region, the 3Cpro coding region, and the 3D(RdRp).

In some embodiments, the SVV genome comprises a 2A coding region. In some embodiments, the 2A coding region is located between the VP2 coding region or a truncation thereof and the heterologous polynucleotide. In some embodiments, the 2A coding region is located between the heterologous polynucleotide and the 2B coding region.

In some embodiments, the SVV derived replicon comprises one or more heterologous polynucleotides. In some embodiments, the heterologous polynucleotide of the replicon comprises at least 500 bp, at least 1000 bp, at least 1500 bp, at least 2000 bp, at least 2500 bp, or at least 3000 bp. In some embodiments, the one or more heterologous polynucleotides comprises at least 500 bp, at least 1000 bp, at least 1500 bp, at least 2000 bp, at least 2500 bp, or at least 3000 bp in total.

In some embodiments, the SVV derived replicon comprises a sequence having at least 70%, at least 75%, at least 80%, at least 85%, at least 90%, at least 93%, at least 95%, at least 97%, at least 98%, at least 99%, at least 99.5%, or 100% sequence identity to any one of SEQ ID NOs: 20, 22, 24, 26, 28, 30, 32, 34, 36, 38, 40, 42, 44, 46, 48, 50, 52, 54, 56, 58, and 60.

In some embodiments, the SVV derived replicon comprises an SVV genome and a heterologous polynucleotide; wherein the SVV genome comprises a deletion between nucleotide 1261 and 3477, inclusive of the endpoints, and according to the numbering of SEQ ID NO: 1, wherein the deletion is at least 500 bp, 600 bp, 700 bp, 800 bp, 900 bp, 1000 bp, 1100 bp, 1200 bp, 1300 bp, 1400 bp, 1500 bp, 1600 bp, 1700 bp, 1800 bp, 1900 bp, 2000 bp, 2100 bp, 2200 bp, 2300 bp, or 2400 bp in total length; wherein the SVV genome comprises a CRE comprising a polynucleotide sequence having at least 70%, at least 75%, at least 80%, at least 85%, at least 90%, at least 95%, at least 96%, at least 97%, at least 98%, at least 99%, at least 99.5%, or 100% identity to SEQ ID NO: 149.

In some embodiments, the SVV derived replicon comprises an SVV genome and a heterologous polynucleotide; wherein the SVV genome comprises a deletion between nucleotide 1261 and 3477, inclusive of the endpoints, and according to the numbering of SEQ ID NO: 1, wherein the deletion is at least 500 bp, 600 bp, 700 bp, 800 bp, 900 bp, 1000 bp, 1100 bp, 1200 bp, 1300 bp, 1400 bp, 1500 bp, 1600 bp, 1700 bp, 1800 bp, 1900 bp, 2000 bp, 2100 bp, 2200 bp, 2300 bp, or 2400 bp in total length; wherein the SVV genome comprises a CRE comprising a polynucleotide sequence having at least 90% identity to SEQ ID NO: 149.

In some embodiments, the SVV derived replicon comprises an SVV genome and a heterologous polynucleotide; wherein the SVV genome comprises a deletion between nucleotide 1261 and 3477, inclusive of the endpoints, according to the numbering of SEQ ID NO: 1, wherein the deletion is at least 500 bp, 600 bp, 700 bp, 800 bp, 900 bp, 1000 bp, 1100 bp, 1200 bp, 1300 bp, 1400 bp, 1500 bp, 1600 bp, 1700 bp, 1800 bp, 1900 bp, 2000 bp, 2100 bp, 2200 bp, 2300 bp, or 2400 bp in total length; wherein the SVV genome comprises a polynucleotide sequence having at least 70%, at least 75%, at least 80%, at least 85%, at least 90%, at least 95%, at least 96%, at least 97%, at least 98%, at least 99%, at least 99.5%, or 100% identity to nucleotide 1 to 1260 according to SEQ ID NO: 1; wherein the SVV genome comprises a polynucleotide sequence having at least 70%, at least 75%, at least 80%, at least 85%, at least 90%, at least 95%, at least 96%, at least 97%, at least 98%, at least 99%, at least 99.5%, or 100% identity to nucleotide 3505 to 7310 according to SEQ ID NO: 1; and wherein the SVV genome comprises a CRE comprising a polynucleotide sequence having at least 70%, at least 75%, at least 80%, at least 85%, at least 90%, at least 95%, at least 96%, at least 97%, at least 98%, at least 99%, at least 99.5%, or 100% identity to SEQ ID NO: 149.

In some embodiments, the SVV derived replicon comprises an SVV genome and a heterologous polynucleotide; wherein the SVV genome comprises a deletion between nucleotide 1261 and 3477, inclusive of the endpoints, according to the numbering of SEQ ID NO: 1, wherein the deletion is at least 500 bp, 600 bp, 700 bp, 800 bp, 900 bp, 1000 bp, 1100 bp, 1200 bp, 1300 bp, 1400 bp, 1500 bp, 1600 bp, 1700 bp, 1800 bp, 1900 bp, 2000 bp, 2100 bp, 2200 bp, 2300 bp, or 2400 bp in total length; wherein the SVV genome comprises a polynucleotide sequence having at least 90% identity to nucleotide 1 to 1260 according to SEQ ID NO: 1; wherein the SVV genome comprises a polynucleotide sequence having at least 90% identity to nucleotide 3505 to 7310 according to SEQ ID NO: 1; and wherein the SVV genome comprises a CRE comprising a polynucleotide sequence having at least 90% identity to SEQ ID NO: 149.

### Recombinant Coxsackievirus Replicon

The disclosure provides recombinant RNA replicons comprising a coxsackievirus viral genome, wherein the coxsackievirus genome comprises a deletion or a truncation in one or more coxsackievirus protein coding regions. In some embodiments, the replicon comprises a heterologous polynucleotide.

In some embodiments, the coxsackievirus is selected from CVB3, CVA21, and CVA9. The nucleic acid sequences of exemplary coxsackieviruses are provided as GenBank Reference No. M33854.1 (CVB3; SEQ ID NO: 16), GenBank Reference No. KT161266.1 (CVA21; SEQ ID NO: 17), and GenBank Reference No. D00627.1 (CVA9; SEQ ID NO: 18). In some embodiments, the recombinant RNA replicon described herein encode a chimeric coxsackievirus.

For coxsackievirus viral genome, the VP4 coding region encompasses nucleotide 714 to nucleotide 920 according to SEQ ID NO: 3. The VP2 coding region encompasses nucleotide 921 to nucleotide 1736 according to SEQ ID NO: 3. The VP3 coding region encompasses nucleotide 1737 to nucleotide 2456 according to SEQ ID NO: 3. The VP1 coding region encompasses nucleotide 2457 to nucleotide 3350 according to SEQ ID NO: 3. The 2A coding region encompasses nucleotide 3351 to nucleotide 3797 according to SEQ ID NO: 3. The 2B coding region encompasses nucleotide 3798 to nucleotide 4088 according to SEQ ID NO: 3.

In some embodiments, the recombinant RNA replicon comprises a coxsackievirus genome comprising the 5′ UTR sequence of SEQ ID NO: 4. In such embodiments, the 5′ UTR sequence of SEQ ID NO: 4 unexpectedly increases the production of a functional coxsackievirus compared to other previously described 5′ UTR sequences. In some embodiment, the recombinant RNA replicon comprises a modified CVA21 coxsackievirus genome according to the sequence of SEQ ID NO: 3.

In some embodiments, the coxsackievirus genome of the replicon comprises deletions and/or truncations in one or more VP coding regions. In some embodiments, one, or at least one of the VP4, VP2, VP3 and VP1 coding regions are deleted and/or truncated. In some embodiments, two, or at least two, of the VP coding regions comprising VP4, VP2, VP3 and VP1 are deleted and/or truncated. In some embodiments, three, or at least three, of the VP coding regions comprising VP4, VP2, VP3 and VP1 are deleted and/or truncated. In some embodiments, all of the VP4, VP2, VP3 and VP1 coding regions are deleted and/or truncated.

In some embodiments, the coxsackievirus genome of the replicon comprises, consists essentially of, or consists of, one or more deletions or truncations of the coxsackievirus genome within the region corresponding to nucleotide 714 to nucleotide 3350, inclusive of the endpoints, according to SEQ ID NO: 3. In some embodiments, the coxsackievirus genome of the replicon comprises a deletion of the coxsackievirus genome region corresponding to nucleotide 714 to nucleotide 3350, inclusive of the endpoints, according to SEQ ID NO: 3. In some embodiments, the replicon comprises one or more deletions or truncations within the region corresponding to nucleotide 1000 to nucleotide 3350 according to SEQ ID NO: 3. In some embodiments, the replicon comprises one or more deletions or truncations within the region corresponding to nucleotide 714 to nucleotide 3350, nucleotide 1000 to nucleotide 3350, nucleotide 1500 to nucleotide 3350, nucleotide 2000 to nucleotide 3350, nucleotide 2500 to nucleotide 3350, nucleotide 714 to nucleotide 3000, nucleotide 1000 to nucleotide 3000, nucleotide 1500 to nucleotide 3000, nucleotide 2000 to nucleotide 3000, nucleotide 2500 to nucleotide 3000, nucleotide 714 to nucleotide 2500, nucleotide 1000 to nucleotide 2500, nucleotide 1500 to nucleotide 2500, nucleotide 2000 to nucleotide 2500, nucleotide 714 to nucleotide 2000, nucleotide 1000 to nucleotide 2000, nucleotide 1500 to nucleotide 2000, nucleotide 714 to nucleotide 1500, or nucleotide 1000 to nucleotide 1500, inclusive of the endpoints, according to SEQ ID NO: 3. All ranges are inclusive of the endpoints.

In some embodiments, the coxsackievirus genome of the replicon comprises, consists essentially of, or consists of, one or more deletions or truncations of the coxsackievirus genome within the region corresponding to nucleotide 717 to nucleotide 3332, inclusive of the endpoints, according to SEQ ID NO: 3. In some embodiments, the coxsackievirus genome of the replicon comprises a deletion of the coxsackievirus genome region corresponding to nucleotide 717 to nucleotide 3332, inclusive of the endpoints, according to SEQ ID NO: 3. In some embodiments, the replicon comprises one or more deletions or truncations within the region corresponding to nucleotide 1000 to nucleotide 3332 according to SEQ ID NO: 3. In some embodiments, the replicon comprises one or more deletions or truncations within the region corresponding to nucleotide 717 to nucleotide 3332, nucleotide 1000 to nucleotide 3332, nucleotide 1500 to nucleotide 3332, nucleotide 2000 to nucleotide 3332, nucleotide 2500 to nucleotide 3332, nucleotide 717 to nucleotide 3000, nucleotide 1000 to nucleotide 3000, nucleotide 1500 to nucleotide 3000, nucleotide 2000 to nucleotide 3000, nucleotide 2500 to nucleotide 3000, nucleotide 717 to nucleotide 2500, nucleotide 1000 to nucleotide 2500, nucleotide 1500 to nucleotide 2500, nucleotide 2000 to nucleotide 2500, nucleotide 717 to nucleotide 2000, nucleotide 1000 to nucleotide 2000, nucleotide 1500 to nucleotide 2000, nucleotide 717 to nucleotide 1500, or nucleotide 1000 to nucleotide 1500, inclusive of the endpoints, according to SEQ ID NO: 3. All ranges are inclusive of the endpoints.

In some embodiments, each of the deletion or the truncation comprises 1 or more nucleotides. In some embodiments, each of the deletion or the truncation comprises 10 or more nucleotides. In some embodiments, each of the deletion or the truncation comprises 50 or more nucleotides. In some embodiments, each of the deletion or the truncation comprises 100 or more nucleotides. In some embodiments, each of the deletion or the truncation comprises 500 or more nucleotides. In some embodiments, each of the deletion or the truncation comprises 1000 or more nucleotides. All ranges are inclusive of the endpoints.

In some embodiments, the one or more deletions or truncations comprise at least 500 bp, at least 600 bp, at least 700 bp, at least 800 bp, at least 900 bp, at least 1000 bp, at least 1100 bp, at least 1200 bp, at least 1300 bp, at least 1400 bp, at least 1500 bp, at least 1600 bp, at least 1700 bp, at least 1800 bp, at least 1900 bp, at least 2000 bp, at least 2100 bp, at least 2200 bp, at least 2300 bp, at least 2400 bp, at least 2500 bp, at least 2600 bp, at least 2615 bp, at least 2636 bp, at least 2650 bp, or at least 2700 bp of nucleotides in total. In some embodiments, the one or more deletions or truncations consist of 500 bp, 600 bp, 700 bp, 800 bp, 900 bp, 1000 bp, 1100 bp, 1200 bp, 1300 bp, 1400 bp, 1500 bp, 1600 bp, 1700 bp, 1800 bp, 1900 bp, 2000 bp, 2100 bp, 2200 bp, 2300 bp, 2400 bp, 2500 bp, 2600 bp, 2700 bp, or any values in between, of nucleotides in total. In some embodiments, the one or more deletions or truncations consist of between 500-2700 bp, between 500-2600 bp, between 500-2300 bp, between 500-2000 bp, between 500-1500 bp, between 500-1000 bp, between 1000-2700 bp, between 1000-2600 bp, between 1000-2300 bp, between 1000-2000 bp, between 1000-1500 bp, between 1500-2700 bp, between 1500-2600 bp, between 1500-2300 bp, between 1500-2200 bp, between 1500-2000 bp, between 2000-2700 bp, between 2000-2600 bp, between 2000-2300 bp, or between 2000-2200 bp of nucleotides in total. All ranges are inclusive of the endpoints.

In some embodiments, the coxsackievirus genome of the replicon comprises a 5′ UTR. In some embodiments, a portion of the coxsackievirus genome of the replicon comprising the 5′ UTR has at least 70%, at least 75%, at least 80%, at least 85%, at least 90%, at least 93%, at least 95%, at least 97%, at least 98%, at least 99%, at least 99.5%, or 100% sequence identity to SEQ ID NO: 4. In some embodiments, a portion of the coxsackievirus genome of the replicon comprising the 5′ UTR has about 70%, about 75%, about 80%, about 85%, about 90%, about 93%, about 95%, about 97%, about 98%, about 99%, about 99.5%, or 100% sequence identity to SEQ ID NO: 4. In some embodiments, a portion of the coxsackievirus genome of the replicon comprising the 5′ UTR has at most 1, at most 5, at most 10, or at most 20 nucleotide mutations according to SEQ ID NO: 4.

In some embodiments, the coxsackievirus genome of the replicon comprises one or more of a 2B coding region, a 2C coding region, a 3A coding region, a 3B coding region, a VPg coding region, a 3C coding region, a 3D pol coding region, and a 3′ UTR. In some embodiments, the coxsackievirus genome of the replicon comprises a 2B coding region, a 2C coding region, a 3A coding region, a 3B coding region, a VPg coding region, a 3C coding region, a 3D pol coding region, and a 3′ UTR. In some embodiments, the coxsackievirus genome of the replicon comprises, from 5′ to 3′ direction, the 2B coding region, the 2C coding region, the 3A coding region, the 3B coding region, the VPg coding region, the 3C coding region, the 3D pol coding region, and the 3′ UTR. In some embodiments, a portion of the coxsackievirus genome comprising the 2B coding region, the 2C coding region, the 3A coding region, the 3B coding region, the VPg coding region, the 3C coding region, the 3D pol coding region, and the 3′ UTR has at least 70%, at least 75%, at least 80%, at least 85%, at least 90%, at least 93%, at least 95%, at least 97%, at least 98%, at least 99%, at least 99.5%, or 100% sequence identity to nucleotide 3797 to 7435 according to SEQ ID NO: 3.

In some embodiments, the replicon comprises, from 5′ to 3′, the 5′ UTR and the heterologous polynucleotide. In some embodiments, the replicon comprises, from 5′ to 3′, the heterologous polynucleotide and the 2B coding region. In some embodiments, the recombinant RNA replicon comprises, from 5′ to 3′, the heterologous polynucleotide, the 2B coding region, the 2C coding region, the 3A coding region, the 3B coding region, the VPg coding region, the 3C coding region, the 3D pol coding region, and the 3′ UTR.

In some embodiments, the replicon further comprises a 2A coding region. In some embodiments, the 2A coding region is located between the 5′ UTR and the heterologous polynucleotide. In some embodiments, the 2A coding region is located between the heterologous polynucleotide and the 2B coding region. In some embodiments, the replicon comprises, from 5′ to 3′, the 5′ UTR, the heterologous polynucleotide, and the 2A coding region. In some embodiments, the coxsackievirus genome of the replicon comprises, from 5′ to 3′ direction, the heterologous polynucleotide, the 2A coding region, the 2B coding region, the 2C coding region, the 3A coding region, the 3B coding region, the VPg coding region, the 3C coding region, the 3D pol coding region, and the 3′ UTR.

In some embodiments, the coxsackievirus genome of the replicon comprises, from 5′ to 3′ direction, the 2A coding region, the 2B coding region, the 2C coding region, the 3A coding region, the 3B coding region, the VPg coding region, the 3C coding region, the 3D pol coding region, and the 3′ UTR. In some embodiments, a portion of the coxsackievirus genome comprising the 2A coding region, the 2B coding region, the 2C coding region, the 3A coding region, the 3B coding region, the VPg coding region, the 3C coding region, the 3D pol coding region, and the 3′ UTR has at least 70%, at least 75%, at least 80%, at least 85%, at least 90%, at least 93%, at least 95%, at least 97%, at least 98%, at least 99%, at least 99.5%, or 100% sequence identity to nucleotide 3492 to 7435 according to SEQ ID NO: 3.

In some embodiments, the coxsackievirus derived replicon comprises one or more heterologous polynucleotides. In some embodiments, the heterologous polynucleotide of the replicon has a length of at least 500 bp, at least 1000 bp, at least 1500 bp, at least 2000 bp, at least 2500 bp, or at least 3000 bp. In some embodiments, the one or more heterologous polynucleotides have a total length of at least 500 bp, at least 1000 bp, at least 1500 bp, at least 2000 bp, at least 2500 bp, or at least 3000 bp. All ranges are inclusive of the endpoints.

In some embodiments, the coxsackievirus derived replicon comprises a sequence having at least 70%, at least 75%, at least 80%, at least 85%, at least 90%, at least 93%, at least 95%, at least 97%, at least 98%, at least 99%, at least 99.5%, or 100% sequence identity to SEQ ID NO: 62.

### Heterologous Polynucleotide and Payload Molecules

In some embodiments, the replicon comprises a heterologous polynucleotide encoding one or more payload molecules.

In some embodiments, the heterologous nucleotide is inserted into a viral genome location between the 2A coding region and the 2B coding region of the viral genome of the replicon. In some embodiments, the heterologous nucleotide is inserted into a viral genome location upstream to the 2A coding region of the viral genome of the replicon. In some embodiments, the heterologous nucleotide is inserted into a viral genome location downstream to the 3D (RdRp) or 3D pol coding region.

In some embodiments, the heterologous nucleotide is inserted into a replicon comprising an SVV viral genome. In some embodiments, the heterologous nucleotide is inserted into the region of the viral genome corresponding to nucleotide 1117 to 3479 of SEQ ID NO: 1. In some embodiments, the heterologous nucleotide is inserted into the region of the viral genome corresponding to nucleotide 3504 to 3505 of SEQ ID NO: 1. In some embodiments, the heterologous nucleotide is inserted into the region of the viral genome corresponding to nucleotide 7209 to 7210 of SEQ ID NO: 1.

In some embodiments, the heterologous nucleotide is inserted into a replicon comprising a coxsakievirus viral genome. In some embodiments, the heterologous nucleotide is inserted into the region of the viral genome corresponding to nucleotide 713 to 3351 of SEQ ID NO: 3. In some embodiments, the heterologous nucleotide is inserted into the region of the viral genome corresponding to nucleotide 3797 to 3798 of SEQ ID NO: 3. In some embodiments, the heterologous nucleotide is inserted into the region of the viral genome corresponding to nucleotide 7334 to 7335 of SEQ ID NO: 3.

In some embodiments, one or more miRNA target sequences are inserted into the heterologous polynucleotide encoding the payload molecule. In some embodiments, one or more miRNA target sequences are incorporated into the 3′ or 5′ UTR of the heterologous polynucleotide encoding the payload molecule. In some embodiments, one or more miRNA target sequences are incorporated into the coding region of the heterologous polynucleotide encoding the payload molecule. In such embodiments, translation and subsequent expression of the payload does not occur, or is substantially reduced, in cells where the corresponding miRNA is expressed. In some embodiments, the payload molecule is a protein.

In some embodiments, the payload molecule is a secreted protein. In some embodiments, the secreted protein comprises a signal peptide. In some embodiments, the secreted protein comprises a non-native signal peptide. In some embodiments, the signal peptide facilitates the secretion of the payload molecule. In some embodiments, the secreted protein does not have a signal peptide.

In some embodiments, the heterologous polynucleotide encoding the payload molecule forms a continuous open reading frame with one or more of the viral protein coding regions. Here, continuous open reading frame refers to a sequence of specific nucleotide triplets that can be translated into a continuous polypeptide. In some embodiments, the payload molecule and the viral protein are linked by a cleavage polypeptide. In some embodiments, the viral protein is 2B.

In some embodiments, the payload molecule is a cytotoxic peptide. As used herein, a “cytotoxic peptide” refers to a protein capable of inducing cell death when expressed in a host cell and/or cell death of a neighboring cell when secreted by the host cell. In some embodiments, the cytotoxic peptide is a caspase, p53, diphtheria toxin (DT), Pseudomonas Exotoxin A (PEA), Type I ribozyme inactivating proteins (RIPs) (e.g., saporin and gelonin), Type II RIPs (e.g., ricin), Shiga-like toxin I (Slt1), photosensitive reactive oxygen species (e.g. killer-red). In certain embodiments, the cytotoxic peptide is encoded by a suicide gene resulting in cell death through apoptosis, such as a caspase gene.

In some embodiments, the payload molecule is an immune modulatory peptide. As used herein, an “immune modulatory peptide” is a peptide capable of modulating (e.g., activating or inhibiting) a particular immune receptor and/or pathway. In some embodiments, the immune modulatory peptides can act on any mammalian cell including immune cells, tissue cells, and stromal cells. In a preferred embodiment, the immune modulatory peptide acts on an immune cell such as a T cell, an NK cell, an NKT T cell, a B cell, a dendritic cell, a macrophage, a basophil, a mast cell, or an eosinophil. Exemplary immune modulatory peptides include antigen-binding molecules such as antibodies or antigen binding fragments thereof, cytokines, chemokines, soluble receptors, cell-surface receptor ligands, bipartite polypeptides, and enzymes.

In some embodiments, the payload molecule is a cytokine such as IFNg, GM-CSF, IL-1, IL-2, IL-12, IL-15, IL-18, IL-36γ, TNFα, IFNα, IFNβ, IFNγ, or TNFSF14. In some embodiments, the payload molecule is a chemokine such as CXCL10, CXCL9, CCL21, CCL4, or CCL5. In some embodiments, the payload molecule is a ligand for a cell-surface receptor such as an NKG2D ligand, a neuropilin ligand, Flt3 ligand, a CD47 ligand (e.g., SIRPIα). In some embodiments, the payload molecule is a soluble receptor, such as a soluble cytokine receptor (e.g., IL-13R, TGFβR1, TGFβR2, IL-35R, IL-15R, IL-2R, IL-12R, and interferon receptors) or a soluble innate immune receptor (e.g., Toll-like receptors, complement receptors, etc.). In some embodiments, the payload molecule is a dominant agonist mutant of a protein involved in intracellular RNA and/or DNA sensing (e.g. a dominant agonist mutant of STING, RIG-1, or MDA-5).

In some embodiments, the payload molecule is an antigen-binding molecule such as an antibody or antigen-binding fragments thereof (e.g., a single chain variable fragment (scFv), an F(ab), etc.). In some embodiments, the antigen-binding molecule specifically binds to a cell surface receptor, such as an immune checkpoint receptor (e.g., PD-1, PD-L1, and CTLA4) or additional cell surface receptors involved in cell growth and activation (e.g., OX40, CD200R, CD47, CSF1R, 41BB, CD40, and NKG2D). In some embodiments, the antigen-binding molecule specifically binds to an antigen shown in Table 3 and/or 4.

In some embodiments, the payload molecule is a scorpion polypeptide such as chlorotoxin, BmKn-2, neopladine 1, neopladine 2, and mauriporin. In some embodiments, the payload molecule is a snake polypeptide such as contortrostatin, apoxin-I, bothropstoxin-I, BJcuL, OHAP-1, rhodostomin, drCT-I, CTX-III, B1L, and ACTX-6. In some embodiments, the payload molecule is a spider polypeptide such as a latarcin and hyaluronidase. In some embodiments, the payload molecule is a bee polypeptide such as melittin and apamin. In some embodiments, the payload molecule is a frog polypeptide such as PsT-1, PdT-1, and PdT-2.

In some embodiments, the payload molecule is an enzyme. In some embodiments, the enzyme is capable of modulating the tumor microenvironment by way of altering the extracellular matrix. In such embodiments, the enzyme may include, but is not limited to, a matrix metalloprotease (e.g., MMP9), a collagenase, a hyaluronidase, a gelatinase, or an elastase. In some embodiments, the enzyme is part of a gene directed enzyme prodrug therapy (GDEPT) system, such as herpes simplex virus thymidine kinase, cytosine deaminase, nitroreductase, carboxypeptidase G2, purine nucleoside phosphorylase, or cytochrome P450. In some embodiments, the enzyme is capable of inducing or activating cell death pathways in the target cell (e.g., a caspase). In some embodiments, the enzyme is capable of degrading an extracellular metabolite or message (e.g. arginase or 15-Hydroxyprostaglandin Dehydrogenase).

In some embodiments, the payload molecule is a bipartite polypeptide (bipartite antigen binding molecule). As used herein, a “bipartite polypeptide” refers to a multimeric protein comprised of a first domain capable of binding a cell surface antigen expressed on a non-cancerous effector cell (e.g., a T cell) and a second domain capable of binding a cell-surface antigen expressed by a target cell (e.g., a cancerous cell, a tumor cell, or an effector cell of a different type). In some embodiments, the individual polypeptide domains of a bipartite polypeptide may comprise an antibody or binding fragment thereof (e.g, a single chain variable fragment (scFv) or an F(ab)), a nanobody, a diabody, a flexibody, a DOCK-AND-LOCK™ antibody, or a monoclonal anti-idiotypic antibody (mAb2). In some embodiments, the structure of the bipartite polypeptides may be a dual-variable domain antibody (DVD-Ig™), a Tandab®, a bi-specific T cell engager (BiTE™), a DuoBody®, or a dual affinity retargeting (DART) polypeptide. In some embodiments, the bipartite polypeptide is a BiTE and comprises a domain that specifically binds to an antigen shown in Table 3 and/or 4. Exemplary BiTEs are shown below in Table 2.

TABLE 2
Validated BiTEs used in preclinical and clinical studies

| Target | Name | Target Disease | Clinical Status |
|---|---|---|---|
| CD19 | Blinatumomab/MT-103/MEDI- | NHL, ALL | Phase I/II/III |
| | 538 | | |
| EpCAM | MT110 | Solid tumors | Phase I |
| CEA | MT111/MEDI-565 | GI adenocarcinoma | Phase I |
| PSMA | Pasotuxizumab/BAY2010112/ | Prostate | Phase I |
| | AMG112 | | |
| CD33 | AMG330 | AML | Preclinical |
| EGFR | C-BiTE and P-BiTE antibodies | Colorectal cancer | Preclinical |
| Her2 | FynomAb, COVA420, HER2- | Breast and gastric | Preclinical |
| | BsAb | carcinoma | |
| EphA2 | bscEphA2xCD3 | Multiple solid tumors | Preclinical |
| MCSP | MCSP-BiTE | Melanoma | Preclinical |
| ADAM17 | A300E | Prostate cancer | Preclinical |
| PSCA | CD3-PSCA(MB1) | Prostate cancer | Preclinical |
| 17-A1 | CD3/17-1A-bispecific | Colorectal cancer | Preclinical |
| NKG2D | scFv-NKG2D, huNKG2D- | Multiple solid and liquid | Preclinical |
| ligands | OKT3 | tumors | |
| DLL3 | AMG757 | Small Cell Lung Cancer | Clinical |

In some embodiments, the cell-surface antigen expressed on an effector cell, which the bipartite polypeptide binds to, is selected from Table 3 below. In some embodiments, the bipartite polypeptide binds to CD3 or one of its components. CD3 is a protein complex and T cell co-receptor that is expressed on T lymphocytes as part of the T cell multimolecular receptor (TCR). It comprises CD37, CD36, CD3E, and/or CD34 receptor chains. In some embodiments, the bipartite polypeptide binds to NKp46. NKp46, also known as CD335, belongs to the natural cytotoxicity receptor (NCR) family and is a glycoprotein with 2 Ig-like domains and a short cytoplasmic tail. In some embodiments, the bipartite polypeptide binds to CD16. CD16, also known as FcγRIII, is a cluster of differentiation molecule found on the surface of natural killer cells, neutrophils, monocytes, and macrophages. In some embodiments, the bipartite polypeptide binds to SIRPα. SIRPα, also known as signal regulatory protein a, is a regulatory membrane glycoprotein from SIRP family expressed mainly by myeloid cells and also by stem cells or neurons, which interacts with transmembrane protein CD47.

In some embodiments, the cell-surface antigen expressed on a tumor cell or effector cell is selected from Table 4 below. In some embodiments, the cell-surface antigen expressed on a tumor cell is a tumor antigen. In some embodiments, the tumor antigen is selected from CD19, EpCAM, CEA, PSMA, CD33, EGFR, Her2, EphA2, MCSP, ADAM17, PSCA, 17-A1, an NKGD2 ligand, CSF1R, FAP, GD2, DLL3, or neuropilin. In some embodiments, the tumor antigen is selected from those listed in Table 4.

In some embodiments, the bipartite polypeptide is selected from a molecule binding to DLL3 and an effector cell target antigen, a molecule binding to FAP and an effector cell target antigen, and a molecule binding to EpCAM and an effector cell target antigen. In some embodiments, the effector cell target antigen is selected from Table 3. In some embodiments, the effect cell target antigen is a T cell target antigen. In some embodiments, the effector cell target antigen is CD3. In some embodiments, the effector cell target antigen is CD3ε.

TABLE 3
Exemplary effector cell target antigens

| T cell | | NKT cell | NK Cell | Other |
|---|---|---|---|---|
| CD3 | CD30 | CD3 | CD16 | CD48 |
| CD3γ | CD38 | CD3γ | CD94/NKG2 | LIGHT |
| | | | (e.g., NKG2D) | |
| CD3δ | CD40 | CD3δ | NKp30 | CD44 |
| CD3ε | CD57 | CD3ε | NKp44 | CD45 |
| CD3ξ | CD69 | CD3ξ | NKp46 | IL-1R2 |
| CD2 | CD70 | invariant TCR | KARs | IL-1Rα |
| CD4 | CD73 | | | IL-1Rα2 |
| CD5 | CD81 | | | IL-13Rα2 |
| CD6 | CD82 | | | IL-15Ra |
| CD7 | CD96 | | | CCR5 |
| CD8 | CD134 | | | CCR8 |
| CD16 | CD137 | | | SIRPα |
| CD25 | CD152 | | | |
| CD27 | CD278 | | | |
| CD28 | | | | |

TABLE 4
Exemplary target cell antigens
Target Cell Antigens

| | | | |
|---|---|---|---|
| 8H9 | CRISP3 | Lewis-Y | Fas |
| GnT-V, β1, 6-N | DC-SIGN | LIV-1 (SLC39A6) | SOX2 |
| AFP | DHFR | Livin | STEAP1 |
| ART1 | EGP40 | LAMP1 | SLITRK6 |
| ART4 | EZH2 | MAGEA3 | NaPi2a |
| ABCG2 | EpCAM | MAGEA4 | SOX1 |
| B7-H3 | EphA2 | MAGEB6 | SOX11 |
| B7-H4 | EphA2/Eck | MAGEA1 | SPANXA1 |
| B7-H6 | EGFRvIII | MART-1 | SART-1 |
| BCMA | E-cadherin | MCSP | SSX4 |
| B-cyclin | EGP2 | MME | SSX5 |
| BMI1 | ETA | mesothelin (MSLN) | Survivin |
| CA-125 | ERBB3 | MAPK1 | SSX2 |
| cadherin | ERBB3/4 | MUC16 | TAG72 |
| CABYR | ERBB4 | MUC1 | TEM1 |
| CTAG2 | EPO | MRP-3 | TEM8 |
| CA6 | F3 | MyoD-1 | TSGA10 |
| CAIX | FAR | NCAM | TSSK6 |
| CEA | FBP | nectin 4 | thyroglobulin |
| CEACAM5 | FTHL 17 | Nestin | transferrin receptor |
| CEACAM6 | fetal AchR | NEP | TACSTD2 (TROP2) |
| Cav-1 | FAP | NY-ESO-1 | TMEM97 |
| CD10 | FGFR3 | hHLA-A | TRP-2 |
| CD117 | FR-a | H60 | TULP2 |
| CD123 | Fra-1/Fosl 1 | OLIG2 | TROP2 |
| CD133 | GAGE1 | 5T4 | tyrosinase |
| CD138 | GD2 | p53 | TRP1 |
| CD15 | GD3 | P-Cadherin | UPAR |
| CD171 | Glil | PB | VEGF |
| CD19 | GP100 | P-glycoprotein | VEGF receptors |
| CD20 | GPA33 | PMCT (SLC13A5) | VEGRR2 |
| CD21 | | PRAME | BRAF |
| CD22 | Glypican-3 | PROXl | WT-1 |
| CD30 | HIV gp120 | PSA | XAGE2 |
| CD33 | HLA-A | PSCA | ZNF165 |
| CD37 | HLA-A2 | PSMA | αᵥβ₆ integrin |
| CD38 | HLA-AI | PSC1 | β-catenin |
| CD44v6 | HLA-B | PVRL4 | cathepsin B |
| CD44v7/8 | HLA-C | Ras | CSAG2 |
| CD74 | HMW-MAA | ROR1 | CTAG |
| Cd79b | Her2/Neu | SART2 | EGFR |
| CD124 (IL-4R) | Her3 | SART3 | EGP40 |
| CDH3 | u70/80 | oncofetal variants | EZH2 |
| | | of fibronectin | |
| Ki-67 | LICAM | tenascin | HIV sp120 |
| CSPG4 | ULBP1 | LICAM | kappa light chain |
| CALLA | ULBP2 | Rae-1α | LDHC |
| CSAG2 | ULBP3 | | TRP-1 |
| COX-2 | ULBP6 | Rae-1β | Fas-L |
| Lambda | MICA | Rae-1δ | DLL3 |
| LAYN | MICB | Rae-1γ | MAGEA12 |
| LeuM-1 | Her3 | PDGF | MAGEC2 |
| KDR | EGF | SSX | BAGE |
| CD47 | SIRP1α | SSX2 | BAGE1 |
| | Cyclin A | KKLC1 | GAGE |
| | KMHN1 | SAGE | XAGE |
| | | SPA17 | XAGE1B |

In some embodiments, the bipartite polypeptide specifically binds to a combination of two antigens that are marked as “x” according to Table 5 below. Those “x” marked combinations in Table 5 that have the same antigens indicate that the bipartite polypeptide specifically binds to two different epitopes of the same antigen. In some embodiments, the bipartite polypeptide is a BiTE.

TABLE 5
Combination of two antigens for bipartite polypeptide binding

| | CD3 | CD3γ | CD3δ | CD3ε | CD3ξ | CD2 | CD4 | CD5 |
|---|---|---|---|---|---|---|---|---|
| 8H9 | x | x | x | x | x | x | x | x |
| GnT-V,β1,6- | x | x | x | x | x | x | x | x |
| N | | | | | | | | |
| AFP | x | x | x | x | x | x | x | x |
| ART1 | x | x | x | x | x | x | x | x |
| ART4 | x | x | x | x | x | x | x | x |
| ABCG2 | x | x | x | x | x | x | x | x |
| B7-H3 | x | x | x | x | x | x | x | x |
| B7-H4 | x | x | x | x | x | x | x | x |
| B7-H6 | x | x | x | x | x | x | x | x |
| BCMA | x | x | x | x | x | x | x | x |
| B-cyclin | x | x | x | x | x | x | x | x |
| BMI1 | x | x | x | x | x | x | x | x |
| CA-125 | x | x | x | x | x | x | x | x |
| cadherin | x | x | x | x | x | x | x | x |
| CABYR | x | x | x | x | x | x | x | x |
| CTAG2 | x | x | x | x | x | x | x | x |
| CA6 | x | x | x | x | x | x | x | x |
| CAIX | x | x | x | x | x | x | x | x |
| CEA | x | x | x | x | x | x | x | x |
| CEACAM5 | x | x | x | x | x | x | x | x |
| CEACAM6 | x | x | x | x | x | x | x | x |
| Cav-1 | x | x | x | x | x | x | x | x |
| CD10 | x | x | x | x | x | x | x | x |
| CD117 | x | x | x | x | x | x | x | x |
| CD123 | x | x | x | x | x | x | x | x |
| CD133 | x | x | x | x | x | x | x | x |
| CD138 | x | x | x | x | x | x | x | x |
| CD15 | x | x | x | x | x | x | x | x |
| CD171 | x | x | x | x | x | x | x | x |
| CD19 | x | x | x | x | x | x | x | x |
| CD20 | x | x | x | x | x | x | x | x |
| CD21 | x | x | x | x | x | x | x | x |
| CD22 | x | x | x | x | x | x | x | x |
| CD30 | x | x | x | x | x | x | x | x |
| CD33 | x | x | x | x | x | x | x | x |
| CD37 | x | x | x | x | x | x | x | x |
| CD38 | x | x | x | x | x | x | x | x |
| CD44v6 | x | x | x | x | x | x | x | x |
| CD44v7/8 | x | x | x | x | x | x | x | x |
| CD74 | x | x | x | x | x | x | x | x |
| Cd79b | x | x | x | x | x | x | x | x |
| CD124 (IL- | x | x | x | x | x | x | x | x |
| 4R) | | | | | | | | |
| CDH3 | x | x | x | x | x | x | x | x |
| Ki-67 | x | x | x | x | x | x | x | x |
| CSPG4 | x | x | x | x | x | x | x | x |
| CALLA | x | x | x | x | x | x | x | x |
| CSAG2 | x | x | x | x | x | x | x | x |
| COX-2 | x | x | x | x | x | x | x | x |
| Lambda | x | x | x | x | x | x | x | x |
| LAYN | x | x | x | x | x | x | x | x |
| LeuM-1 | x | x | x | x | x | x | x | x |
| KDR | x | x | x | x | x | x | x | x |
| CD47 | x | x | x | x | x | x | x | x |
| CRISP3 | x | x | x | x | x | x | x | x |
| DC-SIGN | x | x | x | x | x | x | x | x |
| DHFR | x | x | x | x | x | x | x | x |
| EGP40 | x | x | x | x | x | x | x | x |
| EZH2 | x | x | x | x | x | x | x | x |
| EpCAM | x | x | x | x | x | x | x | x |
| EphA2 | x | x | x | x | x | x | x | x |
| EphA2/Eck | x | x | x | x | x | x | x | x |
| EGFRvIII | x | x | x | x | x | x | x | x |
| E-cadherin | x | x | x | x | x | x | x | x |
| EGP2 | x | x | x | x | x | x | x | x |
| ETA | x | x | x | x | x | x | x | x |
| ERBB3 | x | x | x | x | x | x | x | x |
| ERBB3/4 | x | x | x | x | x | x | x | x |
| ERBB4 | x | x | x | x | x | x | x | x |
| EPO | x | x | x | x | x | x | x | x |
| F3 | x | x | x | x | x | x | x | x |
| FAR | x | x | x | x | x | x | x | x |
| FBP | x | x | x | x | x | x | x | x |
| FTHL17 | x | x | x | x | x | x | x | x |
| fetal AchR | x | x | x | x | x | x | x | x |
| FAP | x | x | x | x | x | x | x | x |
| FGFR3 | x | x | x | x | x | x | x | x |
| FR-a | x | x | x | x | x | x | x | x |
| Fra-1/Fosl 1 | x | x | x | x | x | x | x | x |
| GAGE1 | x | x | x | x | x | x | x | x |
| GD2 | x | x | x | x | x | x | x | x |
| GD3 | x | x | x | x | x | x | x | x |
| Glil | x | x | x | x | x | x | x | x |
| GP100 | x | x | x | x | x | x | x | x |
| GPA33 | x | x | x | x | x | x | x | x |
| Glypican-3 | x | x | x | x | x | x | x | x |
| HIV gp120 | x | x | x | x | x | x | x | x |
| HLA-A | x | x | x | x | x | x | x | x |
| HLA-A2 | x | x | x | x | x | x | x | x |
| HLA-AI | x | x | x | x | x | x | x | x |
| HLA-B | x | x | x | x | x | x | x | x |
| HLA-C | x | x | x | x | x | x | x | x |
| HMW-MAA | x | x | x | x | x | x | x | x |
| Her2/Neu | x | x | x | x | x | x | x | x |
| Her3 | x | x | x | x | x | x | x | x |
| u70/80 | x | x | x | x | x | x | x | x |
| LICAM | x | x | x | x | x | x | x | x |
| ULBP1 | x | x | x | x | x | x | x | x |
| ULBP2 | x | x | x | x | x | x | x | x |
| ULBP3 | x | x | x | x | x | x | x | x |
| ULBP6 | x | x | x | x | x | x | x | x |
| MICA | x | x | x | x | x | x | x | x |
| MICB | x | x | x | x | x | x | x | x |
| Her3 | x | x | x | x | x | x | x | x |
| EGF | x | x | x | x | x | x | x | x |
| SIRP1α | x | x | x | x | x | x | x | x |
| Lewis-Y | x | x | x | x | x | x | x | x |
| LIV-1 | x | x | x | x | x | x | x | x |
| (SLC39A6) | | | | | | | | |
| Livin | x | x | x | x | x | x | x | x |
| LAMP1 | x | x | x | x | x | x | x | x |
| MAGEA3 | x | x | x | x | x | x | x | x |
| MAGEA4 | x | x | x | x | x | x | x | x |
| MAGEB6 | x | x | x | x | x | x | x | x |
| MAGEA1 | x | x | x | x | x | x | x | x |
| MART-1 | x | x | x | x | x | x | x | x |
| MCSP | x | x | x | x | x | x | x | x |
| MME | x | x | x | x | x | x | x | x |
| mesothelin | x | x | x | x | x | x | x | x |
| (MSLN) | | | | | | | | |
| MAPK1 | x | x | x | x | x | x | x | x |
| MUC16 | x | x | x | x | x | x | x | x |
| MUC1 | x | x | x | x | x | x | x | x |
| MRP-3 | x | x | x | x | x | x | x | x |
| MyoD-1 | x | x | x | x | x | x | x | x |
| NCAM | x | x | x | x | x | x | x | x |
| nectin 4 | x | x | x | x | x | x | x | x |
| Nestin | x | x | x | x | x | x | x | x |
| NEP | x | x | x | x | x | x | x | x |
| NY-ESO-1 | x | x | x | x | x | x | x | x |
| hHLA-A | x | x | x | x | x | x | x | x |
| H60 | x | x | x | x | x | x | x | x |
| OLIG2 | x | x | x | x | x | x | x | x |
| 5T4 | x | x | x | x | x | x | x | x |
| p53 | x | x | x | x | x | x | x | x |
| P-Cadherin | x | x | x | x | x | x | x | x |
| PB | x | x | x | x | x | x | x | x |
| P- | x | x | x | x | x | x | x | x |
| glycoprotein | | | | | | | | |
| PMCT | x | x | x | x | x | x | x | x |
| (SLC13A5) | | | | | | | | |
| PRAME | x | x | x | x | x | x | x | x |
| PROX1 | x | x | x | x | x | x | x | x |
| PSA | x | x | x | x | x | x | x | x |
| PSCA | x | x | x | x | x | x | x | x |
| PSMA | x | x | x | x | x | x | x | x |
| PSC1 | x | x | x | x | x | x | x | x |
| PVRL4 | x | x | x | x | x | x | x | x |
| Ras | x | x | x | x | x | x | x | x |
| ROR1 | x | x | x | x | x | x | x | x |
| SART2 | x | x | x | x | x | x | x | x |
| SART3 | x | x | x | x | x | x | x | x |
| oncofetal | x | x | x | x | x | x | x | x |
| variants of | | | | | | | | |
| fibronectin | | | | | | | | |
| tenascin | x | x | x | x | x | x | x | x |
| LICAM | x | x | x | x | x | x | x | x |
| Rae-1α | x | x | x | x | x | x | x | x |
| Rae-1β | x | x | x | x | x | x | x | x |
| Rae-1δ | x | x | x | x | x | x | x | x |
| Rae-1γ | x | x | x | x | x | x | x | x |
| PDGF | x | x | x | x | x | x | x | x |
| Fas | x | x | x | x | x | x | x | x |
| SOX2 | x | x | x | x | x | x | x | x |
| STEAP1 | x | x | x | x | x | x | x | x |
| SLITRK6 | x | x | x | x | x | x | x | x |
| NaPi2a | x | x | x | x | x | x | x | x |
| SOX1 | x | x | x | x | x | x | x | x |
| SOX11 | x | x | x | x | x | x | x | x |
| SPANXA1 | x | x | x | x | x | x | x | x |
| SART-1 | x | x | x | x | x | x | x | x |
| SSX4 | x | x | x | x | x | x | x | x |
| SSX5 | x | x | x | x | x | x | x | x |
| Survivin | x | x | x | x | x | x | x | x |
| SSX2 | x | x | x | x | x | x | x | x |
| TAG72 | x | x | x | x | x | x | x | x |
| TEM1 | x | x | x | x | x | x | x | x |
| TEM8 | x | x | x | x | x | x | x | x |
| TSGA10 | x | x | x | x | x | x | x | x |
| TSSK6 | x | x | x | x | x | x | x | x |
| thyroglobulin | x | x | x | x | x | x | x | x |
| transferrin | x | x | x | x | x | x | x | x |
| receptor | | | | | | | | |
| TACSTD2 | x | x | x | x | x | x | x | x |
| (TROP2) | | | | | | | | |
| TMEM97 | x | x | x | x | x | x | x | x |
| TRP-2 | x | x | x | x | x | x | x | x |
| TULP2 | x | x | x | x | x | x | x | x |
| TROP2 | x | x | x | x | x | x | x | x |
| tyrosinase | x | x | x | x | x | x | x | x |
| TRP1 | x | x | x | x | x | x | x | x |
| UPAR | x | x | x | x | x | x | x | x |
| VEGF | x | x | x | x | x | x | x | x |
| VEGF | x | x | x | x | x | x | x | x |
| receptors | | | | | | | | |
| VEGRR2 | x | x | x | x | x | x | x | x |
| BRAF | x | x | x | x | x | x | x | x |
| WT-1 | x | x | x | x | x | x | x | x |
| XAGE2 | x | x | x | x | x | x | x | x |
| ZNF165 | x | x | x | x | x | x | x | x |
| αvβ6 integrin | x | x | x | x | x | x | x | x |
| β-catenin | x | x | x | x | x | x | x | x |
| cathepsin B | x | x | x | x | x | x | x | x |
| CSAG2 | x | x | x | x | x | x | x | x |
| CTAG | x | x | x | x | x | x | x | x |
| EGFR | x | x | x | x | x | x | x | x |
| EGP40 | x | x | x | x | x | x | x | x |
| EZH2 | x | x | x | x | x | x | x | x |
| HIV sp120 | x | x | x | x | x | x | x | x |
| kappa light | x | x | x | x | x | x | x | x |
| chain | | | | | | | | |
| LDHC | x | x | x | x | x | x | x | x |
| TRP-1 | x | x | x | x | x | x | x | x |
| Fas-L | x | x | x | x | x | x | x | x |
| DLL3 | x | x | x | x | x | x | x | x |
| MAGEA12 | x | x | x | x | x | x | x | x |
| MAGEC2 | x | x | x | x | x | x | x | x |
| BAGE | x | x | x | x | x | x | x | x |
| BAGE1 | x | x | x | x | x | x | x | x |
| GAGE | x | x | x | x | x | x | x | x |
| XAGE | x | x | x | x | x | x | x | x |
| XAGE1B | x | x | x | x | x | x | x | x |
| SSX | x | x | x | x | x | x | x | x |
| SSX2 | x | x | x | x | x | x | x | x |
| KKLC1 | x | x | x | x | x | x | x | x |
| SAGE | x | x | x | x | x | x | x | x |
| SPA17 | x | x | x | x | x | x | x | x |
| Cyclin A | x | x | x | x | x | x | x | x |
| KMHN1 | x | x | x | x | x | x | x | x |
| | CD6 | CD7 | CD8 | CD16 | CD25 | CD27 | CD28 | CD30 |
| 8H9 | x | x | x | x | x | x | x | x |
| GnT-V,β1,6- | x | x | x | x | x | x | x | x |
| N | | | | | | | | |
| AFP | x | x | x | x | x | x | x | x |
| ART1 | x | x | x | x | x | x | x | x |
| ART4 | x | x | x | x | x | x | x | x |
| ABCG2 | x | x | x | x | x | x | x | x |
| B7-H3 | x | x | x | x | x | x | x | x |
| B7-H4 | x | x | x | x | x | x | x | x |
| B7-H6 | x | x | x | x | x | x | x | x |
| BCMA | x | x | x | x | x | x | x | x |
| B-cyclin | x | x | x | x | x | x | x | x |
| BMI1 | x | x | x | x | x | x | x | x |
| CA-125 | x | x | x | x | x | x | x | x |
| cadherin | x | x | x | x | x | x | x | x |
| CABYR | x | x | x | x | x | x | x | x |
| CTAG2 | x | x | x | x | x | x | x | x |
| CA6 | x | x | x | x | x | x | x | x |
| CAIX | x | x | x | x | x | x | x | x |
| CEA | x | x | x | x | x | x | x | x |
| CEACAM5 | x | x | x | x | x | x | x | x |
| CEACAM6 | x | x | x | x | x | x | x | x |
| Cav-1 | x | x | x | x | x | x | x | x |
| CD10 | x | x | x | x | x | x | x | x |
| CD117 | x | x | x | x | x | x | x | x |
| CD123 | x | x | x | x | x | x | x | x |
| CD133 | x | x | x | x | x | x | x | x |
| CD138 | x | x | x | x | x | x | x | x |
| CD15 | x | x | x | x | x | x | x | x |
| CD171 | x | x | x | x | x | x | x | x |
| CD19 | x | x | x | x | x | x | x | x |
| CD20 | x | x | x | x | x | x | x | x |
| CD21 | x | x | x | x | x | x | x | x |
| CD22 | x | x | x | x | x | x | x | x |
| CD30 | x | x | x | x | x | x | x | x |
| CD33 | x | x | x | x | x | x | x | x |
| CD37 | x | x | x | x | x | x | x | x |
| CD38 | x | x | x | x | x | x | x | x |
| CD44v6 | x | x | x | x | x | x | x | x |
| CD44v7/8 | x | x | x | x | x | x | x | x |
| CD74 | x | x | x | x | x | x | x | x |
| Cd79b | x | x | x | x | x | x | x | x |
| CD124 (IL- | x | x | x | x | x | x | x | x |
| 4R) | | | | | | | | |
| CDH3 | x | x | x | x | x | x | x | x |
| Ki-67 | x | x | x | x | x | x | x | x |
| CSPG4 | x | x | x | x | x | x | x | x |
| CALLA | x | x | x | x | x | x | x | x |
| CSAG2 | x | x | x | x | x | x | x | x |
| COX-2 | x | x | x | x | x | x | x | x |
| Lambda | x | x | x | x | x | x | x | x |
| LAYN | x | x | x | x | x | x | x | x |
| LeuM-1 | x | x | x | x | x | x | x | x |
| KDR | x | x | x | x | x | x | x | x |
| CD47 | x | x | x | x | x | x | x | x |
| CRISP3 | x | x | x | x | x | x | x | x |
| DC-SIGN | x | x | x | x | x | x | x | x |
| DHFR | x | x | x | x | x | x | x | x |
| EGP40 | x | x | x | x | x | x | x | x |
| EZH2 | x | x | x | x | x | x | x | x |
| EpCAM | x | x | x | x | x | x | x | x |
| EphA2 | x | x | x | x | x | x | x | x |
| EphA2/Eck | x | x | x | x | x | x | x | x |
| EGFRvIII | x | x | x | x | x | x | x | x |
| E-cadherin | x | x | x | x | x | x | x | x |
| EGP2 | x | x | x | x | x | x | x | x |
| ETA | x | x | x | x | x | x | x | x |
| ERBB3 | x | x | x | x | x | x | x | x |
| ERBB3/4 | x | x | x | x | x | x | x | x |
| ERBB4 | x | x | x | x | x | x | x | x |
| EPO | x | x | x | x | x | x | x | x |
| F3 | x | x | x | x | x | x | x | x |
| FAR | x | x | x | x | x | x | x | x |
| FBP | x | x | x | x | x | x | x | x |
| FTHL17 | x | x | x | x | x | x | x | x |
| fetal AchR | x | x | x | x | x | x | x | x |
| FAP | x | x | x | x | x | x | x | x |
| FGFR3 | x | x | x | x | x | x | x | x |
| FR-a | x | x | x | x | x | x | x | x |
| Fra-1/Fosl 1 | x | x | x | x | x | x | x | x |
| GAGE1 | x | x | x | x | x | x | x | x |
| GD2 | x | x | x | x | x | x | x | x |
| GD3 | x | x | x | x | x | x | x | x |
| Glil | x | x | x | x | x | x | x | x |
| GP100 | x | x | x | x | x | x | x | x |
| GPA33 | x | x | x | x | x | x | x | x |
| Glypican-3 | x | x | x | x | x | x | x | x |
| HIV gp120 | x | x | x | x | x | x | x | x |
| HLA-A | x | x | x | x | x | x | x | x |
| HLA-A2 | x | x | x | x | x | x | x | x |
| HLA-AI | x | x | x | x | x | x | x | x |
| HLA-B | x | x | x | x | x | x | x | x |
| HLA-C | x | x | x | x | x | x | x | x |
| HMW-MAA | x | x | x | x | x | x | x | x |
| Her2/Neu | x | x | x | x | x | x | x | x |
| Her3 | x | x | x | x | x | x | x | x |
| u70/80 | x | x | x | x | x | x | x | x |
| LICAM | x | x | x | x | x | x | x | x |
| ULBP1 | x | x | x | x | x | x | x | x |
| ULBP2 | x | x | x | x | x | x | x | x |
| ULBP3 | x | x | x | x | x | x | x | x |
| ULBP6 | x | x | x | x | x | x | x | x |
| MICA | x | x | x | x | x | x | x | x |
| MICB | x | x | x | x | x | x | x | x |
| Her3 | x | x | x | x | x | x | x | x |
| EGF | x | x | x | x | x | x | x | x |
| SIRP1α | x | x | x | x | x | x | x | x |
| Lewis-Y | x | x | x | x | x | x | x | x |
| LIV-1 | x | x | x | x | x | x | x | x |
| (SLC39A6) | | | | | | | | |
| Livin | x | x | x | x | x | x | x | x |
| LAMP1 | x | x | x | x | x | x | x | x |
| MAGEA3 | x | x | x | x | x | x | x | x |
| MAGEA4 | x | x | x | x | x | x | x | x |
| MAGEB6 | x | x | x | x | x | x | x | x |
| MAGEA1 | x | x | x | x | x | x | x | x |
| MART-1 | x | x | x | x | x | x | x | x |
| MCSP | x | x | x | x | x | x | x | x |
| MME | x | x | x | x | x | x | x | x |
| mesothelin | x | x | x | x | x | x | x | x |
| (MSLN) | | | | | | | | |
| MAPK1 | x | x | x | x | x | x | x | x |
| MUC16 | x | x | x | x | x | x | x | x |
| MUC1 | x | x | x | x | x | x | x | x |
| MRP-3 | x | x | x | x | x | x | x | x |
| MyoD-1 | x | x | x | x | x | x | x | x |
| NCAM | x | x | x | x | x | x | x | x |
| nectin 4 | x | x | x | x | x | x | x | x |
| Nestin | x | x | x | x | x | x | x | x |
| NEP | x | x | x | x | x | x | x | x |
| NY-ESO-1 | x | x | x | x | x | x | x | x |
| hHLA-A | x | x | x | x | x | x | x | x |
| H60 | x | x | x | x | x | x | x | x |
| OLIG2 | x | x | x | x | x | x | x | x |
| 5T4 | x | x | x | x | x | x | x | x |
| p53 | x | x | x | x | x | x | x | x |
| P-Cadherin | x | x | x | x | x | x | x | x |
| PB | x | x | x | x | x | x | x | x |
| P- | x | x | x | x | x | x | x | x |
| glycoprotein | | | | | | | | |
| PMCT | x | x | x | x | x | x | x | x |
| (SLC13A5) | | | | | | | | |
| PRAME | x | x | x | x | x | x | x | x |
| PROX1 | x | x | x | x | x | x | x | x |
| PSA | x | x | x | x | x | x | x | x |
| PSCA | x | x | x | x | x | x | x | x |
| PSMA | x | x | x | x | x | x | x | x |
| PSC1 | x | x | x | x | x | x | x | x |
| PVRL4 | x | x | x | x | x | x | x | x |
| Ras | x | x | x | x | x | x | x | x |
| ROR1 | x | x | x | x | x | x | x | x |
| SART2 | x | x | x | x | x | x | x | x |
| SART3 | x | x | x | x | x | x | x | x |
| oncofetal | x | x | x | x | x | x | x | x |
| variants of | | | | | | | | |
| fibronectin | | | | | | | | |
| tenascin | x | x | x | x | x | x | x | x |
| LICAM | x | x | x | x | x | x | x | x |
| Rae-1α | x | x | x | x | x | x | x | x |
| Rae-1β | x | x | x | x | x | x | x | x |
| Rae-1δ | x | x | x | x | x | x | x | x |
| Rae-1γ | x | x | x | x | x | x | x | x |
| PDGF | x | x | x | x | x | x | x | x |
| Fas | x | x | x | x | x | x | x | x |
| SOX2 | x | x | x | x | x | x | x | x |
| STEAP1 | x | x | x | x | x | x | x | x |
| SLITRK6 | x | x | x | x | x | x | x | x |
| NaPi2a | x | x | x | x | x | x | x | x |
| SOX1 | x | x | x | x | x | x | x | x |
| SOX11 | x | x | x | x | x | x | x | x |
| SPANXA1 | x | x | x | x | x | x | x | x |
| SART-1 | x | x | x | x | x | x | x | x |
| SSX4 | x | x | x | x | x | x | x | x |
| SSX5 | x | x | x | x | x | x | x | x |
| Survivin | x | x | x | x | x | x | x | x |
| SSX2 | x | x | x | x | x | x | x | x |
| TAG72 | x | x | x | x | x | x | x | x |
| TEM1 | x | x | x | x | x | x | x | x |
| TEM8 | x | x | x | x | x | x | x | x |
| TSGA10 | x | x | x | x | x | x | x | x |
| TSSK6 | x | x | x | x | x | x | x | x |
| thyroglobulin | x | x | x | x | x | x | x | x |
| transferrin | x | x | x | x | x | x | x | x |
| receptor | | | | | | | | |
| TACSTD2 | x | x | x | x | x | x | x | x |
| (TROP2) | | | | | | | | |
| TMEM97 | x | x | x | x | x | x | x | x |
| TRP-2 | x | x | x | x | x | x | x | x |
| TULP2 | x | x | x | x | x | x | x | x |
| TROP2 | x | x | x | x | x | x | x | x |
| tyrosinase | x | x | x | x | x | x | x | x |
| TRP1 | x | x | x | x | x | x | x | x |
| UPAR | x | x | x | x | x | x | x | x |
| VEGF | x | x | x | x | x | x | x | x |
| VEGF | x | x | x | x | x | x | x | x |
| receptors | | | | | | | | |
| VEGRR2 | x | x | x | x | x | x | x | x |
| BRAF | x | x | x | x | x | x | x | x |
| WT-1 | x | x | x | x | x | x | x | x |
| XAGE2 | x | x | x | x | x | x | x | x |
| ZNF165 | x | x | x | x | x | x | x | x |
| αvβ6 integrin | x | x | x | x | x | x | x | x |
| β-catenin | x | x | x | x | x | x | x | x |
| cathepsin B | x | x | x | x | x | x | x | x |
| CSAG2 | x | x | x | x | x | x | x | x |
| CTAG | x | x | x | x | x | x | x | x |
| EGFR | x | x | x | x | x | x | x | x |
| EGP40 | x | x | x | x | x | x | x | x |
| EZH2 | x | x | x | x | x | x | x | x |
| HIV sp120 | x | x | x | x | x | x | x | x |
| kappa light | x | x | x | x | x | x | x | x |
| chain | | | | | | | | |
| LDHC | x | x | x | x | x | x | x | x |
| TRP-1 | x | x | x | x | x | x | x | x |
| Fas-L | x | x | x | x | x | x | x | x |
| DLL3 | x | x | x | x | x | x | x | x |
| MAGEA12 | x | x | x | x | x | x | x | x |
| MAGEC2 | x | x | x | x | x | x | x | x |
| BAGE | x | x | x | x | x | x | x | x |
| BAGE1 | x | x | x | x | x | x | x | x |
| GAGE | x | x | x | x | x | x | x | x |
| XAGE | x | x | x | x | x | x | x | x |
| XAGE1B | x | x | x | x | x | x | x | x |
| SSX | x | x | x | x | x | x | x | x |
| SSX2 | x | x | x | x | x | x | x | x |
| KKLC1 | x | x | x | x | x | x | x | x |
| SAGE | x | x | x | x | x | x | x | x |
| SPA17 | x | x | x | x | x | x | x | x |
| Cyclin A | x | x | x | x | x | x | x | x |
| KMHN1 | x | x | x | x | x | x | x | x |
| | CD38 | CD40 | CD57 | CD69 | CD70 | CD73 | CD81 | CD82 |
| 8H9 | x | x | x | x | x | x | x | x |
| GnT-V,β1,6- | x | x | x | x | x | x | x | x |
| N | | | | | | | | |
| AFP | x | x | x | x | x | x | x | x |
| ART1 | x | x | x | x | x | x | x | x |
| ART4 | x | x | x | x | x | x | x | x |
| ABCG2 | x | x | x | x | x | x | x | x |
| B7-H3 | x | x | x | x | x | x | x | x |
| B7-H4 | x | x | x | x | x | x | x | x |
| B7-H6 | x | x | x | x | x | x | x | x |
| BCMA | x | x | x | x | x | x | x | x |
| B-cyclin | x | x | x | x | x | x | x | x |
| BMI1 | x | x | x | x | x | x | x | x |
| CA-125 | x | x | x | x | x | x | x | x |
| cadherin | x | x | x | x | x | x | x | x |
| CABYR | x | x | x | x | x | x | x | x |
| CTAG2 | x | x | x | x | x | x | x | x |
| CA6 | x | x | x | x | x | x | x | x |
| CAIX | x | x | x | x | x | x | x | x |
| CEA | x | x | x | x | x | x | x | x |
| CEACAM5 | x | x | x | x | x | x | x | x |
| CEACAM6 | x | x | x | x | x | x | x | x |
| Cav-1 | x | x | x | x | x | x | x | x |
| CD10 | x | x | x | x | x | x | x | x |
| CD117 | x | x | x | x | x | x | x | x |
| CD123 | x | x | x | x | x | x | x | x |
| CD133 | x | x | x | x | x | x | x | x |
| CD138 | x | x | x | x | x | x | x | x |
| CD15 | x | x | x | x | x | x | x | x |
| CD171 | x | x | x | x | x | x | x | x |
| CD19 | x | x | x | x | x | x | x | x |
| CD20 | x | x | x | x | x | x | x | x |
| CD21 | x | x | x | x | x | x | x | x |
| CD22 | x | x | x | x | x | x | x | x |
| CD30 | x | x | x | x | x | x | x | x |
| CD33 | x | x | x | x | x | x | x | x |
| CD37 | x | x | x | x | x | x | x | x |
| CD38 | x | x | x | x | x | x | x | x |
| CD44v6 | x | x | x | x | x | x | x | x |
| CD44v7/8 | x | x | x | x | x | x | x | x |
| CD74 | x | x | x | x | x | x | x | x |
| Cd79b | x | x | x | x | x | x | x | x |
| CD124 (IL- | x | x | x | x | x | x | x | x |
| 4R) | | | | | | | | |
| CDH3 | x | x | x | x | x | x | x | x |
| Ki-67 | x | x | x | x | x | x | x | x |
| CSPG4 | x | x | x | x | x | x | x | x |
| CALLA | x | x | x | x | x | x | x | x |
| CSAG2 | x | x | x | x | x | x | x | x |
| COX-2 | x | x | x | x | x | x | x | x |
| Lambda | x | x | x | x | x | x | x | x |
| LAYN | x | x | x | x | x | x | x | x |
| LeuM-1 | x | x | x | x | x | x | x | x |
| KDR | x | x | x | x | x | x | x | x |
| CD47 | x | x | x | x | x | x | x | x |
| CRISP3 | x | x | x | x | x | x | x | x |
| DC-SIGN | x | x | x | x | x | x | x | x |
| DHFR | x | x | x | x | x | x | x | x |
| EGP40 | x | x | x | x | x | x | x | x |
| EZH2 | x | x | x | x | x | x | x | x |
| EpCAM | x | x | x | x | x | x | x | x |
| EphA2 | x | x | x | x | x | x | x | x |
| EphA2/Eck | x | x | x | x | x | x | x | x |
| EGFRvIII | x | x | x | x | x | x | x | x |
| E-cadherin | x | x | x | x | x | x | x | x |
| EGP2 | x | x | x | x | x | x | x | x |
| ETA | x | x | x | x | x | x | x | x |
| ERBB3 | x | x | x | x | x | x | x | x |
| ERBB3/4 | x | x | x | x | x | x | x | x |
| ERBB4 | x | x | x | x | x | x | x | x |
| EPO | x | x | x | x | x | x | x | x |
| F3 | x | x | x | x | x | x | x | x |
| FAR | x | x | x | x | x | x | x | x |
| FBP | x | x | x | x | x | x | x | x |
| FTHL17 | x | x | x | x | x | x | x | x |
| fetal AchR | x | x | x | x | x | x | x | x |
| FAP | x | x | x | x | x | x | x | x |
| FGFR3 | x | x | x | x | x | x | x | x |
| FR-a | x | x | x | x | x | x | x | x |
| Fra-1/Fosl 1 | x | x | x | x | x | x | x | x |
| GAGE1 | x | x | x | x | x | x | x | x |
| GD2 | x | x | x | x | x | x | x | x |
| GD3 | x | x | x | x | x | x | x | x |
| Glil | x | x | x | x | x | x | x | x |
| GP100 | x | x | x | x | x | x | x | x |
| GPA33 | x | x | x | x | x | x | x | x |
| Glypican-3 | x | x | x | x | x | x | x | x |
| HIV gp120 | x | x | x | x | x | x | x | x |
| HLA-A | x | x | x | x | x | x | x | x |
| HLA-A2 | x | x | x | x | x | x | x | x |
| HLA-AI | x | x | x | x | x | x | x | x |
| HLA-B | x | x | x | x | x | x | x | x |
| HLA-C | x | x | x | x | x | x | x | x |
| HMW-MAA | x | x | x | x | x | x | x | x |
| Her2/Neu | x | x | x | x | x | x | x | x |
| Her3 | x | x | x | x | x | x | x | x |
| u70/80 | x | x | x | x | x | x | x | x |
| LICAM | x | x | x | x | x | x | x | x |
| ULBP1 | x | x | x | x | x | x | x | x |
| ULBP2 | x | x | x | x | x | x | x | x |
| ULBP3 | x | x | x | x | x | x | x | x |
| ULBP6 | x | x | x | x | x | x | x | x |
| MICA | x | x | x | x | x | x | x | x |
| MICB | x | x | x | x | x | x | x | x |
| Her3 | x | x | x | x | x | x | x | x |
| EGF | x | x | x | x | x | x | x | x |
| SIRP1α | x | x | x | x | x | x | x | x |
| Lewis-Y | x | x | x | x | x | x | x | x |
| LIV-1 | x | x | x | x | x | x | x | x |
| (SLC39A6) | | | | | | | | |
| Livin | x | x | x | x | x | x | x | x |
| LAMP1 | x | x | x | x | x | x | x | x |
| MAGEA3 | x | x | x | x | x | x | x | x |
| MAGEA4 | x | x | x | x | x | x | x | x |
| MAGEB6 | x | x | x | x | x | x | x | x |
| MAGEA1 | x | x | x | x | x | x | x | x |
| MART-1 | x | x | x | x | x | x | x | x |
| MCSP | x | x | x | x | x | x | x | x |
| MME | x | x | x | x | x | x | x | x |
| mesothelin | x | x | x | x | x | x | x | x |
| (MSLN) | | | | | | | | |
| MAPK1 | x | x | x | x | x | x | x | x |
| MUC16 | x | x | x | x | x | x | x | x |
| MUC1 | x | x | x | x | x | x | x | x |
| MRP-3 | x | x | x | x | x | x | x | x |
| MyoD-1 | x | x | x | x | x | x | x | x |
| NCAM | x | x | x | x | x | x | x | x |
| nectin 4 | x | x | x | x | x | x | x | x |
| Nestin | x | x | x | x | x | x | x | x |
| NEP | x | x | x | x | x | x | x | x |
| NY-ESO-1 | x | x | x | x | x | x | x | x |
| hHLA-A | x | x | x | x | x | x | x | x |
| H60 | x | x | x | x | x | x | x | x |
| OLIG2 | x | x | x | x | x | x | x | x |
| 5T4 | x | x | x | x | x | x | x | x |
| p53 | x | x | x | x | x | x | x | x |
| P-Cadherin | x | x | x | x | x | x | x | x |
| PB | x | x | x | x | x | x | x | x |
| P- | x | x | x | x | x | x | x | x |
| glycoprotein | | | | | | | | |
| PMCT | x | x | x | x | x | x | x | x |
| (SLC13A5) | | | | | | | | |
| PRAME | x | x | x | x | x | x | x | x |
| PROX1 | x | x | x | x | x | x | x | x |
| PSA | x | x | x | x | x | x | x | x |
| PSCA | x | x | x | x | x | x | x | x |
| PSMA | x | x | x | x | x | x | x | x |
| PSC1 | x | x | x | x | x | x | x | x |
| PVRL4 | x | x | x | x | x | x | x | x |
| Ras | x | x | x | x | x | x | x | x |
| ROR1 | x | x | x | x | x | x | x | x |
| SART2 | x | x | x | x | x | x | x | x |
| SART3 | x | x | x | x | x | x | x | x |
| oncofetal | x | x | x | x | x | x | x | x |
| variants of | | | | | | | | |
| fibronectin | | | | | | | | |
| tenascin | x | x | x | x | x | x | x | x |
| LICAM | x | x | x | x | x | x | x | x |
| Rae-1α | x | x | x | x | x | x | x | x |
| Rae-1β | x | x | x | x | x | x | x | x |
| Rae-1δ | x | x | x | x | x | x | x | x |
| Rae-1γ | x | x | x | x | x | x | x | x |
| PDGF | x | x | x | x | x | x | x | x |
| Fas | x | x | x | x | x | x | x | x |
| SOX2 | x | x | x | x | x | x | x | x |
| STEAP1 | x | x | x | x | x | x | x | x |
| SLITRK6 | x | x | x | x | x | x | x | x |
| NaPi2a | x | x | x | x | x | x | x | x |
| SOX1 | x | x | x | x | x | x | x | x |
| SOX11 | x | x | x | x | x | x | x | x |
| SPANXA1 | x | x | x | x | x | x | x | x |
| SART-1 | x | x | x | x | x | x | x | x |
| SSX4 | x | x | x | x | x | x | x | x |
| SSX5 | x | x | x | x | x | x | x | x |
| Survivin | x | x | x | x | x | x | x | x |
| SSX2 | x | x | x | x | x | x | x | x |
| TAG72 | x | x | x | x | x | x | x | x |
| TEM1 | x | x | x | x | x | x | x | x |
| TEM8 | x | x | x | x | x | x | x | x |
| TSGA10 | x | x | x | x | x | x | x | x |
| TSSK6 | x | x | x | x | x | x | x | x |
| thyroglobulin | x | x | x | x | x | x | x | x |
| transferrin | x | x | x | x | x | x | x | x |
| receptor | | | | | | | | |
| TACSTD2 | x | x | x | x | x | x | x | x |
| (TROP2) | | | | | | | | |
| TMEM97 | x | x | x | x | x | x | x | x |
| TRP-2 | x | x | x | x | x | x | x | x |
| TULP2 | x | x | x | x | x | x | x | x |
| TROP2 | x | x | x | x | x | x | x | x |
| tyrosinase | x | x | x | x | x | x | x | x |
| TRP1 | x | x | x | x | x | x | x | x |
| UPAR | x | x | x | x | x | x | x | x |
| VEGF | x | x | x | x | x | x | x | x |
| VEGF | x | x | x | x | x | x | x | x |
| receptors | | | | | | | | |
| VEGRR2 | x | x | x | x | x | x | x | x |
| BRAF | x | x | x | x | x | x | x | x |
| WT-1 | x | x | x | x | x | x | x | x |
| XAGE2 | x | x | x | x | x | x | x | x |
| ZNF165 | x | x | x | x | x | x | x | x |
| αvβ6 integrin | x | x | x | x | x | x | x | x |
| β-catenin | x | x | x | x | x | x | x | x |
| cathepsin B | x | x | x | x | x | x | x | x |
| CSAG2 | x | x | x | x | x | x | x | x |
| CTAG | x | x | x | x | x | x | x | x |
| EGFR | x | x | x | x | x | x | x | x |
| EGP40 | x | x | x | x | x | x | x | x |
| EZH2 | x | x | x | x | x | x | x | x |
| HIV sp120 | x | x | x | x | x | x | x | x |
| kappa light | x | x | x | x | x | x | x | x |
| chain | | | | | | | | |
| LDHC | x | x | x | x | x | x | x | x |
| TRP-1 | x | x | x | x | x | x | x | x |
| Fas-L | x | x | x | x | x | x | x | x |
| DLL3 | x | x | x | x | x | x | x | x |
| MAGEA12 | x | x | x | x | x | x | x | x |
| MAGEC2 | x | x | x | x | x | x | x | x |
| BAGE | x | x | x | x | x | x | x | x |
| BAGE1 | x | x | x | x | x | x | x | x |
| GAGE | x | x | x | x | x | x | x | x |
| XAGE | x | x | x | x | x | x | x | x |
| XAGEIB | x | x | x | x | x | x | x | x |
| SSX | x | x | x | x | x | x | x | x |
| SSX2 | x | x | x | x | x | x | x | x |
| KKLC1 | x | x | x | x | x | x | x | x |
| SAGE | x | x | x | x | x | x | x | x |
| SPA17 | x | x | x | x | x | x | x | x |
| Cyclin A | x | x | x | x | x | x | x | x |
| KMHN1 | x | x | x | x | x | x | x | x |
| | | | | | | | CD94/ | |
| | | | | | | | NKG2 | |
| | | | | | | invariant | (e.g., | |
| | CD96 | CD134 | CD137 | CD152 | CD278 | TCR | NKG2D) | NKp30 |
| 8H9 | x | x | x | x | x | x | x | x |
| GnT-V,β1,6- | x | x | x | x | x | x | x | x |
| N | | | | | | | | |
| AFP | x | x | x | x | x | x | x | x |
| ART1 | x | x | x | x | x | x | x | x |
| ART4 | x | x | x | x | x | x | x | x |
| ABCG2 | x | x | x | x | x | x | x | x |
| B7-H3 | x | x | x | x | x | x | x | x |
| B7-H4 | x | x | x | x | x | x | x | x |
| B7-H6 | x | x | x | x | x | x | x | x |
| BCMA | x | x | x | x | x | x | x | x |
| B-cyclin | x | x | x | x | x | x | x | x |
| BMI1 | x | x | x | x | x | x | x | x |
| CA-125 | x | x | x | x | x | x | x | x |
| cadherin | x | x | x | x | x | x | x | x |
| CABYR | x | x | x | x | x | x | x | x |
| CTAG2 | x | x | x | x | x | x | x | x |
| CA6 | x | x | x | x | x | x | x | x |
| CAIX | x | x | x | x | x | x | x | x |
| CEA | x | x | x | x | x | x | x | x |
| CEACAM5 | x | x | x | x | x | x | x | x |
| CEACAM6 | x | x | x | x | x | x | x | x |
| Cav-1 | x | x | x | x | x | x | x | x |
| CD10 | x | x | x | x | x | x | x | x |
| CD117 | x | x | x | x | x | x | x | x |
| CD123 | x | x | x | x | x | x | x | x |
| CD133 | x | x | x | x | x | x | x | x |
| CD138 | x | x | x | x | x | x | x | x |
| CD15 | x | x | x | x | x | x | x | x |
| CD171 | x | x | x | x | x | x | x | x |
| CD19 | x | x | x | x | x | x | x | x |
| CD20 | x | x | x | x | x | x | x | x |
| CD21 | x | x | x | x | x | x | x | x |
| CD22 | x | x | x | x | x | x | x | x |
| CD30 | x | x | x | x | x | x | x | x |
| CD33 | x | x | x | x | x | x | x | x |
| CD37 | x | x | x | x | x | x | x | x |
| CD38 | x | x | x | x | x | x | x | x |
| CD44v6 | x | x | x | x | x | x | x | x |
| CD44v7/8 | x | x | x | x | x | x | x | x |
| CD74 | x | x | x | x | x | x | x | x |
| Cd79b | x | x | x | x | x | x | x | x |
| CD124 (IL- | x | x | x | x | x | x | x | x |
| 4R) | | | | | | | | |
| CDH3 | x | x | x | x | x | x | x | x |
| Ki-67 | x | x | x | x | x | x | x | x |
| CSPG4 | x | x | x | x | x | x | x | x |
| CALLA | x | x | x | x | x | x | x | x |
| CSAG2 | x | x | x | x | x | x | x | x |
| COX-2 | x | x | x | x | x | x | x | x |
| Lambda | x | x | x | x | x | x | x | x |
| LAYN | x | x | x | x | x | x | x | x |
| LeuM-1 | x | x | x | x | x | x | x | x |
| KDR | x | x | x | x | x | x | x | x |
| CD47 | x | x | x | x | x | x | x | x |
| CRISP3 | x | x | x | x | x | x | x | x |
| DC-SIGN | x | x | x | x | x | x | x | x |
| DHFR | x | x | x | x | x | x | x | x |
| EGP40 | x | x | x | x | x | x | x | x |
| EZH2 | x | x | x | x | x | x | x | x |
| EpCAM | x | x | x | x | x | x | x | x |
| EphA2 | x | x | x | x | x | x | x | x |
| EphA2/Eck | x | x | x | x | x | x | x | x |
| EGFRvIII | x | x | x | x | x | x | x | x |
| E-cadherin | x | x | x | x | x | x | x | x |
| EGP2 | x | x | x | x | x | x | x | x |
| ETA | x | x | x | x | x | x | x | x |
| ERBB3 | x | x | x | x | x | x | x | x |
| ERBB3/4 | x | x | x | x | x | x | x | x |
| ERBB4 | x | x | x | x | x | x | x | x |
| EPO | x | x | x | x | x | x | x | x |
| F3 | x | x | x | x | x | x | x | x |
| FAR | x | x | x | x | x | x | x | x |
| FBP | x | x | x | x | x | x | x | x |
| FTHL17 | x | x | x | x | x | x | x | x |
| fetal AchR | x | x | x | x | x | x | x | x |
| FAP | x | x | x | x | x | x | x | x |
| FGFR3 | x | x | x | x | x | x | x | x |
| FR-a | x | x | x | x | x | x | x | x |
| Fra-1/Fosl 1 | x | x | x | x | x | x | x | x |
| GAGE1 | x | x | x | x | x | x | x | x |
| GD2 | x | x | x | x | x | x | x | x |
| GD3 | x | x | x | x | x | x | x | x |
| Glil | x | x | x | x | x | x | x | x |
| GP100 | x | x | x | x | x | x | x | x |
| GPA33 | x | x | x | x | x | x | x | x |
| Glypican-3 | x | x | x | x | x | x | x | x |
| HIV gp120 | x | x | x | x | x | x | x | x |
| HLA-A | x | x | x | x | x | x | x | x |
| HLA-A2 | x | x | x | x | x | x | x | x |
| HLA-AI | x | x | x | x | x | x | x | x |
| HLA-B | x | x | x | x | x | x | x | x |
| HLA-C | x | x | x | x | x | x | x | x |
| HMW-MAA | x | x | x | x | x | x | x | x |
| Her2/Neu | x | x | x | x | x | x | x | x |
| Her3 | x | x | x | x | x | x | x | x |
| u70/80 | x | x | x | x | x | x | x | x |
| LICAM | x | x | x | x | x | x | x | x |
| ULBP1 | x | x | x | x | x | x | x | x |
| ULBP2 | x | x | x | x | x | x | x | x |
| ULBP3 | x | x | x | x | x | x | x | x |
| ULBP6 | x | x | x | x | x | x | x | x |
| MICA | x | x | x | x | x | x | x | x |
| MICB | x | x | x | x | x | x | x | x |
| Her3 | x | x | x | x | x | x | x | x |
| EGF | x | x | x | x | x | x | x | x |
| SIRP1α | x | x | x | x | x | x | x | x |
| Lewis-Y | x | x | x | x | x | x | x | x |
| LIV-1 | x | x | x | x | x | x | x | x |
| (SLC39A6) | | | | | | | | |
| Livin | x | x | x | x | x | x | x | x |
| LAMP1 | x | x | x | x | x | x | x | x |
| MAGEA3 | x | x | x | x | x | x | x | x |
| MAGEA4 | x | x | x | x | x | x | x | x |
| MAGEB6 | x | x | x | x | x | x | x | x |
| MAGEA1 | x | x | x | x | x | x | x | x |
| MART-1 | x | x | x | x | x | x | x | x |
| MCSP | x | x | x | x | x | x | x | x |
| MME | x | x | x | x | x | x | x | x |
| mesothelin | x | x | x | x | x | x | x | x |
| (MSLN) | | | | | | | | |
| MAPK1 | x | x | x | x | x | x | x | x |
| MUC16 | x | x | x | x | x | x | x | x |
| MUC1 | x | x | x | x | x | x | x | x |
| MRP-3 | x | x | x | x | x | x | x | x |
| MyoD-1 | x | x | x | x | x | x | x | x |
| NCAM | x | x | x | x | x | x | x | x |
| nectin 4 | x | x | x | x | x | x | x | x |
| Nestin | x | x | x | x | x | x | x | x |
| NEP | x | x | x | x | x | x | x | x |
| NY-ESO-1 | x | x | x | x | x | x | x | x |
| hHLA-A | x | x | x | x | x | x | x | x |
| H60 | x | x | x | x | x | x | x | x |
| OLIG2 | x | x | x | x | x | x | x | x |
| 5T4 | x | x | x | x | x | x | x | x |
| p53 | x | x | x | x | x | x | x | x |
| P-Cadherin | x | x | x | x | x | x | x | x |
| PB | x | x | x | x | x | x | x | x |
| P- | x | x | x | x | x | x | x | x |
| glycoprotein | | | | | | | | |
| PMCT | x | x | x | x | x | x | x | x |
| (SLC13A5) | | | | | | | | |
| PRAME | x | x | x | x | x | x | x | x |
| PROXI | x | x | x | x | x | x | x | x |
| PSA | x | x | x | x | x | x | x | x |
| PSCA | x | x | x | x | x | x | x | x |
| PSMA | x | x | x | x | x | x | x | x |
| PSC1 | x | x | x | x | x | x | x | x |
| PVRL4 | x | x | x | x | x | x | x | x |
| Ras | x | x | x | x | x | x | x | x |
| ROR1 | x | x | x | x | x | x | x | x |
| SART2 | x | x | x | x | x | x | x | x |
| SART3 | x | x | x | x | x | x | x | x |
| oncofetal | x | x | x | x | x | x | x | x |
| variants of | | | | | | | | |
| fibronectin | | | | | | | | |
| tenascin | x | x | x | x | x | x | x | x |
| LICAM | x | x | x | x | x | x | x | x |
| Rae-1α | x | x | x | x | x | x | x | x |
| Rae-1β | x | x | x | x | x | x | x | x |
| Rae-1δ | x | x | x | x | x | x | x | x |
| Rae-1γ | x | x | x | x | x | x | x | x |
| PDGF | x | x | x | x | x | x | x | x |
| Fas | x | x | x | x | x | x | x | x |
| SOX2 | x | x | x | x | x | x | x | x |
| STEAP1 | x | x | x | x | x | x | x | x |
| SLITRK6 | x | x | x | x | x | x | x | x |
| NaPi2a | x | x | x | x | x | x | x | x |
| SOX1 | x | x | x | x | x | x | x | x |
| SOX11 | x | x | x | x | x | x | x | x |
| SPANXA1 | x | x | x | x | x | x | x | x |
| SART-1 | x | x | x | x | x | x | x | x |
| SSX4 | x | x | x | x | x | x | x | x |
| SSX5 | x | x | x | x | x | x | x | x |
| Survivin | x | x | x | x | x | x | x | x |
| SSX2 | x | x | x | x | x | x | x | x |
| TAG72 | x | x | x | x | x | x | x | x |
| TEM1 | x | x | x | x | x | x | x | x |
| TEM8 | x | x | x | x | x | x | x | x |
| TSGA10 | x | x | x | x | x | x | x | x |
| TSSK6 | x | x | x | x | x | x | x | x |
| thyroglobulin | x | x | x | x | x | x | x | x |
| transferrin | x | x | x | x | x | x | x | x |
| receptor | | | | | | | | |
| TACSTD2 | x | x | x | x | x | x | x | x |
| (TROP2) | | | | | | | | |
| TMEM97 | x | x | x | x | x | x | x | x |
| TRP-2 | x | x | x | x | x | x | x | x |
| TULP2 | x | x | x | x | x | x | x | x |
| TROP2 | x | x | x | x | x | x | x | x |
| tyrosinase | x | x | x | x | x | x | x | x |
| TRP1 | x | x | x | x | x | x | x | x |
| UPAR | x | x | x | x | x | x | x | x |
| VEGF | x | x | x | x | x | x | x | x |
| VEGF | x | x | x | x | x | x | x | x |
| receptors | | | | | | | | |
| VEGRR2 | x | x | x | x | x | x | x | x |
| BRAF | x | x | x | x | x | x | x | x |
| WT-1 | x | x | x | x | x | x | x | x |
| XAGE2 | x | x | x | x | x | x | x | x |
| ZNF165 | x | x | x | x | x | x | x | x |
| αvβ6 integrin | x | x | x | x | x | x | x | x |
| β-catenin | x | x | x | x | x | x | x | x |
| cathepsin B | x | x | x | x | x | x | x | x |
| CSAG2 | x | x | x | x | x | x | x | x |
| CTAG | x | x | x | x | x | x | x | x |
| EGFR | x | x | x | x | x | x | x | x |
| EGP40 | x | x | x | x | x | x | x | x |
| EZH2 | x | x | x | x | x | x | x | x |
| HIV sp120 | x | x | x | x | x | x | x | x |
| kappa light | x | x | x | x | x | x | x | x |
| chain | | | | | | | | |
| LDHC | x | x | x | x | x | x | x | x |
| TRP-1 | x | x | x | x | x | x | x | x |
| Fas-L | x | x | x | x | x | x | x | x |
| DLL3 | x | x | x | x | x | x | x | x |
| MAGEA12 | x | x | x | x | x | x | x | x |
| MAGEC2 | x | x | x | x | x | x | x | x |
| BAGE | x | x | x | x | x | x | x | x |
| BAGE1 | x | x | x | x | x | x | x | x |
| GAGE | x | x | x | x | x | x | x | x |
| XAGE | x | x | x | x | x | x | x | x |
| XAGE1B | x | x | x | x | x | x | x | x |
| SSX | x | x | x | x | x | x | x | x |
| SSX2 | x | x | x | x | x | x | x | x |
| KKLC1 | x | x | x | x | x | x | x | x |
| SAGE | x | x | x | x | x | x | x | x |
| SPA17 | x | x | x | x | x | x | x | x |
| Cyclin A | x | x | x | x | x | x | x | x |
| KMHN1 | x | x | x | x | x | x | x | x |
| | | | | | | | | IL- |
| | NKp44 | NKp46 | KARs | CD48 | LIGHT | CD44 | CD45 | 1R2 |
| 8H9 | x | x | x | x | x | x | x | x |
| GnT-V,β1,6- | x | x | x | x | x | x | x | x |
| N | | | | | | | | |
| AFP | x | x | x | x | x | x | x | x |
| ART1 | x | x | x | x | x | x | x | x |
| ART4 | x | x | x | x | x | x | x | x |
| ABCG2 | x | x | x | x | x | x | x | x |
| B7-H3 | x | x | x | x | x | x | x | x |
| B7-H4 | x | x | x | x | x | x | x | x |
| B7-H6 | x | x | x | x | x | x | x | x |
| BCMA | x | x | x | x | x | x | x | x |
| B-cyclin | x | x | x | x | x | x | x | x |
| BMI1 | x | x | x | x | x | x | x | x |
| CA-125 | x | x | x | x | x | x | x | x |
| cadherin | x | x | x | x | x | x | x | x |
| CABYR | x | x | x | x | x | x | x | x |
| CTAG2 | x | x | x | x | x | x | x | x |
| CA6 | x | x | x | x | x | x | x | x |
| CAIX | x | x | x | x | x | x | x | x |
| CEA | x | x | x | x | x | x | x | x |
| CEACAM5 | x | x | x | x | x | x | x | x |
| CEACAM6 | x | x | x | x | x | x | x | x |
| Cav-1 | x | x | x | x | x | x | x | x |
| CD10 | x | x | x | x | x | x | x | x |
| CD117 | x | x | x | x | x | x | x | x |
| CD123 | x | x | x | x | x | x | x | x |
| CD133 | x | x | x | x | x | x | x | x |
| CD138 | x | x | x | x | x | x | x | x |
| CD15 | x | x | x | x | x | x | x | x |
| CD171 | x | x | x | x | x | x | x | x |
| CD19 | x | x | x | x | x | x | x | x |
| CD20 | x | x | x | x | x | x | x | x |
| CD21 | x | x | x | x | x | x | x | x |
| CD22 | x | x | x | x | x | x | x | x |
| CD30 | x | x | x | x | x | x | x | x |
| CD33 | x | x | x | x | x | x | x | x |
| CD37 | x | x | x | x | x | x | x | x |
| CD38 | x | x | x | x | x | x | x | x |
| CD44v6 | x | x | x | x | x | x | x | x |
| CD44v7/8 | x | x | x | x | x | x | x | x |
| CD74 | x | x | x | x | x | x | x | x |
| Cd79b | x | x | x | x | x | x | x | x |
| CD124 (IL- | x | x | x | x | x | x | x | x |
| 4R) | | | | | | | | |
| CDH3 | x | x | x | x | x | x | x | x |
| Ki-67 | x | x | x | x | x | x | x | x |
| CSPG4 | x | x | x | x | x | x | x | x |
| CALLA | x | x | x | x | x | x | x | x |
| CSAG2 | x | x | x | x | x | x | x | x |
| COX-2 | x | x | x | x | x | x | x | x |
| Lambda | x | x | x | x | x | x | x | x |
| LAYN | x | x | x | x | x | x | x | x |
| LeuM-1 | x | x | x | x | x | x | x | x |
| KDR | x | x | x | x | x | x | x | x |
| CD47 | x | x | x | x | x | x | x | x |
| CRISP3 | x | x | x | x | x | x | x | x |
| DC-SIGN | x | x | x | x | x | x | x | x |
| DHFR | x | x | x | x | x | x | x | x |
| EGP40 | x | x | x | x | x | x | x | x |
| EZH2 | x | x | x | x | x | x | x | x |
| EpCAM | x | x | x | x | x | x | x | x |
| EphA2 | x | x | x | x | x | x | x | x |
| EphA2/Eck | x | x | x | x | x | x | x | x |
| EGFRvIII | x | x | x | x | x | x | x | x |
| E-cadherin | x | x | x | x | x | x | x | x |
| EGP2 | x | x | x | x | x | x | x | x |
| ETA | x | x | x | x | x | x | x | x |
| ERBB3 | x | x | x | x | x | x | x | x |
| ERBB3/4 | x | x | x | x | x | x | x | x |
| ERBB4 | x | x | x | x | x | x | x | x |
| EPO | x | x | x | x | x | x | x | x |
| F3 | x | x | x | x | x | x | x | x |
| FAR | x | x | x | x | x | x | x | x |
| FBP | x | x | x | x | x | x | x | x |
| FTHL17 | x | x | x | x | x | x | x | x |
| fetal AchR | x | x | x | x | x | x | x | x |
| FAP | x | x | x | x | x | x | x | x |
| FGFR3 | x | x | x | x | x | x | x | x |
| FR-a | x | x | x | x | x | x | x | x |
| Fra-1/Fosl 1 | x | x | x | x | x | x | x | x |
| GAGE1 | x | x | x | x | x | x | x | x |
| GD2 | x | x | x | x | x | x | x | x |
| GD3 | x | x | x | x | x | x | x | x |
| Glil | x | x | x | x | x | x | x | x |
| GP100 | x | x | x | x | x | x | x | x |
| GPA33 | x | x | x | x | x | x | x | x |
| Glypican-3 | x | x | x | x | x | x | x | x |
| HIV gp120 | x | x | x | x | x | x | x | x |
| HLA-A | x | x | x | x | x | x | x | x |
| HLA-A2 | x | x | x | x | x | x | x | x |
| HLA-AI | x | x | x | x | x | x | x | x |
| HLA-B | x | x | x | x | x | x | x | x |
| HLA-C | x | x | x | x | x | x | x | x |
| HMW-MAA | x | x | x | x | x | x | x | x |
| Her2/Neu | x | x | x | x | x | x | x | x |
| Her3 | x | x | x | x | x | x | x | x |
| u70/80 | x | x | x | x | x | x | x | x |
| LICAM | x | x | x | x | x | x | x | x |
| ULBP1 | x | x | x | x | x | x | x | x |
| ULBP2 | x | x | x | x | x | x | x | x |
| ULBP3 | x | x | x | x | x | x | x | x |
| ULBP6 | x | x | x | x | x | x | x | x |
| MICA | x | x | x | x | x | x | x | x |
| MICB | x | x | x | x | x | x | x | x |
| Her3 | x | x | x | x | x | x | x | x |
| EGF | x | x | x | x | x | x | x | x |
| SIRP1α | x | x | x | x | x | x | x | x |
| Lewis-Y | x | x | x | x | x | x | x | x |
| LIV-1 | x | x | x | x | x | x | x | x |
| (SLC39A6) | | | | | | | | |
| Livin | x | x | x | x | x | x | x | x |
| LAMP1 | x | x | x | x | x | x | x | x |
| MAGEA3 | x | x | x | x | x | x | x | x |
| MAGEA4 | x | x | x | x | x | x | x | x |
| MAGEB6 | x | x | x | x | x | x | x | x |
| MAGEA1 | x | x | x | x | x | x | x | x |
| MART-1 | x | x | x | x | x | x | x | x |
| MCSP | x | x | x | x | x | x | x | x |
| MME | x | x | x | x | x | x | x | x |
| mesothelin | x | x | x | x | x | x | x | x |
| (MSLN) | | | | | | | | |
| MAPK1 | x | x | x | x | x | x | x | x |
| MUC16 | x | x | x | x | x | x | x | x |
| MUC1 | x | x | x | x | x | x | x | x |
| MRP-3 | x | x | x | x | x | x | x | x |
| MyoD-1 | x | x | x | x | x | x | x | x |
| NCAM | x | x | x | x | x | x | x | x |
| nectin 4 | x | x | x | x | x | x | x | x |
| Nestin | x | x | x | x | x | x | x | x |
| NEP | x | x | x | x | x | x | x | x |
| NY-ESO-1 | x | x | x | x | x | x | x | x |
| hHLA-A | x | x | x | x | x | x | x | x |
| H60 | x | x | x | x | x | x | x | x |
| OLIG2 | x | x | x | x | x | x | x | x |
| 5T4 | x | x | x | x | x | x | x | x |
| p53 | x | x | x | x | x | x | x | x |
| P-Cadherin | x | x | x | x | x | x | x | x |
| PB | x | x | x | x | x | x | x | x |
| P- | x | x | x | x | x | x | x | x |
| glycoprotein | | | | | | | | |
| PMCT | x | x | x | x | x | x | x | x |
| (SLC13A5) | | | | | | | | |
| PRAME | x | x | x | x | x | x | x | x |
| PROXI | x | x | x | x | x | x | x | x |
| PSA | x | x | x | x | x | x | x | x |
| PSCA | x | x | x | x | x | x | x | x |
| PSMA | x | x | x | x | x | x | x | x |
| PSC1 | x | x | x | x | x | x | x | x |
| PVRL4 | x | x | x | x | x | x | x | x |
| Ras | x | x | x | x | x | x | x | x |
| ROR1 | x | x | x | x | x | x | x | x |
| SART2 | x | x | x | x | x | x | x | x |
| SART3 | x | x | x | x | x | x | x | x |
| oncofetal | x | x | x | x | x | x | x | x |
| variants of | x | x | x | x | x | x | x | x |
| fibronectin | | | | | | | | |
| tenascin | | | | | | | | |
| LICAM | x | x | x | x | x | x | x | x |
| Rae-1α | x | x | x | x | x | x | x | x |
| Rae-1β | x | x | x | x | x | x | x | x |
| Rae-1δ | x | x | x | x | x | x | x | x |
| Rae-1γ | x | x | x | x | x | x | x | x |
| PDGF | x | x | x | x | x | x | x | x |
| Fas | x | x | x | x | x | x | x | x |
| SOX2 | x | x | x | x | x | x | x | x |
| STEAP1 | x | x | x | x | x | x | x | x |
| SLITRK6 | x | x | x | x | x | x | x | x |
| NaPi2a | x | x | x | x | x | x | x | x |
| SOX1 | x | x | x | x | x | x | x | x |
| SOX11 | x | x | x | x | x | x | x | x |
| SPANXA1 | x | x | x | x | x | x | x | x |
| SART-1 | x | x | x | x | x | x | x | x |
| SSX4 | x | x | x | x | x | x | x | x |
| SSX5 | x | x | x | x | x | x | x | x |
| Survivin | x | x | x | x | x | x | x | x |
| SSX2 | x | x | x | x | x | x | x | x |
| TAG72 | x | x | x | x | x | x | x | x |
| TEM1 | x | x | x | x | x | x | x | x |
| TEM8 | x | x | x | x | x | x | x | x |
| TSGA10 | x | x | x | x | x | x | x | x |
| TSSK6 | x | x | x | x | x | x | x | x |
| thyroglobulin | x | x | x | x | x | x | x | x |
| transferrin | x | x | x | x | x | x | x | x |
| receptor | | | | | | | | |
| TACSTD2 | x | x | x | x | x | x | x | x |
| (TROP2) | | | | | | | | |
| TMEM97 | x | x | x | x | x | x | x | x |
| TRP-2 | x | x | x | x | x | x | x | x |
| TULP2 | x | x | x | x | x | x | x | x |
| TROP2 | x | x | x | x | x | x | x | x |
| tyrosinase | x | x | x | x | x | x | x | x |
| TRP1 | x | x | x | x | x | x | x | x |
| UPAR | x | x | x | x | x | x | x | x |
| VEGF | x | x | x | x | x | x | x | x |
| VEGF | x | x | x | x | x | x | x | x |
| receptors | | | | | | | | |
| VEGRR2 | x | x | x | x | x | x | x | x |
| BRAF | x | x | x | x | x | x | x | x |
| WT-1 | x | x | x | x | x | x | x | x |
| XAGE2 | x | x | x | x | x | x | x | x |
| ZNF165 | x | x | x | x | x | x | x | x |
| αvβ6 integrin | x | x | x | x | x | x | x | x |
| β-catenin | x | x | x | x | x | x | x | x |
| cathepsin B | x | x | x | x | x | x | x | x |
| CSAG2 | x | x | x | x | x | x | x | x |
| CTAG | x | x | x | x | x | x | x | x |
| EGFR | x | x | x | x | x | x | x | x |
| EGP40 | x | x | x | x | x | x | x | x |
| EZH2 | x | x | x | x | x | x | x | x |
| HIV sp120 | x | x | x | x | x | x | x | x |
| kappa light | x | x | x | x | x | x | x | x |
| chain | | | | | | | | |
| LDHC | x | x | x | x | x | x | x | x |
| TRP-1 | x | x | x | x | x | x | x | x |
| Fas-L | x | x | x | x | x | x | x | x |
| DLL3 | x | x | x | x | x | x | x | x |
| MAGEA12 | x | x | x | x | x | x | x | x |
| MAGEC2 | x | x | x | x | x | x | x | x |
| BAGE | x | x | x | x | x | x | x | x |
| BAGE1 | x | x | x | x | x | x | x | x |
| GAGE | x | x | x | x | x | x | x | x |
| XAGE | x | x | x | x | x | x | x | x |
| XAGE1B | x | x | x | x | x | x | x | x |
| SSX | x | x | x | x | x | x | x | x |
| SSX2 | x | x | x | x | x | x | x | x |
| KKLC1 | x | x | x | x | x | x | x | x |
| SAGE | x | x | x | x | x | x | x | x |
| SPA17 | x | x | x | x | x | x | x | x |
| Cyclin A | x | x | x | x | x | x | x | x |
| KMHN1 | x | x | x | x | x | x | x | x |

| | IL- | IL- | IL- | IL- | | | |
|---|---|---|---|---|---|---|---|
| | 1Rα | IRα2 | 13Rα2 | 15Ra | CCR5 | CCR8 | SIRPα |
| 8H9 | x | x | x | x | x | x | x |
| GnT-V,β1,6- | x | x | x | x | x | x | x |
| N | | | | | | | |
| AFP | x | x | x | x | x | x | x |
| ART1 | x | x | x | x | x | x | x |
| ART4 | x | x | x | x | x | x | x |
| ABCG2 | x | x | x | x | x | x | x |
| B7-H3 | x | x | x | x | x | x | x |
| B7-H4 | x | x | x | x | x | x | x |
| B7-H6 | x | x | x | x | x | x | x |
| BCMA | x | x | x | x | x | x | x |
| B-cyclin | x | x | x | x | x | x | x |
| BMI1 | x | x | x | x | x | x | x |
| CA-125 | x | x | x | x | x | x | x |
| cadherin | x | x | x | x | x | x | x |
| CABYR | x | x | x | x | x | x | x |
| CTAG2 | x | x | x | x | x | x | x |
| CA6 | x | x | x | x | x | x | x |
| CAIX | x | x | x | x | x | x | x |
| CEA | x | x | x | x | x | x | x |
| CEACAM5 | x | x | x | x | x | x | x |
| CEACAM6 | x | x | x | x | x | x | x |
| Cav-1 | x | x | x | x | x | x | x |
| CD10 | x | x | x | x | x | x | x |
| CD117 | x | x | x | x | x | x | x |
| CD123 | x | x | x | x | x | x | x |
| CD133 | x | x | x | x | x | x | x |
| CD138 | x | x | x | x | x | x | x |
| CD15 | x | x | x | x | x | x | x |
| CD171 | x | x | x | x | x | x | x |
| CD19 | x | x | x | x | x | x | x |
| CD20 | x | x | x | x | x | x | x |
| CD21 | x | x | x | x | x | x | x |
| CD22 | x | x | x | x | x | x | x |
| CD30 | x | x | x | x | x | x | x |
| CD33 | x | x | x | x | x | x | x |
| CD37 | x | x | x | x | x | x | x |
| CD38 | x | x | x | x | x | x | x |
| CD44v6 | x | x | x | x | x | x | x |
| CD44v7/8 | x | x | x | x | x | x | x |
| CD74 | x | x | x | x | x | x | x |
| Cd79b | x | x | x | x | x | x | x |
| CD124 (IL- | x | x | x | x | x | x | x |
| 4R) | | | | | | | |
| CDH3 | x | x | x | x | x | x | x |
| Ki-67 | x | x | x | x | x | x | x |
| CSPG4 | x | x | x | x | x | x | x |
| CALLA | x | x | x | x | x | x | x |
| CSAG2 | x | x | x | x | x | x | x |
| COX-2 | x | x | x | x | x | x | x |
| Lambda | x | x | x | x | x | x | x |
| LAYN | x | x | x | x | x | x | x |
| LeuM-1 | x | x | x | x | x | x | x |
| KDR | x | x | x | x | x | x | x |
| CD47 | x | x | x | x | x | x | x |
| CRISP3 | x | x | x | x | x | x | x |
| DC-SIGN | x | x | x | x | x | x | x |
| DHFR | x | x | x | x | x | x | x |
| EGP40 | x | x | x | x | x | x | x |
| EZH2 | x | x | x | x | x | x | x |
| EpCAM | x | x | x | x | x | x | x |
| EphA2 | x | x | x | x | x | x | x |
| EphA2/Eck | x | x | x | x | x | x | x |
| EGFRvIII | x | x | x | x | x | x | x |
| E-cadherin | x | x | x | x | x | x | x |
| EGP2 | x | x | x | x | x | x | x |
| ETA | x | x | x | x | x | x | x |
| ERBB3 | x | x | x | x | x | x | x |
| ERBB3/4 | x | x | x | x | x | x | x |
| ERBB4 | x | x | x | x | x | x | x |
| EPO | x | x | x | x | x | x | x |
| F3 | x | x | x | x | x | x | x |
| FAR | x | x | x | x | x | x | x |
| FBP | x | x | x | x | x | x | x |
| FTHL17 | x | x | x | x | x | x | x |
| fetal AchR | x | x | x | x | x | x | x |
| FAP | x | x | x | x | x | x | x |
| FGFR3 | x | x | x | x | x | x | x |
| FR-a | x | x | x | x | x | x | x |
| Fra-1/Fosl 1 | x | x | x | x | x | x | x |
| GAGE1 | x | x | x | x | x | x | x |
| GD2 | x | x | x | x | x | x | x |
| GD3 | x | x | x | x | x | x | x |
| Glil | x | x | x | x | x | x | x |
| GP100 | x | x | x | x | x | x | x |
| GPA33 | x | x | x | x | x | x | x |
| Glypican-3 | x | x | x | x | x | x | x |
| HIV gp120 | x | x | x | x | x | x | x |
| HLA-A | x | x | x | x | x | x | x |
| HLA-A2 | x | x | x | x | x | x | x |
| HLA-AI | x | x | x | x | x | x | x |
| HLA-B | x | x | x | x | x | x | x |
| HLA-C | x | x | x | x | x | x | x |
| HMW-MAA | x | x | x | x | x | x | x |
| Her2/Neu | x | x | x | x | x | x | x |
| Her3 | x | x | x | x | x | x | x |
| u70/80 | x | x | x | x | x | x | x |
| LICAM | x | x | x | x | x | x | x |
| ULBP1 | x | x | x | x | x | x | x |
| ULBP2 | x | x | x | x | x | x | x |
| ULBP3 | x | x | x | x | x | x | x |
| ULBP6 | x | x | x | x | x | x | x |
| MICA | x | x | x | x | x | x | x |
| MICB | x | x | x | x | x | x | x |
| Her3 | x | x | x | x | x | x | x |
| EGF | x | x | x | x | x | x | x |
| SIRP1α | x | x | x | x | x | x | x |
| Lewis-Y | x | x | x | x | x | x | x |
| LIV-1 | x | x | x | x | x | x | x |
| (SLC39A6) | | | | | | | |
| Livin | x | x | x | x | x | x | x |
| LAMP1 | x | x | x | x | x | x | x |
| MAGEA3 | x | x | x | x | x | x | x |
| MAGEA4 | x | x | x | x | x | x | x |
| MAGEB6 | x | x | x | x | x | x | x |
| MAGEA1 | x | x | x | x | x | x | x |
| MART-1 | x | x | x | x | x | x | x |
| MCSP | x | x | x | x | x | x | x |
| MME | x | x | x | x | x | x | x |
| mesothelin | x | x | x | x | x | x | x |
| (MSLN) | | | | | | | |
| MAPK1 | x | x | x | x | x | x | x |
| MUC16 | x | x | x | x | x | x | x |
| MUC1 | x | x | x | x | x | x | x |
| MRP-3 | x | x | x | x | x | x | x |
| MyoD-1 | x | x | x | x | x | x | x |
| NCAM | x | x | x | x | x | x | x |
| nectin 4 | x | x | x | x | x | x | x |
| Nestin | x | x | x | x | x | x | x |
| NEP | x | x | x | x | x | x | x |
| NY-ESO-1 | x | x | x | x | x | x | x |
| hHLA-A | x | x | x | x | x | x | x |
| H60 | x | x | x | x | x | x | x |
| OLIG2 | x | x | x | x | x | x | x |
| 5T4 | x | x | x | x | x | x | x |
| p53 | x | x | x | x | x | x | x |
| P-Cadherin | x | x | x | x | x | x | x |
| PB | x | x | x | x | x | x | x |
| P- | x | x | x | x | x | x | x |
| glycoprotein | | | | | | | |
| PMCT | x | x | x | x | x | x | x |
| (SLC13A5) | | | | | | | |
| PRAME | x | x | x | x | x | x | x |
| PROX1 | x | x | x | x | x | x | x |
| PSA | x | x | x | x | x | x | x |
| PSCA | x | x | x | x | x | x | x |
| PSMA | x | x | x | x | x | x | x |
| PSC1 | x | x | x | x | x | x | x |
| PVRL4 | x | x | x | x | x | x | x |
| Ras | x | x | x | x | x | x | x |
| ROR1 | x | x | x | x | x | x | x |
| SART2 | x | x | x | x | x | x | x |
| SART3 | x | x | x | x | x | x | x |
| oncofetal | x | x | x | x | x | x | x |
| variants of | x | x | x | x | x | x | x |
| fibronectin | | | | | | | |
| tenascin | | | | | | | |
| LICAM | x | x | x | x | x | x | x |
| Rae-1α | x | x | x | x | x | x | x |
| Rae-1β | x | x | x | x | x | x | x |
| Rae-1δ | x | x | x | x | x | x | x |
| Rae-1γ | x | x | x | x | x | x | x |
| PDGF | x | x | x | x | x | x | x |
| Fas | x | x | x | x | x | x | x |
| SOX2 | x | x | x | x | x | x | x |
| STEAP1 | x | x | x | x | x | x | x |
| SLITRK6 | x | x | x | x | x | x | x |
| NaPi2a | x | x | x | x | x | x | x |
| SOX1 | x | x | x | x | x | x | x |
| SOX11 | x | x | x | x | x | x | x |
| SPANXA1 | x | x | x | x | x | x | x |
| SART-1 | x | x | x | x | x | x | x |
| SSX4 | x | x | x | x | x | x | x |
| SSX5 | x | x | x | x | x | x | x |
| Survivin | x | x | x | x | x | x | x |
| SSX2 | x | x | x | x | x | x | x |
| TAG72 | x | x | x | x | x | x | x |
| TEM1 | x | x | x | x | x | x | x |
| TEM8 | x | x | x | x | x | x | x |
| TSGA10 | x | x | x | x | x | x | x |
| TSSK6 | x | x | x | x | x | x | x |
| thyroglobulin | x | x | x | x | x | x | x |
| transferrin | x | x | x | x | x | x | x |
| receptor | | | | | | | |
| TACSTD2 | x | x | x | x | x | x | x |
| (TROP2) | | | | | | | |
| TMEM97 | x | x | x | x | x | x | x |
| TRP-2 | x | x | x | x | x | x | x |
| TULP2 | x | x | x | x | x | x | x |
| TROP2 | x | x | x | x | x | x | x |
| tyrosinase | x | x | x | x | x | x | x |
| TRP1 | x | x | x | x | x | x | x |
| UPAR | x | x | x | x | x | x | x |
| VEGF | x | x | x | x | x | x | x |
| VEGF | x | x | x | x | x | x | x |
| receptors | | | | | | | |
| VEGRR2 | x | x | x | x | x | x | x |
| BRAF | x | x | x | x | x | x | x |
| WT-1 | x | x | x | x | x | x | x |
| XAGE2 | x | x | x | x | x | x | x |
| ZNF165 | x | x | x | x | x | x | x |
| αvβ6 integrin | x | x | x | x | x | x | x |
| β-catenin | x | x | x | x | x | x | x |
| cathepsin B | x | x | x | x | x | x | x |
| CSAG2 | x | x | x | x | x | x | x |
| CTAG | x | x | x | x | x | x | x |
| EGFR | x | x | x | x | x | x | x |
| EGP40 | x | x | x | x | x | x | x |
| EZH2 | x | x | x | x | x | x | x |
| HIV sp120 | x | x | x | x | x | x | x |
| kappa light | x | x | x | x | x | x | x |
| chain | | | | | | | |
| LDHC | x | x | x | x | x | x | x |
| TRP-1 | x | x | x | x | x | x | x |
| Fas-L | x | x | x | x | x | x | x |
| DLL3 | x | x | x | x | x | x | x |
| MAGEA12 | x | x | x | x | x | x | x |
| MAGEC2 | x | x | x | x | x | x | x |
| BAGE | x | x | x | x | x | x | x |
| BAGE1 | x | x | x | x | x | x | x |
| GAGE | x | x | x | x | x | x | x |
| XAGE | x | x | x | x | x | x | x |
| XAGE1B | x | x | x | x | x | x | x |
| SSX | x | x | x | x | x | x | x |
| SSX2 | x | x | x | x | x | x | x |
| KKLC1 | x | x | x | x | x | x | x |
| SAGE | x | x | x | x | x | x | x |
| SPA17 | x | x | x | x | x | x | x |
| Cyclin A | x | x | x | x | x | x | x |
| KMHN1 | x | x | x | x | x | x | x |

In some embodiments, the payload molecule is an antigen. In some embodiments, the antigen is a protein selected from those listed in Table 4 or a portion thereof. In some embodiments, the antigen is a tumor-associated antigen (TAA) or a portion thereof. In some embodiments, the tumor-associated antigen is expressed on the cell surface of tumor cells. In some embodiments, expression of the antigen or a portion thereof induces immune responses against tumor cells. In some embodiments, the tumor-associated antigen is selected from CD19, EpCAM, CEA, PSMA, CD33, EGFR, Her2, EphA2, MCSP, ADAM17, PSCA, 17-A1, an NKGD2 ligand, CSF1R, FAP, GD2, DLL3, neuropilin, Survivin, or a MAGE family protein. In some embodiments, the tumor-associated antigen is Survivin. In some embodiments, the tumor-associated antigen is a MAGE (Melanoma Antigen Gene) family protein. The MAGE family protein comprises MAGE-B1, MAGEA1, MAGEA10, MAGEA11, MAGEA12, MAGEA2B, MAGEA3, MAGEA4, MAGEA6, MAGEA8, MAGEA9, MAGEB1, MAGEB10, MAGEB16, MAGEB18, MAGEB2, MAGEB3, MAGEB4, MAGEB5, MAGEB6, MAGEB6B, MAGEC1, MAGEC2, MAGEC3, MAGED1, MAGED2, MAGED4, MAGEE1, MAGEE2, MAGEF1, MAGEH1, MAGEL2, NDN, NDNL2, or any combination thereof. In some embodiments, the tumor associated antigen is selected from the antigens in Table 6 below. In some embodiments, the replicon encodes two, three, four, five or more tumor associated antigens of the disclosure.

In some embodiments, the payload molecule comprises or consists of a fragment (i.e., peptide fragment) of a tumor-associated antigen (TAA) of the disclosure. In some embodiments, the fragment of the TAA has a length of about 10 amino acids (aa), about 15 aa, about 20 aa, about 30 aa, about 40 aa, about 50 aa, about 60 aa, about 70 aa, about 80 aa, about 90 aa, about 100 aa, or any values in between. In some embodiments, the fragment of the TAA has a length of at least 10 aa, at least 15 aa, at least 20 aa, at least 30 aa, at least 40 aa, at least 50 aa, at least 60 aa, at least 70 aa, at least 80 aa, at least 90 aa, or at least 100 aa. In some embodiments, the replicon comprises two, three, four, five or more payload molecules each comprising or consisting of a fragment of different TAAs. In some embodiments, the replicon comprises two, three, four, five or more payload molecules each comprising or consisting of different fragments of the same TAA. In some embodiments, the replicon comprises two, three, four, five or more copies of the payload molecules each comprising or consisting of the same fragment of the same TAA. In some embodiments, the payload molecule comprises repeats of the same peptide fragment of the TAA, such as 2, 3, 4, 5, 6, 7, 8, 9, 10, or more than 10 repeats of the same peptide fragment.

TABLE 6
Tumor-Associated Antigen
Tumor-Associated Antigen
MAGEA1
MAGEA3
MAGEA4
MAGEA12
MAGEC2
BAGE (B melanoma antigen)
BAGE1 (B melanoma antigen 1)
GAGE (G antigen)
GAGE1 (G antigen 1)
XAGE (X antigen)
XAGE1B (X antigen family member 1B)
CTAG (cancer/testis antigen)
CTAG2 (LAGE1)
CTAGI (NY-ESO-1)
SSX (synovial sarcoma X)
SSX2 (synovial sarcoma X breakpoint 2)
KKLC1 (Kita-kyushu lung cancer antigen 1)
SAGE (sarcoma antigen)
SPA17 (sperm autoantigenic protein 17)
Cyclin A
KMHN1 (CCDC110)
Survivin
EBV-Encoded Latent Membrane Protein 1 (LMP1)
EBV-Encoded Latent Membrane Protein 2 (LMP2)
Viral antigen derived from Human papillomavirus (HPV)
Viral antigen derived from Human papillomavirus (HPV) strain 6
Viral antigen derived from Human papillomavirus (HPV) strain 7
Viral antigen derived from Human papillomavirus (HPV) strain 11
Viral antigen derived from Human papillomavirus (HPV) strain 16
Viral antigen derived from Human papillomavirus (HPV) strain 18
Viral antigen derived from Human papillomavirus (HPV) strain 31
Viral antigen derived from Epstein-Barr virus (EBV)
Viral antigen derived from Viral antigen from Human T-lymphotrophic virus
(HTLV)
Viral antigen derived from Merkel cell polyomavirus (MCV)
Viral antigen derived from Kaposi′s sarcoma-associated herpesvirus
Viral antigen derived from Cytomegalovirus (CMV)

In some embodiments, the payload molecule comprises or consist of a tumor neoantigen. The term “tumor neoantigen” refers to a neoantigen present in a subject's tumor cell or tissue but not in the subject's corresponding normal cell or tissue. Tumor neoantigen may be a peptide or a protein. In some embodiments, the tumor neoantigen is patient-specific or subject-specific. In some embodiments, the replicon encodes multiple payload molecules comprising a tumor neoantigen, such as 2, 3, 4, 5, 6, 7, 8, 9, 10, or more than 10 payload molecules comprising a tumor neoantigen. In some embodiments, the replicon may encode multiple copies of the same tumor neoantigen.

In some embodiments, the payload molecule is a bipartite polypeptide with specific binding to a major histocompatibility complex (MHC)-peptide antigen complex. In some embodiments, the bipartite polypeptide binds specifically to the MHC-peptide antigen complex. In some embodiments, the MHC is a class I MHC. In some embodiments, the peptide antigen is derived from TAA or tumor neoantigen. In some embodiments, the bipartite polypeptide comprises a fragment of a T-cell receptor (TCR) (e.g. the extracellular domain of TCR) that specifically binds to the MHC-peptide antigen complex. In some embodiments, the bipartite polypeptide also binds to one of the effector cell antigens according to Table 3. In some embodiments, the bipartite polypeptide specifically binds to CD3. In some embodiments, the bipartite polypeptide specifically binds to CD3R.

In some embodiments, the recombinant RNA replicon comprises one or more payload molecules, wherein the payload molecules comprise:
a) one or more cytokines comprising IFNγ, GM-CSF, IL-2, IL-12, IL-15, IL-18, IL-23, and IL-36γ;
b) one or more chemokines comprising CXCL10, CCL4, CCL5 and CCL21;
c) one or more antibodies comprising an anti-PD1-VHH-Fc antibody, an anti-CD47-VHH-Fc antibody, and an anti-TGFβ-VHH(or scFv)-Fc antibody;
d) one or more bipartite polypeptides comprising a bipartite polypeptide binding to DLL3 and an effector cell target antigen, a bipartite polypeptide binding to FAP and an effector cell target antigen, and a bipartite polypeptide binding to EpCAM and an effector cell target antigen;
e) one or more tumor-associated antigens comprising survivin, MAGE family proteins, and all antigens according to Table 6;
f) one or more tumor neoantigens;
g) one or more bipartite polypeptides binding to MHC-peptide antigen complex;
h) one or more fusogenic proteins comprising herpes simplex virus (HSV) UL27/glycoprotein B/gB, HSV UL53/glycoprotein K/gK, Respiratory syncytial virus (RSV) F protein, FASTp15, VSV-G, syncitin-1 (from human endogenous retrovirus-W (HERV-W)) or syncitin-2 (from HERVFRDE1), paramyxovirus SV5-F, measles virus-H, measles virus-F, and the glycoprotein from a retrovirus or lentivirus, such as gibbon ape leukemia virus (GALV), murine leukemia virus (MLV), Mason-Pfizer monkey virus (MPMV) and equine infectious anemia virus (EIAV), optionally with the R transmembrane peptide removed (R-versions);
i) one or more other payload molecules comprising IL15R, PGDH, ADA, ADA2, HYAL1, HYAL2, CHIPS, MLKL (or its 4HB domain only), GSDMD (or its L192A mutant, or its amino acids 1-233 fragment, or its amino acids 1-233 fragment with L192A mutation), GSDME (or its amino acid 1-237 fragment), HMGB1 (or its Box B domain only), Melittin (e.g., alpha-Melittin), SMAC/Diablo (or its amino acid 56-239 fragment), Snake LAAO, Snake disintegrin, Leptin, FLT3L, TRAIL, Gasdermin D or a truncation thereof, and Gasdermin E or a truncation thereof;
j) one or more antigens from pathogens comprising Dengue virus, Chikungunya virus, Mycobacterium tuberculosis, Human immunodeficiency viruses, SARS-CoV-2, Coronavirus, Hepatitis B Virus, Togaviridae family virus, Flaviviridae family virus, Influenza A virus, Influenza B virus, and a veterinary virus; or
k) any combination thereof.

Fusogenic proteins are proteins that facilitate the fusion of cell to cell membranes. The payload molecule, or at least one of the payload molecules, encoded by the replicon of the disclosure may be a fusogenic protein comprising herpes simplex virus (HSV) UL27/glycoprotein B/gB, HSV UL53/glycoprotein K/gK, Respiratory syncytial virus (RSV) F protein, FASTp15, VSV-G, syncitin-1 (from human endogenous retrovirus-W (HERV-W)) or syncitin-2 (from HERVFRDE1), paramyxovirus SV5-F, measles virus-H, measles virus-F, and the glycoprotein from a retrovirus or lentivirus, such as gibbon ape leukemia virus (GALV), murine leukemia virus (MLV), Mason-Pfizer monkey virus (MPMV) and equine infectious anemia virus (EIAV), optionally with the R transmembrane peptide removed (R-versions).

In some embodiments, the payload molecule is GM-CSF. In some embodiments, the payload molecule is a GM-CSF polypeptide having at least 70%, at least 75%, at least 80%, at least 85%, at least 90%, at least 95%, at least 98%, at least 99%, or 100%, sequence identity to SEQ ID NO: 81.

In some embodiments, the payload molecule is IL-2. In some embodiments, the payload molecule is a IL-2 polypeptide having at least 70%, at least 75%, at least 80%, at least 85%, at least 90%, at least 95%, at least 98%, at least 99%, or 100%, sequence identity to SEQ ID NO: 82. In some embodiments, the payload molecule is a IL-2 polypeptide having at least 70%, at least 75%, at least 80%, at least 85%, at least 90%, at least 95%, at least 98%, at least 99%, or 100%, sequence identity to SEQ ID NO: 83.

In some embodiments, the payload molecule is IL-12 beta subunit. In some embodiments, the payload molecule is a IL-12 beta subunit polypeptide having at least 70%, at least 75%, at least 80%, at least 85%, at least 90%, at least 95%, at least 98%, at least 99%, or 100%, sequence identity to SEQ ID NO: 84. In some embodiments, the payload molecule is IL-12 alpha subunit. In some embodiments, the payload molecule is a IL-12 alpha subunit polypeptide having at least 70%, at least 75%, at least 80%, at least 85%, at least 90%, at least 95%, at least 98%, at least 99%, or 100%, sequence identity to SEQ ID NO: 85. In some embodiments, the payload molecule is IL-23 alpha subunit. In some embodiments, the payload molecule is a IL-23 alpha subunit polypeptide having at least 70%, at least 75%, at least 80%, at least 85%, at least 90%, at least 95%, at least 98%, at least 99%, or 100%, sequence identity to SEQ ID NO: 86.

In some embodiments, the payload molecule is IL-18. In some embodiments, the payload molecule is an IL-18 polypeptide having at least 70%, at least 75%, at least 80%, at least 85%, at least 90%, at least 95%, at least 98%, at least 99%, or 100%, sequence identity to SEQ ID NO: 87.

In some embodiments, the payload molecule is IL-36γ. In some embodiments, the payload molecule is a IL-36γ polypeptide having at least 70%, at least 75%, at least 80%, at least 85%, at least 90%, at least 95%, at least 98%, at least 99%, or 100%, sequence identity to SEQ ID NO: 88. In some embodiments, the payload molecule is an IL-36γ polypeptide having at least 70%, at least 75%, at least 80%, at least 85%, at least 90%, at least 95%, at least 98%, at least 99%, or 100%, sequence identity to SEQ ID NO: 89.

In some embodiments, the payload molecule is CXCL10. In some embodiments, the payload molecule is a CXCL10 polypeptide having at least 70%, at least 75%, at least 80%, at least 85%, at least 90%, at least 95%, at least 98%, at least 99%, or 100%, sequence identity to SEQ ID NO: 90. In some embodiments, the payload molecule is a CXCL10 polypeptide having at least 70%, at least 75%, at least 80%, at least 85%, at least 90%, at least 95%, at least 98%, at least 99%, or 100%, sequence identity to SEQ ID NO: 91.

In some embodiments, the payload molecule is CCL4. In some embodiments, the payload molecule is a CCL4 polypeptide having at least 70%, at least 75%, at least 80%, at least 85%, at least 90%, at least 95%, at least 98%, at least 99%, or 100%, sequence identity to SEQ ID NO: 92.

In some embodiments, the payload molecule is CCL5. In some embodiments, the payload molecule is a CCL5 polypeptide having at least 70%, at least 75%, at least 80%, at least 85%, at least 90%, at least 95%, at least 98%, at least 99%, or 100%, sequence identity to SEQ ID NO: 93.

In some embodiments, the payload molecule is CCL21. In some embodiments, the payload molecule is a CCL21 polypeptide having at least 70%, at least 75%, at least 80%, at least 85%, at least 90%, at least 95%, at least 98%, at least 99%, or 100%, sequence identity to SEQ ID NO: 94.

In some embodiments, the payload molecule is anti-PD1-VHH-Fc. In some embodiments, the payload molecule is an anti-PD1-VHH-Fc(hIgG4) polypeptide having at least 70%, at least 75%, at least 80%, at least 85%, at least 90%, at least 95%, at least 98%, at least 99%, or 100%, sequence identity to SEQ ID NO: 95.

In some embodiments, the payload molecule is an anti-DLL3 bipartite polypeptide. In some embodiments, the anti-DLL3 bipartite polypeptide is an anti-DLL3 Bi-specific T-cell engager (BiTE). In some embodiments, the anti-DLL3 bipartite polypeptide or anti-DLL3 Bi-specific T-cell engager (BiTE) comprises a first domain capable of binding a cell surface antigen of an effector cell and a second domain capable of binding to DLL3. In some embodiments, the first domain binds to CD3. In some embodiments, the second domain (binding to DLL3) is an scFv or a nanobody (VHH). In some embodiments, the DLL3 binding domain is selected from those described in International PCT Application No. PCT/US2021/030836, which is incorporated herein by reference in its entirety. In some embodiments, the DLL3 antigen comprise an amino acid sequence having at least 70%, at least 75%, at least 80%, at least 85%, at least 90%, at least 95%, at least 98%, at least 99%, or 100%, sequence identity to SEQ ID NO: 96.

In some embodiments, the payload molecule comprises an anti-FAP heavy chain variable region. In some embodiments, the payload molecule comprises an anti-FAP heavy chain variable region polypeptide having at least 70%, at least 75%, at least 80%, at least 85%, at least 90%, at least 95%, at least 98%, at least 99%, or 100%, sequence identity to SEQ ID NO: 97. In some embodiments, the payload molecule comprises an anti-FAP light chain variable region. In some embodiments, the payload molecule comprises an anti-FAP light chain variable region polypeptide having at least 70%, at least 75%, at least 80%, at least 85%, at least 90%, at least 95%, at least 98%, at least 99%, or 100%, sequence identity to SEQ ID NO: 98.

In some embodiments, the payload molecule comprises an anti-CD3 heavy chain variable region. In some embodiments, the payload molecule comprises an anti-CD3 heavy chain variable region polypeptide having at least 70%, at least 75%, at least 80%, at least 85%, at least 90%, at least 95%, at least 98%, at least 99%, or 100%, sequence identity to SEQ ID NO: 99. In some embodiments, the payload molecule comprises an anti-CD3 light chain variable region. In some embodiments, the payload molecule comprises an anti-CD3 light chain variable region polypeptide having at least 70%, at least 75%, at least 80%, at least 85%, at least 90%, at least 95%, at least 98%, at least 99%, or 100%, sequence identity to SEQ ID NO: 100.

In some embodiments, the payload molecule is blinatumomab. In some embodiments, the payload molecule is a blinatumomab-like polypeptide having at least 70%, at least 75%, at least 80%, at least 85%, at least 90%, at least 95%, at least 98%, at least 99%, or 100%, sequence identity to SEQ ID NO: 101.

In some embodiments, the payload molecule is MT 110. In some embodiments, the payload molecule is a MT110-like polypeptide having at least 70%, at least 75%, at least 80%, at least 85%, at least 90%, at least 95%, at least 98%, at least 99%, or 100%, sequence identity to SEQ ID NO: 102.

In some embodiments, the payload molecule is pasotuxizumab. In some embodiments, the payload molecule is a pasotuxizumab-like polypeptide having at least 70%, at least 75%, at least 80%, at least 85%, at least 90%, at least 95%, at least 98%, at least 99%, or 100%, sequence identity to SEQ ID NO: 103.

In some embodiments, the payload molecule is AMG330. In some embodiments, the payload molecule is an AMG330-like polypeptide having at least 70%, at least 75%, at least 80%, at least 85%, at least 90%, at least 95%, at least 98%, at least 99%, or 100%, sequence identity to SEQ ID NO: 104.

In some embodiments, the payload molecule is COVA420 heavy chain. In some embodiments, the payload molecule is a COVA420 heavy chain-like polypeptide having at least 70%, at least 75%, at least 80%, at least 85%, at least 90%, at least 95%, at least 98%, at least 99%, or 100%, sequence identity to SEQ ID NO: 105. In some embodiments, the payload molecule is COVA420 light chain. In some embodiments, the payload molecule is a COVA420 light chain-like polypeptide having at least 70%, at least 75%, at least 80%, at least 85%, at least 90%, at least 95%, at least 98%, at least 99%, or 100%, sequence identity to SEQ ID NO: 106.

In some embodiments, the payload molecule is survivin. In some embodiments, the payload molecule is a survivin polypeptide having at least 70%, at least 75%, at least 80%, at least 85%, at least 90%, at least 95%, at least 98%, at least 99%, or 100%, sequence identity to SEQ ID NO: 107.

In some embodiments, the payload molecule is IFNγ. In some embodiments, the payload molecule is an IFNγ polypeptide having at least 70%, at least 75%, at least 80%, at least 85%, at least 90%, at least 95%, at least 98%, at least 99%, or 100%, sequence identity to SEQ ID NO: 113.

In some embodiments, the payload molecule is IL-15. In some embodiments, the payload molecule is an IL-15 polypeptide having at least 70%, at least 75%, at least 80%, at least 85%, at least 90%, at least 95%, at least 98%, at least 99%, or 100%, sequence identity to SEQ ID NO: 114.

In some embodiments, the payload molecule is IL15R. In some embodiments, the IL15R comprises IL15RA and/or IL15RB. In some embodiments, the IL15RA has at least 70%, at least 75%, at least 80%, at least 85%, at least 90%, at least 95%, at least 98%, at least 99%, or 100%, sequence identity to SEQ ID NO: 115. In some embodiments, the IL15RB polypeptide has at least 70%, at least 75%, at least 80%, at least 85%, at least 90%, at least 95%, at least 98%, at least 99%, or 100%, sequence identity to SEQ ID NO: 116.

In some embodiments, the payload molecule is PGDH. In some embodiments, the payload molecule is a PGDH polypeptide having at least 70%, at least 75%, at least 80%, at least 85%, at least 90%, at least 95%, at least 98%, at least 99%, or 100%, sequence identity to SEQ ID NO: 117.

In some embodiments, the payload molecule is ADA2. In some embodiments, the payload molecule is an ADA2 polypeptide having at least 70%, at least 75%, at least 80%, at least 85%, at least 90%, at least 95%, at least 98%, at least 99%, or 100%, sequence identity to SEQ ID NO: 118.

In some embodiments, the payload molecule is HYAL1. In some embodiments, the payload molecule is an HYAL 1 polypeptide having at least 70%, at least 75%, at least 80%, at least 85%, at least 90%, at least 95%, at least 98%, at least 99%, or 100%, sequence identity to SEQ ID NO: 119.

In some embodiments, the payload molecule is HYAL2. In some embodiments, the payload molecule is an HYAL2 polypeptide having at least 70%, at least 75%, at least 80%, at least 85%, at least 90%, at least 95%, at least 98%, at least 99%, or 100%, sequence identity to SEQ ID NO: 120.

In some embodiments, the payload molecule is MLKL. In some embodiments, the payload molecule is an MLKL polypeptide having at least 70%, at least 75%, at least 80%, at least 85%, at least 90%, at least 95%, at least 98%, at least 99%, or 100%, sequence identity to SEQ ID NO: 121. In some embodiments, the payload molecule comprises or consists of MLKL 41113 domain.

In some embodiments, the payload molecule is GSDMD. In some embodiments, the payload molecule is a GSDMD polypeptide having at least 70%, at least 75%, at least 80%, at least 85%, at least 90%, at least 95%, at least 98%, at least 99%, or 100%, sequence identity to SEQ ID NO: 122. In some embodiments, the payload molecule is a GSDMD 1-233 fragment and/or L192A mutant.

In some embodiments, the payload molecule is GSDME. In some embodiments, the payload molecule is a GSDME polypeptide having at least 70%, at least 75%, at least 80%, at least 85%, at least 90%, at least 95%, at least 98%, at least 99%, or 100%, sequence identity to SEQ ID NO: 123. In some embodiments, the payload molecule is a GSDME 1-237 fragment.

In some embodiments, the payload molecule is HMGB1. In some embodiments, the payload molecule is an HMGB1 polypeptide having at least 70%, at least 75%, at least 80%, at least 85%, at least 90%, at least 95%, at least 98%, at least 99%, or 100%, sequence identity to SEQ ID NO: 124. In some embodiments, the payload molecule comprises or consists of HMGB1 Box B domain.

In some embodiments, the payload molecule is Melittin. In some embodiments, the payload molecule is a Melittin polypeptide having at least 70%, at least 75%, at least 80%, at least 85%, at least 90%, at least 95%, at least 98%, at least 99%, or 100%, sequence identity to SEQ ID NO: 125.

In some embodiments, the payload molecule is SMAC/Diablo. In some embodiments, the payload molecule is an SMAC/Diablo polypeptide having at least 70%, at least 75%, at least 80%, at least 85%, at least 90%, at least 95%, at least 98%, at least 99%, or 100%, sequence identity to SEQ ID NO: 126. In some embodiments, the payload molecule comprises or consists of SMAC/Diablo amino acid 56-239 fragment.

In some embodiments, the payload molecule is Snake LAAO. In some embodiments, the payload molecule is a Snake LAAO polypeptide having at least 70%, at least 75%, at least 80%, at least 85%, at least 90%, at least 95%, at least 98%, at least 99%, or 100%, sequence identity to SEQ ID NO: 127.

In some embodiments, the payload molecule is Leptin. In some embodiments, the payload molecule is a Leptin polypeptide having at least 70%, at least 75%, at least 80%, at least 85%, at least 90%, at least 95%, at least 98%, at least 99%, or 100%, sequence identity to SEQ ID NO: 128.

In some embodiments, the payload molecule is FLT3L. In some embodiments, the payload molecule is a FLT3L polypeptide having at least 70%, at least 75%, at least 80%, at least 85%, at least 90%, at least 95%, at least 98%, at least 99%, or 100%, sequence identity to SEQ ID NO: 129.

In some embodiments, the payload molecule is TRAIL. In some embodiments, the payload molecule is a TRAIL polypeptide having at least 70%, at least 75%, at least 80%, at least 85%, at least 90%, at least 95%, at least 98%, at least 99%, or 100%, sequence identity to SEQ ID NO: 130.

In some embodiments, the payload molecule is MAGEA1. In some embodiments, the payload molecule is an MAGEA1 polypeptide having at least 70%, at least 75%, at least 80%, at least 85%, at least 90%, at least 95%, at least 98%, at least 99%, or 100%, sequence identity to SEQ ID NO: 131.

In some embodiments, the payload molecule is MAGEA3. In some embodiments, the payload molecule is an MAGEA3 polypeptide having at least 70%, at least 75%, at least 80%, at least 85%, at least 90%, at least 95%, at least 98%, at least 99%, or 100%, sequence identity to SEQ ID NO: 132.

In some embodiments, the payload molecule is MAGEA4. In some embodiments, the payload molecule is an MAGEA4 polypeptide having at least 70%, at least 75%, at least 80%, at least 85%, at least 90%, at least 95%, at least 98%, at least 99%, or 100%, sequence identity to SEQ ID NO: 133.

In some embodiments, the payload molecule is MAGEA12. In some embodiments, the payload molecule is an MAGEA12 polypeptide having at least 70%, at least 75%, at least 80%, at least 85%, at least 90%, at least 95%, at least 98%, at least 99%, or 100%, sequence identity to SEQ ID NO: 134.

In some embodiments, the payload molecule is MAGEC2. In some embodiments, the payload molecule is an MAGEC2 polypeptide having at least 70%, at least 75%, at least 80%, at least 85%, at least 90%, at least 95%, at least 98%, at least 99%, or 100%, sequence identity to SEQ ID NO: 135.

In some embodiments, the payload molecule is BAGE1 (B melanoma antigen 1). In some embodiments, the payload molecule is an BAGE1 (B melanoma antigen 1) polypeptide having at least 70%, at least 75%, at least 80%, at least 85%, at least 90%, at least 95%, at least 98%, at least 99%, or 100%, sequence identity to SEQ ID NO: 136.

In some embodiments, the payload molecule is GAGE1 (G antigen 1). In some embodiments, the payload molecule is an GAGE1 (G antigen 1) polypeptide having at least 70%, at least 75%, at least 80%, at least 85%, at least 90%, at least 95%, at least 98%, at least 99%, or 100%, sequence identity to SEQ ID NO: 137.

In some embodiments, the payload molecule is XAGE1B (X antigen family member 1B). In some embodiments, the payload molecule is an XAGE1B (X antigen family member 1B) polypeptide having at least 70%, at least 75%, at least 80%, at least 85%, at least 90%, at least 95%, at least 98%, at least 99%, or 100%, sequence identity to SEQ ID NO: 138.

In some embodiments, the payload molecule is CTAG2 (LAGE1). In some embodiments, the payload molecule is a CTAG2 (LAGE1) polypeptide having at least 70%, at least 75%, at least 80%, at least 85%, at least 90%, at least 95%, at least 98%, at least 99%, or 100%, sequence identity to SEQ ID NO: 139.

In some embodiments, the payload molecule is CTAG1 (NY-ESO-1). In some embodiments, the payload molecule is a CTAG1 (NY-ESO-1) polypeptide having at least 70%, at least 75%, at least 80%, at least 85%, at least 90%, at least 95%, at least 98%, at least 99%, or 100%, sequence identity to SEQ ID NO: 140.

In some embodiments, the payload molecule is SSX2 (synovial sarcoma X breakpoint 2). In some embodiments, the payload molecule is an SSX2 (synovial sarcoma X breakpoint 2) polypeptide having at least 70%, at least 75%, at least 80%, at least 85%, at least 90%, at least 95%, at least 98%, at least 99%, or 100%, sequence identity to SEQ ID NO: 141.

In some embodiments, the payload molecule is KKLC1 (Kita-kyushu lung cancer antigen 1). In some embodiments, the payload molecule is a KKLC1 (Kita-kyushu lung cancer antigen 1) polypeptide having at least 70%, at least 75%, at least 80%, at least 85%, at least 90%, at least 95%, at least 98%, at least 99%, or 100%, sequence identity to SEQ ID NO: 142.

In some embodiments, the payload molecule is SAGE (sarcoma antigen). In some embodiments, the payload molecule is a SAGE (sarcoma antigen) polypeptide having at least 70%, at least 75%, at least 80%, at least 85%, at least 90%, at least 95%, at least 98%, at least 99%, or 100%, sequence identity to SEQ ID NO: 143.

In some embodiments, the payload molecule is SPA17 (sperm autoantigenic protein 17). In some embodiments, the payload molecule is a SPA17 (sperm autoantigenic protein 17) polypeptide having at least 70%, at least 75%, at least 80%, at least 85%, at least 90%, at least 95%, at least 98%, at least 99%, or 100%, sequence identity to SEQ ID NO: 144.

In some embodiments, the payload molecule is Cyclin A. In some embodiments, the payload molecule is a Cyclin A polypeptide having at least 70%, at least 75%, at least 80%, at least 85%, at least 90%, at least 95%, at least 98%, at least 99%, or 100%, sequence identity to SEQ ID NO: 145.

In some embodiments, the payload molecule is KMHN1 (CCDC110). In some embodiments, the payload molecule is a KMHN1 (CCDC110) polypeptide having at least 70%, at least 75%, at least 80%, at least 85%, at least 90%, at least 95%, at least 98%, at least 99%, or 100%, sequence identity to SEQ ID NO: 146.

In some embodiments, the payload molecule is LMP-1. In some embodiments, the payload molecule is a LMP-1 polypeptide having at least 70%, at least 75%, at least 80%, at least 85%, at least 90%, at least 95%, at least 98%, at least 99%, or 100%, sequence identity to SEQ ID NO: 147.

In some embodiments, the payload molecule is LMP-2. In some embodiments, the payload molecule is a LMP-2 polypeptide having at least 70%, at least 75%, at least 80%, at least 85%, at least 90%, at least 95%, at least 98%, at least 99%, or 100%, sequence identity to SEQ ID NO: 148.

In some embodiments, the payload molecule is an antigen that is not encoded by a subject's own genome. In some embodiments, the payload molecule is an antigen that is expressed by a pathogenic microorganism. Pathogenic microorganisms comprise bacteria, viruses, parasites and fungi. In some embodiments, the payload molecule is an antigen from one of the pathogens comprising Dengue virus, Chikungunya virus, Mycobacterium tuberculosis, Human immunodeficiency virus, SARS-CoV-2, Coronavirus, Hepatitis B virus, Togaviridae family virus, Flaviviridae family virus, Influenza A virus, Influenza B virus and a veterinary virus.

### Cleavage Polypeptides

In some embodiments, one or more cleavage polypeptides are operably linked to the payload molecule. The presence of such cleavage polypeptides allows separation of the payload molecule from the rest of the polypeptide encoded by the replicon. In some embodiments, the replicon comprises a heterologous polynucleotide encoding two or more payload molecules operably linked to one or more cleavage polypeptides, which allows separation of the payload molecules. In some embodiments, additional peptide linker (such as a Glycine-Serine linker) may be present between the payload molecule and the cleavage polypeptide.

The cleavage polypeptides comprise 2A family self-cleaving peptides, 3C cleavage site, furin site, IGSF1, and HIV-1 protease site. It shall be noted that more than one cleavage polypeptides can be operably linked to the payload molecule, and different cleavage polypeptides can be used in the same replicon. For example, different cleavage polypeptides can be operably linked to the N-terminus and C-terminus of a payload molecule. In addition, two or more cleavage polypeptides can be joined together or linked consecutively to form a longer cleavage polypeptide which may possess improved cleavage property.

In some embodiments, the cleavage polypeptide comprises or consists of a 2A family self-cleaving peptide. Self-cleaving peptides are found in members of the Picornaviridae virus family, including aphthoviruses such as foot-and-mouth disease virus (FMDV), equine rhinitis A virus (ERAV), Thosea asigna virus (TaV) and porcine teschovirus-1 (PTV-1) (Donnelly, M L, et al., J. Gen. Virol., 82, 1027-101 (2001); Ryan, M D, et al., J. Gen. Virol., 72, 2727-2732 (2001) and cardioviruses such as Theilovirus (e.g., Theiler's murine encephalomyelitis) and encephalomyocarditis viruses. The 2A peptides derived from FMDV, ERAV, PTV-1, and TaV are sometimes referred to herein as “F2A”, “E2A”, “P2A”, and “T2A”, respectively. Aphthovirus 2A polypeptides typically contain a Dx1Ex2NPG (SEQ ID NO: 63) motif, where xl is often valine or isoleucine. Without wishing to be bound by any particular theory, the 2A sequence is believed to mediate ‘ribosomal skipping’ between the proline and glycine, impairing normal peptide bond formation between the P and G without affecting downstream translation. Exemplary 2A self-cleaving peptides can be found in Table 7 below. Additional exemplary 2A self-cleaving peptides can be found in U.S. Pat. No. 9,497,943 and Souza-Moreira et al., FEMS Yeast Res. 2018 Aug. 1; 18(5), which are incorporated by reference herein. In some embodiments, the cleavage polypeptide comprises one of the 2A self-cleaving peptides according to Table 7. In some embodiments, the cleavage polypeptide comprises an amino acid sequence consisting of at most 1, at most 2, at most 3 or at most 4 mutations according to one of the 2A self-cleaving peptides in Table 7.

TABLE 7
Exemplary 2A self-cleaving peptides

| Name | Sequence | SEQ ID NO |
|---|---|---|
| T2A | EGRGSLLTCGDVEEN | 64 |
| | PGP | |
| P2A | ATNFSLLKQAGDVEE | 65 |
| | NPGP | |
| E2A | QCTNYALLKLAGDVE | 66 |
| | SNPGP | |
| F2A | VKQTLNFDLLKLAGD | 67 |
| | VESNPGP | |
| SVV 2A | SGDIETNPGP | 68 |

In some embodiments, the cleavage polypeptide comprises or consists of a SVV 2A self-cleaving peptide. In some embodiments, the SVV 2A self-cleaving peptide has the amino acid sequence of SGDIETNPGP (SEQ ID NO: 68). In some embodiments, the SVV 2A self-cleaving peptide has an amino acid sequence consisting of at most 1, at most 2, or at most 3 mutations according to SGDIETNPGP (SEQ ID NO: 68).

In some embodiments, the cleavage polypeptide comprises or consists of a Coxsackievirus 2A cleavage site. In some embodiments, the Coxsackievirus 2A cleavage site has the amino acid sequence of GFGHQ (SEQ ID NO: 69). In some embodiments, the Coxsackievirus 2A cleavage site has an amino acid sequence consisting of at most 1, at most 2, or at most 3 mutations according to GFGHQ (SEQ ID NO: 69).

In some embodiments, the cleavage polypeptide comprises or consists of 3C cleavage sites. In some embodiments, the 3C cleavage site is a SVV 3C cleavage site having amino acid sequence IVYELQGP (SEQ ID NO: 70). In some embodiments, the 3C cleavage site has an amino acid sequence consisting of at most 1, at most 2, or at most 3 mutations according to IVYELQGP (SEQ ID NO: 70). In some embodiments, the cleavage polypeptide comprises a fusin site and a 3C cleavage site. In some embodiments, the cleavage polypeptide comprises or consists of an amino acid sequence of RRKRIVYELQGP (SEQ ID NO: 71). In some embodiments, the 3C cleavage site has an amino acid sequence consisting of at most 1, at most 2, at most 3 or at most 4 mutations according to RRKRIVYELQGP (SEQ ID NO: 71).

In some embodiments, the cleavage polypeptide comprises or consists of one or more cleavage sites that can be cleaved by a protease produced by a mammalian cell. In some embodiments, the protease is a furin protease. In some embodiments, the cleavage polypeptide comprises or consists of one furin site. In some embodiments, the cleavage polypeptide comprises or consists of two or more furin sites. In some embodiments, the furin site has a consensus sequence of Arg-X-X-Arg (SEQ ID NO: 72). In some embodiments, the furin site has a consensus sequence of Arg-X-Lys/Arg-Arg (SEQ ID NO: 73). In some embodiments, the furin site has the amino acid sequence of RRKR (SEQ ID NO: 74). In some embodiments, the cleavage polypeptide comprises one or more GS linker (amino acid sequence Gly-Ser). In some embodiments, the cleavage polypeptide comprises one or more GSG linkers (amino acid sequence Gly-Ser-Gly). In some embodiments, the cleavage polypeptide adopts the configuration of “GSG linker-2A peptide”. In some embodiments, the cleavage polypeptide adopts the configuration of “furin site-2A peptide”. In some embodiments, the cleavage polypeptide adopts the configuration of “furin site-GSG linker-2A peptide”.

In some embodiments, the cleavage polypeptide comprises, or consists of, an IGSF1 polypeptide. In some embodiments, the IGSF1 polypeptide comprises or consists of the amino acid sequence of NEAIRLSLIMQLVALLLVVLWIRWKCRRLRIREAWLLGTAQGVTMLFIVTALLCCGLCNG (SEQ ID NO: 75). In some embodiments, the IGSF1 polypeptide comprises or consists of an amino acid sequence having at least 70%, at least 75%, at least 80%, at least 85%, at least 90%, at least 95%, or at least 98% identity to SEQ ID NO: 75. In some embodiments, the cleavage polypeptide comprises one or more furin sites in addition to the IGSF1 polypeptide. In some embodiments, the cleavage polypeptide comprises, or consists of, a furin-site containing peptide having an amino acid sequence of GSRRKRGSRRKRGS (SEQ ID NO: 76). In some embodiments, the cleavage polypeptide comprises, or consists of, the amino acid sequence of GSRRKRGSRRKRGSNEAIRLSLIMQLVALLLVVLWIRWKCRRLRIREAWLLGTAQGVTMLFI VTALLCCGLCNG (SEQ ID NO: 77). In some embodiments, the cleavage polypeptide comprises, or consist of, an amino acid sequence having at least 70%, at least 75%, at least 80%, at least 85%, at least 90%, at least 95%, or at least 98% identity to SEQ ID NO: 77. In some embodiments, two of the payload molecules are operably linked to a cleavage polypeptide comprising an IGSF polypeptide. In some embodiments, two of the payload molecules are operably linked to a cleavage polypeptide comprising an IGSF polypeptide and one or more furin sites. In some embodiments, two of the payload molecules are operably linked to a cleavage polypeptide comprising or consisting of the amino acid sequence of SEQ ID NO: 77.

In some embodiments, the cleavage polypeptide comprises or consists of one or more cleavage site that can be recognized by a non-mammalian protease. In some embodiments, the non-mammalian protease is an HIV protease. In some embodiments, the cleavage polypeptide comprises, or consists of, an HIV protease site. In some embodiments, the HIV protease site comprises or consists of a PR cleavage sequence having the amino acid sequence of IFLETS (SEQ ID NO: 78). In some embodiments, the HIV protease site comprises or consists of a PR cleavage sequence having at most one, at most two, or at most three mutations or conservative mutations according to IFLETS (SEQ ID NO: 78). In some embodiments, the cleavage polypeptide comprises a GS linker and a PR cleavage sequence. In some embodiments, the cleavage polypeptide comprises, or consists of, an amino acid sequence of GSGIFLETS (SEQ ID NO: 79). In some embodiments, the cleavage polypeptide comprises, or consists of, an amino acid sequence having at most one, at most two, at most three or at most four mutations or conservative mutations according to GSGIFLETS (SEQ ID NO: 79).

In some embodiments, the heterologous nucleic acid comprises an HIV protease coding sequence. In some embodiments, the HIV protease comprises or consists of an amino acid sequence having at least 70%, at least 75%, at least 80%, at least 85%, at least 90%, at least 95%, at least 98% identity, at least 99% identity or 100% identity to SEQ ID NO: 80:

(SEQ ID NO: 80)
QITLWQRPLVTIKIGGQLKEALLDTGADDTV
LEEMSLPGRWKPKMIGGIGGFIKVRQYDQIL
IEICGHKAIGTVLVGPTPVNIIGRNLLTQIG
CTLNF

In some embodiments, the heterologous polynucleotide comprises a coding region that encodes a payload molecule operably linked to one or more cleavage polypeptides. In some embodiments, the payload molecule is operably linked to two cleavage polypeptides. In some embodiments, at least one cleavage polypeptide flanks the N-terminus of the payload molecule and/or at least one cleavage polypeptide flanks the C-terminus of the payload molecule. In some embodiments, the cleavage peptides and the payload molecule adopt the configuration of:

N′-cleavage polypeptide 1-payload molecule-cleavage polypeptide 2-C′.

In some embodiments, additional cleavage polypeptide may be present at the N′ and/or C′ terminus of the configuration described above in this paragraph. In some embodiments, the additional cleavage polypeptide comprises a 2A self-cleaving peptide. In some embodiments, the cleavage polypeptide 2 at the C-terminus comprises or consists of a T2A self-cleaving peptide. In some embodiments, the cleavage polypeptide 1 at the N-terminus comprises or consists of a 2A self-cleaving peptide. In some embodiments, additional peptide linker (such as a Glycine-Serine linker) may be present between the payload molecule and the cleavage polypeptide.

### Co-Expression of Multiple Payload Molecules

In some embodiments, the disclosure provides recombinant RNA replicons comprising heterologous nucleotides encoding two or more payload molecules. In some embodiments, the recombinant RNA replicon of the present disclosure enables expression of two or more payload molecules from one replicon.

In some embodiments, the two or more payload molecules are encoded by a continuous heterologous polynucleotide. In some embodiments, at least one of the payload molecules is encoded by a second heterologous polynucleotide. In some embodiments, the two or more heterologous polynucleotide are inserted into different locations of the viral genome.

In some embodiments, at least one of the payload molecules is a secreted protein. In some embodiments, the secreted protein comprises a native signal peptide or a non-native signal peptide. In some embodiments, two of the payload molecules are secreted proteins. In some embodiments, at least two of the payload molecules are secreted proteins. In some embodiments, all of the payload molecules are secreted proteins. In some embodiments, at least one of the payload molecules is a secreted protein comprising a native signal peptide sequence for secretion. In some embodiments, at least one of the payload molecules is a secreted protein comprising a non-native signal peptide sequence for secretion. In some embodiments, at least one of the payload molecules is a secreted protein without signal peptide sequence.

In some embodiments, each of the payload molecule is operably linked to a cleavage polypeptide at its C-terminus. In some embodiments, each of the payload molecule is operably linked to cleavage polypeptides at both its N-terminus and its C-terminus.

In some embodiments, the heterologous polynucleotide comprises a coding region that encodes two or more payload molecules operably linked to a cleavage polypeptide. In some embodiments, the two or more payload molecules and the cleavage polypeptide adopts the configuration of:

N′-payload molecule 1-cleavage polypeptide-payload molecule 2-C′.

In some embodiments, additional cleavage polypeptide may be present at the N′ and/or C′ terminus of the configuration described above in this paragraph. In some embodiments, the additional cleavage polypeptide comprises a 2A self-cleaving peptide. In some embodiments, a T2A self-cleaving peptide flanks the C-terminus of payload molecule 2. In some embodiments, additional peptide linker (such as a Glycine-Serine linker) may be present between the payload molecule and the cleavage polypeptide.

In some embodiments, the heterologous polynucleotide comprises a coding region that encodes two payload molecules operably linked to a cleavage polypeptide comprising or consisting of an IGSF polypeptide. In some embodiments, the IGSF1 polypeptide has the amino acid sequence of:

(SEQ ID NO: 75)
NEAIRLSLIMQLVALLLVVLWIRWKCRRLR
IREAWLLGTAQGVTMLFIVTALLCCGLCNG.

In some embodiments, the IGSF1 polypeptide has at least 70%, at least 75%, at least 80%, at least 85%, at least 90%, at least 95%, or at least 98% identity to SEQ ID NO: 75. In some embodiments, the cleavage polypeptide comprises a furin-site containing peptide having an amino acid sequence of GSRRKRGSRRKRGS (SEQ ID NO: 76). In some embodiments, the cleavage polypeptide comprises, or consists of, an amino acid sequence of:

(SEQ ID NO: 77)
GSRRKRGSRRKRGSNEAIRLSLIMQLVALLLVVLWIR
WKCRRLRIREAWLLGTAQGVTMLFIVTALLCCGLCNG.

In some embodiments, the cleavage polypeptide comprises, or consist of, an amino acid sequence having at least 70%, at least 75%, at least 80%, at least 85%, at least 90%, at least 95%, or at least 98% identity to SEQ ID NO: 77.

In some embodiments, the heterologous polynucleotide comprises a coding region that encodes two payload molecules operably linked to a cleavage polypeptide comprising or consisting of one or more HIV protease site. In some embodiments, the HIV protease site comprises or consists of a PR cleavage sequence having the amino acid sequence of IFLETS (SEQ ID NO: 78), or an amino acid sequence having at most one, at most two, or at most three mutations or conservative mutations according to IFLETS (SEQ ID NO: 78). In some embodiments, the cleavage polypeptide comprises a GS linker and a PR cleavage sequence. In some embodiments, the cleavage polypeptide comprises, or consists of, an amino acid sequence of GSGIFLETS (SEQ ID NO: 79). In some embodiments, the cleavage polypeptide comprises, or consists of, an amino acid sequence having at most one, at most two, at most three or at most four mutations or conservative mutations according to GSGIFLETS (SEQ ID NO: 79).

In some embodiments, the heterologous nucleic acid further comprises an HIV protease coding region. In some embodiments, the HIV protease is operably linked to the one or more payload molecule by a cleavage polypeptide comprising or consisting of an HIV protease sites. In some embodiments, the HIV protease is located in between two payload molecule.

In some embodiments, the heterologous nucleic acid comprises a coding region that encodes two payload molecules and the HIV protease. In some embodiments, the heterologous nucleic acid comprises a coding region that encodes a polypeptide adopting the configuration of:

N′-Payload molecule 1-HIV protease site-HIV protease-HIV protease site-Payload molecule 2-C′.

In some embodiments, additional cleavage polypeptide maybe present at the N′ and/or C′ terminus of the configuration described above in this paragraph. In some embodiments, the additional cleavage polypeptide comprises an HIV protease site. In some embodiments, the additional cleavage polypeptide comprises a 2A self-cleaving peptide. In some embodiments, a T2A self-cleaving peptide flanks the C-terminus of payload molecule 2. In some embodiments, additional peptide linker (such as a Glycine-Serine linker) may be present between the payload molecule and the HIV protease site.

In some embodiments, the heterologous nucleic acid comprises a coding region that encodes three payload molecules and the HIV protease. In some embodiments, the heterologous nucleic acid comprises a coding region that encodes a polypeptide adopting the configuration of:

N′-Payload molecule 1-HIV protease site-HIV protease-HIV protease site-Payload molecule 2-HIV protease site-Payload molecule 3-C′.

In some embodiments, additional cleavage polypeptides maybe present at the N′ and/or C′ terminus of the configuration described above in this paragraph. In some embodiments, the additional cleavage polypeptide comprises an HIV protease site. In some embodiments, the additional cleavage polypeptide comprises a 2A self-cleaving peptide. In some embodiments, a T2A self-cleaving peptide flanks the C-terminus of payload molecule 3. In some embodiments, additional peptide linker (such as a Glycine-Serine linker) may be present between the payload molecule and the HIV protease site.

In some embodiments, the two or more payload molecules are selected from the group consisting of a fluorescent protein, an enzyme, a cytokine, a chemokine, an antigen-binding molecule capable of binding to a cell surface receptor, and a ligand for a cell-surface receptor. In some embodiments, at least one of the payload molecules is a secreted protein. In some embodiments, the two or more payload molecules are secreted proteins. In some embodiments, the payload molecules are selected from the payload molecules described in the “Heterologous Polynucleotide and Payload Molecules” section of the present disclosure.

In some embodiments, the two or more payload molecules comprise:
a. IL-2 and IL-36γ;
b. CXCL10 and an antigen binding molecule binding to FAP and CD3;
c. IL-2 and an antigen binding molecule binding to DLL3 and CD3;
d. IL-36γ and an antigen binding molecule binding to DLL3 and CD3; or e. IL-2, IL-36γ and an antigen binding molecule binding to DLL3 and CD3

In some embodiments, the two or more payload molecules comprise anti-DLL3 bipartite polypeptide, anti-FAP bipartite polypeptide, anti-PD1-VHH-Fc antibody, IL-2, IL-12, IL-18, L-23, IL-36γ, CCL21, CXCL10, or any combinations thereof. In some embodiments, the anti-DLL3 bipartite polypeptide is an anti-DLL3/anti-CD3 bipartite polypeptide. In some embodiments, the anti-FAP bipartite polypeptide is an anti-FAP/anti-CD3 bipartite polypeptide.

In some embodiments, the replicon of the disclosure, or the heterologous polynucleotide of the replicon, comprises coding region for two, or at least two payload molecules according to one of the payload molecule combinations listed in Table 8 below. In some embodiments, the replicon is a SVV derived replicon. In some embodiments, the replicon is a CVA21 derived replicon. Each combination of two payload molecules is marked as “x” according to Table 8 below. Those “x” marked combinations in Table 8 that have the same payload molecules indicate that the replicon comprises two copies of the payload molecule.

TABLE 8
Two Payload Molecule Combinations

| | | GM- | | | | | | | | | |
|---|---|---|---|---|---|---|---|---|---|---|---|
| | IFNg | CSF | IL-2 | IL-12 | IL-15 | IL-18 | IL-23 | IL-36γ | CXCL10 | CCL4 | CCL5 |
| IFNg | X | X | X | X | X | X | X | X | X | X | X |
| GM-CSF | X | X | X | X | X | X | X | X | X | X | X |
| IL-2 | X | X | X | X | X | X | X | X | X | X | X |
| IL-12 | X | X | X | X | X | X | X | X | X | X | X |
| IL-15 | X | X | X | X | X | X | X | X | X | X | X |
| IL-18 | X | X | X | X | X | X | X | X | X | X | X |
| IL-23 | X | X | X | X | X | X | X | X | X | X | X |
| IL-36γ | X | X | X | X | X | X | X | X | X | X | X |
| CXCL10 | X | X | X | X | X | X | X | X | X | X | X |
| CCL4 | X | X | X | X | X | X | X | X | X | X | X |
| CCL5 | X | X | X | X | X | X | X | X | X | X | X |
| CCL21 | X | X | X | X | X | X | X | X | X | X | X |
| anti-PD1- | X | X | X | X | X | X | X | X | X | X | X |
| VHH-Fc | | | | | | | | | | | |
| anti-TGFb- | X | X | X | X | X | X | X | X | X | X | X |
| VHH(or | | | | | | | | | | | |
| scFv)-Fc | | | | | | | | | | | |
| anti-CD47- | X | X | X | X | X | X | X | X | X | X | X |
| VHH-Fc | | | | | | | | | | | |
| anti-DLL3 | X | X | X | X | X | X | X | X | X | X | X |
| bipartite | | | | | | | | | | | |
| polypeptide | | | | | | | | | | | |
| anti-FAP | X | X | X | X | X | X | X | X | X | X | X |
| bipartite | | | | | | | | | | | |
| polypeptide | | | | | | | | | | | |
| anti-EpCAM | X | X | X | X | X | X | X | X | X | X | X |
| bipartite | | | | | | | | | | | |
| polypeptide | | | | | | | | | | | |
| Survivin | X | X | X | X | X | X | X | X | X | X | X |
| MAGE family | X | X | X | X | X | X | X | X | X | X | X |
| protein | | | | | | | | | | | |
| IL15R | X | X | X | X | X | X | X | X | X | X | X |
| PGDH | X | X | X | X | X | X | X | X | X | X | X |
| ADA | X | X | X | X | X | X | X | X | X | X | X |
| ADA2 | X | X | X | X | X | X | X | X | X | X | X |
| HYAL1 | X | X | X | X | X | X | X | X | X | X | X |
| HYAL2 | X | X | X | X | X | X | X | X | X | X | X |
| CHIPS | X | X | X | X | X | X | X | X | X | X | X |
| MLKL (or its | X | X | X | X | X | X | X | X | X | X | X |
| 4HB domain | | | | | | | | | | | |
| only) | | | | | | | | | | | |
| GSDMD (or | X | X | X | X | X | X | X | X | X | X | X |
| its L192A | | | | | | | | | | | |
| mutant, or its | | | | | | | | | | | |
| amino acids | | | | | | | | | | | |
| 1-233 | | | | | | | | | | | |
| fragment, or | | | | | | | | | | | |
| its amino | | | | | | | | | | | |
| acids 1-233 | | | | | | | | | | | |
| fragment with | | | | | | | | | | | |
| L192A | | | | | | | | | | | |
| mutation) | | | | | | | | | | | |
| GSDME (or | X | X | X | X | X | X | X | X | X | X | X |
| its amino acid | | | | | | | | | | | |
| 1-237 | | | | | | | | | | | |
| fragment) | | | | | | | | | | | |
| HMGB1 (or | X | X | X | X | X | X | X | X | X | X | X |
| its Box B | | | | | | | | | | | |
| domain only) | | | | | | | | | | | |
| Melittin (e.g., | X | X | X | X | X | X | X | X | X | X | X |
| alpha- | | | | | | | | | | | |
| Melittin) | | | | | | | | | | | |
| SMAC/Diablo | X | X | X | X | X | X | X | X | X | X | X |
| (or its amino | | | | | | | | | | | |
| acid 56-239 | | | | | | | | | | | |
| fragment) | | | | | | | | | | | |
| Snake LAAO | X | X | X | X | X | X | X | X | X | X | X |
| Snake | X | X | X | X | X | X | X | X | X | X | X |
| disintegrin | | | | | | | | | | | |
| Leptin | X | X | X | X | X | X | X | X | X | X | X |
| FLT3L | X | X | X | X | X | X | X | X | X | X | X |
| TRAIL | X | X | X | X | X | X | X | X | X | X | X |
| Gasdermin D | X | X | X | X | X | X | X | X | X | X | X |
| or a truncation | | | | | | | | | | | |
| thereof | | | | | | | | | | | |
| Gasdermin E | X | X | X | X | X | X | X | X | X | X | X |
| or a truncation | | | | | | | | | | | |
| thereof | | | | | | | | | | | |
| Tumor | X | X | X | X | X | X | X | X | X | X | X |
| associated | | | | | | | | | | | |
| antigen | | | | | | | | | | | |
| Tumor | X | X | X | X | X | X | X | X | X | X | X |
| neoantigen | | | | | | | | | | | |
| Bipartite | X | X | X | X | X | X | X | X | X | X | X |
| polypeptide | | | | | | | | | | | |
| binding to | | | | | | | | | | | |
| MHC-peptide | | | | | | | | | | | |
| antigen | | | | | | | | | | | |
| complex | | | | | | | | | | | |
| Fusogenic | X | X | X | X | X | X | X | X | X | X | X |
| protein | | | | | | | | | | | |

| | | anti- | anti- | anti- | | | anti- | |
|---|---|---|---|---|---|---|---|---|
| | | PD1- | TGFb- | CD47- | anti-DLL3 | anti-FAP | EpCAM | |
| | | VHH- | VHH(or | VHH- | bipartite | bipartite | bipartite | |
| | CCL21 | Fc | scFv)-Fc | Fc | polypeptide | polypeptide | polypeptide | Survivin |
| IFNg | X | X | X | X | X | X | X | X |
| GM-CSF | X | X | X | X | X | X | X | X |
| IL-2 | X | X | X | X | X | X | X | X |
| IL-12 | X | X | X | X | X | X | X | X |
| IL-15 | X | X | X | X | X | X | X | X |
| IL-18 | X | X | X | X | X | X | X | X |
| IL-23 | X | X | X | X | X | X | X | X |
| IL-36γ | X | X | X | X | X | X | X | X |
| CXCL10 | X | X | X | X | X | X | X | X |
| CCL4 | X | X | X | X | X | X | X | X |
| CCL5 | X | X | X | X | X | X | X | X |
| CCL21 | X | X | X | X | X | X | X | X |
| anti-PD1- | X | X | X | X | X | X | X | X |
| VHH-Fc | | | | | | | | |
| anti-TGFb- | X | X | X | X | X | X | X | X |
| VHH(or | | | | | | | | |
| scFv)-Fc | | | | | | | | |
| anti-CD47- | X | X | X | X | X | X | X | X |
| VHH-Fc | | | | | | | | |
| anti-DLL3 | X | X | X | X | X | X | X | X |
| bipartite | | | | | | | | |
| polypeptide | | | | | | | | |
| anti-FAP | X | X | X | X | X | X | X | X |
| bipartite | | | | | | | | |
| polypeptide | | | | | | | | |
| anti-EpCAM | X | X | X | X | X | X | X | X |
| bipartite | | | | | | | | |
| polypeptide | | | | | | | | |
| Survivin | X | X | X | X | X | X | X | X |
| MAGE family | X | X | X | X | X | X | X | X |
| protein | | | | | | | | |
| IL15R | X | X | X | X | X | X | X | X |
| PGDH | X | X | X | X | X | X | X | X |
| ADA | X | X | X | X | X | X | X | X |
| ADA2 | X | X | X | X | X | X | X | X |
| HYAL1 | X | X | X | X | X | X | X | X |
| HYAL2 | X | X | X | X | X | X | X | X |
| CHIPS | X | X | X | X | X | X | X | X |
| MLKL (or its | X | X | X | X | X | X | X | X |
| 4HB domain | | | | | | | | |
| only) | | | | | | | | |
| GSDMD (or | X | X | X | X | X | X | X | X |
| its L192A | | | | | | | | |
| mutant, or its | | | | | | | | |
| amino acids | | | | | | | | |
| 1-233 | | | | | | | | |
| fragment, or | | | | | | | | |
| its amino | | | | | | | | |
| acids 1-233 | | | | | | | | |
| fragment with | | | | | | | | |
| L192A | | | | | | | | |
| mutation) | | | | | | | | |
| GSDME (or | X | X | X | X | X | X | X | X |
| its amino acid | | | | | | | | |
| 1-237 | | | | | | | | |
| fragment) | | | | | | | | |
| HMGB1 (or | X | X | X | X | X | X | X | X |
| its Box B | | | | | | | | |
| domain only) | | | | | | | | |
| Melittin (e.g., | X | X | X | X | X | X | X | X |
| alpha- | | | | | | | | |
| Melittin) | | | | | | | | |
| SMAC/Diablo | X | X | X | X | X | X | X | X |
| (or its amino | | | | | | | | |
| acid 56-239 | | | | | | | | |
| fragment) | | | | | | | | |
| Snake LAAO | X | X | X | X | X | X | X | X |
| Snake | X | X | X | X | X | X | X | X |
| disintegrin | | | | | | | | |
| Leptin | X | X | X | X | X | X | X | X |
| FLT3L | X | X | X | X | X | X | X | X |
| TRAIL | X | X | X | X | X | X | X | X |
| Gasdermin D | X | X | X | X | X | X | X | X |
| or a truncation | | | | | | | | |
| thereof | | | | | | | | |
| Gasdermin E | X | X | X | X | X | X | X | X |
| or a truncation | | | | | | | | |
| thereof | | | | | | | | |
| Tumor | X | X | X | X | X | X | X | X |
| associated | | | | | | | | |
| antigen | | | | | | | | |
| Tumor | X | X | X | X | X | X | X | X |
| neoantigen | | | | | | | | |
| Bipartite | X | X | X | X | X | X | X | X |
| polypeptide | | | | | | | | |
| binding to | | | | | | | | |
| MHC-peptide | | | | | | | | |
| antigen | | | | | | | | |
| complex | | | | | | | | |
| Fusogenic | X | X | X | X | X | X | X | X |
| protein | | | | | | | | |

| | MAGE | | | | | | | | MLKL |
|---|---|---|---|---|---|---|---|---|---|
| | family | | | | | | | | (or its 4HB |
| | protein | IL15R | PGDH | ADA | ADA2 | HYAL1 | HYAL2 | CHIPS | domain only) |
| IFNg | X | X | X | X | X | X | X | X | X |
| GM-CSF | X | X | X | X | X | X | X | X | X |
| IL-2 | X | X | X | X | X | X | X | X | X |
| IL-12 | X | X | X | X | X | X | X | X | X |
| IL-15 | X | X | X | X | X | X | X | X | X |
| IL-18 | X | X | X | X | X | X | X | X | X |
| IL-23 | X | X | X | X | X | X | X | X | X |
| IL-36γ | X | X | X | X | X | X | X | X | X |
| CXCL10 | X | X | X | X | X | X | X | X | X |
| CCL4 | X | X | X | X | X | X | X | X | X |
| CCL5 | X | X | X | X | X | X | X | X | X |
| CCL21 | X | X | X | X | X | X | X | X | X |
| anti-PD1- | X | X | X | X | X | X | X | X | X |
| VHH-Fc | | | | | | | | | |
| anti-TGFb- | X | X | X | X | X | X | X | X | X |
| VHH(or | | | | | | | | | |
| scFv)-Fc | | | | | | | | | |
| anti-CD47- | X | X | X | X | X | X | X | X | X |
| VHH-Fc | | | | | | | | | |
| anti-DLL3 | X | X | X | X | X | X | X | X | X |
| bipartite | | | | | | | | | |
| polypeptide | | | | | | | | | |
| anti-FAP | X | X | X | X | X | X | X | X | X |
| bipartite | | | | | | | | | |
| polypeptide | | | | | | | | | |
| anti-EpCAM | X | X | X | X | X | X | X | X | X |
| bipartite | | | | | | | | | |
| polypeptide | | | | | | | | | |
| Survivin | X | X | X | X | X | X | X | X | X |
| MAGE family | X | X | X | X | X | X | X | X | X |
| protein | | | | | | | | | |
| IL15R | X | X | X | X | X | X | X | X | X |
| PGDH | X | X | X | X | X | X | X | X | X |
| ADA | X | X | X | X | X | X | X | X | X |
| ADA2 | X | X | X | X | X | X | X | X | X |
| HYAL1 | X | X | X | X | X | X | X | X | X |
| HYAL2 | X | X | X | X | X | X | X | X | X |
| CHIPS | X | X | X | X | X | X | X | X | X |
| MLKL (or its | X | X | X | X | X | X | X | X | X |
| 4HB domain | | | | | | | | | |
| only) | | | | | | | | | |
| GSDMD (or | X | X | X | X | X | X | X | X | X |
| its L192A | | | | | | | | | |
| mutant, or its | | | | | | | | | |
| amino acids | | | | | | | | | |
| 1-233 | | | | | | | | | |
| fragment, or | | | | | | | | | |
| its amino | | | | | | | | | |
| acids 1-233 | | | | | | | | | |
| fragment with | | | | | | | | | |
| L192A | | | | | | | | | |
| mutation) | | | | | | | | | |
| GSDME (or | X | X | X | X | X | X | X | X | X |
| its amino acid | | | | | | | | | |
| 1-237 | | | | | | | | | |
| fragment) | | | | | | | | | |
| HMGB1 (or | X | X | X | X | X | X | X | X | X |
| its Box B | | | | | | | | | |
| domain only) | | | | | | | | | |
| Melittin (e.g., | X | X | X | X | X | X | X | X | X |
| alpha- | | | | | | | | | |
| Melittin) | | | | | | | | | |
| SMAC/Diablo | X | X | X | X | X | X | X | X | X |
| (or its amino | | | | | | | | | |
| acid 56-239 | | | | | | | | | |
| fragment) | | | | | | | | | |
| Snake LAAO | X | X | X | X | X | X | X | X | X |
| Snake | X | X | X | X | X | X | X | X | X |
| disintegrin | | | | | | | | | |
| Leptin | X | X | X | X | X | X | X | X | X |
| FLT3L | X | X | X | X | X | X | X | X | X |
| TRAIL | X | X | X | X | X | X | X | X | X |
| Gasdermin D | X | X | X | X | X | X | X | X | X |
| or a truncation | | | | | | | | | |
| thereof | | | | | | | | | |
| Gasdermin E | X | X | X | X | X | X | X | X | X |
| or a truncation | | | | | | | | | |
| thereof | | | | | | | | | |
| Tumor | X | X | X | X | X | X | X | X | X |
| associated | | | | | | | | | |
| antigen | | | | | | | | | |
| Tumor | X | X | X | X | X | X | X | X | X |
| neoantigen | | | | | | | | | |
| Bipartite | X | X | X | X | X | X | X | X | X |
| polypeptide | | | | | | | | | |
| binding to | | | | | | | | | |
| MHC-peptide | | | | | | | | | |
| antigen | | | | | | | | | |
| complex | | | | | | | | | |
| Fusogenic | X | X | X | X | X | X | X | X | X |
| protein | | | | | | | | | |

| | GSDMD (or | | | | | | | |
|---|---|---|---|---|---|---|---|---|
| | its L192A | | | | | | | |
| | mutant, or | | | | | | | |
| | its amino | | | | | | | |
| | acids 1-233 | | | | | | | |
| | fragment, | | | | SMAC/ | | | |
| | or its amino | | HMGB1 | | Diablo | | | |
| | acids 1-233 | GSDME | (or its | Melittin | (or its | | | |
| | fragment | (or its amino | Box B | (e.g., | amino acid | | Snake | |
| | with L192A | acid 1-237 | domain | alpha- | 56-239 | Snake | dis- | |
| | mutation) | fragment) | only) | Melittin) | fragment) | LAAO | integrin | Leptin |
| IFNg | X | X | X | X | X | X | X | X |
| GM-CSF | X | X | X | X | X | X | X | X |
| IL-2 | X | X | X | X | X | X | X | X |
| IL-12 | X | X | X | X | X | X | X | X |
| IL-15 | X | X | X | X | X | X | X | X |
| IL-18 | X | X | X | X | X | X | X | X |
| IL-23 | X | X | X | X | X | X | X | X |
| IL-36γ | X | X | X | X | X | X | X | X |
| CXCL10 | X | X | X | X | X | X | X | X |
| CCL4 | X | X | X | X | X | X | X | X |
| CCL5 | X | X | X | X | X | X | X | X |
| CCL21 | X | X | X | X | X | X | X | X |
| anti-PD1- | X | X | X | X | X | X | X | X |
| VHH-Fc | | | | | | | | |
| anti-TGFb- | X | X | X | X | X | X | X | X |
| VHH(or | | | | | | | | |
| scFv)-Fc | | | | | | | | |
| anti-CD47- | X | X | X | X | X | X | X | X |
| VHH-Fc | | | | | | | | |
| anti-DLL3 | X | X | X | X | X | X | X | X |
| bipartite | | | | | | | | |
| polypeptide | | | | | | | | |
| anti-FAP | X | X | X | X | X | X | X | X |
| bipartite | | | | | | | | |
| polypeptide | | | | | | | | |
| anti-EpCAM | X | X | X | X | X | X | X | X |
| bipartite | | | | | | | | |
| polypeptide | | | | | | | | |
| Survivin | X | X | X | X | X | X | X | X |
| MAGE family | X | X | X | X | X | X | X | X |
| protein | | | | | | | | |
| IL15R | X | X | X | X | X | X | X | X |
| PGDH | X | X | X | X | X | X | X | X |
| ADA | X | X | X | X | X | X | X | X |
| ADA2 | X | X | X | X | X | X | X | X |
| HYAL1 | X | X | X | X | X | X | X | X |
| HYAL2 | X | X | X | X | X | X | X | X |
| CHIPS | X | X | X | X | X | X | X | X |
| MLKL (or its | X | X | X | X | X | X | X | X |
| 4HB domain | | | | | | | | |
| only) | | | | | | | | |
| GSDMD (or | X | X | X | X | X | X | X | X |
| its L192A | | | | | | | | |
| mutant, or its | | | | | | | | |
| amino acids | | | | | | | | |
| 1-233 | | | | | | | | |
| fragment, or | | | | | | | | |
| its amino | | | | | | | | |
| acids 1-233 | | | | | | | | |
| fragment with | | | | | | | | |
| L192A | | | | | | | | |
| mutation) | | | | | | | | |
| GSDME (or | X | X | X | X | X | X | X | X |
| its amino acid | | | | | | | | |
| 1-237 | | | | | | | | |
| fragment) | | | | | | | | |
| HMGB1 (or | X | X | X | X | X | X | X | X |
| its Box B | | | | | | | | |
| domain only) | | | | | | | | |
| Melittin (e.g., | X | X | X | X | X | X | X | X |
| alpha- | | | | | | | | |
| Melittin) | | | | | | | | |
| SMAC/Diablo | X | X | X | X | X | X | X | X |
| (or its amino | | | | | | | | |
| acid 56-239 | | | | | | | | |
| fragment) | | | | | | | | |
| Snake LAAO | X | X | X | X | X | X | X | X |
| Snake | X | X | X | X | X | X | X | X |
| disintegrin | | | | | | | | |
| Leptin | X | X | X | X | X | X | X | X |
| FLT3L | X | X | X | X | X | X | X | X |
| TRAIL | X | X | X | X | X | X | X | X |
| Gasdermin D | X | X | X | X | X | X | X | X |
| or a truncation | | | | | | | | |
| thereof | | | | | | | | |
| Gasdermin E | X | X | X | X | X | X | X | X |
| or a truncation | | | | | | | | |
| thereof | | | | | | | | |
| Tumor | X | X | X | X | X | X | X | X |
| associated | | | | | | | | |
| antigen | | | | | | | | |
| Tumor | X | X | X | X | X | X | X | X |
| neoantigen | | | | | | | | |
| Bipartite | X | X | X | X | X | X | X | X |
| polypeptide | | | | | | | | |
| binding to | | | | | | | | |
| MHC-peptide | | | | | | | | |
| antigen | | | | | | | | |
| complex | | | | | | | | |
| Fusogenic | X | X | X | X | X | X | X | X |
| protein | | | | | | | | |

| | | | | | | | Bipartite | |
|---|---|---|---|---|---|---|---|---|
| | | | | | | | polypeptide | |
| | | | | | | | binding to | |
| | | | Gasdermin | Gasdermin | | | MHC- | |
| | | | D or a | E or a | Tumor | Tumor | peptide | |
| | | | truncation | truncation | associated | neo- | antigen | Fusogenic |
| | FLT3L | TRAIL | thereof | thereof | antigen | antigen | complex | protein |
| IFNg | X | X | X | X | X | X | X | X |
| GM-CSF | X | X | X | X | X | X | X | X |
| IL-2 | X | X | X | X | X | X | X | X |
| IL-12 | X | X | X | X | X | X | X | X |
| IL-15 | X | X | X | X | X | X | X | X |
| IL-18 | X | X | X | X | X | X | X | X |
| IL-23 | X | X | X | X | X | X | X | X |
| IL-36γ | X | X | X | X | X | X | X | X |
| CXCL10 | X | X | X | X | X | X | X | X |
| CCL4 | X | X | X | X | X | X | X | X |
| CCL5 | X | X | X | X | X | X | X | X |
| CCL21 | X | X | X | X | X | X | X | X |
| anti-PD1- | X | X | X | X | X | X | X | X |
| VHH-Fc | | | | | | | | |
| anti-TGFb- | X | X | X | X | X | X | X | X |
| VHH(or | | | | | | | | |
| scFv)-Fc | | | | | | | | |
| anti-CD47- | X | X | X | X | X | X | X | X |
| VHH-Fc | | | | | | | | |
| anti-DLL3 | X | X | X | X | X | X | X | X |
| bipartite | | | | | | | | |
| polypeptide | | | | | | | | |
| anti-FAP | X | X | X | X | X | X | X | X |
| bipartite | | | | | | | | |
| polypeptide | | | | | | | | |
| anti-EpCAM | X | X | X | X | X | X | X | X |
| bipartite | | | | | | | | |
| polypeptide | | | | | | | | |
| Survivin | X | X | X | X | X | X | X | X |
| MAGE family | X | X | X | X | X | X | X | X |
| protein | | | | | | | | |
| IL15R | X | X | X | X | X | X | X | X |
| PGDH | X | X | X | X | X | X | X | X |
| ADA | X | X | X | X | X | X | X | X |
| ADA2 | X | X | X | X | X | X | X | X |
| HYAL1 | X | X | X | X | X | X | X | X |
| HYAL2 | X | X | X | X | X | X | X | X |
| CHIPS | X | X | X | X | X | X | X | X |
| MLKL (or its | X | X | X | X | X | X | X | X |
| 4HB domain | | | | | | | | |
| only) | | | | | | | | |
| GSDMD (or | X | X | X | X | X | X | X | X |
| its L192A | | | | | | | | |
| mutant, or its | | | | | | | | |
| amino acids 1- | | | | | | | | |
| 233 fragment, | | | | | | | | |
| or its amino | | | | | | | | |
| acids 1-233 | | | | | | | | |
| fragment with | | | | | | | | |
| L192A | | | | | | | | |
| mutation) | | | | | | | | |
| GSDME (or | X | X | X | X | X | X | X | X |
| its amino acid | | | | | | | | |
| 1-237 | | | | | | | | |
| fragment) | | | | | | | | |
| HMGB1 (or | X | X | X | X | X | X | X | X |
| its Box B | | | | | | | | |
| domain only) | | | | | | | | |
| Melittin (e.g., | X | X | X | X | X | X | X | X |
| alpha- | | | | | | | | |
| Melittin) | | | | | | | | |
| SMAC/Diablo | X | X | X | X | X | X | X | X |
| (or its amino | | | | | | | | |
| acid 56-239 | | | | | | | | |
| fragment) | | | | | | | | |
| Snake LAAO | X | X | X | X | X | X | X | X |
| Snake | X | X | X | X | X | X | X | X |
| disintegrin | | | | | | | | |
| Leptin | X | X | X | X | X | X | X | X |
| FLT3L | X | X | X | X | X | X | X | X |
| TRAIL | X | X | X | X | X | X | X | X |
| Gasdermin D | X | X | X | X | X | X | X | X |
| or a truncation | | | | | | | | |
| thereof | | | | | | | | |
| Gasdermin E | X | X | X | X | X | X | X | X |
| or a truncation | | | | | | | | |
| thereof | | | | | | | | |
| Tumor | X | X | X | X | X | X | X | X |
| associated | | | | | | | | |
| antigen | | | | | | | | |
| Tumor | X | X | X | X | X | X | X | X |
| neoantigen | | | | | | | | |
| Bipartite | X | X | X | X | X | X | X | X |
| polypeptide | | | | | | | | |
| binding to | | | | | | | | |
| MHC-peptide | | | | | | | | |
| antigen | | | | | | | | |
| complex | | | | | | | | |
| Fusogenic | X | X | X | X | X | X | X | X |
| protein | | | | | | | | |

In some embodiments, the replicon of the disclosure, or the heterologous polynucleotide of the replicon, comprises coding region for three, or at least three payload molecules according to one of the payload molecule combinations listed in Table 9 below. In some embodiments, the replicon is a SVV derived replicon. In some embodiments, the replicon is a CVA21 derived replicon.

TABLE 9
Three Payload Molecules Combination

| # | Payload Molecule Combination |
|---|---|
| 1 | anti-DLL3 bipartite polypeptide, anti- |
| | FAP bipartite polypeptide, anti-PD1- |
| | VHH-Fc antibody |
| 2 | anti-DLL3 c, anti-FAP bipartite |
| | polypeptide, IL-2 |
| 3 | anti-DLL3 bipartite polypeptide, anti- |
| | FAP bipartite polypeptide, IL-12 |
| 4 | anti-DLL3 bipartite polypeptide, anti- |
| | FAP bipartite polypeptide, IL-18 |
| 5 | anti-DLL3 bipartite polypeptide, anti- |
| | FAP bipartite polypeptide, IL-23 |
| 6 | anti-DLL3 bipartite polypeptide, anti- |
| | FAP bipartite polypeptide, IL-36γ |
| 7 | anti-DLL3 bipartite polypeptide, anti- |
| | FAP bipartite polypeptide, CCL21 |
| 8 | anti-DLL3 bipartite polypeptide, anti- |
| | FAP bipartite polypeptide, CXCL10 |
| 9 | anti-DLL3 bipartite polypeptide, anti- |
| | PD1-VHH-Fc antibody, IL-2 |
| 10 | anti-DLL3 bipartite polypeptide, anti- |
| | PD1-VHH-Fc antibody, IL-12 |
| 11 | anti-DLL3 bipartite polypeptide, anti- |
| | PD1-VHH-Fc antibody, IL-18 |
| 12 | anti-DLL3 bipartite polypeptide, anti- |
| | PD1-VHH-Fc antibody, IL-23 |
| 13 | anti-DLL3 bipartite polypeptide, anti- |
| | PD1-VHH-Fc antibody, IL-36γ |
| 14 | anti-DLL3 bipartite polypeptide, anti- |
| | PD1-VHH-Fc antibody, CCL21 |
| 15 | anti-DLL3 bipartite polypeptide, anti- |
| | PD1-VHH-Fc antibody, CXCL10 |
| 16 | anti-DLL3 bipartite polypeptide, IL-2, |
| | IL-12 |
| 17 | anti-DLL3 bipartite polypeptide, IL-2, |
| | IL-18 |
| 18 | anti-DLL3 bipartite polypeptide, IL-2, |
| | IL-23 |
| 19 | anti-DLL3 bipartite polypeptide, IL-2, |
| | IL-36γ |
| 20 | anti-DLL3 bipartite polypeptide, IL-2, |
| | CCL21 |
| 21 | anti-DLL3 bipartite polypeptide, IL-2, |
| | CXCL10 |
| 22 | anti-DLL3 bipartite polypeptide, IL-12, |
| | IL-18 |
| 23 | anti-DLL3 bipartite polypeptide, IL-12, |
| | IL-23 |
| 24 | anti-DLL3 bipartite polypeptide, IL-12, |
| | IL-36γ |
| 25 | anti-DLL3 bipartite polypeptide, IL-12, |
| | CCL21 |
| 26 | anti-DLL3 bipartite polypeptide, IL-12, |
| | CXCL10 |
| 27 | anti-DLL3 bipartite polypeptide, IL-18, |
| | IL-23 |
| 28 | anti-DLL3 bipartite polypeptide, IL-18, |
| | IL-36γ |
| 29 | anti-DLL3 bipartite polypeptide, IL-18, |
| | CCL21 |
| 30 | anti-DLL3 bipartite polypeptide, IL-18, |
| | CXCL10 |
| 31 | anti-DLL3 bipartite polypeptide, IL-23, |
| | IL-36γ |
| 32 | anti-DLL3 bipartite polypeptide, IL-23, |
| | CCL21 |
| 33 | anti-DLL3 bipartite polypeptide, IL-23, |
| | CXCL10 |
| 34 | anti-DLL3 bipartite polypeptide, IL-36γ, |
| | CCL21 |
| 35 | anti-DLL3 bipartite polypeptide, IL-36γ, |
| | CXCL10 |
| 36 | anti-DLL3 bipartite polypeptide, CCL21, |
| | CXCL10 |
| 37 | anti-FAP bipartite polypeptide, anti-PD1- |
| | VHH-Fc antibody, IL-2 |
| 38 | anti-FAP bipartite polypeptide, anti-PD1- |
| | VHH-Fc antibody, IL-12 |
| 39 | anti-FAP bipartite polypeptide, anti-PD1- |
| | VHH-Fc antibody, IL-18 |
| 40 | anti-FAP bipartite polypeptide, anti-PD1- |
| | VHH-Fc antibody, IL-23 |
| 41 | anti-FAP bipartite polypeptide, anti-PD1- |
| | VHH-Fc antibody, IL-36γ |
| 42 | anti-FAP bipartite polypeptide, anti-PD1- |
| | VHH-Fc antibody, CCL21 |
| 43 | anti-FAP bipartite polypeptide, anti-PD1- |
| | VHH-Fc antibody, CXCL10 |
| 44 | anti-FAP bipartite polypeptide, IL-2, |
| | IL-12 |
| 45 | anti-FAP bipartite polypeptide, IL-2, |
| | IL-18 |
| 46 | anti-FAP bipartite polypeptide, IL-2, |
| | IL-23 |
| 47 | anti-FAP bipartite polypeptide, IL-2, |
| | IL-36γ |
| 48 | anti-FAP bipartite polypeptide, IL-2, |
| | CCL21 |
| 49 | anti-FAP bipartite polypeptide, IL-2, |
| | CXCL10 |
| 50 | anti-FAP bipartite polypeptide, IL-12, |
| | IL-18 |
| 51 | anti-FAP bipartite polypeptide, IL-12, |
| | IL-23 |
| 52 | anti-FAP bipartite polypeptide, IL-12, |
| | IL-36γ |
| 53 | anti-FAP bipartite polypeptide, IL-12, |
| | CCL21 |
| 54 | anti-FAP bipartite polypeptide, IL-12, |
| | CXCL10 |
| 55 | anti-FAP bipartite polypeptide, IL-18, |
| | IL-23 |
| 56 | anti-FAP bipartite polypeptide, IL-18, |
| | IL-36γ |
| 57 | anti-FAP bipartite polypeptide, IL-18, |
| | CCL21 |
| 58 | anti-FAP bipartite polypeptide, IL-18, |
| | CXCL10 |
| 59 | anti-FAP bipartite polypeptide, IL-23, |
| | IL-36γ |
| 60 | anti-FAP bipartite polypeptide, IL-23, |
| | CCL21 |
| 61 | anti-FAP bipartite polypeptide, IL-23, |
| | CXCL10 |
| 62 | anti-FAP bipartite polypeptide, IL-36γ, |
| | CCL21 |
| 63 | anti-FAP bipartite polypeptide, IL-36γ, |
| | CXCL10 |
| 64 | anti-FAP bipartite polypeptide, |
| | CCL21, CXCL10 |
| 65 | anti-PD1-VHH-Fc antibody, IL-2, |
| | IL-12 |
| 66 | anti-PD1-VHH-Fc antibody, IL-2, |
| | IL-18 |
| 67 | anti-PD1-VHH-Fc antibody, IL-2, |
| | IL-23 |
| 68 | anti-PD1-VHH-Fc antibody, IL-2, |
| | IL-36γ |
| 69 | anti-PD1-VHH-Fc antibody, IL-2, |
| | CCL21 |
| 70 | anti-PD1-VHH-Fc antibody, IL-2, |
| | CXCL10 |
| 71 | anti-PD1-VHH-Fc antibody, IL-12, |
| | IL-18 |
| 72 | anti-PD1-VHH-Fc antibody, IL-12, |
| | IL-23 |
| 73 | anti-PD1-VHH-Fc antibody, IL-12, |
| | IL-36γ |
| 74 | anti-PD1-VHH-Fc antibody, IL-12, |
| | CCL21 |
| 75 | anti-PD1-VHH-Fc antibody, IL-12, |
| | CXCL10 |
| 76 | anti-PD1-VHH-Fc antibody, IL-18, |
| | IL-23 |
| 77 | anti-PD1-VHH-Fc antibody, IL-18, |
| | IL-36γ |
| 78 | anti-PD1-VHH-Fc antibody, IL-18, |
| | CCL21 |
| 79 | anti-PD1-VHH-Fc antibody, IL-18, |
| | CXCL10 |
| 80 | anti-PD1-VHH-Fc antibody, IL-23, |
| | IL-36γ |
| 81 | anti-PD1-VHH-Fc antibody, IL-23, |
| | CCL21 |
| 82 | anti-PD1-VHH-Fc antibody, IL-23, |
| | CXCL10 |
| 83 | anti-PD1-VHH-Fc antibody, IL-36γ, |
| | CCL21 |
| 84 | anti-PD1-VHH-Fc antibody, IL-36γ, |
| | CXCL10 |
| 85 | anti-PD1-VHH-Fc antibody, |
| | CCL21, CXCL10 |
| 86 | IL-2, IL-12, IL-18 |
| 87 | IL-2, IL-12, IL-23 |
| 88 | IL-2, IL-12, IL-36γ |
| 89 | IL-2, IL-12, CCL21 |
| 90 | IL-2, IL-12, CXCL10 |
| 91 | IL-2, IL-18, IL-23 |
| 92 | IL-2, IL-18, IL-36γ |
| 93 | IL-2, IL-18, CCL21 |
| 94 | IL-2, IL-18, CXCL10 |
| 95 | IL-2, IL-23, IL-36γ |
| 96 | IL-2, IL-23, CCL21 |
| 97 | IL-2, IL-23, CXCL10 |
| 98 | IL-2, IL-36γ, CCL21 |
| 99 | IL-2, IL-36γ, CXCL10 |
| 100 | IL-2, CCL21, CXCL10 |
| 101 | IL-12, IL-18, IL-23 |
| 102 | IL-12, IL-18, IL-36γ |
| 103 | IL-12, IL-18, CCL21 |
| 104 | IL-12, IL-18, CXCL10 |
| 105 | IL-12, IL-23, IL-36γ |
| 106 | IL-12, IL-23, CCL21 |
| 107 | IL-12, IL-23, CXCL10 |
| 108 | IL-12, IL-36γ, CCL21 |
| 109 | IL-12, IL-36γ, CXCL10 |
| 110 | IL-12, CCL21, CXCL10 |
| 111 | IL-18, IL-23, IL-36γ |
| 112 | IL-18, IL-23, CCL21 |
| 113 | IL-18, IL-23, CXCL10 |
| 114 | IL-18, IL-36γ, CCL21 |
| 115 | IL-18, IL-36γ, CXCL10 |
| 116 | IL-18, CCL21, CXCL10 |
| 117 | IL-23, IL-36γ, CCL21 |
| 118 | IL-23, IL-36γ, CXCL10 |
| 119 | IL-23, CCL21, CXCL10 |
| 120 | IL-36γ, CCL21, CXCL10 |

In some embodiments, the anti-DLL3 bipartite polypeptide in Table 8 or Table 9 binds to DLL3 and one of the effector cell antigens listed in Table 3. In some embodiments, the anti-DLL3 bipartite polypeptide in Table 8 or Table 9 binds to DLL3 and one of the effector cell antigens selected from CD3, NKp46 and CD16. In some embodiments, the anti-DLL3 bipartite polypeptide in Table 8 or Table 9 is a BiTE.

In some embodiments, the anti-FAP bipartite polypeptide in Table 8 or Table 9 binds to FAP and one of the effector cell antigens listed in Table 3. In some embodiments, the anti-FAP bipartite polypeptide in Table 8 or Table 9 binds to FAP and one of the effector cell antigens selected from CD3, NKp46 and CD16. In some embodiments, the anti-FAP bipartite polypeptide in Table 8 or Table 9 is a BiTE.

In some embodiments, the anti-EpCAM bipartite polypeptide in Table 8 or Table 9 binds to EpCAM and one of the effector cell antigens listed in Table 3. In some embodiments, the anti-EpCAM bipartite polypeptide in Table 8 or Table 9 binds to EpCAM and one of the effector cell antigens selected from CD3, NKp46 and CD16. In some embodiments, the anti-EpCAM bipartite polypeptide in Table 8 or Table 9 is a BiTE.

In some embodiments, the Various Seneca Valley virus (SVV) derived recombinant RNA replicons comprise a heterologous polynucleotide encoding one or more immunomodulatory proteins (e.g., anti-DLL3 Bi-specific T-cell engager (BiTE)). In some embodiments, the SVV derived recombinant RNA replicons further comprise coding regions for one or more cytokines (e.g., IL-2, IL-12, IL-36γ) and/or one or more chemokines (e.g., CCL21, CCL4). In some embodiments, the SVV derived recombinant RNA replicons comprise coding regions of two or more payload molecules according to Table 10 below.

TABLE 10
Payload Molecules for SVV derived Replicon

| SVV derived Replicon | Payload Molecules |
|---|---|
| Replicon Construct#A1: | anti-DLL3 BiTE, IL-2 |
| Replicon Construct#A2: | anti-DLL3 BiTE, IL-12 |
| Replicon Construct#A3: | anti-DLL3 BiTE, IL-36γ |
| Replicon Construct#A4: | anti-DLL3 BiTE, CCL21 |
| Replicon Construct#A5: | anti-DLL3 BiTE, CCL4 |
| Replicon Construct#A6: | anti-DLL3 BiTE, IL-2, IL-12 |
| Replicon Construct#A7: | anti-DLL3 BiTE, IL-2, IL-36γ |
| Replicon Construct#A8: | anti-DLL3 BiTE, IL-2, CCL21 |
| Replicon Construct#A9: | anti-DLL3 BiTE, IL-2, CCL4 |
| Replicon Construct#A10: | anti-DLL3 BiTE, IL-12, IL-36γ |
| Replicon Construct#A11: | anti-DLL3 BiTE, IL-12, CCL21 |
| Replicon Construct#A12: | anti-DLL3 BiTE, IL-12, CCL4 |
| Replicon Construct#A13: | anti-DLL3 BiTE, IL-36γ, CCL21 |
| Replicon Construct#A14: | anti-DLL3 BiTE, IL-36γ, CCL4 |
| Replicon Construct#A15: | anti-DLL3 BiTE, CCL21, CCL4 |

In some embodiments, the Coxsackievirus A21 (CVA21)-derived recombinant RNA replicons comprise a heterologous polynucleotide encoding one or more immunomodulatory proteins (e.g., anti-DLL3 Bi-specific T-cell engager (BiTE)). In some embodiments, the CVA21 derived recombinant RNA replicons further comprise coding regions for one or more cytokines (e.g., IL-2, IL-12, IL-36γ) and/or one or more chemokines (e.g., CCL21, CCL4). In some embodiments, the CVA21 derived recombinant RNA replicons comprise coding regions of two or more payload molecules according to Table 11 below.

TABLE 11
Payload Molecules for CVA21 derived Replicon

| CVA21 derived Replicon | Payload Molecules |
|---|---|
| Replicon Construct#C1: | anti-DLL3 BiTE, IL-2 |
| Replicon Construct#C2: | anti-DLL3 BiTE, IL-12 |
| Replicon Construct#C3: | anti-DLL3 BiTE, IL-36γ |
| Replicon Construct#C4: | anti-DLL3 BiTE, CCL21 |
| Replicon Construct#C5: | anti-DLL3 BiTE, CCL4 |
| Replicon Construct#C6: | anti-DLL3 BiTE, IL-2, IL-12 |
| Replicon Construct#C7: | anti-DLL3 BiTE, IL-2, IL-36γ |
| Replicon Construct#C8: | anti-DLL3 BiTE, IL-2, CCL21 |
| Replicon Construct#C9: | anti-DLL3 BiTE, IL-2, CCL4 |
| Replicon Construct#C10: | anti-DLL3 BiTE, IL-12, IL-36γ |
| Replicon Construct#C11: | anti-DLL3 BiTE, IL-12, CCL21 |
| Replicon Construct#C12: | anti-DLL3 BiTE, IL-12, CCL4 |
| Replicon Construct#C13: | anti-DLL3 BiTE, IL-36γ, CCL21 |
| Replicon Construct#C14: | anti-DLL3 BiTE, IL-36γ, CCL4 |
| Replicon Construct#C15: | anti-DLL3 BiTE, CCL21, CCL4 |

### Internal Ribosome Entry Site

In some embodiments, the recombinant RNA replicon comprises an IRES outside of the 5′ UTR. In some embodiments, the IRES is located between 5′ UTR and 2B coding region. In some embodiments, the IRES is located between 2A coding region and 2B coding region. In some embodiments, the IRES is located between the payload molecule coding sequence and 2B coding region. In some embodiments, the IRES is located between a CRE and 2B coding region. In some embodiments, the IRES is located between 5′ UTR and the heterologous polynucleotide. In some embodiments, the IRES is located between the CRE and the heterologous polynucleotide. In some embodiments, the IRES is located between a VP coding region and the heterologous polynucleotide. In some embodiments, the IRES is located between 2A coding region and the heterologous polynucleotide. In some embodiments, the IRES is an EMCV IRES. In some embodiments, the replicon is a replicon comprising a SVV genome.

### Trans-Encapsidation

In some embodiments, the recombinant RNA replicon of the disclosure can be trans-encapsidated by another recombinant RNA molecule encoding an oncolytic virus (e.g., an RNA viral genome). Such recombinant RNA molecule may comprise a viral genome (e.g., a synthetic viral genomes). In some embodiments, such recombinant RNA molecules or RNA viral genome is capable of producing an infectious, lytic virus when introduced into a cell by a non-viral delivery vehicle and does not require additional exogenous genes or proteins to be present in the cell in order to replicate and produce an infectious virus. In some embodiments, such RNA viral genome comprises all the VP coding regions. The expressed viral proteins then mediate viral replication and assembly into an infectious viral particle (which may comprise a capsid protein, an envelope protein, and/or a membrane protein) comprising the RNA viral genome. In some embodiments, the recombinant RNA replicon of the disclosure can be trans-encapsidated by the capsid proteins expressed from such RNA viral genome. In some embodiments, the recombinant RNA replicon can be trans-encapsidated when the recombinant RNA replicon and the RNA viral genome are present in the same cell (e.g., by delivering them into the cell via the particle). As such, the recombinant RNA replicon and the RNA viral genome described herein, when introduced into the same host cell, can produce two groups of viral particles-one group comprises the recombinant RNA replicon, the other group comprises the RNA viral genome, both of which are capable of infecting another host cell.

miRNA Target Sequence (miR-TS) Cassette

In some embodiments, the recombinant RNA replicon comprises one or more microRNA (miRNA) target sequence (miR-TS) cassettes, wherein the miR-TS cassette comprises one or more miRNA target sequences, and wherein expression of one or more of the corresponding miRNAs in a cell inhibits replication of the replicon in the cell. In some embodiments, the one or more miRNAs are selected from miR-124, miR-1, miR-143, miR-128, miR-219, miR-219a, miR-122, miR-204, miR-217, miR-137, and miR-126. In some embodiments, the miR-TS cassette comprises one or more copies of a miR-124 target sequence, one or more copies of a miR-1 target sequence, and one or more copies of a miR-143 target sequence. In some embodiments, the miR-TS cassette comprises one or more copies of a miR-128 target sequence, one or more copies of a miR-219a target sequence, and one or more copies of a miR-122 target sequence. In some embodiments, the miR-TS cassette comprises one or more copies of a miR-128 target sequence, one or more copies of a miR-204 target sequence, and one or more copies of a miR-219 target sequence. In some embodiments, the miR-TS cassette comprises one or more copies of a miR-217 target sequence, one or more copies of a miR-137 target sequence, and one or more copies of a miR-126 target sequence.

In some embodiments, the recombinant RNA replicon comprises one or more miR-TS cassettes is incorporated into the 5′ untranslated region (UTR) or 3′ UTR of one or more essential viral genes (protein coding regions). In some embodiments, the recombinant RNA replicon comprises one or more miR-TS cassettes is incorporated into the 5′ untranslated region (UTR) or 3′ UTR of one or more non-essential genes. In some embodiments, the recombinant RNA replicon comprises one or more miR-TS cassettes is incorporated 5′ or 3′ of one or more essential viral genes.

### Methods of Producing Recombinant RNA Replicons

In some embodiments, the recombinant RNA replicons of the disclosure are produced in vitro using one or more DNA vector templates comprising a polynucleotide encoding the recombinant RNA replicons. The term “vector” is used herein to refer to a nucleic acid molecule capable transferring, encoding, or transporting another nucleic acid molecule. The transferred nucleic acid is generally inserted into the vector nucleic acid molecule. A vector may include sequences that direct autonomous replication in a cell and/or may include sequences sufficient to allow integration into host cell DNA. In some embodiments, the recombinant RNA replicon of the disclosure is produced using one or more viral vectors.

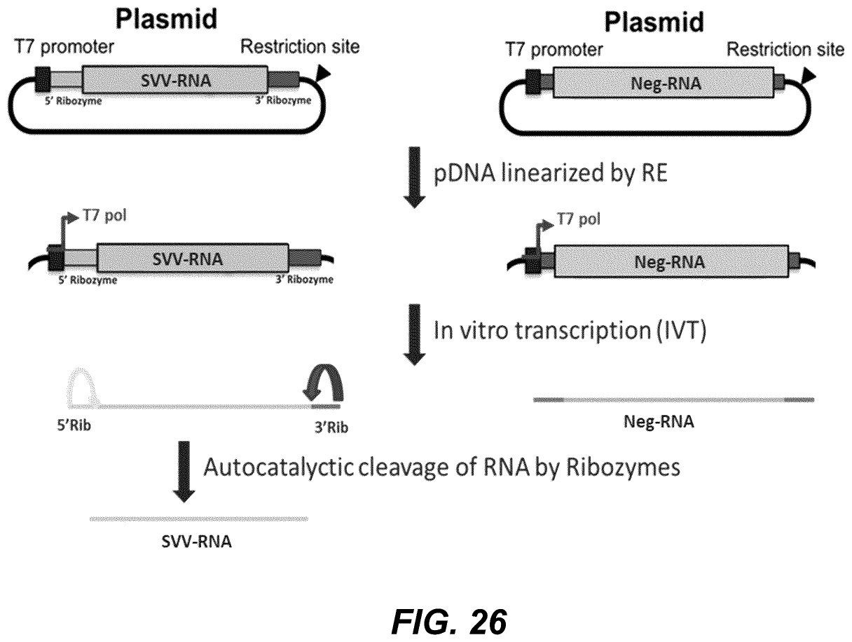

In some embodiments, the recombinant RNA replicons of the disclosure are produced by introducing a polynucleotide encoding the recombinant RNA replicon (e.g., by means of transfection, transduction, electroporation, and the like) into a suitable host cell in vitro. Suitable host cells include insect and mammalian cell lines. The host cells are cultured for an appropriate amount of time to allow expression of the polynucleotides and production of the recombinant RNA replicons. The recombinant RNA replicons are then isolated from the host cell and formulated for therapeutic use (e.g., encapsulated in a particle). A schematic of the in vitro synthesis of the recombinant RNA replicons with 3′ and 5′ ribozymes is shown in FIG. 26 (using SVV derived replicon as example but it applies to other replicons as well). The same schematic applies to the synthesis of recombinant RNA replicons using other combinations of junctional cleavage sequences.

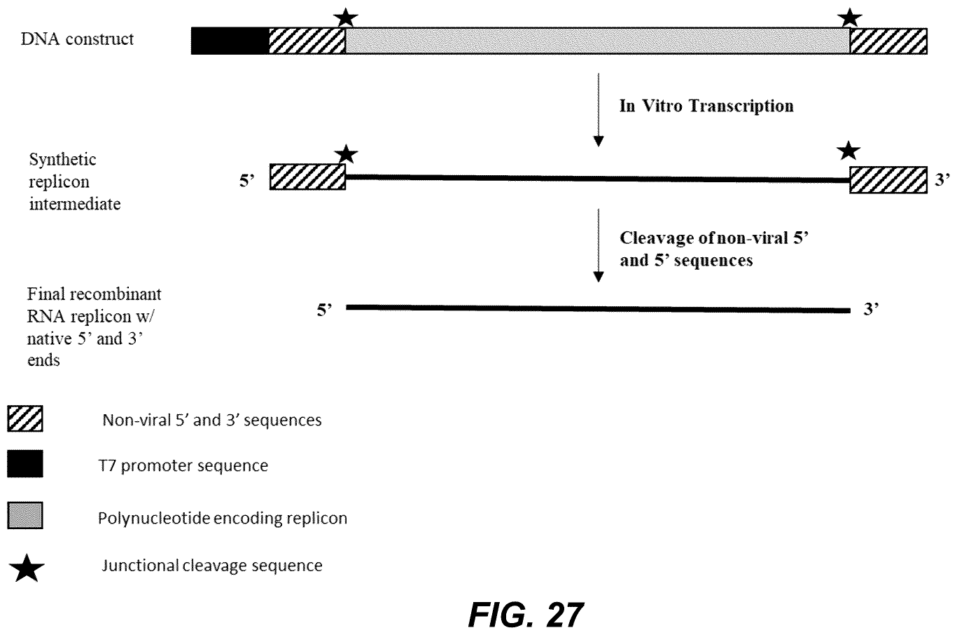

In some embodiments, the replication of the recombinant RNA replicons of the disclosure require discrete 5′ and 3′ ends that are native to the viral genome of the replicon. The RNA transcripts produced by T7 RNA polymerase in vitro or by mammalian RNA Pol II contain mammalian 5′ and 3′ UTRs do not contain the discrete, native ends required for production of an infectious RNA virus. For example, the T7 RNA polymerase requires a guanosine residue on the 5′ end of the template polynucleotide in order to initiate transcription. However, SVV begins with a uridine residue on its 5′ end. Thus, the T7 leader sequence, which is required for in vitro transcription of the replicon comprising the SVV viral genome, must be removed to generate the native 5′ SVV terminus required for production of a functional replicon. Therefore, in some embodiments, polynucleotides suitable for use in the production of the recombinant RNA replicons of the disclosure require additional non-viral 5′ and 3′ sequences that enable generation of the discrete 5′ and 3′ ends native to the virus. Such sequences are referred to herein as junctional cleavage sequences (JCS). In some embodiments, the junctional cleavage sequences act to cleave the T7 RNA polymerase or Pol II-encoded RNA transcript at the junction of the viral RNA and the mammalian mRNA sequence such that the non-viral RNA polynucleotides are removed from the transcript in order to maintain the endogenous 5′ and 3′ discrete ends of the viral genome (See schematic shown in FIG. 27). In some embodiments, the junctional cleavage sequences act to generate the appropriate ends during the linearization of the DNA plasmid encoding the recombinant RNA replicons (e.g., the use of 3′ restriction enzyme recognition sequences to produce the appropriate 3′ end upon linearization of the plasmid template and prior to in vitro transcription of the recombinant RNA replicons).

The nature of the junctional cleavage sequences and the removal of the non-viral RNA from the viral genome transcript can be accomplished by a variety of methods. For example, in some embodiments, the junctional cleavage sequences are targets for RNA interference (RNAi) molecules. “RNA interference molecule” as used herein refers to an RNA polynucleotide that mediates degradation of a target mRNA sequence through endogenous gene silencing pathways (e.g., Dicer and RNA-induced silencing complex (RISC)). Exemplary RNA interference agents include micro RNAs (miRNAs), artificial miRNA (amiRNAs), short hairpin RNAs (shRNAs), and small interfering RNAs (siRNAs). Further, any system for cleaving an RNA transcript at a specific site currently known the art or to be defined in the future can be used to generate the discrete ends native to the virus.

In some embodiments, the RNAi molecule is a miRNA. A miRNA refers to a naturally-occurring, small non-coding RNA molecule of about 18-25 nucleotides in length that is at least partially complementary to a target mRNA sequence. In animals, genes for miRNAs are transcribed to a primary miRNA (pri-miRNA), which is double stranded and forms a stem-loop structure. Pri-miRNAs are then cleaved in the nucleus by a microprocessor complex comprising the class 2 RNase III, Drosha, and the microprocessor subunit, DCGR8, to form a 70-100 nucleotide precursor miRNA (pre-miRNA). The pre-miRNA forms a hairpin structure and is transported to the cytoplasm where it is processed by the RNase III enzyme, Dicer, into a miRNA duplex of ˜18-25 nucleotides. Although either strand of the duplex may potentially act as a functional miRNA, typically one strand of the miRNA is degraded and only one strand is loaded onto the Argonaute (AGO) nuclease to produce the effector RNA-induced silencing complex (RISC) in which the miRNA and its mRNA target interact (Wahid et al., 1803:11, 2010, 1231-1243). In some embodiments, the 5′ and/or 3′ junctional cleavage sequences are miRNA target sequences.

In some embodiments, the RNAi molecule is an artificial miRNA (amiRNA) derived from a synthetic miRNA-embedded in a Pol II transcript. (See e.g., Liu et al., Nucleic Acids Res (2008) 36:9; 2811-2834; Zeng et al., Molecular Cell (2002), 9; 1327-1333; Fellman et al., Cell Reports (2013) 5; 1704-1713). In some embodiments, the 5′ and/or 3′ junctional cleavage sequences are amiRNA target sequences.

In some embodiments, the RNAi molecule is an siRNA molecule. siRNAs refer to double stranded RNA molecules typically about 21-23 nucleotides in length. The duplex siRNA molecule is processed in the cytoplasm by the associates with a multi protein complex called the RNA-induced silencing complex (RISC), during which the “passenger” sense strand is enzymatically cleaved from the duplex. The antisense “guide” strand contained in the activated RISC then guides the RISC to the corresponding mRNA by virtue of sequence complementarity and the AGO nuclease cuts the target mRNA, resulting in specific gene silencing. In some embodiments, the siRNA molecule is derived from an shRNA molecule. shRNAs are single stranded artificial RNA molecules ˜ 50-70 nucleotides in length that form stem-loop structures. Expression of shRNAs in cells is accomplished by introducing a DNA polynucleotide encoding the shRNA by plasmid or viral vector. The shRNA is then transcribed into a product that mimics the stem-loop structure of a pre-miRNA, and after nuclear export the hair-pin is processed by Dicer to form a duplex siRNA molecule which is then further processed by the RISC to mediate target-gene silencing. In some embodiments, the 5′ and/or 3′ junctional cleavage sequences are siRNA target sequences.

In some embodiments, the junctional cleavage sequences are guide RNA (gRNA) target sequences. In such embodiments, gRNAs can be designed and introduced with a Cas endonuclease with RNase activity (e.g., Cas13) to mediate cleavage of the viral genome transcript at the precise junctional site. In some embodiments, the 5′ and/or 3′ junctional cleavage sequences are gRNA target sequences.

In some embodiments, the junctional cleavage sequences are pri-miRNA-encoding sequences. Upon transcription of the polynucleotide encoding the viral genome (e.g., the recombinant RNA molecule), these sequences form the pri-miRNA stem-loop structure which is then cleaved in the nucleus by Drosha to cleave the transcript at the precise junctional site. In some embodiments, the 5′ and/or 3′ junctional cleavage sequences are pri-mRNA target sequences.

In some embodiments, the junctional cleavage sequences are primer binding sequences that facilitate cleavage by the endoribonuclease, RNAseH. In such embodiments, a primer that anneals to the 5′ and/or 3′ junctional cleavage sequence is added to the in vitro reaction along with an RNAseH enzyme. RNAseH specifically hydrolyzes the phosphodiester bonds of RNA which is hybridized to DNA, therefore enabling cleavage of the recombinant RNA replicon intermediates at the precise junctional cleavage sequence to produce the required 5′ and 3′ native ends.

In some embodiments, the junctional cleavage sequences are restriction enzyme recognition sites and result in the generation of discrete ends of viral transcripts during linearization of the plasmid template runoff RNA synthesis with T7 RNA Polymerase. In some embodiments, the junctional cleavage sequences are Type IIS restriction enzyme recognition sites. Type IIS restriction enzymes comprise a specific group of enzymes which recognize asymmetric DNA sequences and cleave at a defined distance outside of their recognition sequence, usually within 1 to 20 nucleotides. Exemplary Type IIS restriction enzymes include AcuI, AlwI, BaeI, BbsI, BbvI, BccI, BceAI, BcgI, BciVI, BcoDI, BfuAI, BmrI, BpmI, BpuEI, BsaI, BsaXI, BseRI, BsgI, BsmAI, BsmBi, BsmFI, BsmI, BspCNI, BspMI, BspQI, BsrDI, BsrI, BtgZI, BtsCI, BstI, CaspCI, EarI, EciI, Esp3I, FauI, FokI, HgaI, HphI, HpyAV, MbolI, MlyI, MmeI, MnlL, NmeAIII, PleI, SapI, and SfaNI. The recognition sequences for these Type IIS restriction enzymes are known in the art. See the New England Biolabs website located at neb.com/tools-and-resources/selection-charts/type-iis-restriction-enzymes. In some embodiments, the junctional cleavage sequence is a SapI restriction enzyme recognition site.

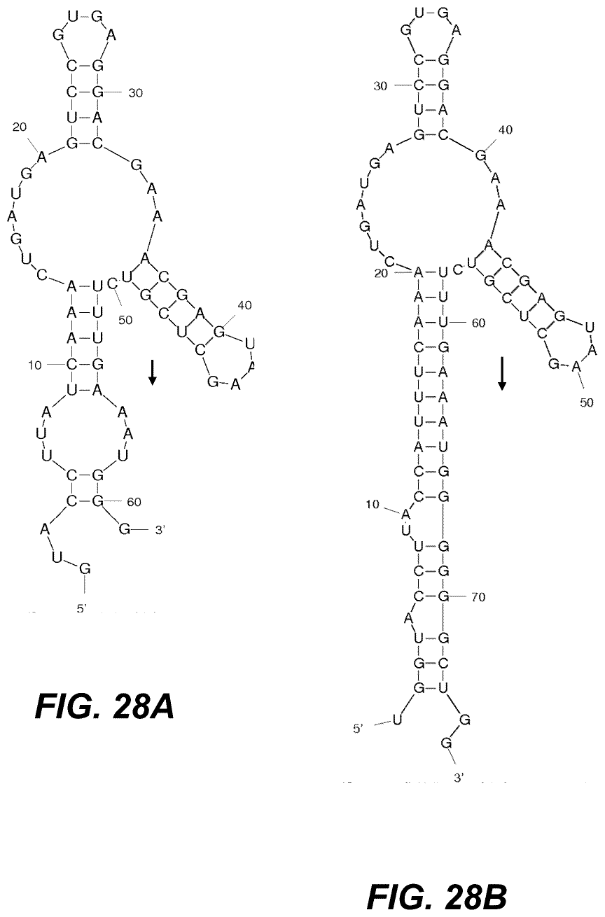

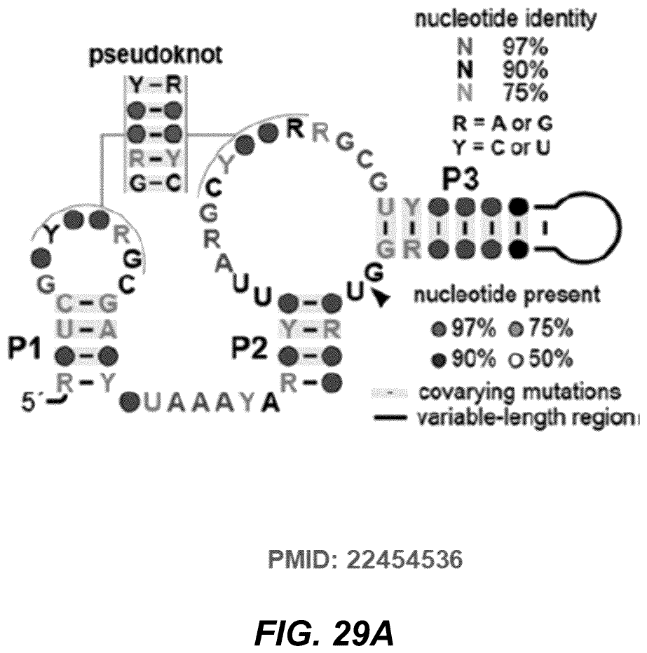

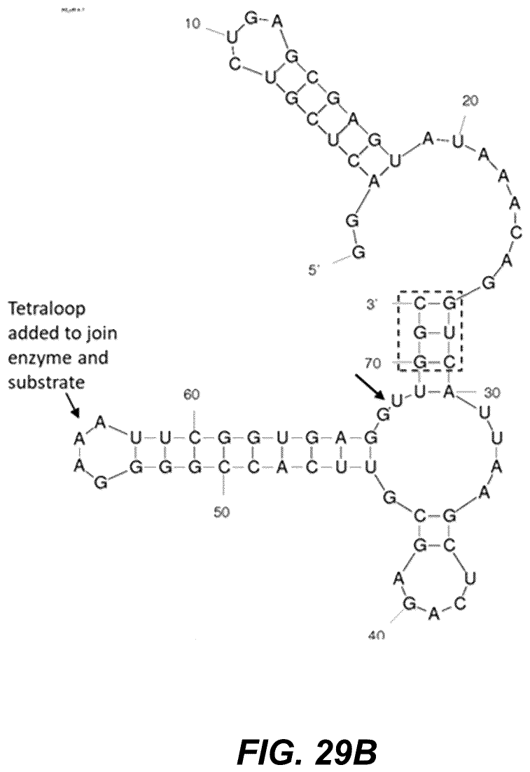

In some embodiments, the junctional cleavage sequences are ribozyme-encoding sequences and mediate self-cleavage of the recombinant RNA replicons intermediates to produce the native discrete 5′ and 3′ ends of required for the final recombinant RNA replicons and subsequent production of infectious RNA viruses. Exemplary ribozymes include the Hammerhead ribozyme (e.g., the Hammerhead ribozymes shown in FIGS. 28A-28B), the Varkud satellite (VS) ribozyme, the hairpin ribozyme, the GIR1 branching ribozyme, the glmS ribozyme, the twister ribozyme, the twister sister ribozyme, the pistol ribozyme (e.g., Pistol 1 and Pistol 2 shown in FIGS. 29A-29B), the hatchet ribozyme, and the Hepatitis delta virus ribozyme. In some embodiments, the 5′ and/or 3′ junctional cleavage sequences are ribozyme encoding sequences.

In some embodiments, the junctional cleavage sequences are sequences encoding ligand-inducible self-cleaving ribozymes, referred to as “aptazymes”. Aptazymes are ribozyme sequences that contain an integrated aptamer domain specific for a ligand. Ligand binding to the apatmer domain triggers activation of the enzymatic activity of the ribozyme, thereby resulting in cleavage of the RNA transcript. Exemplary aptazymes include theophylline-dependent aptazymes (e.g., hammerhead ribozyme linked to a theophylline-dependent apatmer), tetracycline-dependent aptazymes (e.g., hammerhead ribozyme linked to a Tet-dependent aptamer), guanine-dependent aptazymes (e.g., hammerhead ribozyme linked to a guanine-dependent aptamer). In some embodiments, the 5′ and/or 3′ junctional cleavage sequences are aptazyme-encoding sequences.

In some embodiments, the junctional cleavage sequences are target sequences for an RNAi molecule (e.g., an siRNA molecule, an shRNA molecule, an miRNA molecule, or an amiRNA molecule), a gRNA molecule, or an RNAseH primer. In such embodiments, the junctional cleavage sequence is at least partially complementary to the sequence of the RNAi molecule, gRNA molecule, or primer molecule. Methods of sequence alignment for comparison and determination of percent sequence identity and percent complementarity are well known in the art. Optimal alignment of sequences for comparison can be conducted, e.g., by the homology alignment algorithm of Needleman and Wunsch, (1970) J. Mol. Biol. 48:443, by the search for similarity method of Pearson and Lipman, (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), by manual alignment and visual inspection (see, e.g., Brent et al., (2003) Current Protocols in Molecular Biology), by use of algorithms know in the art including the BLAST and BLAST 2.0 algorithms, which are described in Altschul et al., (1977) Nuc. Acids Res. 25:3389-3402; and Altschul et al., (1990) J. Mol. Biol. 215:403-410, respectively. Software for performing BLAST analyses is publicly available through the National Center for Biotechnology Information.

In some embodiments, the 5′ junctional cleavage sequence and 3′ junctional cleavage sequence are from the same group (e.g., are both RNAi target sequences, both ribozyme-encoding sequences, etc.). For example, in some embodiments, the junctional cleavage sequences are RNAi target sequences (e.g., siRNA, shRNA, amiRNA, or miRNA target sequences) and are incorporated into the 5′ and 3′ ends of the polynucleotide encoding the recombinant RNA replicon. In such embodiments, the 5′ and 3′ RNAi target sequence may be the same (i.e., targets for the same siRNA, amiRNA, or miRNA) or different (i.e., the 5′ sequence is a target for one siRNA, shmiRNA, or miRNA and the 3′ sequence is a target for another siRNA, amiRNA, or miRNA). In some embodiments, the junctional cleavage sequences are guide RNA target sequences and are incorporated into the 5′ and 3′ ends of the polynucleotide encoding the recombinant RNA replicon. In such embodiments, the 5′ and 3′ gRNA target sequences may be the same (i.e., targets for the same gRNA) or different (i.e., the 5′ sequence is a target for one gRNA and the 3′ sequence is a target for another gRNA). In some embodiments, the junctional cleavage sequences are pri-mRNA-encoding sequences and are incorporated into the 5′ and 3′ ends of the polynucleotide encoding the recombinant RNA replicon. In some embodiments, the junctional cleavage sequences are ribozyme-encoding sequences and are incorporated immediately 5′ and 3′ of the polynucleotide sequence encoding the recombinant RNA replicon.

In some embodiments, the 5′ junctional cleavage sequence and 3′ junctional cleavage sequence are from the same group but are different variants or types. For example, in some embodiments, the 5′ and 3′ junctional cleavage sequences may be target sequences for an RNAi molecule, wherein the 5′ junctional cleavage sequence is an siRNA target sequence and the 3′ junctional cleavage sequence is a miRNA target sequence (or vis versa). In some embodiments, the 5′ and 3′ junctional cleavage sequences may be ribozyme-encoding sequences, wherein the 5′ junctional cleavage sequence is a hammerhead ribozyme-encoding sequence and the 3′ junctional cleavage sequence is a hepatitis delta virus ribozyme-encoding sequence.

In some embodiments, the 5′ junctional cleavage sequence and 3′ junctional cleavage sequence are different types. For example, in some embodiments, the 5′ junctional cleavage sequence is an RNAi target sequence (e.g., an siRNA, an amiRNA, or a miRNA target sequence) and the 3′ junctional cleavage sequence is a ribozyme sequence, an aptazyme sequence, a pri-miRNA sequence, or a gRNA target sequence. In some embodiments, the 5′ junctional cleavage sequence is a ribozyme sequence and the 3′ junctional cleavage sequence is an RNAi target sequence (e.g., an siRNA, an amiRNA, or a miRNA target sequence), an aptazyme sequence, a pri-miRNA-encoding sequence, or a gRNA target sequence. In some embodiments, the 5′ junctional cleavage sequence is an aptazyme sequence and the 3′ junctional cleavage sequence is an RNAi target sequence (e.g., an siRNA, an amiRNA, or a miRNA target sequence), a ribozyme sequence, a pri-miRNA sequence, or a gRNA target sequence. In some embodiments, the 5′ junctional cleavage sequence is a pri-miRNA sequence and the 3′ junctional cleavage sequence is an RNAi target sequence (e.g., an siRNA, an amiRNA, or a miRNA target sequence), a ribozyme sequence, an aptazyme sequence, or a gRNA target sequence. In some embodiments, the 5′ junctional cleavage sequence is a gRNA target sequence and the 3′ junctional cleavage sequence is an RNAi target sequence (e.g., an siRNA, an amiRNA, or a miRNA target sequence), a ribozyme sequence, a pri-miRNA sequence, or an aptazyme sequence.

Exemplary arrangements of the junctional cleavage sequences relative to the polynucleotide encoding the recombinant RNA replicon are shown below in Tables 12 and 13.

TABLE 12
Symmetrical Junctional Cleavage Sequence (JSC) Arrangements

| 5′ | JCS | | JCS | 3′ |
|---|---|---|---|---|
| | siRNA TS | replicon | siRNA TS | |
| | miR TS | replicon | miR TS | |
| | AmiR TS | replicon | AmiR TS | |
| | gRNA TS | replicon | gRNA TS | |
| | pri-miR | replicon | pri-miR | |
| | ribozyme | replicon | ribozyme | |
| | aptazyme | replicon | aptazyme | |
| | RNAseH primer TS | replicon | RNAseH primer TS | |

TABLE 13
Asymmetrical JCS Arrangements

| 5′ | JCS | | JCS | 3′ |
|---|---|---|---|---|
| | siRNA TS | replicon | miR TS | |
| | siRNA TS | replicon | AmiR TS | |
| | siRNA TS | replicon | gRNA TS | |
| | siRNA TS | replicon | pri-miR | |
| | siRNA TS | replicon | ribozyme | |
| | siRNA TS | replicon | aptazyme | |
| | siRNA TS | replicon | RNAseH primer TS | |
| | siRNA TS | replicon | Restr Enz RS | |
| | miR TS | replicon | siRNA TS | |
| | miR TS | replicon | AmiR TS | |
| | miR TS | replicon | gRNA TS | |
| | miR TS | replicon | pri-miR | |
| | miR TS | replicon | ribozyme | |
| | miR TS | replicon | aptazyme | |
| | miR TS | replicon | RNAseH primer TS | |
| | miR TS | replicon | Restr Enz RS | |
| | AmiR TS | replicon | siRNA TS | |
| | AmiR TS | replicon | miR TS | |
| | AmiR TS | replicon | gRNA TS | |
| | AmiR TS | replicon | pri-miR | |
| | AmiR TS | replicon | ribozyme | |
| | AmiR TS | replicon | aptazyme | |
| | AmiR TS | replicon | RNAseH primer TS | |
| | AmiR TS | replicon | Restr Enz RS | |
| | gRNA TS | replicon | siRNA TS | |
| | gRNA TS | replicon | miR TS | |
| | gRNA TS | replicon | AmiR TS | |
| | gRNA TS | replicon | pri-miR | |
| | gRNA TS | replicon | ribozyme | |
| | gRNA TS | replicon | aptazyme | |
| | gRNA TS | replicon | RNAseH primer TS | |
| | gRNA TS | replicon | Restr Enz RS | |
| | pri-miR | replicon | siRNA TS | |
| | pri-miR | replicon | miR TS | |
| | pri-miR | replicon | AmiR TS | |
| | pri-miR | replicon | gRNA TS | |
| | pri-miR | replicon | ribozyme | |
| | pri-miR | replicon | aptazyme | |
| | pri-miR | replicon | RNAseH primer TS | |
| | pri-miR | replicon | Restr Enz RS | |
| | ribozyme | replicon | siRNA TS | |
| | ribozyme | replicon | miR TS | |
| | ribozyme | replicon | AmiR TS | |
| | ribozyme | replicon | gRNA TS | |
| | ribozyme | replicon | pri-miR | |
| | ribozyme | replicon | aptazyme | |
| | ribozyme | replicon | RNAseH primer TS | |
| | ribozyme | replicon | Restr Enz RS | |
| | aptazyme | replicon | siRNA TS | |
| | aptazyme | replicon | miR TS | |
| | aptazyme | replicon | AmiR TS | |
| | aptazyme | replicon | gRNA TS | |
| | aptazyme | replicon | pri-miR | |
| | aptazyme | replicon | ribozyme | |
| | aptazyme | replicon | RNAseH primer TS | |
| | aptazyme | replicon | Restr Enz RS | |
| | RNAseH primer TS | replicon | siRNA TS | |
| | RNAseH primer TS | replicon | miR TS | |
| | RNAseH primer TS | replicon | AmiR TS | |
| | RNAseH primer TS | replicon | gRNA TS | |
| | RNAseH primer TS | replicon | pri-miR | |
| | RNAseH primer TS | replicon | ribozyme | |
| | RNAseH primer TS | replicon | aptazyme | |
| | RNAseH primer TS | replicon | Restr Enz RS | |

In some embodiments, the recombinant RNA replicons of the disclosure are produced in vitro by In vitro RNA transcription (See schematic in FIG. 27). The recombinant RNA replicons are then purified and formulated for therapeutic use (e.g., encapsulated into a lipid nanoparticle). In some embodiments, the DNA polynucleotide comprises, from 5′ to 3′: (i) a promoter sequence (e.g., a T7 polymerase promoter); (ii) a 5′ ribozyme sequence; (iii) a polynucleotide encoding the recombinant RNA replicon; and (iv) a 3′ ribozyme sequence. In some embodiments, the DNA polynucleotide comprises, from 5′ to 3′: (i) a promoter sequence (e.g., a T7 polymerase promoter); (ii) a 5′ Hammerhead ribozyme sequence (e.g., a wild type HHR or a modified HHR such as that provided in FIGS. 28A-28B); (iii) a polynucleotide encoding the recombinant RNA replicon; and (iv) a 3′ hepatitis delta virus ribozyme sequence.

In some embodiments, the DNA polynucleotide comprises, from 5′ to 3′: (i) a promoter sequence (e.g., a T7 polymerase promoter); (ii) a 5′ Hammerhead ribozyme sequence (e.g., a wild type HHR or a modified HHR such as that provided in FIGS. 28A-28B); (iii) a polynucleotide encoding a recombinant RNA replicon; and (iv) a 3′ hepatitis delta virus ribozyme sequence. In some embodiments, the DNA polynucleotide comprises, from 5′ to 3′: (i) a promoter sequence (e.g., a T7 polymerase promoter); (ii) a 5′ Hammerhead ribozyme sequence (e.g., a wild type HHR or a modified HHR such as that provided in FIGS. 28A-28B); (iii) a polynucleotide encoding a recombinant RNA replicon; and (iv) a 3′ hepatitis delta virus ribozyme sequence.

In some embodiments, the DNA polynucleotide comprises, from 5′ to 3′: (i) a promoter sequence (e.g., a T7 polymerase promoter); (ii) a 5′ ribozyme sequence; (iii) a polynucleotide encoding the recombinant RNA replicon; and (iv) a 3′ restriction enzyme recognition site. In some embodiments, the DNA polynucleotide comprises, from 5′ to 3′: (i) a promoter sequence (e.g., a T7 polymerase promoter); (ii) a 5′ Hammerhead ribozyme sequence (e.g., a wild type HHR or a modified HHR such as that provided in FIGS. 28A-28B); (iii) a polynucleotide encoding the recombinant RNA replicon; and (iv) a 3′ SapI restriction enzyme recognition site.

In some embodiments, the DNA polynucleotide comprises, from 5′ to 3′: (i) a promoter sequence (e.g., a T7 polymerase promoter); (ii) a 5′ Hammerhead ribozyme sequence (e.g., a wild type HHR or a modified HHR such as that provided in FIGS. 28A-28B); (iii) a polynucleotide encoding recombinant RNA replicon; and (iv) a 3′ SapI restriction enzyme recognition site.

In some embodiments, the DNA polynucleotide comprises, from 5′ to 3′: (i) a promoter sequence (e.g., a T7 polymerase promoter); (ii) a 5′ Pistol ribozyme sequence (e.g., a Pistol 1 or a Pistol 2 ribozyme sequence shown in FIGS. 29A-29B); (iii) a polynucleotide encoding the recombinant RNA replicon; and (iv) a 3′ SapI restriction enzyme recognition site. In some embodiments, the DNA polynucleotide comprises, from 5′ to 3′: (i) a promoter sequence (e.g., a T7 polymerase promoter); (ii) a 5′ Pistol 1 ribozyme sequence; (iii) a polynucleotide encoding a recombinant RNA replicon; and (iv) a 3′ SapI restriction enzyme recognition site. In some embodiments, the DNA polynucleotide comprises, from 5′ to 3′: (i) a promoter sequence (e.g., a T7 polymerase promoter); (ii) a 5′ Pistol 2 ribozyme sequence; (iii) a polynucleotide encoding a wild type SVV genome; and (iv) a 3′ SapI restriction enzyme recognition site. In some embodiments, the DNA polynucleotide comprises a nucleic acid sequence that is at least 95%, 96%, 97%, 98%, or 99% identical to SEQ ID NO: 15. In some embodiments, the DNA polynucleotide comprises or consists of SEQ ID NO: 15.

In some embodiments, the DNA polynucleotide comprises, from 5′ to 3′: (i) a promoter sequence (e.g., a T7 polymerase promoter); (ii) a 5′ RNAseH primer binding site; (iii) a polynucleotide encoding the recombinant RNA replicon; and (iv) a 3′ restriction enzyme recognition site. In some embodiments, the DNA vector comprises a polynucleotide comprising, from 5′ to 3′: (i) a promoter sequence (e.g., a T7 polymerase promoter); (ii) a 5′ RNAseH primer binding site; (iii) a polynucleotide encoding recombinant RNA replicons; and (iv) a 3′SapI restriction enzyme recognition site.

### Particles Comprising Recombinant RNA Replicons

In some embodiments, the recombinant RNA replicons of the disclosure are encapsulated in “particles.” As used herein, a particle refers to a non-tissue derived composition of matter such as liposomes, lipoplexes, nanoparticles, nanocapsules, microparticles, microspheres, lipid particles, exosomes, vesicles, and the like. In some embodiments, the particles are non-proteinaceous and non-immunogenic. In such embodiments, encapsulation of the recombinant RNA replicons of the disclosure allows for delivery of a viral genome without the induction of a systemic, anti-viral immune response and mitigates the effects of neutralizing anti-viral antibodies. Further, encapsulation of the recombinant RNA replicons of the disclosure shields the genomes from degradation and facilitates the introduction into target host cells. In some embodiments, the particles are nanoparticles. In some embodiments, the particles are lipid nanoparticles. In some embodiments, the particles are exosomes.

The disclosure provides particles comprising a recombinant RNA replicon of the disclosure. In some embodiments, the particle is a lipid nanoparticle. In some embodiments, the particles further comprise a second recombinant RNA molecule encoding an oncolytic virus. In some embodiments, the second recombinant RNA molecule encoding an oncolytic virus comprises a RNA viral genome (e.g., a RNA viral genome of an oncolytic virus). In some embodiments, the oncolytic virus is a picornavirus. In some embodiments, the picornavirus is selected from a senecavirus, a cardiovirus, and an enterovirus. In some embodiments, the picornavirus is a Seneca Valley Virus (SVV). In some embodiments, the picornavirus is a Coxsackievirus. In some embodiments, the picornavirus is an encephalomyocarditis virus (EMCV). In some embodiments, the RNA viral genome comprises intact VP1, VP2, VP3 and VP4 coding regions. In some embodiments, the RNA viral genome comprising intact VP1, VP2, VP3 and VP4 coding regions belongs to the same viral species or the same viral genus as the viral genome of the replicon. In some embodiments, the recombinant RNA replicon can be trans-encapsidated by the capsid proteins expressed from the RNA viral genome comprising intact VP coding regions. In some embodiments, the recombinant RNA replicon can be trans-encapsidated when the recombinant RNA replicon and the RNA viral genome are present in the same cell (e.g., by delivering them into the cell via the particle).

In some embodiments, the particle is biodegradable in a subject. In such embodiments, multiple doses of the particles can be administered to a subject without an accumulation of particles in the subject. Examples of suitable particles include polystyrene particles, poly(lactic-co-glycolic acid) PLGA particles, polypeptide-based cationic polymer particles, cyclodextrin particles, chitosan particles, lipid based particles, poly(β-amino ester) particles, low-molecular-weight polyethylenimine particles, polyphosphoester particles, disulfide cross-linked polymer particles, polyamidoamine particles, polyethylenimine (PEI) particles, and PLURIONICS stabilized polypropylene sulfide particles.

In some embodiments, the polynucleotides of the disclosure are encapsulated in inorganic particles. In some embodiments, the inorganic particles are gold nanoparticles (GNP), gold nanorods (GNR), magnetic nanoparticles (MNP), magnetic nanotubes (MNT), carbon nanohorns (CNH), carbon fullerenes, carbon nanotubes (CNT), calcium phosphate nanoparticles (CPNP), mesoporous silica nanoparticles (MSN), silica nanotubes (SNT), or a starlike hollow silica nanoparticles (SHNP).

In some embodiments, the particles of the disclosure are nanoscopic in size, in order to enhance solubility, avoid possible complications caused by aggregation in vivo and to facilitate pinocytosis. In some embodiments, the particle has an average diameter of about less than about 1000 nm. In some embodiments, the particle has an average diameter of less than about 500 nm. In some embodiments, the particle has an average diameter of between about 30 and about 100 nm, between about 50 and about 100 nm, or between about 75 and about 100 nm. In some embodiments, the particle has an average diameter of between about 30 and about 75 nm or between about 30 and about 50 nm. In some embodiments, the particle has an average diameter between about 100 and about 500 nm. In some embodiments, the particle has an average diameter between about 200 and 400 nm. In some embodiments, the particle has an average size of about 350 nm.

### Exosomes

In some embodiments, the recombinant RNA replicons of the disclosure are encapsulated in exosomes. Exosomes are small membrane vesicles of endocytic origin that are released into the extracellular environment following fusion of multivesicular bodies with the plasma membrane of the parental cell (e.g., the cell from which the exosome is released, also referred to herein as a donor cell). The surface of an exosome comprises a lipid bilayer derived from the parental cell's cell membrane and can further comprise membrane proteins expressed on the parental cell surface. In some embodiments, exosomes may also contain cytosol from the parental cell. Exosomes are produced by many different cell types including epithelial cells, B and T lymphocytes, mast cells (MC), and dendritic cells (DC) and have been identified in blood plasma, urine, bronchoalveolar lavage fluid, intestinal epithelial cells, and tumor tissues. Because the composition of an exosome is dependent on the parental cell type from which they are derived, there are no “exosome-specific” proteins. However, many exosomes comprise proteins associated with the intracellular vesicles from which the exosome originated in the parental cells (e.g., proteins associated with and/or expressed by endosomes and lysosomes). For example, exosomes can be enriched in antigen presentation molecules such as major histocompatibility complex I and II (MHC-I and MHC-II), tetraspanins (e.g., CD63), several heat shock proteins, cytoskeletal components such as actins and tubulins, proteins involved in intracellular membrane fusion, cell-cell interactions (e.g. CD54), signal transduction proteins, and cytosolic enzymes.

Exosomes may mediate transfer of cellular proteins from one cell (e.g., a parental cells) to a target or recipient cell by fusion of the exosomal membrane with the plasma membrane of the target cell. As such, modifying the material that is encapsulated by the exosome provides a mechanism by which exogenous agents, such as the polynucleotides described herein, may be introduced to a target cell. Exosomes that have been modified to contain one or more exogenous agents (e.g., a polynucleotide described herein) are referred to herein as “modified exosomes”. In some embodiments, modified exosomes are produced by introduction of the exogenous agent (e.g., a polynucleotide described herein) are introduced into a parental cell. In such embodiments, an exogenous nucleic acid is introduced into the parental, exosome-producing cells such that the exogenous nucleic acid itself, or a transcript of the exogenous nucleic acid is incorporated into the modified exosomes produced from the parental cell. The exogenous nucleic acids can be introduced to the parental cell by means known in the art, for example transduction, transfection, transformation, and/or microinjection of the exogenous nucleic acids.

In some embodiments, modified exosomes are produced by directly introducing recombinant RNA replicons of the disclosure into an exosome. In some embodiments, recombinant RNA replicons of the disclosure is introduced into an intact exosome. “Intact exosomes” refer to exosomes comprising proteins and/or genetic material derived from the parental cell from which they are produced. Methods for obtaining intact exosomes are known in the art (See e.g., Alvarez-Erviti L. et al., Nat Biotechnol. 2011 April; 29(4):34-5; Ohno S, et al., Mol Ther 2013 January; 21(1):185-91; and EP Patent Publication No. 2010663).

In some embodiments, recombinant RNA replicons are introduced into empty exosomes. “Empty exosomes” refer to exosomes that lack proteins and/or genetic material (e.g., DNA or RNA) derived from the parental cell. Methods to produce empty exosomes (e.g., lacking parental cell-derived genetic material) are known in the art including UV-exposure, mutation/deletion of endogenous proteins that mediate loading of nucleic acids into exosomes, as well as electroporation and chemical treatments to open pores in the exosomal membranes such that endogenous genetic material passes out of the exosome through the open pores. In some embodiments, empty exosomes are produced by opening the exosomes by treatment with an aqueous solution having a pH from about 9 to about 14 to obtain exosomal membranes, removing intravesicular components (e.g., intravesicular proteins and/or nucleic acids), and reassembling the exosomal membranes to form empty exosomes. In some embodiments, intravesicular components (e.g., intravesicular proteins and/or nucleic acids) are removed by ultracentrifugation or density gradient ultracentrifugation. In some embodiments, the membranes are reassembled by sonication, mechanical vibration, extrusion through porous membranes, electric current, or combinations of one or more of these techniques. In particular embodiments, the membranes are reassembled by sonication.

In some embodiments, loading of intact or empty exosomes with the recombinant RNA replicons described herein to produce a modified exosome can be achieved using conventional molecular biology techniques such as in vitro transformation, transfection, and/or microinjection. In some embodiments, the exogenous agents (e.g., the polynucleotides described herein) are introduced directly into intact or empty exosomes by electroporation. In some embodiments, the exogenous agents (e.g., the polynucleotides described herein) are introduced directly into intact or empty exosomes by lipofection (e.g., transfection). Lipofection kits suitable for use in the production of exosome according to the present disclosure are known in the art and are commercially available (e.g., FuGENE® HD Transfection Reagent from Roche, and LIPOFECTAMINE™ 2000 from Invitrogen). In some embodiments, the exogenous agents (e.g., the polynucleotides described herein) are introduced directly into intact or empty exosomes by transformation using heat shock. In such embodiments, exosomes isolated from parental cells are chilled in the presence of divalent cations such as Ca²⁺ (in CaCl₂)) in order to permeabilize the exosomal membrane. The exosomes can then be incubated with the exogenous nucleic acids and briefly heat shocked (e.g., incubated at 42° C. for 30-120 seconds). In particular embodiments, loading of empty exosomes with exogenous agents (e.g., the polynucleotides described herein) can be achieved by mixing or co-incubation of the agents with the exosomal membranes after the removal of intravesicular components. The modified exosomes reassembled from the exosomal membranes will, therefore, incorporate the exogenous agents into the intravesicular space. Additional methods for producing exosome encapsulated nucleic acids are known in the art (See e.g., U.S. Pat. Nos. 9,889,210; 9,629,929; and 9,085,778; International PCT Publication Nos. WO 2017/161010 and WO 2018/039119).

Exosomes can be obtained from numerous different parental cells, including cell lines, bone-marrow derived cells, and cells derived from primary patient samples. Exosomes released from parental cells can be isolated from supernatants of parental cell cultures by means known in the art. For example, physical properties of exosomes can be employed to separate them from a medium or other source material, including separation on the basis of electrical charge (e.g., electrophoretic separation), size (e.g., filtration, molecular sieving, etc.), density (e.g., regular or gradient centrifugation) and Svedberg constant (e.g., sedimentation with or without external force, etc). Alternatively, or additionally, isolation can be based on one or more biological properties, and include methods that can employ surface markers (e.g., for precipitation, reversible binding to solid phase, FACS separation, specific ligand binding, non-specific ligand binding, etc.). Analysis of exosomal surface proteins can be determined by flow cytometry using fluorescently labeled antibodies for exosome-associated proteins such as CD63. Additional markers for characterizing exosomes are described in International PCT Publication No. WO 2017/161010. In yet further contemplated methods, the exosomes can also be fused using chemical and/or physical methods, including PEG-induced fusion and/or ultrasonic fusion.

In some embodiments, size exclusion chromatography can be utilized to isolate the exosomes. In some embodiments, the exosomes can be further isolated after chromatographic separation by centrifugation techniques (of one or more chromatography fractions), as is generally known in the art. In some embodiments, the isolation of exosomes can involve combinations of methods that include, but are not limited to, differential centrifugation as previously described (See Raposo, G. et al., J. Exp. Med. 183, 1161-1172 (1996)), ultracentrifugation, size-based membrane filtration, concentration, and/or rate zonal centrifugation.

In some embodiments, the exosomal membrane comprises one or more of phospholipids, glycolipids, fatty acids, sphingolipids, phosphoglycerides, sterols, cholesterols, and phosphatidylserine. In addition, the membrane can comprise one or more polypeptides and one or more polysaccharides, such as glycans. Exemplary exosomal membrane compositions and methods for modifying the relative amount of one or more membrane component are described in International PCT Publication No. WO 2018/039119.

In some embodiments, the particles are exosomes and have a diameter between about 30 and about 100 nm, between about 30 and about 200 nm, or between about 30 and about 500 nm. In some embodiments, the particles are exosomes and have a diameter between about 10 nm and about 100 nm, between about 20 nm and about 100 nm, between about 30 nm and about 100 nm, between about 40 nm and about 100 nm, between about 50 nm and about 100 nm, between about 60 nm and about 100 nm, between about 70 nm and about 100 nm, between about 80 nm and about 100 nm, between about 90 nm and about 100 nm, between about 100 nm and about 200 nm, between about 100 nm and about 150 nm, between about 150 nm and about 200 nm, between about 100 nm and about 250 nm, between about 250 nm and about 500 nm, or between about 10 nm and about 1000 nm. In some embodiments, the particles are exosomes and have a diameter between about 20 nm and 300 nm, between about 40 nm and 200 nm, between about 20 nm and 250 nm, between about 30 nm and 150 nm, or between about 30 nm and 100 nm.

### Lipid Nanoparticles

In some embodiments, the recombinant RNA replicons described herein are encapsulated in a lipid nanoparticle (LNP). In certain embodiments, the LNP comprises one or more lipids such as such as triglycerides (e.g. tristearin), diglycerides (e.g. glycerol bahenate), monoglycerides (e.g. glycerol monostearate), fatty acids (e.g. stearic acid), steroids (e.g. cholesterol), and waxes (e.g. cetyl palmitate). In some embodiments, the LNP comprises one or more cationic lipids and one or more helper lipids. In some embodiments, the LNP comprises one or more cationic lipids, a cholesterol, and one or more neutral lipids

Cationic lipids refer to any of a number of lipid species that carry a net positive charge at a selected pH, such as physiological pH. Such lipids include, but are not limited to 1,2-DiLinoleyloxy-N,N-dimethylaminopropane (DLinDMA), 1,2-Dilinolenyloxy-N,N-dimethylaminopropane (DLenDMA), dioctadecyldimethylammonium (DODMA), distearyldimethylammonium (DSDMA), N,N-dioleyl-N,N-dimethylammonium chloride (DODAC); N-(2,3-dioleyloxy)propyl)-N,N,N-trimethylammonium chloride (DOTMA); N,N-distearyl-N,N-dimethylammonium bromide (DDAB); N-(2,3-dioleoyloxy)propyl)-N,N,N-trimethylammonium chloride (DOTAP); 3-(N-(N′,N′-dimethylaminoethane)-carbamoyl)cholesterol (DC-Chol), and N-(1,2-dimyristyloxyprop-3-yl)-N,N-dimethyl-N-hydroxyethyl ammonium bromide (DMRIE). For example, cationic lipids that have a positive charge at below physiological pH include, but are not limited to, DODAP, DODMA, and DMDMA. In some embodiments, the cationic lipids comprise Cis alkyl chains, ether linkages between the head group and alkyl chains, and 0 to 3 double bonds. Such lipids include, e.g., DSDMA, DLinDMA, DLenDMA, and DODMA. The cationic lipids may comprise ether linkages and pH titratable head groups. Such lipids include, e.g., DODMA.

In some embodiments, the cationic lipids comprise a protonatable tertiary amine head group. Such lipids are referred to herein as ionizable lipids. Ionizable lipids refer to lipid species comprising an ionizable amine head group and typically comprising a pKa of less than about 7. Therefore, in environments with an acidic pH, the ionizable amine head group is protonated such that the ionizable lipid preferentially interacts with negatively charged molecules (e.g., nucleic acids such as the recombinant polynucleotides described herein) thus facilitating nanoparticle assembly and encapsulation. Therefore, in some embodiments, ionizable lipids can increase the loading of nucleic acids into lipid nanoparticles. In environments where the pH is greater than about 7 (e.g., physiologic pH of ≈7.4), the ionizable lipid comprises a neutral charge. When particles comprising ionizable lipids are taken up into the low pH environment of an endosome (e.g., pH <7), the ionizable lipid is again protonated and associates with the anionic endosomal membranes, promoting release of the contents encapsulated by the particle. In some embodiments, the LNP comprises an ionizable lipid, e.g., a 7.SS-cleavable and pH-responsive Lipid Like Material (such as the COATSOME® SS-Series).

In some embodiments, the cationic lipid is an ionizable lipid selected from DLinDMA, DLin-KC2-DMA, DLin-MC3-DMA (MC3), COATSOME® SS-LC (former name: SS-18/4PE-13), COATSOME® SS-EC (former name: SS-33/4PE-15), COATSOME® SS-OC, COATSOME® SS-OP, Di((Z)-non-2-en-1-yl)9-((4-dimethylamino)butanoyl)oxy) heptadecanedioate (L-319), or N-(2,3-dioleoyloxy)propyl)-N,N,N-trimethylammonium chloride (DOTAP). In some embodiments, the cationic ionizable lipid is DLin-MC3-DMA (MC3). In some embodiments, the cationic ionizable lipid is COATSOME® SS-LC. In some embodiments, the cationic ionizable lipid is COATSOME® SS-EC. In some embodiments, the cationic ionizable lipid is COATSOME® SS-OC. In some embodiments, the cationic ionizable lipid is COATSOME® SS-OP. In some embodiments, the cationic ionizable lipid is L-319. In some embodiments, the cationic ionizable lipid is DOTAP.

In some embodiments, the LNPs comprise one or more non-cationic helper lipids (neutral lipids). Exemplary neutral helper lipids include (1,2-dilauroyl-sn-glycero-3-phosphoethanolamine) (DLPE), 1,2-diphytanoyl-sn-glycero-3-phosphoethanolamine (DiPPE), 1,2-distearoyl-sn-glycero-3-phosphocholine (DSPC), 1,2-dipalmitoyl-sn-glycero-3-phosphocholine (DPPC), 1,2-dioleyl-sn-glycero-3-phosphoethanolamine (DOPE), 1,2-dipalmitoyl-sn-glycero-3-phosphoethanolamine (DPPE), 1,2-dimyristoyl-sn-glycero-3-phosphoethanolamine (DMPE), (1,2-dioleoyl-sn-glycero-3-phospho-(1′-rac-glycerol) (DOPG), 1,2-dioleoyl-sn-glycero-3-phosphocholine (DOPC), 1,2-distearoyl-sn-glycero-3-phosphoethanolamine (DSPE), ceramides, sphingomyelins, and cholesterol. In some embodiments, the one or more helper lipids are selected from 1,2-distearoyl-sn-glycero-3-phosphocholine (DSPC); 1,2-dilauroyl-sn-glycero-3-phosphoethanolamine (DLPE); 1,2-dioleoyl-sn-glycero-3-phosphocholine (DOPC); 1,2-dioleoyl-sn-glycero-3-phosphoethanolamine (DOPE); and cholesterol. In some embodiments, the LNPs comprise DSPC. In some embodiments, the LNPs comprise DOPC. In some embodiments, the LNPs comprise DLPE. In some embodiments, the LNPs comprise DOPE.

The use and inclusion of polyethylene glycol (PEG)-modified phospholipids and derivatized lipids such as derivatized ceramides (PEG-CER), including N-octanoyl-sphingosine-1-[succinyl(methoxy polyethylene glycol)-2000] (C8 PEG-2000 ceramide) in the liposomal and pharmaceutical compositions described herein is also contemplated, preferably in combination with one or more of the compounds and lipids disclosed herein.

In some embodiments, the lipid nanoparticles may further comprise one or more of PEG-modified lipids that comprise a poly(ethylene)glycol chain of up to 5 kDa in length covalently attached to a lipid comprising one or more C6-C20 alkyls. In some embodiments, the LNPs further comprise 1,2-Distearoyl-sn-glycero-3-phosphoethanolamine-Poly(ethylene glycol) (DSPE-PEG), or 1,2-distearoyl-sn-glycero-3-phosphoethanolamine-N-[amino(polyethylene glycol)] (DSPE-PEG-amine). In some embodiments, the LNPs further comprise a PEG-modified lipid selected from 1,2-distearoyl-sn-glycero-3-phosphoethanolamine-N-[amino(polyethyleneglycol)-5000] (DSPE-PEG5K); 1,2-dipalmitoyl-rac-glycerol methoxypolyethylene glycol-2000 (DPG-PEG2K); 1,2-distearoyl-rac-glycero-3-methylpolyoxyethylene-5000 (DSG-PEG5K); 1,2-distearoyl-rac-glycero-3-methylpolyoxyethylene-2000 (DSG-PEG2K); 1,2-dimyristoyl-rac-glycero-3-methylpolyoxyethylene-5000 (DMG-PEG5K); and 1,2-dimyristoyl-rac-glycero-3-methylpolyoxyethylene-2000 (DMG-PEG2K). In some embodiments, the LNPs further comprise DSPE-PEG5K. In some embodiments, the LNPs further comprise DPG-PEG2K. In some embodiments, the LNPs further comprise DSG-PEG2K. In some embodiments, the LNPs further comprise DMG-PEG2K. In some embodiments, the LNPs further comprise DSG-PEG5K. In some embodiments, the LNPs further comprise DMG-PEG5K. In some embodiments, the PEG-modified lipid comprises about 0.1% to about 1% of the total lipid content in a lipid nanoparticle. In some embodiments, the PEG-modified lipid comprises about 0.1%, about 0.2% about 0.3%, about 0.4%, about 0.5%, about 0.6%, about 0.7%, about 0.8%, about 0.9%, about 1.0%, about 1.5%, about 2.0%, about 2.5%, or about 3.0% of the total lipid content in the lipid nanoparticle.

In some embodiments, the lipid is modified with a cleavable PEG lipid. Examples of PEG derivatives with cleavable bonds include those modified with peptide bonds (Kulkarni et al. (2014). Mmp-9 responsive PEG cleavable nanovesicles for efficient delivery of chemotherapeutics to pancreatic cancer. Mol Pharmaceutics 11:2390-9; Lin et al. (2015). Drug/dye-loaded, multifunctional peg-chitosan-iron oxide nanocomposites for methotraxate synergistically self-targeted cancer therapy and dual model imaging. ACS Appl Mater Interfaces 7:11908-20.), disulfide keys (Yan et al (2014). A method to accelerate the gelation of disulfide-crosslinked hydrogels. Chin J Polym Sci 33:118-27; Wu & Yan (2015). Copper nanopowder catalyzed cross-coupling of diaryl disulfides with aryl iodides in PEG-400. Synlett 26:537-42), vinyl ether bonds, hydrazone bonds (Kelly et al. (2016). Polymeric prodrug combination to exploit the therapeutic potential of antimicrobial peptides against cancer cells. Org Biomol Chem 14:9278-86.), and ester bonds (Xu et al. (2008). Esterase-catalyzed dePEGylation of pH-sensitive vesicles modified with cleavable PEG-lipid derivatives. J Control Release 130:238-45). See also, Fang et al., (2017) Cleaveable PEGylation: a strategy for overcoming the “PEG dilemma” in efficient drug delivery. Drug Delivery 24:2, 22-32.

In some embodiments, the PEG lipid is an activated PEG lipid. Exemplary activated PEG lipids include PEG-NH2, PEG-MAL, PEG-NHS, and PEG-ALD. Such functionalized PEG lipids are useful in the conjugation of targeting moieties to lipid nanoparticles to direct the particles to a particular target cell or tissue (e.g., by the attachment of antigen-binding molecules, peptides, glycans, etc.).

In some embodiments, the LNP comprises a cationic lipid and one or more helper lipids, wherein the cationic lipid is DOTAP. In some embodiments, the LNP comprises a cationic lipid and one or more helper lipids, wherein the cationic lipid is DLin-MC3-DMA (MC3). In some embodiments, the LNP comprises a cationic lipid and one or more helper lipids, wherein the cationic lipid is COATSOME® SS-EC. In some embodiments, the LNP comprises a cationic lipid and one or more helper lipids, wherein the cationic lipid is COATSOME® SS-LC. In some embodiments, the LNP comprises a cationic lipid and one or more helper lipids, wherein the cationic lipid is COATSOME® SS-OC. In some embodiments, the LNP comprises a cationic lipid and one or more helper lipids, wherein the cationic lipid is COATSOME® SS-OP. In some embodiments, the LNP comprises a cationic lipid and one or more helper lipids, wherein the cationic lipid is L-319.

In some embodiments, the LNP comprises a cationic lipid and one or more helper lipids, wherein the one or more helper lipids comprises cholesterol. In some embodiments, the LNP comprises a cationic lipid and one or more helper lipids, wherein the one or more helper lipids comprises DLPE. In some embodiments, the LNP comprises a cationic lipid and one or more helper lipids, wherein the one or more helper lipids comprises DSPC. In some embodiments, the LNP comprises a cationic lipid and one or more helper lipids, wherein the one or more helper lipids comprises DOPE. In some embodiments, the LNP comprises a cationic lipid and one or more helper lipids, wherein the one or more helper lipids comprises DOPC.

In some embodiments, the LNP comprises a cationic lipid and at least two helper lipids, wherein the cationic lipid is DOTAP, and the at least two helper lipids comprise cholesterol and DLPE. In some embodiments, the LNP comprises a cationic lipid and at least two helper lipids, wherein the cationic lipid is MC3, and the at least two helper lipids comprise cholesterol and DSPC. In some embodiments, the at least two helper lipids comprise cholesterol and DOPE. In some embodiments, the at least two helper lipids comprise cholesterol and DSPC. In some embodiments, the LNP comprises a cationic lipid and at least three helper lipids, wherein the cationic lipid is DOTAP, and the at least three helper lipids comprise cholesterol, DLPE, and DSPE. In some embodiments, the LNP comprises a cationic lipid and at least three helper lipids, wherein the cationic lipid is MC3, and the at least three helper lipids comprise cholesterol, DSPC, and DMG. In some embodiments, the at least three helper lipids comprise cholesterol, DOPE, and DSPE. In some embodiments, the at least three helper lipids comprise cholesterol, DSPC, and DMG. In some embodiments, the LNP comprises DOTAP, cholesterol, and DLPE. In some embodiments, the LNP comprises MC3, cholesterol, and DSPC. In some embodiments, the LNP comprises DOTAP, cholesterol, and DOPE. In some embodiments, the LNP comprises DOTAP, cholesterol, DLPE, and DSPE. In some embodiments, the LNP comprises MC3, cholesterol, DSPC, and DMG. In some embodiments, the LNP comprises DOTAP, cholesterol, DLPE, and DSPE-PEG. In some embodiments, the LNP comprises MC3, cholesterol, DSPC, and DMG-PEG. In some embodiments, the LNP comprises DOTAP, cholesterol, DOPE, and DSPE. In some embodiments, the LNP comprises DOTAP, cholesterol, DOPE, and DSPE-PEG. In some embodiments, the LNP comprises SS-OC, DSPC, cholesterol, and DPG-PEG (e.g., DPG-PEG2K).

In some embodiments, the LNP comprises DOTAP, cholesterol (Chol), and DLPE, wherein the ratio of DOTAP:Chol:DLPE (as a percentage of total lipid content) is about 50:35:15. In some embodiments, the LNP comprises DOTAP, cholesterol (Chol), and DLPE, wherein the ratio of DOTAP:Chol:DOPE (as a percentage of total lipid content) is about 50:35:15. In some embodiments, the LNP comprises DOTAP, cholesterol (Chol), DLPE, DSPE-PEG, wherein the ratio of DOTP:Chol:DLPE (as a percentage of total lipid content) is about 50:35:15 and wherein the particle comprises about 0.2% DSPE-PEG. In some embodiments, the LNP comprises MC3, cholesterol (Chol), DSPC, and DMG-PEG, wherein the ratio of MC3:Chol:DSPC:DMG-PEG (as a percentage of total lipid content) is about 49:38.5:11:1.5.

In some embodiments, the LNP comprises SS-OC, DSPC, cholesterol (Chol), and DPG-PEG2K), wherein the ratio of SS-OC:DSPC:Chol:DPG-PEG2K (as a percentage of total lipid content) is about A:B:C:D, wherein A=40%-60%, B=10%-25%, C=20%-30%, and D=0%-3% and wherein A+B+C+D=100%. In some embodiments, the LNP comprises SS-OC, DSPC, cholesterol (Chol), and DPG-PEG2K, wherein the ratio of SS-OC:DSPC:Chol:DPG-PEG2K (as a percentage of total lipid content) is about A:B:C:D, wherein A=45%-50%, B=20%-25%, C=25%-30%, and D=0%-1% and wherein A+B+C+D=100%. In some embodiments, the LNP comprises SS-OC, DSPC, cholesterol (Chol), and DPG-PEG2K, wherein the ratio of SS-OC:DSPC:Chol:DPG-PEG2K (as a percentage of total lipid content) is about 49:22:28.5:0.5.

In some embodiments, the LNP comprises SS-OC, DSPC, cholesterol (Chol), and DPG-PEG2K, wherein the ratio of SS-OC:DSPC:Chol:DPG-PEG2K (as a percentage of total lipid content) is about A:B:C:D, wherein A=40%-60%, B=10%-30%, C=20%-45%, and D=0%-3% and wherein A+B+C+D=100%. In some embodiments, the LNP comprises SS-OC, DSPC, cholesterol (Chol), and DPG-PEG2K, wherein the ratio of SS-OC:DSPC:Chol:DPG-PEG2K (as a percentage of total lipid content) is about A:B:C:D, wherein A=40%-60%, B=10%-30%, C=25%-45%, and D=0%-3% and wherein A+B+C+D=100%. In some embodiments, the LNP comprises SS-OC, DSPC, cholesterol (Chol), and DPG-PEG2K, wherein the ratio of SS-OC:DSPC:Chol:DPG-PEG2K (as a percentage of total lipid content) is about A:B:C:D, wherein A=45%-55%, B=10%-20%, C=30%-40%, and D=1%-2% and wherein A+B+C+D=100%. In some embodiments, the LNP comprises SS-OC, DSPC, cholesterol (Chol), and DPG-PEG2K, wherein the ratio of SS-OC:DSPC:Chol:DPG-PEG2K (as a percentage of total lipid content) is about A:B:C:D, wherein A=45%-50%, B=10%-15%, C=35%-40%, and D=1%-2% and wherein A+B+C+D=100%. In some embodiments, the LNP comprises SS-OC, DSPC, cholesterol (Chol), and DPG-PEG2K, wherein the ratio of SS-OC:DSPC:Chol:DPG-PEG2K (as a percentage of total lipid content) is 49:11:38.5:1.5.

In some embodiments, the LNP comprises SS-OC, DSPC, cholesterol (Chol), and DPG-PEG2K, wherein the ratio of SS-OC:DSPC:Chol:DPG-PEG2K (as a percentage of total lipid content) is about A:B:C:D, wherein A=45%-65%, B=5%-20%, C=20%-45%, and D=0%-3% and wherein A+B+C+D=100%. In some embodiments, the LNP comprises SS-OC, DSPC, cholesterol (Chol), and DPG-PEG2K, wherein the ratio of SS-OC:DSPC:Chol:DPG-PEG2K (as a percentage of total lipid content) is about A:B:C:D, wherein A=50%-60%, B=5%-15%, C=30%-45%, and D=0%-3% and wherein A+B+C+D=100%. In some embodiments, the LNP comprises SS-OC, DSPC, cholesterol (Chol), and DPG-PEG2K, wherein the ratio of SS-OC:DSPC:Chol:DPG-PEG2K (as a percentage of total lipid content) is about A:B:C:D, wherein A=55%-60%, B=5%-15%, C=30%-40%, and D=1%-2% and wherein A+B+C+D=100%. In some embodiments, the LNP comprises SS-OC, DSPC, cholesterol (Chol), and DPG-PEG2K, wherein the ratio of SS-OC:DSPC:Chol:DPG-PEG2K (as a percentage of total lipid content) is about A:B:C:D, wherein A=55%-60%, B=5%-10%, C=30%-35%, and D=1%-2% and wherein A+B+C+D=100%. In some embodiments, the LNP comprises SS-OC, DSPC, cholesterol (Chol), and DPG-PEG2K, wherein the ratio of SS-OC:DSPC:Chol:DPG-PEG2K (as a percentage of total lipid content) is 58:7:33.5:1.5.

In some embodiments, the nanoparticle is coated with a glycosaminoglycan (GAG) in order to modulate or facilitate uptake of the nanoparticle by target cells. The GAG may be heparin/heparin sulfate, chondroitin sulfate/dermatan sulfate, keratin sulfate, or hyaluronic acid (HA). In a particular embodiment, the surface of the nanoparticle is coated with HA and targets the particles for uptake by tumor cells. In some embodiments, the lipid nanoparticle is coated with an arginine-glycine-aspartate tri-peptide (RGD peptides) (See Ruoslahti, Advanced Materials, 24, 2012, 3747-3756; and Bellis et al., Biomaterials, 32(18), 2011, 4205-4210).

In some embodiments, the LNPs have an average size of about 50 nm to about 500 nm. For example, in some embodiments, the LNPs have an average size of about 50 nm to about 200 nm, about 100 nm to about 200 nm, about 150 nm to about 200 nm, about 50 nm to about 150 nm, about 100 nm to about 150 nm, about 150 nm to about 500 nm, about 200 nm to about 500 nm, about 300 nm to about 500 nm, about 350 nm to about 500 nm, about 400 nm to about 500 nm, about 425 nm to about 500 nm, about 450 nm to about 500 nm, or about 475 nm to about 500 nm. In some embodiments, the plurality of LNPs have an average size of about 50 nm to about 120 nm. In some embodiments, the plurality of LNPs have an average size of about 50 nm, 60 nm, 70 nm, 80 nm, 90 nm, 100 nm, 110 nm, or about 120 nm. In some embodiments, the plurality of LNPs have an average size of about 100 nm.

In some embodiments, the LNPs have a neutral charge (e.g., an average zeta-potential of between about 0 mV and 1 mV). In some embodiments, the LNPs have an average zeta-potential of between about 40 mV and about −40 mV. In some embodiments, the LNPs have an average zeta-potential of between about 40 mV and about 0 mV. In some embodiments, the LNPs have an average zeta-potential of between about 35 mV and about 0 mV, about 30 mV and about 0 mV, about 25 mV to about 0 mV, about 20 mV to about 0 mV, about 15 mV to about 0 mV, about 10 mV to about 0 mV, or about 5 mV to about 0 mV. In some embodiments, the LNPs have an average zeta-potential of between about 20 mV and about −40 mV. In some embodiments, the LNPs have an average zeta-potential of between about 20 mV and about −20 mV. In some embodiments, the LNPs have an average zeta-potential of between about 10 mV and about −20 mV. In some embodiments, the LNPs have an average zeta-potential of between about 10 mV and about −10 mV. In some embodiments, the LNPs have an average zeta-potential of about 10 mV, about 9 mV, about 8 mV, about 7 mV, about 6 mV, about 5 mV, about 4 mV, about 3 mV, about 2 mV, about 1 mV, about 0 mV, about −1 mV, about −2 mV, about −3 mV, about −4 mV, about −5 mV, about −6 mV, about −7 mV, about −8 mV, about −9 mV, about −9 mV or about −10 mV.

In some embodiments, the LNPs have an average zeta-potential of between about 0 mV and −20 mV. In some embodiments, the LNPs have an average zeta-potential of less than about −20 mV. For example in some embodiments, the LNPs have an average zeta-potential of less than about less than about −30 mV, less than about 35 mV, or less than about −40 mV. In some embodiments, the LNPs have an average zeta-potential of between about −50 mV to about-20 mV, about −40 mV to about −20 mV, or about −30 mV to about −20 mV. In some embodiments, the LNPs have an average zeta-potential of about 0 mV, about −1 mV, about −2 mV, about −3 mV, about −4 mV, about −5 mV, about −6 mV, about −7 mV, about −8 mV, about −9 mV, about −10 mV, about −11 mV, about −12 mV, about −13 mV, about −14 mV, about −15 mV, about −16 mV, about −17 mV, about −18 mV, about −19 mV, about −20 mV, about −21 mV, about −22 mV, about −23 mV, about −24 mV, about −25 mV, about −26 mV, about −27 mV, about −28 mV, about −29 mV, about −30 mV, about −31 mV, about −32 mV, about −33 mV, about −34 mV, about −35 mV, about −36 mV, about −37 mV, about −38 mV, about −39 mV, or about −40 mV.

In some embodiments, the lipid nanoparticles comprise a recombinant nucleic acid molecule described herein and comprise a ratio of lipid (L) to nucleic acid (N) of about 3:1 (L:N). In some embodiments, the lipid nanoparticles comprise a recombinant nucleic acid molecule described herein and comprise an L:N ratio about 4:1, about 5:1, about 6:1, about 7:1, about 8:1, about 9:1, or about 10:1. In some embodiments, the lipid nanoparticles comprise a recombinant nucleic acid molecule described herein and comprise a ratio of lipid (L) to nucleic acid (N) of about 7:1. In some embodiments, the lipid nanoparticles comprise a recombinant nucleic acid molecule described herein and comprise an L:N ratio about 4.5:1, about 4.6:1, about 4.7:1, about 4.8:1, about 4.9:1, about 5:1, about 5.1:1, about 5.2:1, about 5.3:1, about 5.4:1, or about 5.5:1. In some embodiments, the lipid nanoparticles comprise a recombinant nucleic acid molecule described herein and comprise an L:N ratio about 6.5:1, 6.6:1, 6.7:1, 6.8:1, 6.9:1, 7:1, 7.1:1, 7.2:1, 7.3:1, 7.4:1, and 7.5:1.

In some embodiments, the LNP comprises a lipid formulation selected from one of the formulations listed in Table 14.

TABLE 14
Exemplary Replicon Containing Lipid Nanoparticles

| Formulation | | Ionizable | Cholesterol | Helper | PEGylated | |
|---|---|---|---|---|---|---|
| ID | Buffer | lipid (%) | (%) | lipid (%) | lipid (%) | N:P |
| 70001-5C | PB, pH 5.8 | MC3 | Cholesterol | DSPC | DSPE-PEG5K | 7 |
| | | (49%) | (39.8%) | (11%) | (0.2%) | |
| 70009-1.C | PB, pH 5.8 | MC3 | Cholesterol | DSPC | DSPE-PEG5K | 7 |
| | | (49%) | (39.8%) | (11%) | (0.2%) | |
| 70009-2.C | PB, pH 5.8 | MC3 | Cholesterol | DSPC | DSPE-PEG5K | 7 |
| | | (49%) | (39.8%) | (11%) | (0.2%) | |
| 70009-3.C | PB, pH 5.8 | MC3 | Cholesterol | DSPC | DSPE-PEG5K | 7 |
| | | (49%) | (39.8%) | (11%) | (0.2%) | |
| 70032-1.C | PB, pH 5.8 | MC3 | Cholesterol | DSPC | DSPE-PEG5K | 7 |
| | | (49%) | (39.8%) | (11%) | (0.2%) | |
| 70032-2.C | PB, pH 5.8 | MC3 | Cholesterol | DSPC | DMG-PEG2K | 7 |
| | | (49%) | (38.5%) | (11%) | (1.5%) | |
| 70032-3.C | PB, pH 5.8 | MC3 | Cholesterol | DSPC | DSPE-PEG5K | 5 |
| | | (49%) | (39.8%) | (11%) | (0.2%) | |
| 70032-4.C | PB, pH 5.8 | MC3 | Cholesterol | DSPC | DSPE-PEG5K | 3 |
| | | (49%) | (39.8%) | (11%) | (0.2%) | |
| 70032-5.C | PB, pH 7.4 | DOTAP | Cholesterol | DLPE | DSPE-PEG5K | 5.33 |
| | | (50%) | (34.8%) | (15%) | (0.2%) | |
| 70032-6C | PB, pH 5.8 | MC3 | Cholesterol | DSPC | DSPE-PEG5K | 7 |
| | | (49%) | (39.8%) | (11%) | (0.2%) | |
| 70041-2.C | MB, pH 3.0 | SS-EC | Cholesterol | DSPC | DSPE-PEG5K | 7 |
| | | (49%) | (39.8%) | (11%) | (0.2%) | |
| 70041-3.C | MB, pH 3.0 | SS-LC | Cholesterol | DSPC | DSPE-PEG5K | 7 |
| | | (49%) | (39.8%) | (11%) | (0.2%) | |
| 70041-4.C | MB, pH 3.0 | SS-OC | Cholesterol | DSPC | DSPE-PEG5K | 7 |
| | | (49%) | (39.8%) | (11%) | (0.2%) | |
| 70046-4.C | MB, pH 3.0 | SS-EC | Cholesterol | DOPC | DSG-PEG5K | 9 |
| | | (56%) | (28.0%) | (9%) | (7%) | |
| 70046-5.C | MB, pH 3.0 | SS-LC | Cholesterol | DOPC | DSG-PEG5K | 9 |
| | | (56%) | (28.0%) | (9%) | (7%) | |
| 70046-6.C | MB, pH 3.0 | SS-OC | Cholesterol | DOPC | DSG-PEG5K | 9 |
| | | (56%) | (28.0%) | (9%) | (7%) | |
| 70053-1.C | PB, pH 5.8 | MC3 | Cholesterol | DSPC | DSPE-PEG5K | 7 |
| | | (49%) | (39.8%) | (11%) | (0.2%) | |
| 70053-2.C | MB, pH 3.0 | SS-LC | Cholesterol | DSPC | DSPE-PEG5K | 7 |
| | | (49%) | (39.8%) | (11%) | (0.2%) | |
| 70059-1.C | MB, pH 3.0 | SS-EC | Cholesterol | DSPC | DSPE-PEG5K | 7 |
| | | (49%) | (39.8%) | (11%) | (0.2%) | |
| 70059-2.C | MB, pH 3.0 | SS-EC | Cholesterol | DSPC | DSPE-PEG5K | 7 |
| | | (49%) | (39.5%) | (11%) | (0.5%) | |
| 70059-3.C | MB, pH 3.0 | SS-EC | Cholesterol | DSPC | DSPE-PEG5K | 7 |
| | | (49%) | (39.0%) | (11%) | (1.0%) | |
| 70065-2.C | MB, pH 3.0 | SS-LC | Cholesterol | DSPC | DMG-PEG2K | 7 |
| | | (49%) | (39.5%) | (11%) | (0.5%) | |
| 70065-3.C | MB, pH 3.0 | SS-LC | Cholesterol | DSPC | DMG-PEG2K | 7 |
| | | (49%) | (39.0%) | (11%) | (1.0%) | |
| 70065-4.C | MB, pH 3.0 | SS-LC | Cholesterol | DSPC | DMG-PEG2K | 7 |
| | | (49%) | (38.5%) | (11%) | (1.5%) | |
| 70065-5.C | MB, pH 3.0 | SS-LC | Cholesterol | DSPC | DMG-PEG5K | 7 |
| | | (49%) | (39.5%) | (11%) | (0.5%) | |
| 70065-6.C | MB, pH 3.0 | SS-LC | Cholesterol | DSPC | DMG-PEG5K | 7 |
| | | (49%) | (39.0%) | (11%) | (1.0%) | |
| 70070-1.C | PB, pH 5.8 | MC3 | Cholesterol | DSPC | DSPE-PEG5K | 7 |
| | | (49%) | (39.8%) | (11%) | (0.2%) | |
| 70070-4.C | PB, pH 5.8 | MC3 | Cholesterol | DSPC | DMG-PEG2K | 7 |
| | | (49%) | (39.8%) | (11%) | (0.2%) | |
| 70070-5.C | PB, pH 5.8 | MC3 | Cholesterol | DSPC | DMG-PEG2K | 7 |
| | | (49%) | (38.0%) | (11%) | (2.0%) | |
| 70070-6.C | PB, pH 5.8 | MC3 | Cholesterol | DSPC | DMG-PEG2K | 7 |
| | | (49%) | (35.0%) | (11%) | (5.0%) | |
| 70077-3.C | PB, pH 5.8 | MC3 | Cholesterol | DSPC | DSPE-PEG5K | 7 |
| | | (49%) | (39.8%) | (11%) | (0.2%) | |
| 70077-4.C | MB, pH 3.0 | SS-OC | Cholesterol | DSPC | DSPE-PEG5K | 7 |
| | | (49%) | (39.8%) | (11%) | (0.2%) | |
| 70077-5.C | MB, pH 3.0 | SS-OC | Cholesterol | DSPC | DSG-PEG2K | 7 |
| | | (49%) | (48.5%) | (2%) | (0.5%) | |
| 70077-6.C | MB, pH 3.0 | SS-OC | Cholesterol | DSPC | DPG-PEG2K | 7 |
| | | (49%) | (48.0%) | (2%) | (1.0%) | |
| 70077-7.C | MB, pH 3.0 | SS-OC | Cholesterol | DSPC | DMG-PEG2K | 7 |
| | | (49%) | (47.0%) | (2%) | (2.0%) | |
| 70077-8.C | MB, pH 3.0 | SS-OC | Cholesterol | DSPC | DMG-PEG2K | 7 |
| | | (49%) | (38.5%) | (12%) | (0.5%) | |
| 70077-9.C | MB, pH 3.0 | SS-OC | Cholesterol | DSPC | DSG-PEG2K | 7 |
| | | (49%) | (37.0%) | (12%) | (2.0%) | |
| 70077-10.C | MB, pH 3.0 | SS-OC | Cholesterol | DSPC | DPG-PEG2K | 7 |
| | | (49%) | (28.5%) | (22%) | (0.5%) | |
| 70077-11.C | MB, pH 3.0 | SS-OC | Cholesterol | DSPC | DMG-PEG2K | 7 |
| | | (49%) | (28.0%) | (22%) | (1.0%) | |
| 70087-1.C | PB, pH 5.8 | MC3 | Cholesterol | DSPC | DSPE-PEG5K | 7 |
| | | (49%) | (39.8%) | (11%) | (0.2%) | |
| 70087-2.C | MB, pH 3.0 | SS-OC | Cholesterol | DSPC | DPG-PEG2K | 7 |
| | | (60%) | (28.5%) | (11%) | (0.5%) | |
| 70087-3.C | MB, pH 3.0 | SS-OC | Cholesterol | DSPC | DSG-PEG2K | 7 |
| | | (60%) | (28.5%) | (11%) | (0.5%) | |
| 70087-4.C | MB, pH 3.0 | SS-OC | Cholesterol | DSPC | DMG-PEG2K | 7 |
| | | (60%) | (28.5%) | (11%) | (0.5%) | |
| 70087-5.C | MB, pH 3.0 | SS-OC | Cholesterol | DSPC | DPG-PEG2K | 7 |
| | | (60%) | (27.0%) | (11%) | (1.5%) | |
| 80010-1.C | PB, pH 5.8 | MC3 | Cholesterol | DSPC | DSPE-PEG5K | 7 |
| | | (49%) | (39.8%) | (11%) | (0.2%) | |
| 80010-2.C | MB, pH 3.0 | SS-OC | Cholesterol | DSPC | DPG-PEG2K | 7 |
| | | (49%) | (28.5%) | (22%) | (0.5%) | |
| 80010-3.C | MB, pH 3.0 | SS-OC | Cholesterol | DSPC | DMG-PEG2K | 7 |
| | | (49%) | (28.5%) | (22%) | (0.5%) | |
| 80010-4.C | MB, pH 3.0 | SS-OC | Cholesterol | DSPC | DSG-PEG2K | 7 |
| | | (49%) | (28.5%) | (22%) | (0.5%) | |
| 80010-5.C | MB, pH 3.0 | SS-OC | Cholesterol | DSPC | DPG-PEG2K | 7 |
| | | (49%) | (28.0%) | (22%) | (1.0%) | |
| 80016-1.C | PB, pH 5.8 | MC3 | Cholesterol | DSPC | DSPE-PEG5K | 7 |
| | | (49%) | (39.8%) | (11%) | (0.2%) | |
| 80016-2.C | MB, pH 3.0 | MC3 | Cholesterol | DSPC | DPG-PEG2K | 7 |
| | | (49%) | (28.5%) | (22%) | (0.5%) | |
| 80016-3.C | MB, pH 3.0 | SS-OC | Cholesterol | DSPC | DPG-PEG2K | 5.5 |
| | | (49%) | (26.5%) | (22%) | (0.5%) | |
| 80016-6.C | MB, pH 3.0 | SS-OC | Cholesterol | DSPC | DPG-PEG2K | 7 |
| | | (49%) | (28.5%) | (22%) | (0.5%) | |
| 80016-7.C | MB, pH 3.0 | SS-OC | Cholesterol | DSPC | DPG-PEG2K | 7 |
| | | (49%) | (27.5%) | (22%) | (1.5%) | |
| 80016-9.C | MB, pH 3.0 | L-319 | Cholesterol | DSPC | DMG-PEG2K | 7 |
| | | (49%) | (28.5%) | (22%) | (0.2%) | |
| 80016-10.C | MB, pH 3.0 | L-319 | Cholesterol | DSPC | DMG-PEG2K | 7 |
| | | (49%) | (27.5%) | (22%) | (1.5%) | |
| 80016-11.C | MB, pH 3.0 | L-319 | Cholesterol | DSPC | DMG-PEG2K | 7 |
| | | (49%) | (26.5%) | (22%) | (2.5%) | |
| 80033-1.C | MB, pH 3.0 | SS-OC | Cholesterol | DSPC | DPG-PEG2K | 7 |
| | | (49%) | (28.5%) | (22%) | (0.5%) | |
| 80033-2.C | MB, pH 3.0 | SS-LC | Cholesterol | DSPC | DPG-PEG2K | 7 |
| | | (49%) | (28.5%) | (22%) | (0.5%) | |
| 80033-3.C | MB, pH 3.0 | SS-OP | Cholesterol | DSPC | DPG-PEG2K | 7 |
| | | (49%) | (28.5%) | (22%) | (0.5%) | |
| 80048-1.C | MB, pH 3.0 | SS-OC | Cholesterol | DSPC | DPG-PEG2K | 9 |
| | | (49%) | (35.5%) | (15%) | (0.5%) | |
| 80048-2.C | MB, pH 3.0 | SS-OC | Cholesterol | DSPC | DPG-PEG2K | 5 |
| | | (49%) | (28.5%) | (22%) | (0.5%) | |
| 80048-3.C | MB, pH 3.0 | SS-OC | Cholesterol | DSPC | DPG-PEG2K | 7 |
| | | (49%) | (21.5%) | (29%) | (0.5%) | |
| 80048-4.C | MB, pH 3.0 | SS-OC | Cholesterol | DOPE | DPG-PEG2K | 5 |
| | | (49%) | (21.5%) | (29%) | (0.5%) | |
| 80048-5.C | MB, pH 3.0 | SS-OC | Cholesterol | DOPE | DPG-PEG2K | 9 |
| | | (49%) | (28.5%) | (22%) | (0.5%) | |
| 80048-6.C | MB, pH 3.0 | SS-OC | Cholesterol | DOPE | DPG-PEG2K | 7 |
| | | (49%) | (35.5%) | (15%) | (0.5%) | |
| 80048-7.C | MB, pH 3.0 | SS-OC | Cholesterol | DLPE | DPG-PEG2K | 5 |
| | | (49%) | (35.5%) | (15%) | (0.5%) | |
| 80048-8.C | MB, pH 3.0 | SS-OC | Cholesterol | DLPE | DPG-PEG2K | 7 |
| | | (49%) | (28.5%) | (22%) | (0.5%) | |
| 80048-9.C | MB, pH 3.0 | SS-OC | Cholesterol | DLPE | DPG-PEG2K | 9 |
| | | (49%) | (21.5%) | (29%) | (0.5%) | |
| 80059.1.C | MB, pH 3.0 | SS-OC | Cholesterol | DLPE | DPG-PEG2K | 7 |
| | | (49%) | (28.5%) | (22%) | (0.5%) | |
| 80059-2.C | MB, pH 3.0 | SS-OC | Cholesterol | DLPE | DPG-PEG2K | 7 |
| | | (49%) | (28.5%) | (22%) | (0.5%) | |
| 80130-1.C | MB, pH 3.0 | SS-OC | Cholesterol | DSPC | DPG-PEG2K | 7 |
| | | (49%) | (28.5%) | (22%) | (0.5%) | |
| 80130-2.C | MB, pH 3.0 | SS-OC | Cholesterol | DSPC | DPG-PEG2K | 7 |
| | | (49%) | (28.5%) | (22%) | (0.5%) | |
| 80130-3.C | MB, pH 3.0 | SS-LC | Cholesterol | DSPC | DPG-PEG2K | 7 |
| | | (49%) | (28.5%) | (22%) | (0.5%) | |
| 80139-1.C | MB, pH 3.0 | SS-OC | Cholesterol | DSPC | DPG-PEG2K | 7 |
| | | (49%) | (28.5%) | (22%) | (0.5%) | |

### Therapeutic Compositions and Methods of Use

One aspect of the disclosure relates to therapeutic compositions comprising the recombinant RNA replicons described herein, or particles comprising recombinant RNA replicons described herein, and methods for the treatment of cancer. Compositions described herein can be formulated in any manner suitable for a desired delivery route. Typically, formulations include all physiologically acceptable compositions including derivatives or prodrugs, solvates, stereoisomers, racemates, or tautomers thereof with any pharmaceutically acceptable carriers, diluents, and/or excipients.

In some embodiments, the LNP comprising the recombinant RNA replicon (and optionally the RNA viral genome) is capable of producing oncolytic viruses when administered to a subject, wherein the encoded oncolytic virus is capable of reducing the size of a tumor that is remote from the site of administration. For example, intravenous administration of the LNPs may results in replicon replication in tumor tissue and reduction of tumor size. In some embodiments, the LNPs of the disclosure are capable of localizing to tumors or cancerous tissues that are remote from the site of LNP administration. Such effects enable the use of the LNP-encapsulated replicons of the disclosure in the treatment of tumors that are not easily accessible and therefore not suitable for intratumoral delivery of treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, or emulsifier which has been approved by the United States Food and Drug Administration as being acceptable for use in humans or domestic animals. Exemplary pharmaceutically acceptable carriers include, but are not limited to, to sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations.

“Pharmaceutically acceptable salt” includes both acid and base addition salts. Pharmaceutically-acceptable salts include the acid addition salts (formed with the free amino groups of the protein) and which are formed with inorganic acids such as, for example, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, ptoluenesulfonic acid, trifluoroacetic acid, undecylenic acid, and the like. Salts formed with the free carboxyl groups can also be derived from inorganic bases such as, for example, sodium, potassium, lithium, ammonium, calcium, magnesium, iron, zinc, copper, manganese, aluminum salts, and the like. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline, and caffeine.

The present disclosure provides methods of killing a cancerous cell or a target cell comprising exposing the cell to an RNA polynucleotide or particle described herein, or composition thereof, under conditions sufficient for the intracellular delivery of the composition to the cancerous cell. As used herein “killing a cancerous cell” refer to the death of a cancerous cell by means of apoptosis or necrosis. Killing of a cancerous cell may be determined by methods known in the art including but not limited to, tumor size measurements, cell counts, and flow cytometry for the detection of cell death markers such as Annexin V and incorporation of propidium iodide.

The present disclosure further provides methods of treating or preventing cancer in a subject in need thereof wherein an effective amount of the therapeutic compositions described herein is administered to the subject. The route of administration will vary, naturally, with the location and nature of the disease being treated, and may include, for example intradermal, transdermal, subdermal, parenteral, nasal, intravenous, intramuscular, intranasal, subcutaneous, percutaneous, intratracheal, intraperitoneal, intratumoral, perfusion, lavage, direct injection, and oral administration. The encapsulated polynucleotide compositions described herein are useful in the treatment of metastatic cancers, wherein systemic administration may be necessary to deliver the compositions to multiple organs and/or cell types. Therefore, in some embodiments, the compositions described herein are administered systemically

The present disclosure further provides methods of immunizing a subject against a disease wherein an effective amount of a therapeutic composition described herein is administered to the subject. The route of administration will vary, naturally, with the location and nature of immunization agent, and may include, for example intradermal, transdermal, subdermal, parenteral, nasal, intravenous, intramuscular, intranasal, subcutaneous, percutaneous, intratracheal, intraperitoneal, intratumoral, perfusion, lavage, direct injection, and oral administration.

The present disclosure further provides a particle of the disclosure, a vector of the disclosure, a recombinant RNA replicon of the disclosure, or compositions thereof, for use as a medicament. In some embodiments, the medicament is for the killing a cancerous cell. In some embodiments, the medicament is for treating cancer. In some embodiments, the medicament is for immunization against a disease.

An “effective amount” or an “effective dose,” used interchangeably herein, refers to an amount and or dose of the compositions described herein that results in an improvement or remediation of the symptoms of the disease or condition. The improvement is any improvement or remediation of the disease or condition, or symptom of the disease or condition. The improvement is an observable or measurable improvement or may be an improvement in the general feeling of well-being of the subject. Thus, one of skill in the art realizes that a treatment may improve the disease condition but may not be a complete cure for the disease. Improvements in subjects may include, but are not limited to, decreased tumor burden, decreased tumor cell proliferation, increased tumor cell death, activation of immune pathways, increased time to tumor progression, decreased cancer pain, increased survival, or improvements in the quality of life. The effective amount of a particular agent may therefore be represented in a variety of ways based on the nature of the agent, such as mass/volume, #of cells/volume, particles/volume, (mass of the agent)/(mass of the subject), #of cells/(mass of subject), or particles/(mass of subject). The effective amount of a particular agent may also be expressed as the half-maximal effective concentration (EC₅₀), which refers to the concentration of an agent that results in a magnitude of a particular physiological response that is half-way between a reference level and a maximum response level.

In some embodiments, administration of an effective dose may be achieved with administration a single dose of a composition described herein. As used herein, “dose” refers to the amount of a composition delivered at one time. In some embodiments, the dose of the recombinant RNA molecules is measured as the 50% Tissue culture Infective Dose (TCID₅₀). In some embodiments, the TCID₅₀ is at least about 10³-10⁹ TCID₅₀/mL, for example, at least about 10³ TCID₅₀/mL, about 10⁴ TCID₅₀/mL, about 10⁵ TCID₅₀/mL, about 10⁶ TCID₅₀/mL, about 10⁷ TCID₅₀/mL, about 108 TCID₅₀/mL, or about 10⁹ TCID₅₀/mL. In some embodiments, a dose may be measured by the number of particles in a given volume (e.g., particles/mL). In some embodiments, a dose may be further refined by the genome copy number of the RNA polynucleotides described herein present in each particle (e.g., #of particles/mL, wherein each particle comprises at least one genome copy of the polynucleotide). In some embodiments, delivery of an effective dose may require administration of multiple doses of a composition described herein. As such, administration of an effective dose may require the administration of at least 2, 3, 4, 5, 6, 7, 8, 9, 10, 20, 30, 40, 50, or more doses of a composition described herein.

In embodiments wherein multiple doses of a composition described herein are administered, each dose need not be administered by the same actor and/or in the same geographical location. Further, the dosing may be administered according to a predetermined schedule. For example, the predetermined dosing schedule may comprise administering a dose of a composition described herein daily, every other day, weekly, bi-weekly, monthly, bi-monthly, annually, semi-annually, or the like. The predetermined dosing schedule may be adjusted as necessary for a given patient (e.g., the amount of the composition administered may be increased or decreased and/or the frequency of doses may be increased or decreased, and/or the total number of doses to be administered may be increased or decreased).

### Definitions

In the present description, any concentration range, percentage range, ratio range, or integer range is to be understood to include the value of any integer within the recited range and, when appropriate, fractions thereof (such as one tenth and one hundredth of an integer), unless otherwise indicated. It should be understood that the terms “a” and “an” as used herein refer to “one or more” of the enumerated components unless otherwise indicated. The use of the alternative (e.g., “or”) should be understood to mean either one, both, or any combination thereof of the alternatives. As used herein, the terms “include” and “comprise” are used synonymously. As used herein, “plurality” may refer to one or more components (e.g., one or more miRNA target sequences).

As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). In some embodiments, the term “approximately” or “about” refers to a range of values that fall within 10% in either direction (greater than or less than) of the stated reference value unless otherwise stated or otherwise evident from the context (except where such number would exceed 100% of a possible value).

“Decrease” or “reduce” refers to a decrease or a reduction in a particular value of at least 5%, for example, 5, 6, 7, 8, 9, 10, 15, 20, 25, 30, 35, 40, 45, 50, 55, 60, 65, 70, 75, 80, 85, 90, 95, 99, or 100% as compared to a reference value. A decrease or reduction in a particular value may also be represented as a fold-change in the value compared to a reference value, for example, at least 1, 2, 3, 4, 5, 6, 7, 8, 9, 10, 15, 20, 30, 40, 50, 60, 70, 80, 90, 100, 200, 500, 1000-fold, or more, decrease as compared to a reference value.

“Increase” refers to an increase in a particular value of at least 5%, for example, 5, 6, 7, 8, 9, 10, 15, 20, 25, 30, 35, 40, 45, 50, 55, 60, 65, 70, 75, 80, 85, 90, 95, 99, 100, 200, 300, 400, 500% or more as compared to a reference value. An increase in a particular value may also be represented as a fold-change in the value compared to a reference value, for example, at least 1-fold, 2, 3, 4, 5, 6, 7, 8, 9, 10, 15, 20, 30, 40, 50, 60, 70, 80, 90, 100, 200, 500, 1000-fold or more, increase as compared to the level of a reference value.

The term “sequence identity” refers to the percentage of bases or amino acids between two polynucleotide or polypeptide sequences that are the same, and in the same relative position. As such one polynucleotide or polypeptide sequence has a certain percentage of sequence identity compared to another polynucleotide or polypeptide sequence. For sequence comparison, typically one sequence acts as a reference sequence, to which test sequences are compared. The term “reference sequence” refers to a molecule to which a test sequence is compared. The term “mutation” refers to the substitution, deletion or addition of nucleic acids or amino acids. The term “conservative mutation” refers to the substitution of a single amino acid or a small number of amino acids in a polypeptide where the new amino acid has a chemical and physical property (charge, hydrophilicity, etc.) that is similar to the substituted amino acid.

“Complementary” refers to the capacity for pairing, through base stacking and specific hydrogen bonding, between two sequences comprising naturally or non-naturally occurring (e.g., modified as described above) bases (nucleotides) or analogs thereof. For example, if a base at one position of a nucleic acid is capable of hydrogen bonding with a base at the corresponding position of a target, then the bases are considered to be complementary to each other at that position. Nucleic acids can comprise universal bases, or inert abasic spacers that provide no positive or negative contribution to hydrogen bonding. Base pairings may include both canonical Watson-Crick base pairing and non-Watson-Crick base pairing (e.g., Wobble base pairing and Hoogsteen base pairing). It is understood that for complementary base pairings, adenosine-type bases (A) are complementary to thymidine-type bases (T) or uracil-type bases (U), that cytosine-type bases (C) are complementary to guanosine-type bases (G), and that universal bases such as 3-nitropyrrole or 5-nitroindole can hybridize to and are considered complementary to any A, C, U, or T. Nichols et al., Nature, 1994; 369:492-493 and Loakes et al., Nucleic Acids Res., 1994; 22:4039-4043. Inosine (I) has also been considered in the art to be a universal base and is considered complementary to any A, C, U, or T. See Watkins and SantaLucia, Nucl. Acids Research, 2005; 33 (19): 6258-6267.

An “expression cassette” or “expression construct” refers to a polynucleotide sequence operably linked to a promoter.

“Operably linked” refers to a juxtaposition wherein the components so described are in a relationship permitting them to function in their intended manner. For instance, a promoter is operably linked to a polynucleotide sequence if the promoter affects the transcription or expression of the polynucleotide sequence; a cleavage polypeptide is operably linked to a payload molecule if it allows the separation of the payload molecule (e.g., from the rest of the polypeptide) under certain desirable conditions.

The term “subject” includes animals, such as mammals. In some embodiments, the mammal is a primate. In some embodiments, the mammal is a human. In some embodiments, subjects are livestock such as cattle, sheep, goats, cows, swine, and the like; or domesticated animals such as dogs and cats. In some embodiments (e.g., particularly in research contexts) subjects are rodents (e.g., mice, rats, hamsters), rabbits, primates, or swine such as inbred pigs and the like. The terms “subject” and “patient” are used interchangeably herein. In some embodiments, the methods of the present disclosure are employed to treat a human subject. The methods of the present disclosure may also be employed to treat non-human primates (e.g., monkeys, baboons, and chimpanzees), mice, rats, bovines, horses, cats, dogs, pigs, rabbits, goats, deer, sheep, ferrets, gerbils, guinea pigs, hamsters, bats, birds, and reptiles.

As used herein “prevention” or “prophylaxis” can mean complete prevention of the symptoms of a disease, a delay in onset of the symptoms of a disease, or a lessening in the severity of subsequently developed disease symptoms.

“Cancer” herein refers to or describes the physiological condition in mammals that is typically characterized by unregulated cell growth. Examples of cancer include but are not limited to carcinoma, lymphoma, blastoma, sarcoma (including liposarcoma, osteogenic sarcoma, angiosarcoma, endotheliosarcoma, leiomyosarcoma, chordoma, lymphangiosarcoma, lymphangioendotheliosarcoma, rhabdomyosarcoma, fibrosarcoma, myxosarcoma, and chondrosarcoma), neuroendocrine tumors, mesothelioma, synovioma, schwannoma, meningioma, adenocarcinoma, melanoma, and leukemia or lymphoid malignancies. More particular examples of such cancers include squamous cell cancer (e.g., epithelial squamous cell cancer), lung cancer including small-cell lung cancer, non-small cell lung cancer, adenocarcinoma of the lung and squamous carcinoma of the lung, small cell lung carcinoma, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer (e.g., renal cell carcinoma), neuroendocrine cancer, prostate cancer (e.g., Castration resistant neuroendocrine prostate cancer), vulvar cancer, thyroid cancer, B-cell chronic lymphocytic leukemia, diffuse large B-cell lymphoma (DLBCL), marginal zone lymphoma (MZL), Merkel cell carcinoma, hepatic carcinoma, anal carcinoma, penile carcinoma, testicular cancer, esophageal cancer, tumors of the biliary tract, Ewing's tumor, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, testicular tumor, lung carcinoma, bladder carcinoma, epithelial carcinoma, glioma (e.g., malignant glioma), astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, multiple myeloma, Waldenstrom's macroglobulinemia, myelodysplastic disease, heavy chain disease, neuroendocrine tumors, Schwannoma, and other carcinomas, as well as head and neck cancer. In some embodiments, the cancer is a neuroendocrine cancer. Furthermore, benign (i.e., noncancerous) hyperproliferative diseases, disorders and conditions, including benign prostatic hypertrophy (BPH), meningioma, schwannoma, neurofibromatosis, keloids, myoma and uterine fibroids and others may also be treated using the disclosure disclosed herein.

“Administration” refers herein to introducing an agent or composition into a subject.

“Treating” as used herein refers to delivering an agent or composition to a subject to affect a physiologic outcome. In some embodiments, treating refers to the treatment of a disease in a mammal, e.g., in a human, including (a) inhibiting the disease, i.e., arresting disease development or preventing disease progression; (b) relieving the disease, i.e., causing regression of the disease state; and (c) curing the disease.

“Population” of cells refers to any number of cells greater than 1, but is preferably at least 1×10³ cells, at least 1×10⁴ cells, at least 1×10⁵ cells, at least 1×10⁶ cells, at least 1×10⁷ cells, at least 1×10⁸ cells, at least 1×10⁹ cells, at least 1×10¹⁰ cells, or more cells. A population of cells may refer to an in vitro population (e.g., a population of cells in culture) or an in vivo population (e.g., a population of cells residing in a particular tissue).

“Effector function” refers to functions of an immune cell related to the generation, maintenance, and/or enhancement of an immune response against a target cell or target antigen.

The terms “microRNA,” “miRNA,” and “miR” are used interchangeably herein and refer to small non-coding endogenous RNAs of about 21-25 nucleotides in length that regulate gene expression by directing their target messenger RNAs (mRNA) for degradation or translational repression.

The term “composition” as used herein refers to a formulation of a recombinant RNA molecule or a particle-encapsulated recombinant RNA molecule described herein that is capable of being administered or delivered to a subject or cell.

The term “replication-competent viral genome” refers to a viral genome encoding all of the viral genes necessary for viral replication and production of an infectious viral particle.

The term “oncolytic virus” refers to a virus that has been modified to, or naturally, preferentially infect cancer cells.

The term “vector” is used herein to refer to a nucleic acid molecule capable of transferring or transporting another nucleic acid molecule.

The term “replicon” refers to a nucleic acid that is capable of directing the generation of copies of itself. As used herein, the term “replicon” includes RNA as well as DNA. Generally, a viral replicon contains at least a part of the genome of the virus. A viral replicon may contain an incomplete viral genome yet is still capable of directing the generation of copies of itself.

The term “upstream”, when used in reference to nucleic acid, refers to a nucleotide sequence that is located toward 5′ with respect to the reference nucleotide sequence, and when used in reference to polypeptide, refers to an amino acid sequence that is located towards N-term with respect to the reference amino acid sequence. The term “downstream”, when used in reference to nucleic acid, refers to a nucleotide sequence that is located toward 3′ with respect to the reference nucleotide sequence, and when used in reference to polypeptide, refers to an amino acid sequence that is located towards C-term with respect to the reference amino acid sequence.

The term “cis-acting replication element” refers to a portion of the RNA genome of an RNA virus or replicon which must be present in cis, that is, present as part of each viral strand as a necessary condition for replication. In some embodiments, the cis-acting replication element is composed of one or more segments of viral RNA.

The terms “corresponding to” or “correspond to”, as used herein in relation to the amino acid or nucleic acid position(s), refer to the position(s) in a first polypeptide/polynucleotide sequence that aligns with a given amino acid/nucleic acid in a reference polypeptide/polynucleotide sequence when the first and the reference polypeptide/polynucleotide sequences are aligned. Alignment is performed by one of skill in the art using software designed for this purpose, for example, Clustal Omega version 1.2.4 with the default parameters for that version.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

### FURTHER NUMBERED EMBODIMENTS

Further numbered embodiments of the present disclosure are provided as follows:

Embodiment 1. A recombinant RNA replicon comprising:
a picornavirus genome, wherein the picornavirus genome comprises a deletion or a truncation in one or more protein coding regions; and
a heterologous polynucleotide.

Embodiment 2. The recombinant RNA replicon of Embodiment 1, wherein the picornavirus genome comprises the deletion or the truncation in one or more VP coding regions.

Embodiment 3. The recombinant RNA replicon of Embodiment 1 or 2, wherein the picornavirus genome comprises the deletion or the truncation in each of the VP1, VP3 and VP2 coding regions.

Embodiment 4. The recombinant RNA replicon of any one of Embodiments 1-3, wherein the picornavirus genome comprises the deletion of the VP1 and VP3 coding regions and the truncation of the VP2 coding region.

Embodiment 5. The recombinant RNA replicon of any one of Embodiments 1-4, wherein the picornavirus is selected from a senecavirus, a cardiovirus, and an enterovirus.

Embodiment 6. The recombinant RNA replicon of any one of Embodiments 1-5, wherein the deletion or the truncation comprises at least 500 bp, at least 1000 bp, at least 1500 bp, at least 2000 bp, at least 2500 bp, or at least 3000 bp.

Embodiment 7. The recombinant RNA replicon of Embodiments 6, wherein the deletion or the truncation comprises at least 2000 bp.

Embodiment 8. The recombinant RNA replicon of any one of Embodiments 1-7, wherein a site of the deletion or a site of the truncation comprises the heterologous polynucleotide

Embodiment 9. The recombinant RNA replicon of any one of Embodiments 1-7, wherein the heterologous polynucleotide is inserted between a 2A coding region and a 2B coding region.

Embodiment 10. The recombinant RNA replicon of any one of Embodiments 1-7, wherein the heterologous polynucleotide is inserted between a 3D coding region and a 3′ untranslated region (UTR).

Embodiment 11. The recombinant RNA replicon of any one of Embodiments 1-10, wherein the heterologous polynucleotide comprises at least 1000 bp, at least 2000 bp, or at least 3000 bp.

Embodiment 12. The recombinant RNA replicon of any one of Embodiments 1-11, wherein the picornavirus is a Seneca Valley Virus (SVV).

Embodiment 13. The recombinant RNA replicon of Embodiment 12, wherein the deletion or the truncation comprises one or more nucleotides between nucleotide 1261 and 3477, inclusive of the endpoints, according to the numbering of SEQ ID NO: 1.

Embodiment 14. The recombinant RNA replicon of Embodiment 12, wherein the deletion or the truncation comprises nucleotide 1261 to 3477, inclusive of the endpoints, according to the numbering of SEQ ID NO: 1.

Embodiment 15. The recombinant RNA replicon of Embodiments 12 or 13, wherein the deletion or the truncation comprises at least 500 bp, at least 1000 bp, at least 1500 bp, or at least 2000 bp.

Embodiment 16. The recombinant RNA replicon of Embodiment 15, wherein the deletion or the truncation comprises at least 2000 bp.

Embodiment 17. The recombinant RNA replicon of any one of Embodiments 12 to 16, wherein the SVV genome comprises a 5′ leader protein coding sequence.

Embodiment 18. The recombinant RNA replicon of any one of Embodiments 12 to 17, wherein the SVV genome comprises a VP4 coding region.

Embodiment 19. The recombinant RNA replicon of any one of Embodiments 12 to 18, wherein the SVV genome comprises a VP2 coding region or a truncation thereof.

Embodiment 20. The recombinant RNA replicon of Embodiment 19, wherein the SVV genome comprises, from 5′ to 3′ direction, the 5′ leader protein coding sequence, the VP4 coding region, and the VP2 coding region or a truncation thereof.

Embodiment 21. The recombinant RNA replicon of Embodiment 20, wherein a portion of the SVV genome comprising the 5′ leader protein coding sequence, the VP4 coding region, and the VP2 coding region or a truncation thereof has at least 90% sequence identity to nucleotide 1 to 1260 of SEQ ID NO: 1.

Embodiment 22. The recombinant RNA replicon of Embodiment 20 or 21, wherein the SVV genome comprises, from 5′ to 3′ direction, the 5′ leader protein coding sequence, the VP4 coding region, the VP2 coding region or a truncation thereof, and the heterologous polynucleotide.

Embodiment 23. The recombinant RNA replicon of any one of Embodiments 1-22, wherein the SVV genome comprises a cis-acting replication element (CRE).

Embodiment 24. The recombinant RNA replicon of Embodiment 23, wherein the CRE comprises between 10-200 bp.

Embodiment 25. The recombinant RNA replicon of Embodiment 23 or 24, wherein the CRE comprises one or more nucleotides within the region corresponding to nucleotide 1000 to nucleotide 1260 according to SEQ ID NO: 1.

Embodiment 26. The recombinant RNA replicon of Embodiment 23 or 24, wherein the CRE comprises one or more nucleotides within the region corresponding to nucleotide 1117 to nucleotide 1260 according to SEQ ID NO: 1.

Embodiment 27. The recombinant RNA replicon of any one of Embodiments 23-26, wherein the CRE comprises a polynucleotide sequence having at least 70%, at least 75%, at least 80%, at least 85%, at least 90%, at least 95%, at least 96%, at least 97%, at least 98%, at least 99%, or 100% identity to SEQ ID NO: 149.

Embodiment 28. The recombinant RNA replicon of any one of Embodiments 12-27, wherein the SVV genome further comprises a 2A coding region.

Embodiment 29. The recombinant RNA replicon of Embodiment 28, wherein the 2A coding region is located between the VP2 coding region or a truncation thereof and the heterologous polynucleotide.

Embodiment 30. The recombinant RNA replicon of any one of Embodiments 12-29, wherein the SVV genome comprises one or more of a 2B coding region, a 2C coding region, a 3A coding region, a 3B coding region, a 3Cpro coding region, and a 3D(RdRp) coding region.

Embodiment 31. The recombinant RNA replicon of any one of Embodiments 12-29, wherein the SVV genome comprises a 2B coding region, a 2C coding region, a 3A coding region, a 3B coding region, a 3Cpro coding region, and a 3D(RdRp) coding region.

Embodiment 32. The recombinant RNA replicon of Embodiment 31, wherein the SVV genome comprises, from 5′ to 3′, the 2B coding region, the 2C coding region, the 3A coding region, the 3B coding region, the 3Cpro coding region, and the 3D(RdRp) coding region.

Embodiment 33. The recombinant RNA replicon of Embodiment 32, wherein a portion of the SVV genome comprising the 2B coding region, the 2C coding region, the 3A coding region, the 3B coding region, the 3Cpro coding region, and the 3D(RdRp) coding region has at least 90% sequence identity to nucleotide 3505 to 7310 according to SEQ ID NO: 1.

Embodiment 34. The recombinant RNA replicon of any one of Embodiments 30-33, wherein the SVV genome comprises, from 5′ to 3′, the heterologous polynucleotide and the 2B coding region.

Embodiment 35. The recombinant RNA replicon of any one of Embodiments 1 to 11, wherein the picornavirus is a coxsackievirus.

Embodiment 36. The recombinant RNA replicon of Embodiment 35, wherein the deletion or the truncation comprises one or more nucleotides between nucleotide 717 to 3332, inclusive of the endpoints, according to the numbering of SEQ ID NO: 3.

Embodiment 37. The recombinant RNA replicon of Embodiment 35, wherein the deletion or the truncation comprises nucleotide 717 to 3332, inclusive of the endpoints, according to the numbering of SEQ ID NO: 3.

Embodiment 38. The recombinant RNA replicon of Embodiment 35 or 36, wherein the deletion or the truncation comprises at least 500 bp, at least 1000 bp, at least 1500 bp, at least 2000 bp, or at least 2600 bp.

Embodiment 39. The recombinant RNA replicon of any one of Embodiments 35 to 38, wherein the coxsackievirus genome comprises a 5′ UTR.

Embodiment 40. The recombinant RNA replicon of any one of Embodiments 35 to 39, wherein a portion of the coxsackievirus genome comprising the 5′ UTR has at least 90% sequence identity to SEQ ID NO: 4.

Embodiment 41. The recombinant RNA replicon of any one of Embodiments 35 to 40, wherein the coxsackievirus genome comprises one or more of a 2A coding region, a 2B coding region, a 2C coding region, a 3A coding region, a 3B coding region, a VPg coding region, a 3C coding region, a 3D pol coding region, and a 3′ UTR.

Embodiment 42. The recombinant RNA replicon of any one of Embodiments 35 to 40, wherein the coxsackievirus genome comprises a 2A coding region, a 2B coding region, a 2C coding region, a 3A coding region, a 3B coding region, a VPg coding region, a 3C coding region, a 3D pol coding region, and a 3′ UTR.

Embodiment 43. The recombinant RNA replicon of Embodiment 42, wherein the coxsackievirus genome comprises, from 5′ to 3′ direction, the 2A coding region, the 2B coding region, the 2C coding region, the 3A coding region, the 3B coding region, the VPg coding region, the 3C coding region, the 3D pol coding region, and the 3′ UTR.

Embodiment 44. The recombinant RNA replicon of Embodiment 42, wherein a portion of the coxsackievirus genome comprising the 2A coding region, the 2B coding region, the 2C coding region, the 3A coding region, the 3B coding region, the VPg coding region, the 3C coding region, the 3D pol coding region, and the 3′ UTR has at least 90% sequence identity to nucleotide 3492 to 7435 in SEQ ID NO: 3.

Embodiment 45. The recombinant RNA replicon of any one of Embodiments 41 to 44, wherein the coxsackievirus genome comprises, from 5′ to 3′, the 5′ UTR, the heterologous polynucleotide, and the 2A coding region.

Embodiment 46. The recombinant RNA replicon of any one of Embodiments 1 to 11, wherein the picornavirus is an encephalomyocarditis virus (EMCV).

Embodiment 47. The recombinant RNA replicon of any one of Embodiments 9 and 11-46, wherein the recombinant RNA replicon comprises an internal ribosome entry site (IRES) inserted between the heterologous polynucleotide and the 2B coding region.

Embodiment 48. The recombinant RNA replicon of any one of Embodiments 1 to 47, wherein the heterologous polynucleotide encodes one or more payload molecules.

Embodiment 49. The recombinant RNA replicon of any one of Embodiments 1 to 47, wherein the heterologous polynucleotide encodes two or more payload molecules.

Embodiment 50. The recombinant RNA replicon of Embodiment 49, wherein the two or more payload molecules are operably linked by one or more cleavage polypeptides.

Embodiment 51. The recombinant RNA replicon of Embodiment 50, wherein the cleavage polypeptide comprises a 2A family self-cleaving peptide, a 3C cleavage site, a furin site, an IGSF1 polypeptide, or a HIV protease site.

Embodiment 52. The recombinant RNA replicon of Embodiment 51, wherein the cleavage polypeptide comprises an IGSF1 polypeptide, and wherein the IGSF1 polypeptide comprises an amino acid sequence having at least 90% identity to SEQ ID NO: 75.

Embodiment 53. The recombinant RNA replicon of Embodiment 51, wherein the cleavage polypeptide comprises an HIV protease site.

Embodiment 54. The recombinant RNA replicon of Embodiment 51, wherein the cleavage polypeptide comprises a 2A family self-cleaving peptide.

Embodiment 55. The recombinant RNA replicon of any one of Embodiments 50 to 54, wherein the cleavage polypeptide comprises a furin site.

Embodiment 56. The recombinant RNA replicon of any one of Embodiments 50 to 55, wherein the heterologous polynucleotide encodes a polypeptide comprising the two or more payload molecules and the cleavage polypeptide comprising, from N-terminus to C-terminus: N′-payload molecule 1-cleavage polypeptide-payload molecule 2-C′.

Embodiment 57. The recombinant RNA replicon of Embodiment 53, wherein the heterologous polynucleotide further comprises a coding region that encodes an HIV protease, and wherein the heterologous polynucleotide comprises a coding region that encodes a polypeptide comprising, from N-terminus to C-terminus: N′-Payload molecule 1-HIV protease site-HIV protease-HIV protease site-Payload molecule 2-C′.

Embodiment 58. The recombinant RNA replicon of Embodiment 57, wherein the heterologous polynucleotide further comprises a coding region that encodes a third payload molecule, and wherein the heterologous polynucleotide comprises a coding region that encodes a polypeptide comprising, from N-terminus to C-terminus:

N′-Payload molecule 1-HIV protease site-HIV protease-HIV protease site-Payload molecule 2-HIV protease site-Payload molecule 3-C′.

Embodiment 59. The recombinant RNA replicon of any one of Embodiments 56 to 58, further comprising a cleavage polypeptide at the C-terminus of the encoded polypeptide.

Embodiment 60. The recombinant RNA replicon of any one of Embodiment 48 to 59, wherein the payload molecules are selected from a fluorescent protein, an enzyme, a cytokine, a chemokine, an antigen, an antigen-binding molecule capable of binding to a cell surface receptor, and a ligand for a cell-surface receptor.

Embodiment 61. The recombinant RNA replicon of any one of Embodiment 48 to 59, wherein the payload molecules are selected from:
a) one or more cytokines comprising IFN□, GM-CSF, IL-2, IL-12, IL-15, IL-18, IL-23, and IL-36γ;
b) one or more chemokines comprising CXCL10, CCL4, CCL5, and CCL21;
c) one or more antibodies comprising an anti-PD1-VHH-Fc antibody, an anti-CD47-VHH-Fc antibody, and an anti-TGFβ-VHH(or scFv)-Fc antibody;
d) one or more bipartite polypeptides comprising a bipartite polypeptide binding to DLL3 and an effector cell target antigen, a bipartite polypeptide binding to FAP and an effector cell target antigen, and a bipartite polypeptide binding to EpCAM and an effector cell target antigen;
e) one or more tumor-associated antigens comprising survivin, MAGE family proteins, and all antigens according to Table 6;
f) one or more tumor neoantigens;
g) one or more bipartite polypeptides binding to MHC-peptide antigen complex;
h) one or more fusogenic proteins comprising herpes simplex virus (HSV) UL27/glycoprotein B/gB, HSV UL53/glycoprotein K/gK, Respiratory syncytial virus (RSV) F protein, FASTp15, VSV-G, syncitin-1 (from human endogenous retrovirus-W (HERV-W)) or syncitin-2 (from HERVFRDE1), paramyxovirus SV5-F, measles virus-H, measles virus-F, and the glycoprotein from a retrovirus or lentivirus, such as gibbon ape leukemia virus (GALV), murine leukemia virus (MLV), Mason-Pfizer monkey virus (MPMV) and equine infectious anemia virus (EIAV), optionally with the R transmembrane peptide removed (R-versions);
i) one or more other payload molecules comprising IL15R, PGDH, ADA, ADA2, HYAL1, HYAL2, CHIPS, MLKL (or its 4HB domain only), GSDMD (or its L192A mutant, or its amino acids 1-233 fragment, or its amino acids 1-233 fragment with L192A mutation), GSDME (or its amino acid 1-237 fragment), HMGB1 (or its Box B domain only), Melittin (e.g., alpha-Melittin), SMAC/Diablo (or its amino acid 56-239 fragment), Snake LAAO, Snake disintegrin, Leptin, FLT3L, TRAIL, Gasdermin D or a truncation thereof, and Gasdermin E or a truncation thereof,
j) one or more antigens from pathogens comprising Dengue virus, Chikungunya virus, Mycobacterium tuberculosis, Human immunodeficiency viruses, SARS-CoV-2, Coronavirus, Hepatitis B Virus, Togaviridae family virus, Flaviviridae family virus, Influenza A virus, Influenza B virus, and a veterinary virus; or
k) any combination thereof.

Embodiment 62. The recombinant RNA replicon of any one of Embodiments 49 to 59, wherein the two or more payload molecules are selected from the group consisting of a fluorescent protein, an enzyme, a cytokine, a chemokine, an antigen-binding molecule capable of binding to a cell surface receptor, and a ligand for a cell-surface receptor.

Embodiment 63. The recombinant RNA replicon of any one of Embodiments 49 to 59, wherein the heterologous polynucleotide encodes two or more payload molecules comprising:
a. IL-2 and IL-36γ;
b. CXCL10 and an antigen binding molecule binding to FAP and CD3;
c. IL-2 and an antigen binding molecule binding to DLL3 and CD3;
d. IL-36γ and an antigen binding molecule binding to DLL3 and CD3; or
e. IL-2, IL-36γ and an antigen binding molecule binding to DLL3 and CD3.

Embodiment 64. The recombinant RNA replicon of any one of Embodiments 1 to 63, further comprising a microRNA (miRNA) target sequence (miR-TS) cassette comprising one or more miRNA target sequences.

Embodiment 65. The recombinant RNA replicon of Embodiment 64, wherein the one or more miRNAs comprise miR-124, miR-1, miR-143, miR-128, miR-219, miR-219a, miR-122, miR-204, miR-217, miR-137, and miR-126.

Embodiment 66. A recombinant DNA molecule comprising, from 5′ to 3′, a promoter sequence, a 5′ junctional cleavage sequence, a polynucleotide sequence encoding the recombinant RNA replicon of any one of Embodiments 1-65, and a 3′ junctional cleavage sequence.

Embodiment 67. The recombinant DNA molecule of Embodiment 66, wherein the promoter sequence is a T7 promoter sequence.

Embodiment 68. The recombinant DNA molecule of Embodiment 66 or 67, wherein the 5′ junctional cleavage sequence is a ribozyme sequence and the 3′ junctional cleavage sequence is a ribozyme sequence.

Embodiment 69. The recombinant DNA molecule of Embodiment 68, wherein the 5′ ribozyme sequence is a hammerhead ribozyme sequence and wherein the 3′ ribozyme sequence is a hepatitis delta virus ribozyme sequence.

Embodiment 70. The recombinant DNA molecule of Embodiment 66 or 67, wherein the 5′ junctional cleavage sequence is a ribozyme sequence and the 3′ junctional cleavage sequence is a restriction enzyme recognition sequence.

Embodiment 71. The recombinant DNA molecule of Embodiment 70, wherein the 5′ ribozyme sequence is a hammerhead ribozyme sequence, a Pistol ribozyme sequence, or a modified Pistol ribozyme sequence.

Embodiment 72. The recombinant DNA molecule of Embodiment 70 or 71, wherein 3′ restriction enzyme recognition sequence is a Type IIS restriction enzyme recognition sequence.

Embodiment 73. The recombinant DNA molecule of Embodiment 72, wherein the Type IIS recognition sequence is a SapI recognition sequence.

Embodiment 74. The recombinant DNA molecule of Embodiment 66 or 67, wherein the 5′ junctional cleavage sequence is an RNAseH primer binding sequence and the 3′ junctional cleavage sequence is a restriction enzyme recognition sequence.

Embodiment 75. A method of producing the recombinant RNA replicon of any one of Embodiments 1-65, comprising in vitro transcription of the DNA molecule of any one of Embodiments 66-74 and purification of the resulting recombinant RNA replicon.

Embodiment 76. A composition comprising an effective amount of the recombinant RNA replicon of any one of Embodiments 1-65, and a carrier suitable for administration to a mammalian subject.

Embodiment 77. A vector comprising the recombinant RNA replicon of any one of Embodiments 1-65.

Embodiment 78. The vector of Embodiment 77, wherein the vector is a viral vector.

Embodiment 79. The vector of Embodiment 77, wherein the vector is a non-viral vector.

Embodiment 80. A particle comprising the recombinant RNA replicon of any one of Embodiments 1-65.

Embodiment 81. The particle of Embodiment 80, wherein the particle is selected from the group consisting of a nanoparticle, an exosome, a liposome, and a lipoplex.

Embodiment 82. The particle of Embodiment 81, wherein the nanoparticle is a lipid nanoparticle (LNP) comprising a cationic lipid, one or more helper lipids, and a phospholipid-polymer conjugate.

Embodiment 83. The particle of Embodiment 82, wherein the cationic lipid is selected from DLinDMA, DLin-KC2-DMA, DLin-MC3-DMA (MC3), COATSOME® SS-LC (former name: SS-18/4PE-13), COATSOME® SS-EC (former name: SS-33/4PE-15), COATSOME® SS-OC, COATSOME® SS-OP, Di((Z)-non-2-en-1-yl)9-((4-dimethylamino)butanoyl)oxy)heptadecanedioate (L-319), or N-(2,3-dioleoyloxy)propyl)-N,N,N-trimethylammonium chloride (DOTAP).

Embodiment 84. The particle of Embodiment 82 or 83, wherein the helper lipid is selected from 1,2-distearoyl-sn-glycero-3-phosphocholine (DSPC); 1,2-dilauroyl-sn-glycero-3-phosphoethanolamine (DLPE); 1,2-dioleoyl-sn-glycero-3-phosphocholine (DOPC); 1,2-dioleoyl-sn-glycero-3-phosphoethanolamine (DOPE); and cholesterol.

Embodiment 85. The particle of Embodiment 82, wherein the cationic lipid is 1,2-dioleoyl-3-trimethylammonium-propane (DOTAP), and wherein the neutral lipid is 1,2-Dilauroyl-sn-glycero-3-phosphoethanolamine (DLPE) or 1,2-Dioleoyl-sn-glycero-3-phosphoethanolamine (DOPE).

Embodiment 86. The particle of any one of Embodiments 82-85, wherein the PEG-lipid is selected from 1,2-distearoyl-sn-glycero-3-phosphoethanolamine-N-[amino(polyethyleneglycol)] (DSPE-PEG); 1,2-dipalmitoyl-rac-glycerol methoxypolyethylene glycol (DPG-PEG); 1,2-distearoyl-rac-glycero-3-methylpolyoxyethylene (DSG-PEG); 1,2-distearoyl-rac-glycero-3-methylpolyoxyethylene (DSG-PEG); 1,2-dimyristoyl-rac-glycero-3-methylpolyoxyethylene (DMG-PEG); and 1,2-dimyristoyl-rac-glycero-3-methylpolyoxyethylene (DMG-PEG), or 1,2-distearoyl-sn-glycero-3-phosphoethanolamine-N-[amino(polyethylene glycol)] (DSPE-PEG-amine).

Embodiment 87. The particle of any one of Embodiments 82-86, wherein the PEG-lipid is selected from 1,2-distearoyl-sn-glycero-3-phosphoethanolamine-N-[amino(polyethyleneglycol)-5000] (DSPE-PEG5K); 1,2-dipalmitoyl-rac-glycerol methoxypolyethylene glycol-2000 (DPG-PEG2K); 1,2-distearoyl-rac-glycero-3-methylpolyoxyethylene-5000 (DSG-PEG5K); 1,2-distearoyl-rac-glycero-3-methylpolyoxyethylene-2000 (DSG-PEG2K); 1,2-dimyristoyl-rac-glycero-3-methylpolyoxyethylene-5000 (DMG-PEG5K); and 1,2-dimyristoyl-rac-glycero-3-methylpolyoxyethylene-2000 (DMG-PEG2K).

Embodiment 88. The particle of Embodiment 82, wherein the cationic lipid comprises COATSOME® SS-OC, wherein the one or more helper lipids comprise cholesterol (Chol) and DSPC, and wherein the phospholipid-polymer conjugate comprises DPG-PEG2000.

Embodiment 89. The particle of Embodiment 88, wherein the ratio of SS-OC:DSPC:Chol:DPG-PEG2K (as a percentage of total lipid content) is A:B:C:D, wherein:
a. A=40%-60%, B=10%-25%, C=20%-30%, and D=0%-3% and wherein A+B+C+D=100%;
b. A=45%-50%, B=20%-25%, C=25%-30%, and D=0%-1% and wherein A+B+C+D=100%
c. A=40%-60%, B=10%-30%, C=20%-45%, and D=0%-3% and wherein A+B+C+D=100%;
d. A=40%-60%, B=10%-30%, C=25%-45%, and D=0%-3% and wherein A+B+C+D=100%;
e. A=45%-55%, B=10%-20%, C=30%-40%, and D=1%-2% and wherein A+B+C+D=100%;
f. A=45%-50%, B=10%-15%, C=35%-40%, and D=1%-2% and wherein A+B+C+D=100%;
g. A=45%-65%, B=5%-20%, C=20%-45%, and D=0%-3% and wherein A+B+C+D=100%;
h. A=50%-60%, B=5%-15%, C=30%-45%, and D=0%-3% and wherein A+B+C+D=100%;
i. A=55%-60%, B=5%-15%, C=30%-40%, and D=1%-2% and wherein A+B+C+D=100%;
j. A=55%-60%, B=5%-10%, C=30%-35%, and D=1%-2% and wherein A+B+C+D=100%.

Embodiment 90. The particle of Embodiment 88, wherein the ratio of SS-OC:DSPC:Chol:DPG-PEG2K (as a percentage of total lipid content) is:
a. about 49:22:28.5:0.5;
b. about 49:11:38.5:1.5; or
c. about 58:7:33.5:1.5.

Embodiment 91. The particle of Embodiment 88, wherein the ratio of SS-OC:DSPC:Chol:DPG-PEG2K (as a percentage of total lipid content) is about 49:22:28.5:0.5.

Embodiment 92. The particle of Embodiment 82, wherein the cationic lipid is 1,2-dioleoyl-3-trimethylammonium-propane (DOTAP), and wherein the neutral lipid is 1,2-Dilauroyl-sn-glycero-3-phosphoethanolamine (DLPE) or 1,2-Dioleoyl-sn-glycero-3-phosphoethanolamine (DOPE).

Embodiment 93. The particle of Embodiment 82 or 92, further comprising a PEG-lipid, wherein the PEG-lipid is 1, 2-Distearoyl-sn-glycero-3-phosphoethanolamine-Poly(ethylene glycol) (DSPE-PEG) or 1,2-distearoyl-sn-glycero-3-phosphoethanolamine-N-[amino(polyethylene glycol)] (DSPE-PEG-amine).

Embodiment 94. The particle of any one of Embodiments 80-93, further comprising a second recombinant RNA molecule encoding an oncolytic virus.

Embodiment 95. The particle of Embodiment 94, wherein the oncolytic virus is a picornavirus.

Embodiment 96. The particle of Embodiment 95, wherein the picornavirus is selected from a senecavirus, a cardiovirus, and an enterovirus.

Embodiment 97. The particle of Embodiment 95, wherein the picornavirus is a Seneca Valley Virus (SVV).

Embodiment 98. The particle of Embodiment 95, wherein the picornavirus is a Coxsackievirus.

Embodiment 99. The particle of Embodiment 95, wherein the picornavirus is an encephalomyocarditis virus (EMCV).

Embodiment 100. A therapeutic composition comprising a plurality of lipid nanoparticles according to any one of Embodiments 82-99.

Embodiment 101. The therapeutic composition of Embodiment 100 wherein the plurality of LNPs have an average size of about 50 nm to about 120 nm.

Embodiment 102. The therapeutic composition of Embodiment 100 wherein the plurality of LNPs have an average size of about 100 nm.

Embodiment 103. The therapeutic composition of any one of Embodiments 100-102, wherein the plurality of LNPs have an average zeta-potential of between about 20 mV to about −20 mV, about 10 mV to about −10 mV, about 5 mV to about −5 mV, or about 20 mV to about −40 mV, −50 mV to about-20 mV, about −40 mV to about −20 mV, or about −30 mV to about −20 mV.

Embodiment 104. The therapeutic composition of Embodiment 103, wherein the plurality of LNPs have an average zeta-potential of about −30 mV, about −31 mV, about-32 mV, about −33 mV, about −34 mV, about −35 mV, about −36 mV, about −37 mV, about −38 mV, about −39 mV, or about −40 mV.

Embodiment 105. A method of killing a cancerous cell comprising exposing the cancerous cell to the particle of any one of Embodiments 80-97, the vector of any one of Embodiments 77-79, the recombinant RNA replicon of any one of Embodiments 1-65, or compositions thereof.

Embodiment 106. The method of Embodiment 105, wherein the method is performed in vivo, in vitro, or ex vivo.

Embodiment 107. A method of treating a cancer in a subject comprising administering to the subject suffering from the cancer an effective amount of the particle of any one of Embodiments 80-97, the vector of any one of Embodiments 77-79, the recombinant RNA replicon of any one of Embodiments 1-65, or compositions thereof.

Embodiment 108. The method of Embodiment 107, wherein the particle, the recombinant RNA replicon, or composition thereof is administered intravenously, intranasally, as an inhalant, or is injected directly into a tumor.

Embodiment 109. The method of Embodiment 107 or 108, wherein the particle, the recombinant RNA replicon, or composition thereof is administered to the subject repeatedly.

Embodiment 110. The method of any of Embodiments 107-109, wherein the subject is a mouse, a rat, a rabbit, a cat, a dog, a horse, a non-human primate, or a human.

Embodiment 111. The method of any of Embodiments 107-110, wherein the cancer is selected from lung cancer, breast cancer, ovarian cancer, cervical cancer, prostate cancer, testicular cancer, colorectal cancer, colon cancer, pancreatic cancer (e.g., Castration resistant neuroendocrine prostate cancer), liver cancer, gastric cancer, head and neck cancer, thyroid cancer, malignant glioma, glioblastoma, melanoma, B-cell chronic lymphocytic leukemia, diffuse large B-cell lymphoma (DLBCL), sarcoma, a neuroblastoma, a neuroendocrine cancer, a rhabdomyosarcoma, a medulloblastoma, a bladder cancer, marginal zone lymphoma (MZL), Merkel cell carcinoma, and renal cell carcinoma.

Embodiment 112. The method of Embodiment 111, wherein:
a. the lung cancer is small cell lung cancer or non-small cell lung cancer;
b. the liver cancer is hepatocellular carcinoma (HCC); and/or
c. the prostate cancer is treatment-emergent neuroendocrine prostate cancer.

Embodiment 113. The method of Embodiments 111, wherein the cancer is a neuroendocrine cancer.

Embodiment 114. A method of immunizing a subject against a disease, comprising administering to the subject an effective amount of the particle of any one of Embodiments 80-97, the vector of any one of Embodiments 77-79, the recombinant RNA replicon of any one of Embodiments 1-65, or compositions thereof.

Embodiment 115. The method of Embodiment 114, wherein the particle, the recombinant RNA replicon, or composition thereof is administered intravenously, intramuscularly, intradermally, intranasally, or as an inhalant.

Embodiment 116. The method of Embodiment 114 or 115, wherein the particle, the recombinant RNA replicon, or composition thereof is administered to the subject repeatedly.

Embodiment 117. The method of any one of Embodiments 114 to 116, wherein the disease is an infectious disease.

Embodiment 118. The method of Embodiment 117, wherein the infectious disease is caused by one of the pathogens comprising Dengue virus, Chikungunya virus, Mycobacterium tuberculosis, Human immunodeficiency virus, SARS-CoV-2, Coronavirus, Hepatitis B virus, Togaviridae family virus, Flaviviridae family virus, Influenza A virus, Influenza B virus and a veterinary virus.

Embodiment 119. A recombinant RNA replicon comprising a picornavirus genome and a heterologous polynucleotide.

Embodiment 120. The recombinant RNA replicon of Embodiment 119, wherein the heterologous polynucleotide is inserted between a 2A coding region and a 2B coding region.

Embodiment 121. The recombinant RNA replicon of Embodiment 119, wherein the heterologous polynucleotide is inserted between a 5′ UTR and a 2A coding region.

Embodiment 122. The recombinant RNA replicon of Embodiment 119, wherein the heterologous polynucleotide is inserted between a 3D coding region and a 3′ UTR.

Embodiment 123. The recombinant RNA replicon of any one of Embodiments 119-122, wherein the picornavirus is selected from a senecavirus, a cardiovirus, and an enterovirus.

### EXAMPLES

The following examples are given for the purpose of illustrating various embodiments of the disclosure and are not meant to limit the present disclosure in any fashion. The present examples; along with the methods described herein are presently representative of preferred embodiments; are exemplary; and are not intended as limitations on the scope of the disclosure. Changes therein and other uses which are encompassed within the spirit of the disclosure as defined by the scope of the claims will occur to those skilled in the art.

### Example 1: Insertion of Heterologous Polynucleotide Reduces SVV Viral Replication

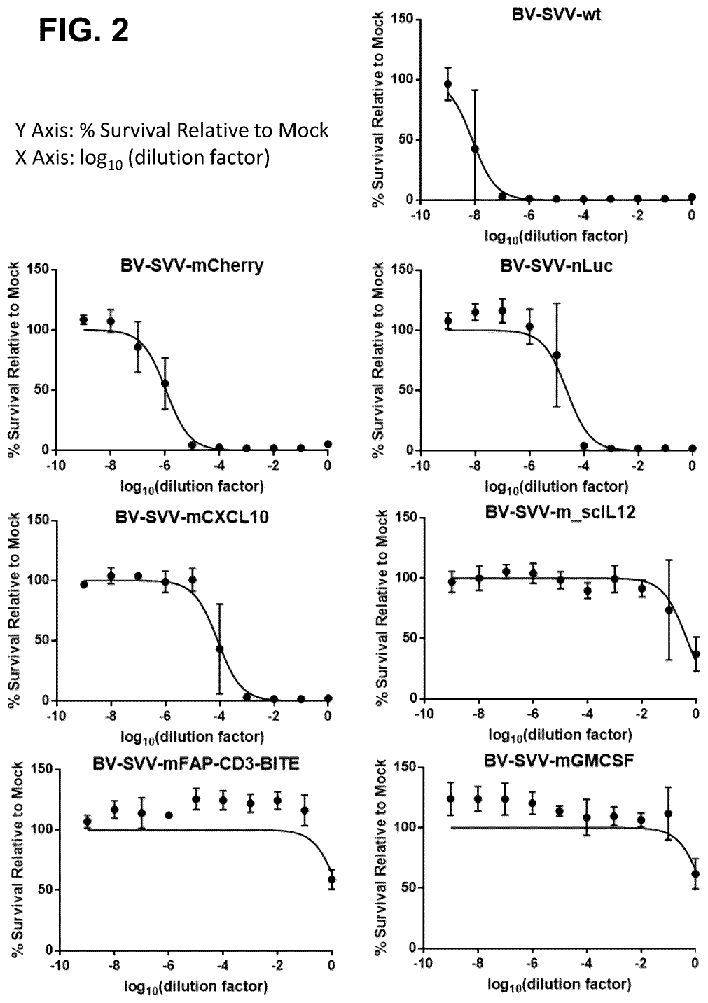

Experiments were performed to assess the ability of viral replication of Seneca Valley Virus (SVV) with heterologous polynucleotides of varying lengths inserted into the viral genome (Table 15). Briefly, NCI-H1299 cells were transfected with 0.015 pmol of plasmid encoding the recombinant SVV viral genome on Day 1. Cells were harvested and supernatant were filtered to collect viruses on Day 4. On Day 5, NCI-H446 cells were infected with the collected viruses and CTG assay was performed to estimate viral replication rate. The results are shown in FIG. 2 and Table 15. Insertion of the heterologous polynucleotides caused reduction of viral replication rate.

TABLE 15
Viral Replication of SVV Comprising
Heterologous Polynucleotides

| | Payload | |
|---|---|---|
| | Size | |
| Construct | (bp) | IC50 |
| BV-SVV-wt | n/a | 8.03E−09 |
| BV-SVV-mCherry | 702 | 1.09E−06 |
| BV-SVV-nLuc | 513 | 2.36E−05 |
| BV-SVV-mCXCL10 | 294 | 8.55E−05 |
| BV-SVV-m_scIL12 | 1629 | 4.43E−01 |
| BV-SVV-mFAP-CD3-BiTE | 1518 | 1.80E+00 |
| BV-SVV-mGMCSF | 423 | 1.90E+00 |

### Example 2: Identification of SVV Cis-Acting Replication Element (CRE) within VP2 Region

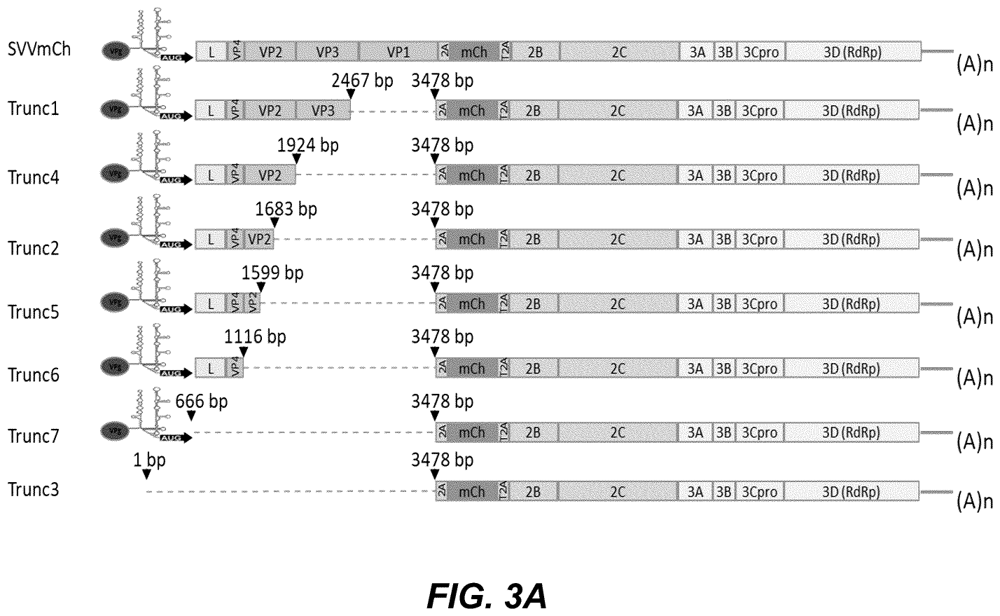

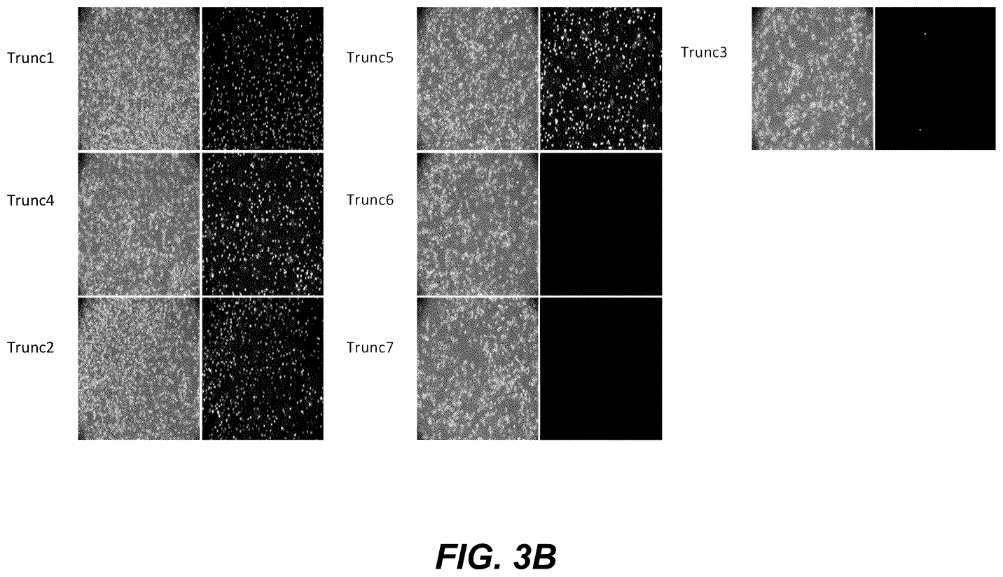

Experiments were performed to assess whether deletions/truncations within the viral genome region encoding one or more VP proteins affect SVV viral replication. SVV derived recombinant RNA replicons comprising an mCherry reporter gene and deletions and/or truncations in the regions encoding VP proteins were generated according to Table 16 and FIG. 3A. Corresponding recombinant RNA replicons were generated via in vitro T7 transcription. NCI-H1299 cells were transfected with the resultant RNA, and mCherry expression were evaluated 24 hours after the transfection. The results showed that deletions between 1599 bp-3478 bp has minimal effect on SVV viral replication, whereas a deletion of the nucleotides between 1116 bp-1599 bp (within the VP2 coding region) greatly reduced SVV viral replication (FIG. 3B). Therefore, a cis-acting replication element (or at least a part of the cis-acting replication element) is present between 1116 bp-1599 bp of SVV viral genome.

TABLE 16
SVV Replicons with Deletion in the VP Coding Region

| | | SEQ ID NO: | | |
|---|---|---|---|---|
| | | (for DNA | | |
| | SEQ ID NO: | vector | | Replicon |
| Replicon | (for Replicon) | templates) | Deletion | Length |
| SVVmCh | 20 | 19 | 0 bp | 8.0 kb |
| Trunc1 | 22 | 21 | 1011 bp | 7.1 kb |
| Trunc4 | 28 | 27 | 1554 bp | 6.5 kb |
| Trunc2 | 24 | 23 | 1795 bp | 6.3 kb |
| Trunc5 | 30 | 29 | 1878 bp | 6.2 kb |
| Trunc6 | 32 | 31 | 2362 bp | 5.7 kb |
| Trunc7 | 34 | 33 | 2812 bp | 5.3 kb |
| Trunc3 | 26 | 25 | 3478 bp | 4.6 kb |

### Example 3: Trunc5 Replicon is Trans-Encapsidated by SVVwt with Minimal Efficacy Loss

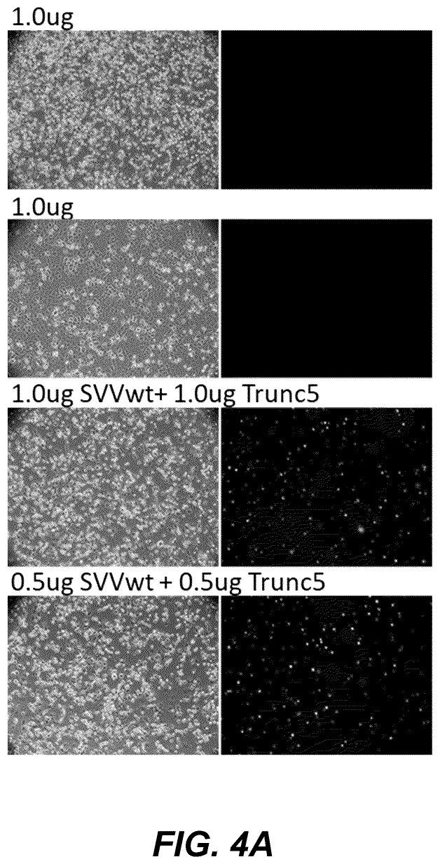

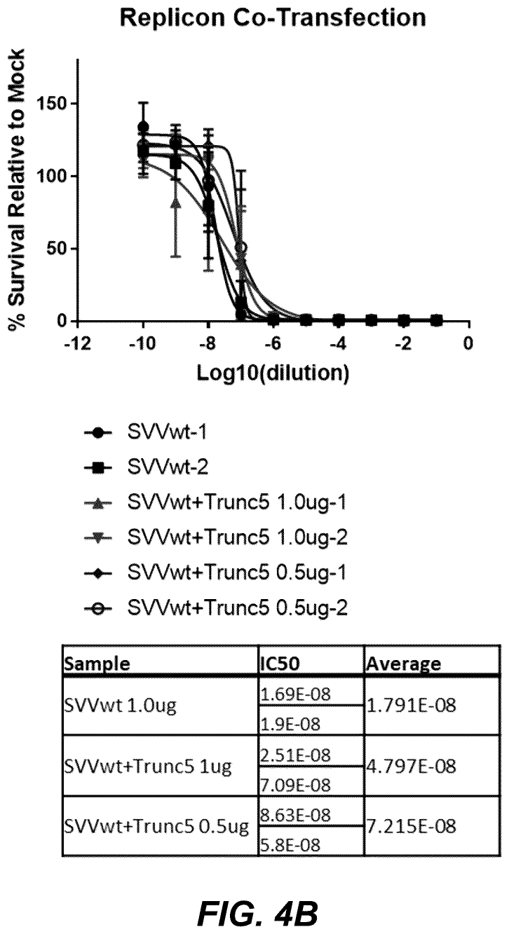

Experiments were performed to assess whether Trunc5 replicon can retain efficacy after trans-encapsidation by wildtype SVV and determine fitness cost to wildtype SVV. Briefly, Trunc5 replicon and/or wildtype SVV viral genome were linearized with NotI restriction enzyme and in vitro transcribed (IVT) with the HiScribe T7 RNA Synthesis Kit (NEB). NCI-H1299 cells were co-transfected with 0.5 or 1 ug of each or both resultant RNA molecules using Lipofectamine RNAiMax (Invitrogen). At 48 hours post transfection the supernatant was collected and filtered through a 0.45 um filter. 100 ul of the filtered supernatant was transferred onto a fresh monolayer of H1299 cells and expression of mCherry was observed at 24 hours post infection (FIG. 4A). Expression of mCherry was detected after co-transfection with SVVwt but not after transfection with replicon alone, demonstrating that Trunc5 was trans-encapsidated. Viral titer from filtered supernatants from SVVwt transfection versus SVVwt and Trunc5-SVV replicon co-transfection were compared using an IC50 assay performed in NCI-H446 cells (FIG. 4B). Co-transfection of SVVwt with Trunc5 results in minimal reduction of viral titer.

### Example 4: Trunc10 Maintains CRE and Maximizes Payload Capacity

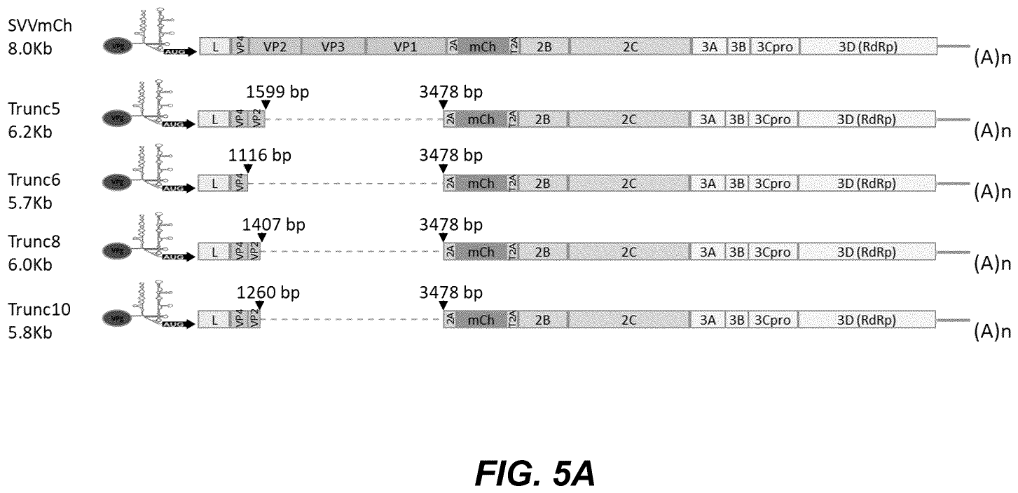

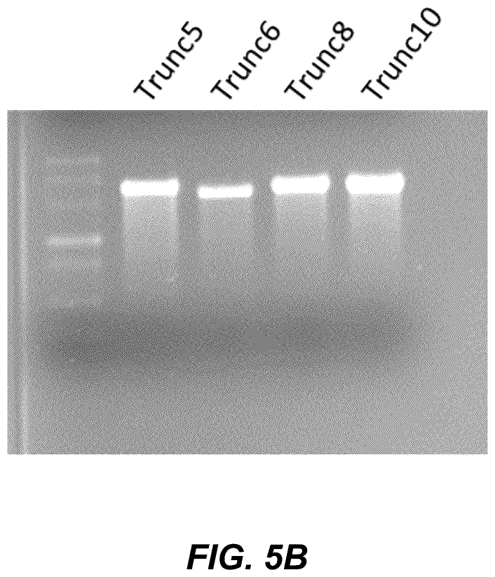

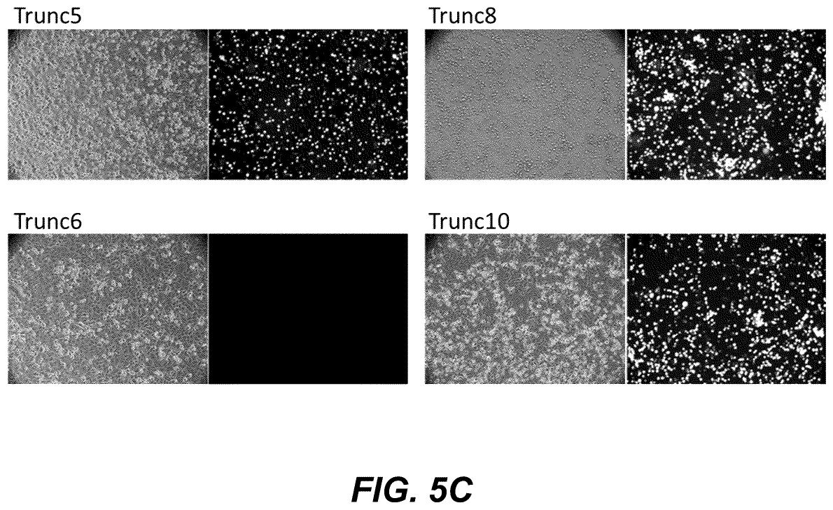

Further experiments were performed to narrow down the location of CRE and analyze the length of tolerable deletion within the SVV VP coding regions. SVV derived recombinant RNA replicons comprising an mCherry reporter gene and deletions and/or truncations in the regions encoding VP proteins were generated according to Table 17 and FIG. 5A. Experiments were performed according to the protocol described in Example 2. FIG. 5B shows the RNA molecules generated via in vitro T7 RNA synthesis, and FIG. 5C shows mCherry signal of various replicon. The results showed that a deletion between 1260 bp-3478 bp (Trunc10 replicon) has minimal impact on SVV viral replication.

TABLE 17
SVV Replicons with Deletion in the VP Coding Region

| | | SEQ ID NO: | | |
|---|---|---|---|---|
| | | (for DNA | | |
| | SEQ ID NO: | vector | | Replicon |
| Replicon | (for Replicon) | templates) | Deletion | Length |
| SVVmCh | 20 | 19 | 0 bp | 8.0 kb |
| Trunc5 | 30 | 29 | 1878 bp | 6.2 kb |
| Trunc6 | 32 | 31 | 2362 bp | 5.7 kb |
| Trunc8 | 36 | 35 | 2071 bp | 6.0 kb |
| Trunc10 | 38 | 37 | 2218 bp | 5.8 kb |

### Example 5: SVV Replicons with Single Payload are Replication and Trans-Encapsidation Competent

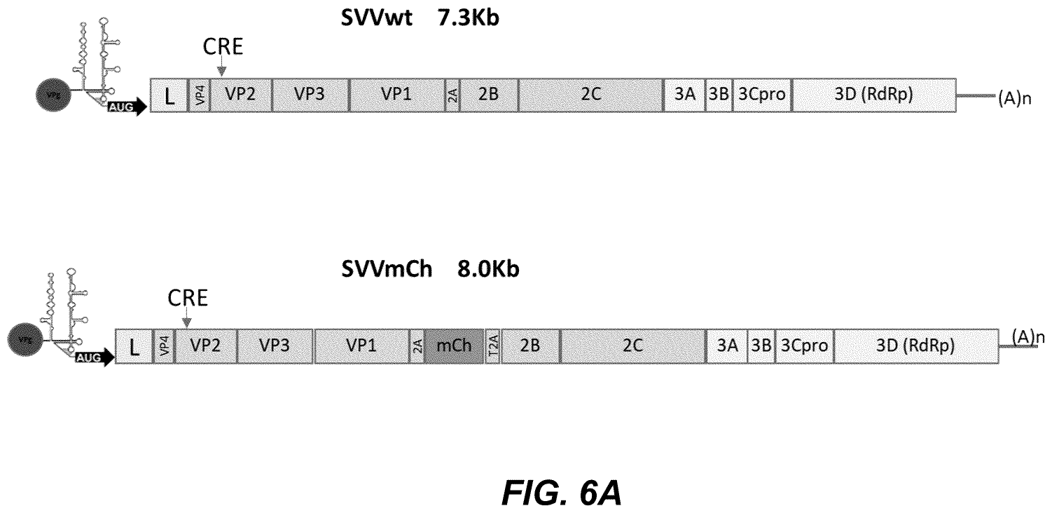

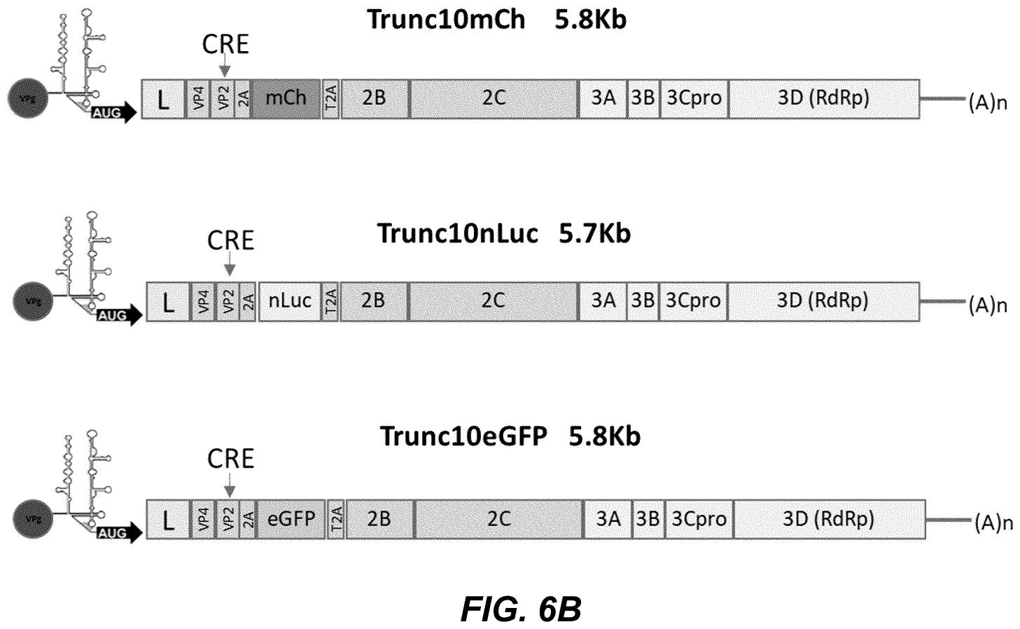

Replicons were constructed for in-vitro and in-vivo testing of competency (FIGS. 6A-6B). Various payloads (mCherry, nano-Luciferase, or eGFP) were inserted into the replicon as shown in FIG. 6B, and the resultant replicons were tested according to protocols in Examples 2-4. The result showed that all these replicons are replication and trans-encapsidation competent.

### Example 6: SVV-Trunc10 Replicon with Murine IL-2 Payload

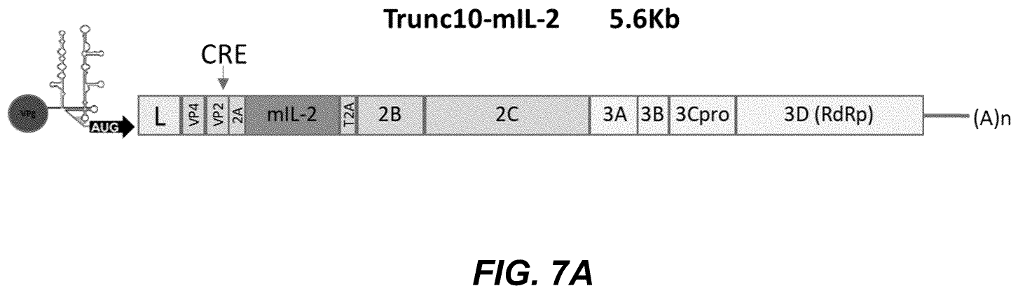

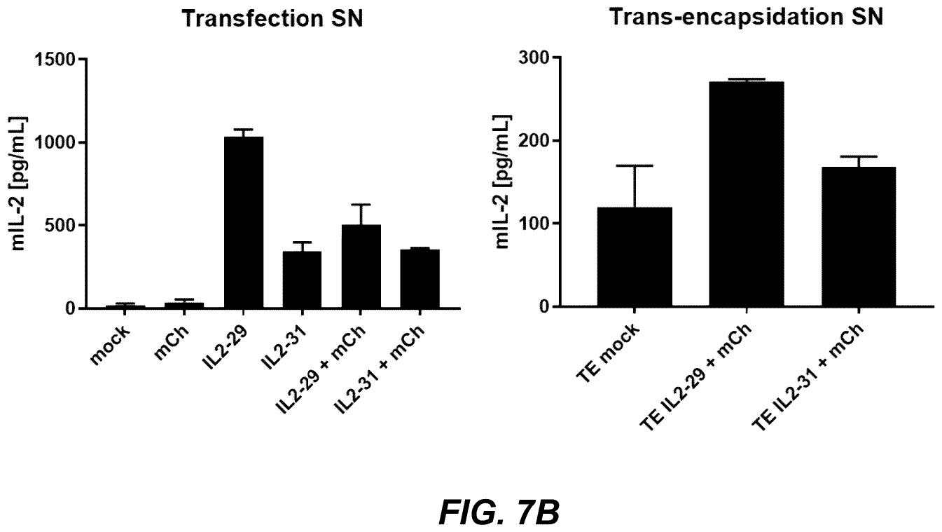

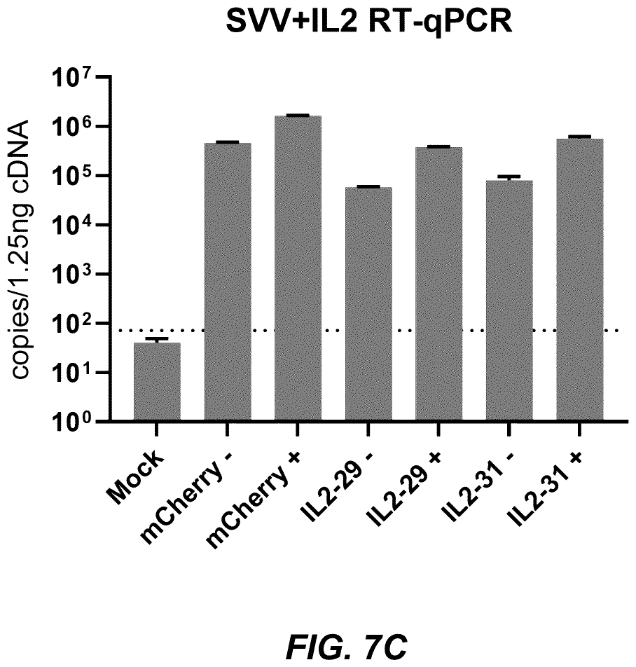

Experiments were conducted to test the expression of murine IL-2 payload protein via the SVV-Trunc10 replicon. FIG. 7A shows the construction of SVV-replicon Trunc10 carrying a transgene encoding murine TL-2 payload. The replicon and SVV-mCherry templates were linearized with NotI restriction enzyme and in vitro transcribed (IVT) with the HiScribe T7 RNA Synthesis Kit (NEB). H1299 cells were transfected using Lipofectamine RNAiMax (Invitrogen) with 1 ug of Replicon RNA or 1 ug of Replicon RNA plus 1 ug SVVmCherry. At 48 hours post transfection the supernatant was collected and filtered through a 0.45 um filter and RNA was collected in QIAzol (Qiagen). 100 ul of the filtered supernatant was transferred onto a fresh monolayer of H1299 cells and supernatant was collected at 48 hours post infection. Expression of murine IL-2 was detected with a mIL-2 ELISA (R&D) (FIG. 7B). In addition, viral RNA was isolated and analyzed with a positive and negative strand specific taqman assay (FIG. 7C). The results showed that the SVV-Trunc10-mIL-2 replicon expresses and secretes mIL-2 after transfection and trans-encapsidation, and is competent for positive and negative strand viral RNA synthesis.

### Example 7: SVV-Trunc10 Replicon with Single Chain mIL-12 Payload

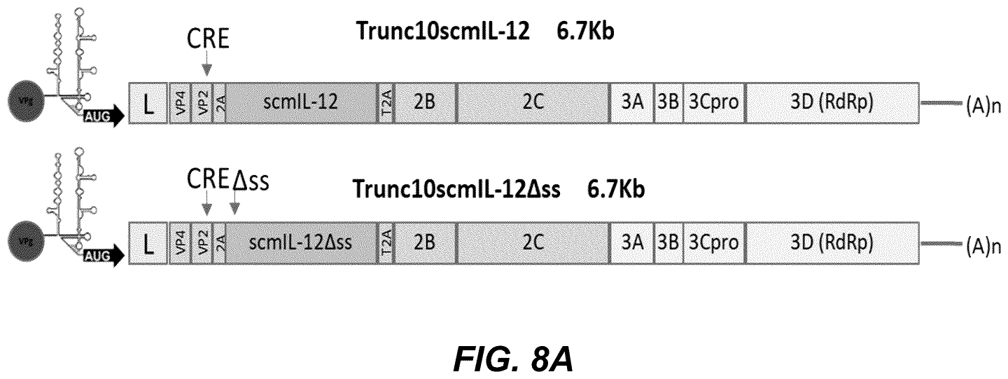

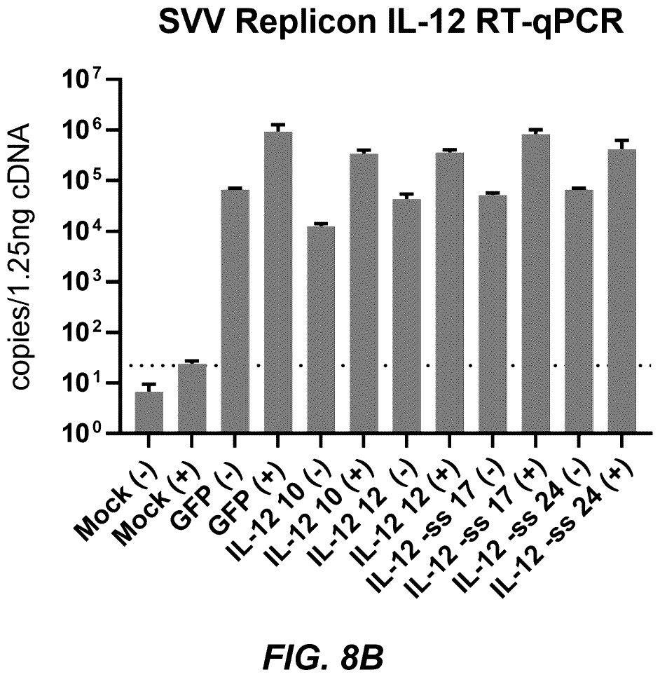

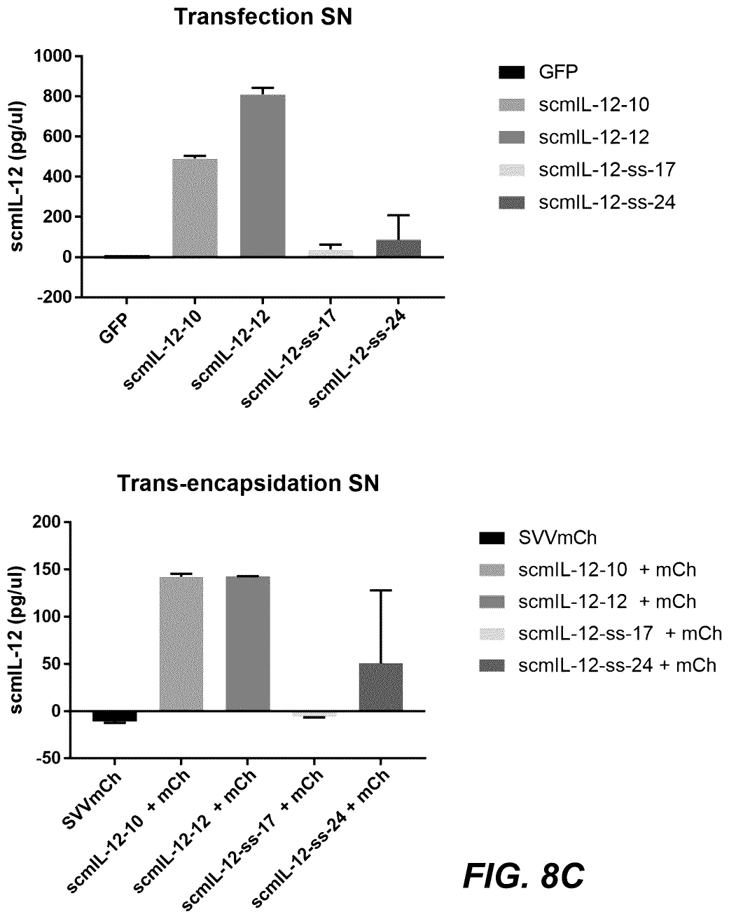

Experiments were conducted to test the expression of single chain mIL-12 (scmIL-12) payload protein via the SVV-Trunc10 replicon. FIG. 8A depicts the construction of SVV-replicon Trunc10 carrying a transgene encoding single chain mIL-12 (scmIL-12), with and without a signal sequence. The replicon and SVV-mCherry templates were linearized with NotI restriction enzyme and in vitro transcribed (IVT) with the HiScribe T7 RNA Synthesis Kit (NEB). H1299 cells were transfected using Lipofectamine RNAiMax (Invitrogen) with 1 ug of Replicon RNA or 1 ug of Replicon RNA plus 1 ug SVVmCherry. At 48 hours post transfection the supernatant was collected and filtered through a 0.45 um filter and RNA was collected in QIAzol (Qiagen). 100 ul of the filtered supernatant was transferred onto a fresh monolayer of H1299 cells and supernatant was collected at 48 hours post infection. RNA was isolated and analyzed with a positive and negative strand specific taqman assay (FIG. 8B), which showed that the replicons are competent for positive and negative strand viral RNA synthesis, and payload secretion is not correlated with positive or negative viral RNA synthesis. Expression of murine IL-12 was detected with a mIL-2 ELISA (R&D) (FIG. 8C), which showed that the Trunc10-scmIL-12 replicon expressed and secreted mIL-12 after transfection and trans-encapsidation. Deletion of the signal sequence reduced IL-12 secretion. Overall, the results showed that the Trunc10-scmIL-12 and Trunc10-scmIL-12Δss replicons are competent for positive and negative strand viral RNA synthesis, however the intact signal sequence facilitates expression and secretion of mIL-12 after transfection and trans-encapsidation.

### Example 8: SVV-Trunc10-hIL-36γ Replicon

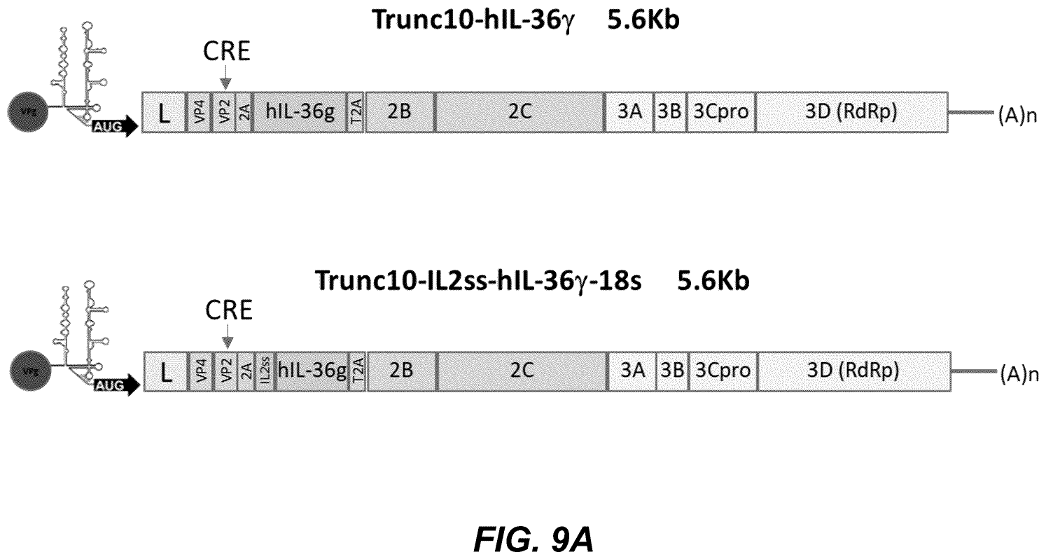

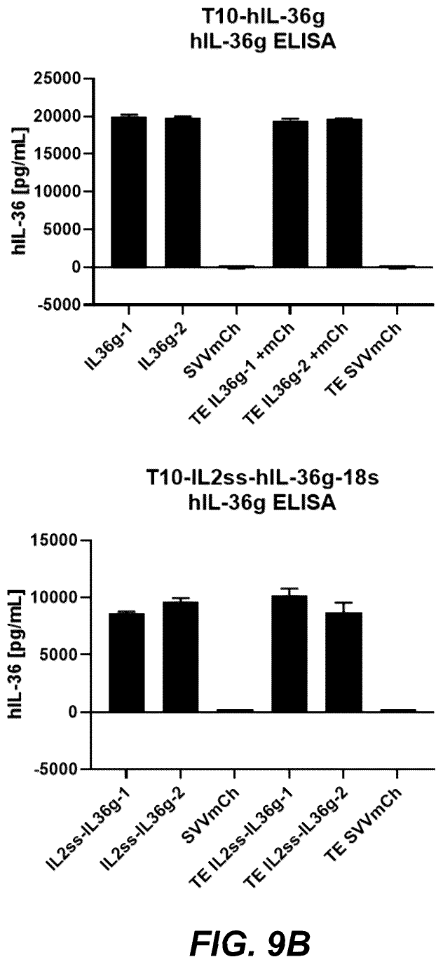

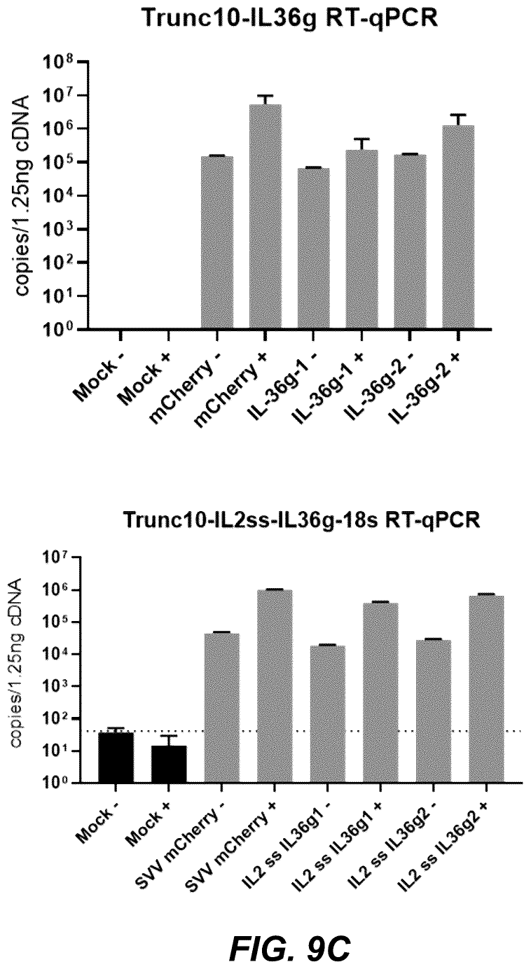

Experiments were conducted to test the expression of human IL-36γ payload protein via the SVV-Trunc10 replicon. FIG. 9A depicts the construction of SVV-replicon Trunc10 carrying a transgene encoding human IL-36γ, with the native signal sequence or with the IL2 signal sequence. The replicon and SVV-mCherry templates were linearized with NotI restriction enzyme and in vitro transcribed (IVT) with the HiScribe T7 RNA Synthesis Kit (NEB). H1299 cells were transfected using Lipofectamine RNAiMax (Invitrogen) with 1 ug of Replicon RNA or 1 ug of Replicon RNA plus 1 ug SVVmCherry. At 48 hours post transfection the supernatant was collected and filtered through a 0.45 um filter and RNA was collected in QIAzol reagent (Qiagen). 100 ul of the filtered supernatant was transferred onto a fresh monolayer of H1299 cells and supernatant was collected at 48 hours post infection. Expression of hIL-36γ was detected with an hIL-36γ ELISA (R&D). Both replicons express and secrete hIL-36γ after transfection and trans-encapsidation, and addition of the IL-2 signal sequence does not improve expression or secretion (FIG. 9B). RNA was isolated and analyzed with a positive and negative strand specific taqman assay. The results (FIG. 9C) showed that both replicons are competent for positive and negative strand viral RNA synthesis. Overall, the results demonstrated that the SVV-Trunc10-hIL-36γ and SVV-Trunc10-IL2ss-hIL-36γ-18S replicons express and secrete hIL-36γ after transfection and trans-encapsidation, and are competent for positive and negative strand viral RNA synthesis.

### Example 9: Second IRES in Dicistronic Replicons does not Improve Replicon Function

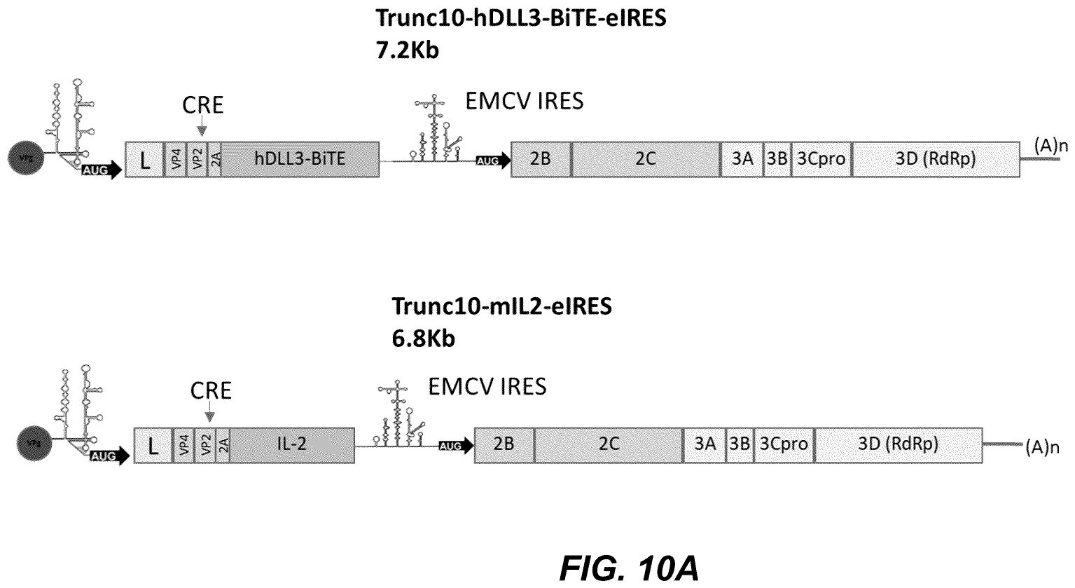

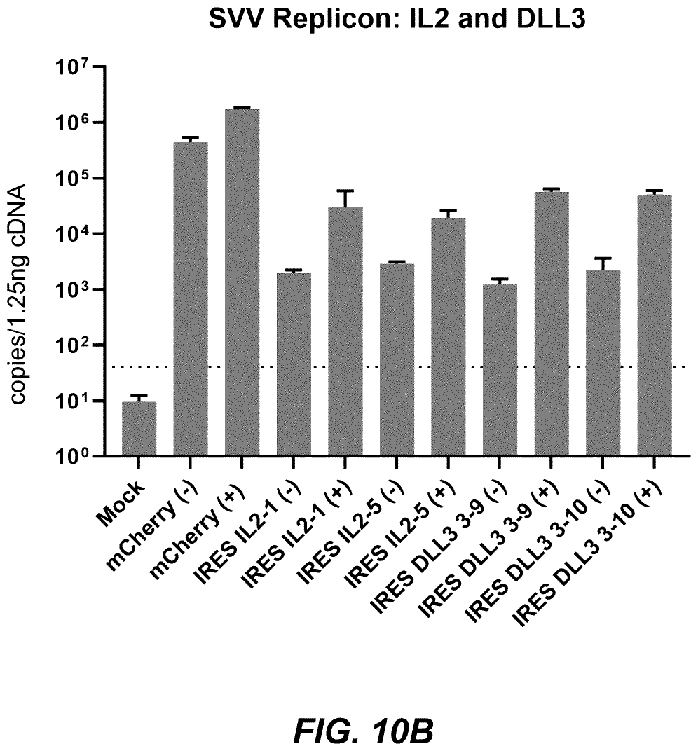

FIG. 10A depicts construction of dicistronic replicons incorporated with a second encephalomyocarditis virus (EMCV) IRES downstream of a single payload. The effect of the second IRES on replicon function improvement were tested. The replicon and SVV-mCherry templates were linearized with NotI restriction enzyme and in vitro transcribed (IVT) with the HiScribe T7 RNA Synthesis Kit (NEB). H1299 cells were transfected using Lipofectamine RNAiMax (Invitrogen) with 1 ug of Replicon RNA or 1 ug SVVmCherry. At 48 hours post transfection RNA was collected with QIAzol (Qiagen). RNA was isolated and analyzed with a positive and negative strand specific taqman assay (FIG. 10B). The results showed that negative strand synthesis of the replicon was impaired by the addition of the second ECMV IRES, and incorporation of a second IRES downstream of the hDLL3-BiTE or mIL-2 payloads did not improve viral RNA synthesis.

### Example 10: Multiple furinT2A Sites are not Effective for Payload Expression in Dicistronic Dual Payload Replicons

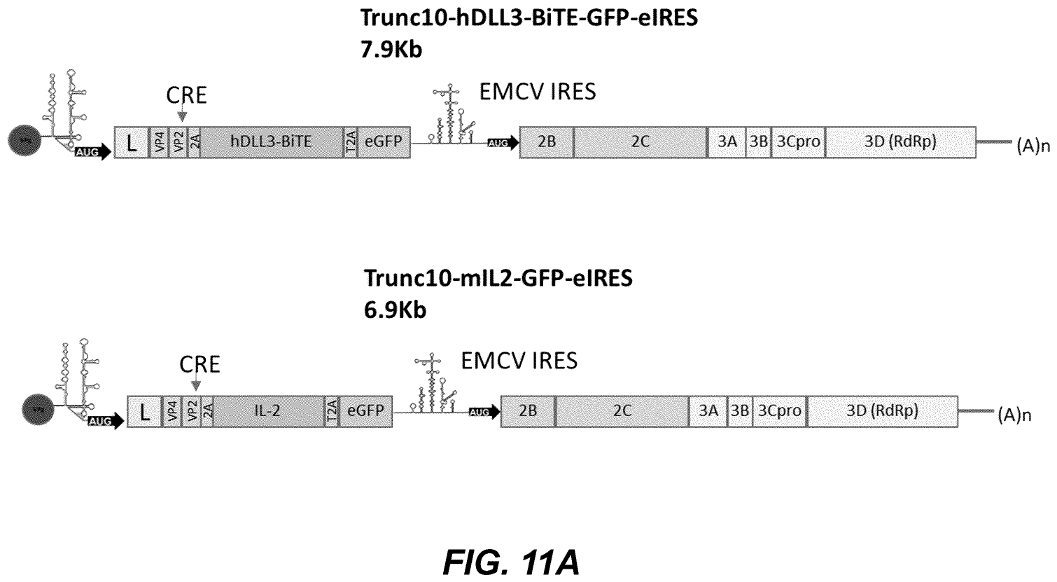

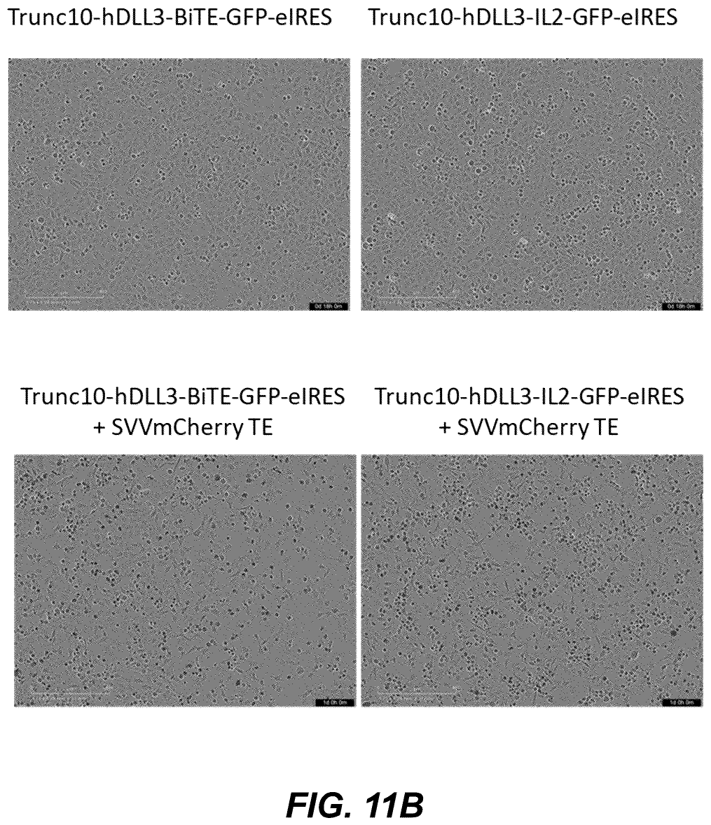

FIG. 11A depicts construction of dicistronic dual payload replicons incorporated with a second encephalomyocarditis virus (EMCV) IRES downstream of multiple payloads separated by a furin-T2A site between the first payload and the second payload (eGFP). The replicon and SVV-mCherry templates were linearized with NotI restriction enzyme and in vitro transcribed (IVT) with the HiScribe T7 RNA Synthesis Kit (NEB). H1299 cells were transfected using Lipofectamine RNAiMax (Invitrogen) with 1 ug of Replicon RNA or 1 ug of replicon RNA plus 1 ug SVVmCherry. At 48 hours post transfection cells were observed for GFP expression and the supernatant from co-transfection of replicon and SVVmCherry was collected and filtered through a 0.45 um filter. 100 ul of the filtered supernatant was transferred onto a fresh monolayer of H1299 cells and cells were examined for mCherry and GFP expression 24 hours post infection (FIG. 11B). Minimal expression of GFP was detected after transfection of either Trunc10-hDLL3-BiTE-GFP-eIRES or Trunc10-mIL2-GFP-eIRES or after trans-encapsidation, indicating that the dicistronic replicon has impaired expression of the second payload.

### Example 11: Dual Payload Replicon with Second Payload Incorporated at the 3′ End of the Replicon is not Efficient for Replication

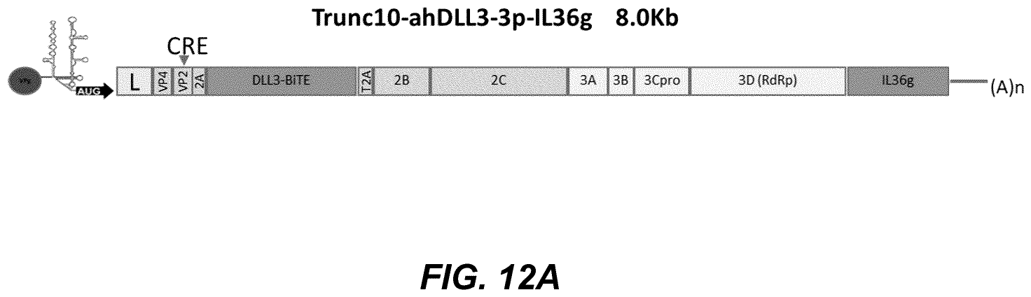

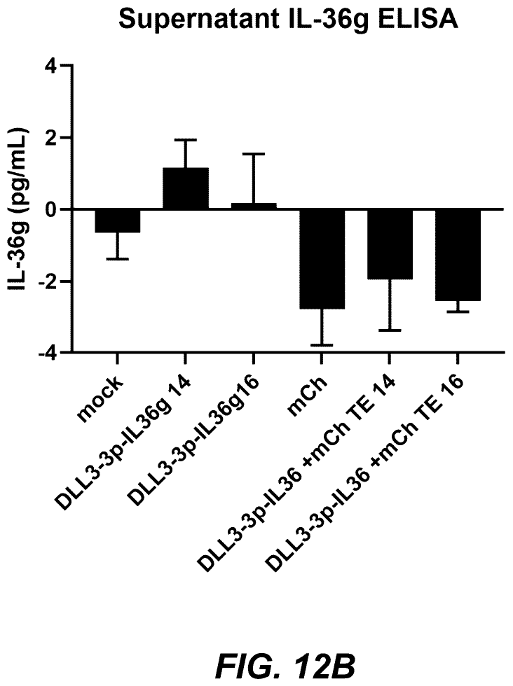

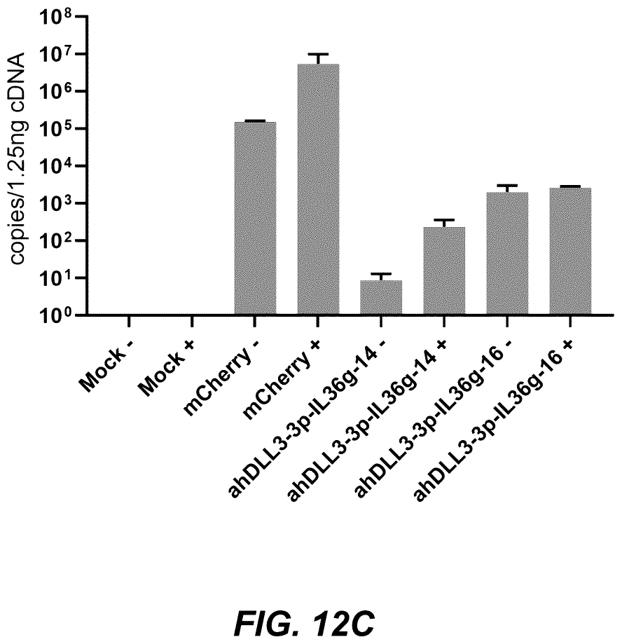

FIG. 12A depicts construction of a dual payload replicon incorporated with a second payload at the 3′ end of the replicon between the RdRp and the 3′UTR. The replicon and SVV-mCherry templates were linearized with NotI restriction enzyme and in vitro transcribed (IVT) with the HiScribe T7 RNA Synthesis Kit (NEB). H1299 cells were transfected using Lipofectamine RNAiMax (Invitrogen) with 1 ug of Replicon RNA or 1 ug of Replicon RNA plus 1 ug SVVmCherry. At 48 hours post transfection the supernatant was collected and filtered through a 0.45 um filter and RNA was collected in QIAzol reagent (Qiagen). 100 ul of the filtered supernatant was transferred onto a fresh monolayer of H1299 cells and supernatant was collected at 48 hours post infection. Expression of hIL-36γ was detected with an hIL-36γ ELISA (R&D). The replicon secreted less than 2 pg/mL of hIL-36 after transfection and the hIL-36 secretion was not detectable after trans-encapsidation (FIG. 12B). RNA was isolated and analyzed with a positive and negative strand specific taqman assay, which showed that the replicon was defective for positive and negative strand viral RNA synthesis (FIG. 12C). The results demonstrated that payload insertion at the 3′ end results in very low expression of IL-36γ after transfection, inhibits trans-encapsidation, and reduces positive and negative strand synthesis.

### Example 12: Expression of 1DLT176-MTT10-DLL3-VHH-CD3 Using Trunc10 Replicon

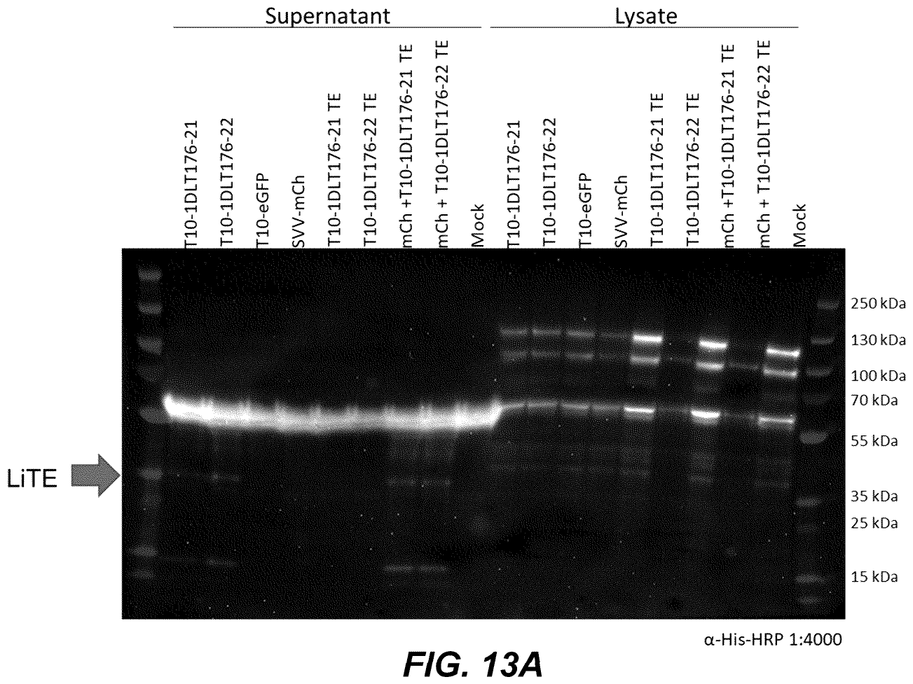

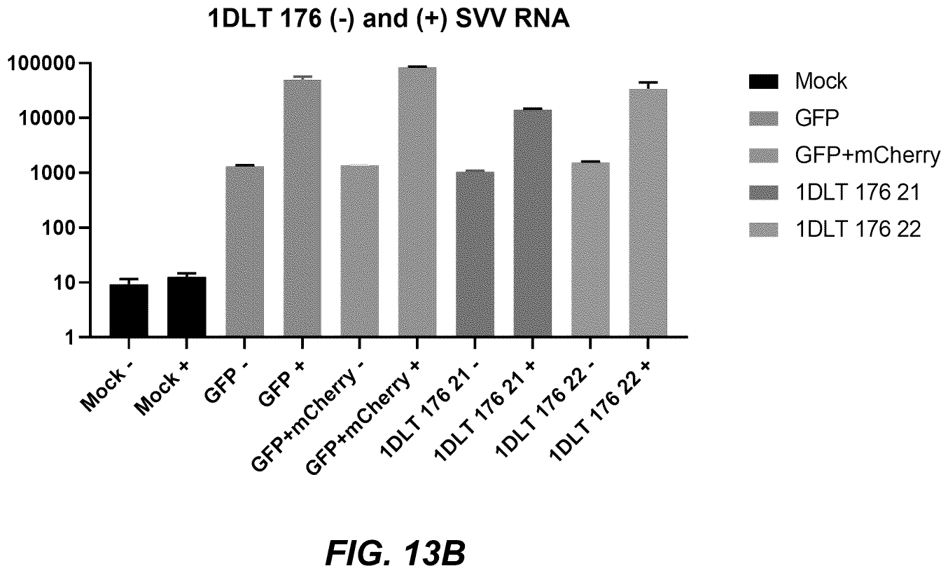

Single payload replicon for expression of his-tagged 1DLT176-MTT10-DLL3-VHH-CD3 LiTE was constructed. The replicon and SVV-mCherry templates were linearized with NotI restriction enzyme and in vitro transcribed (IVT) with the HiScribe T7 RNA Synthesis Kit (NEB). H1299 cells were transfected using Lipofectamine RNAiMax (Invitrogen) with 1 ug of Replicon RNA or 1 ug of Replicon RNA plus 1 ug SVVmCherry. At 48 hours post transfection the supernatant was collected and filtered through a 0.45 um filter and RNA was collected in QIAzol reagent (Qiagen). 100 ul of the filtered supernatant was transferred onto a fresh monolayer of H1299 cells and supernatant was collected at 48 hours post infection. Expression of his-tagged 1DLT176-MTT10-DLL3-VHH-CD3 LiTE was detected with an anti-His western blot. Specific bands correlated with LiTE expression were indicated with an arrow and detected in supernatant of transfected and trans-encapsidated samples (FIG. 13A). RNA is isolated and analyzed with a positive and negative strand specific taqman assay (FIG. 13B). The results suggested that the Trunc10-1DLT176-MTT10-DLL3-VHH-CD3 replicon is competent for LiTE payload expression, and for positive and negative strand viral RNA synthesis.

### Example 13: Expression of rDLL3-αCD3-H/L-BiTE and T10-rDLL3-αCD3-L/H-BiTE Using Trunc10 Replicon

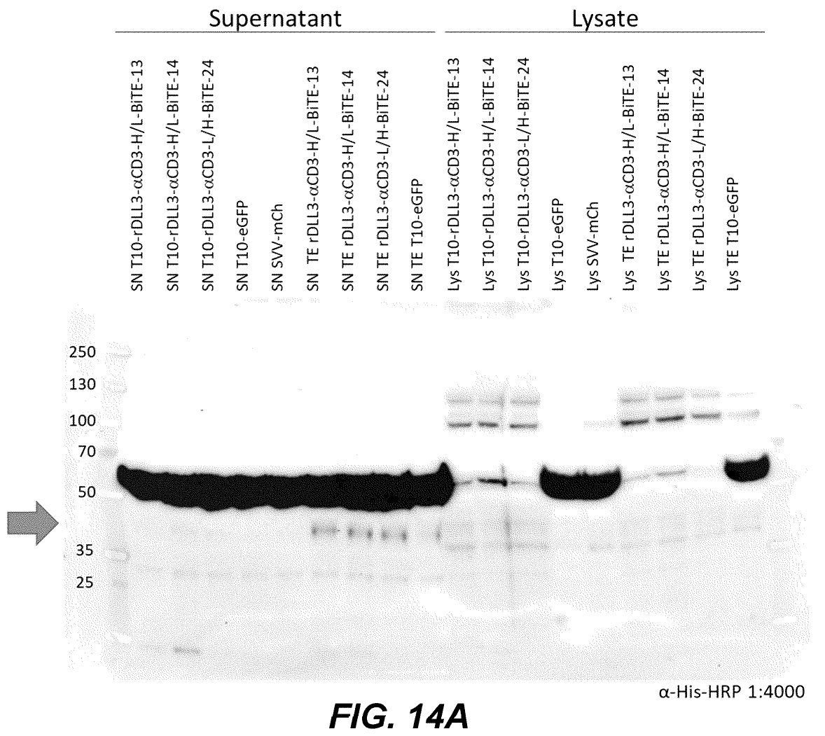

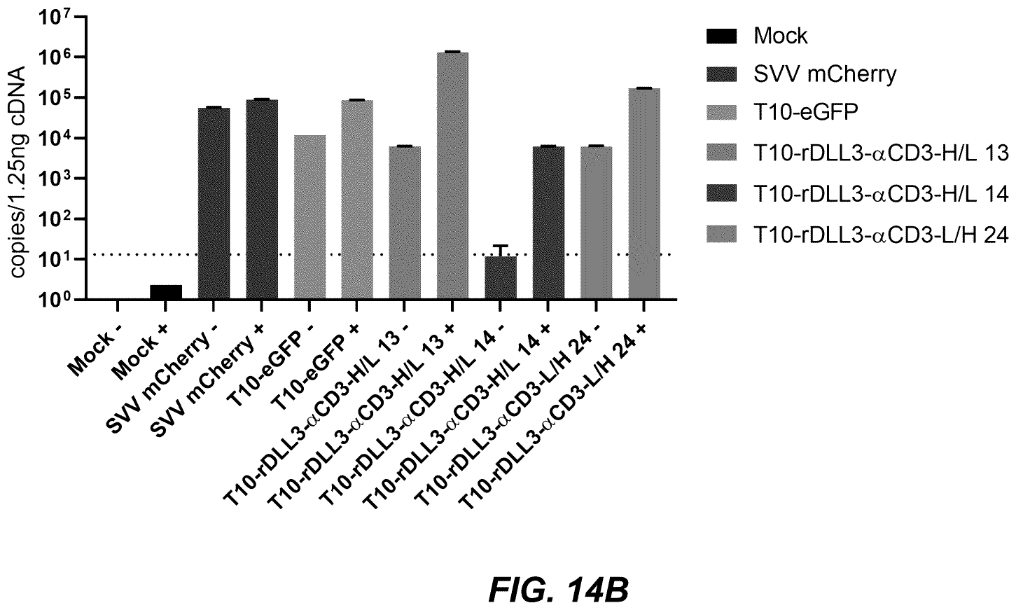

Single payload replicons for expression of his-tagged rDLL3-αCD3-BiTE were constructed. The H/L is oriented with heavy chain followed by light chain, while the reverse is true for L/H. A. The replicon and SVV-mCherry templates were linearized with NotI restriction enzyme and in vitro transcribed (IVT) with the HiScribe T7 RNA Synthesis Kit (NEB). H1299 cells were transfected using Lipofectamine RNAiMax (Invitrogen) with 1 ug of Replicon RNA or 1 ug of Replicon RNA plus 1 ug SVVmCherry. At 48 hours post transfection the supernatant was collected and filtered through a 0.45 um filter and RNA was collected in QIAzol reagent (Qiagen). 100 ul of the filtered supernatant was transferred onto a fresh monolayer of H1299 cells and supernatant was collected at 48 hours post infection. Expression of his-tagged rDLL3-αCD3-BiTE was detected with an anti-His western blot (FIG. 14A). Specific bands correlated with LiTE expression is indicated with an arrow and detected in supernatant of transfected and trans-encapsidated samples. RNA was isolated and analyzed with a positive and negative strand specific taqman assay (FIG. 14B). The results demonstrated that both of the Trunc10-rDLL3-αCD3 BiTE expressing replicons are competent for BiTE payload expression, trans-encapsidation, and for positive and negative strand viral RNA synthesis. The orientation of heavy and light chain does not affect replicon function.

### Example 14: Alternate Cleavage Peptides for Expressing Multiple Payloads Using Trunc10 Replicon

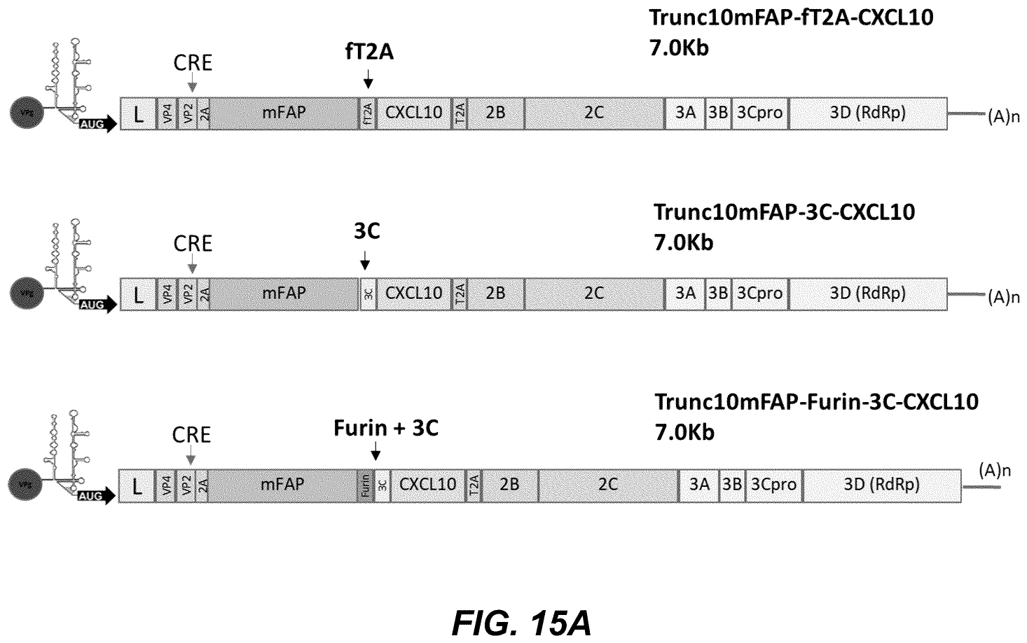

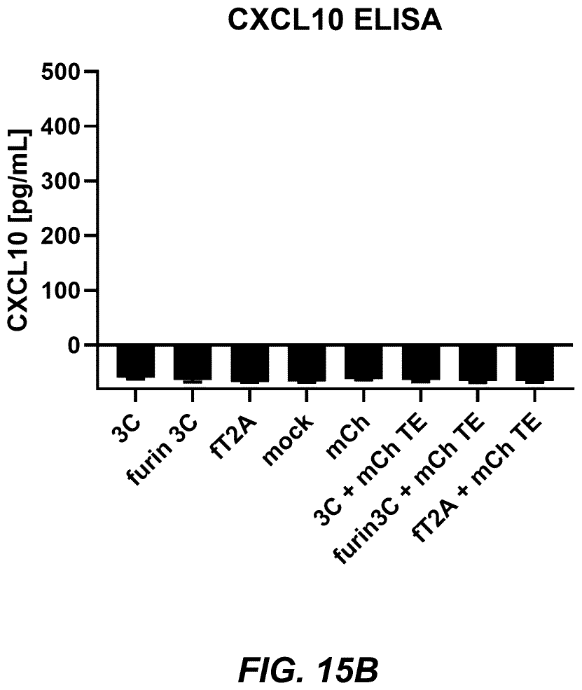

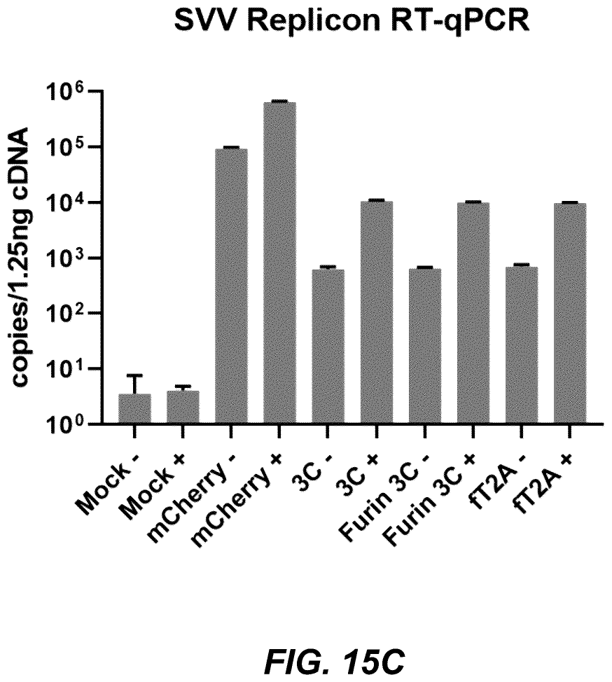

Trunc10 replicon comprising alternate cleavage peptides (3C, or furin-3C, or furinT2A) between his-tagged mFAP and CXCL10 were constructed to test whether any of these alternative cleavage peptides enables efficient expression of multiple payloads from a single replicon (FIG. 15A and SEQ ID NOs: 40, 42, 44). The replicon templates (SEQ ID NOs: 39, 41, 43) and SVV-mCherry templates were linearized with NotI restriction enzyme and in vitro transcribed (IVT) with the HiScribe T7 RNA Synthesis Kit (NEB). H1299 cells were transfected using Lipofectamine RNAiMax (Invitrogen) with 1 ug of Replicon RNA or 1 ug of Replicon RNA plus 1 ug SVVmCherry. At 48 hours post transfection the supernatant was collected and filtered through a 0.45 um filter and RNA was collected in QIAzol reagent (Qiagen). 100 ul of the filtered supernatant was transferred onto a fresh monolayer of H1299 cells and supernatant was collected at 48 hours post infection. Expression of CXCL10 was analyzed with a CXCL10 specific ELISA (R&D). Expression of CXCL10 was not detected after transfection or trans-encapsidation (FIG. 15B). RNA is isolated and analyzed with a positive and negative strand specific taqman assay (FIG. 15C), which showed that all these replicons are deficient for positive and negative strand viral RNA synthesis. Therefore, compared to the original furinT2A site, 3C, fT2A and furin3C cleavage sites do not promote dual payload expression or negative or positive strand synthesis.

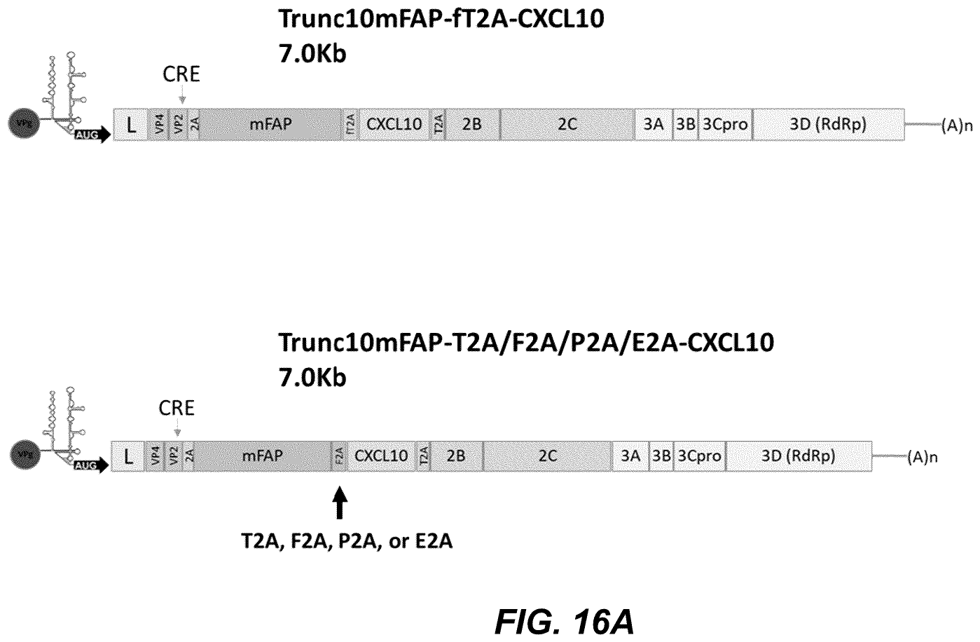

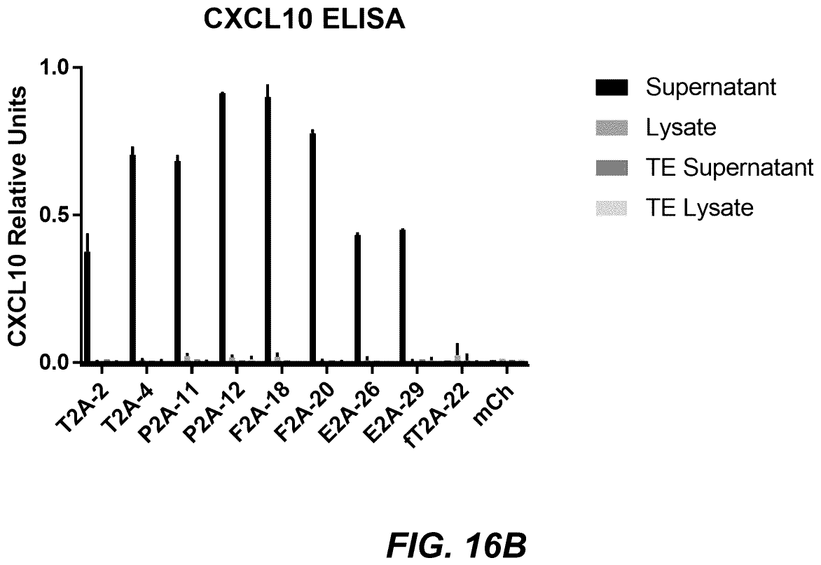

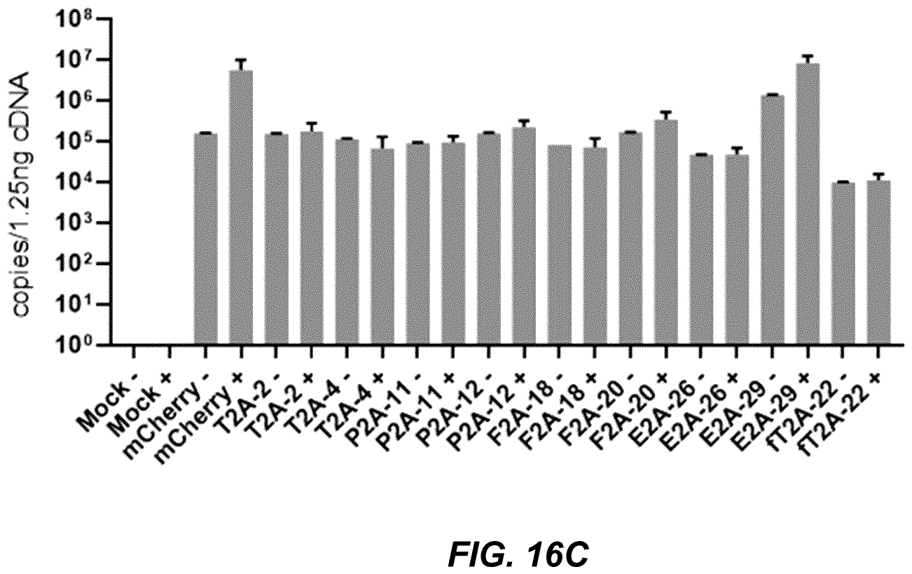

Trunc10 replicon comprising alternate cleavage peptides (T2A, P2A, F2A, or E2A) between his-tagged mFAP and CXCL10 were constructed to test whether any of these alternative cleavage peptides enables efficient expression of multiple payloads from a single replicon (FIG. 16A and SEQ ID NOs: 48, 50, 52, 54). The replicon templates (SEQ ID NOs: 47, 49, 51, 53) and SVV-mCherry template were linearized with NotI restriction enzyme and in vitro transcribed (IVT) with the HiScribe T7 RNA Synthesis Kit (NEB). H1299 cells were transfected using Lipofectamine RNAiMax (Invitrogen) with 1 ug of Replicon RNA or 1 ug of Replicon RNA plus 1 ug SVVmCherry. At 48 hours post transfection the supernatant was collected and filtered through a 0.45 um filter and RNA is collected in QIAzol reagent (Qiagen). 100 ul of the filtered supernatant was transferred onto a fresh monolayer of H1299 cells and supernatant was collected at 48 hours post infection. Expression of CXCL10 was examined with a CXCL10 specific ELISA (R&D) (FIG. 16B), which showed that expression of CXCL10 is enhanced from the T2A, P2A, F2A, and E2A replicons compared to the furin-T2A replicon, but CXCL10 expression is not detected after trans-encapsidation. RNA is isolated and analyzed with a positive and negative strand specific taqman assay (FIG. 16C), which showed that all replicons are competent for positive and negative strand viral RNA synthesis which is improved compared to the furin-T2A replicon.

### Example 15: An IGSF1 Internal Domain Linker with a N Terminal Furin Site Allows Secretion of 2 Polypeptides from a Single ORF

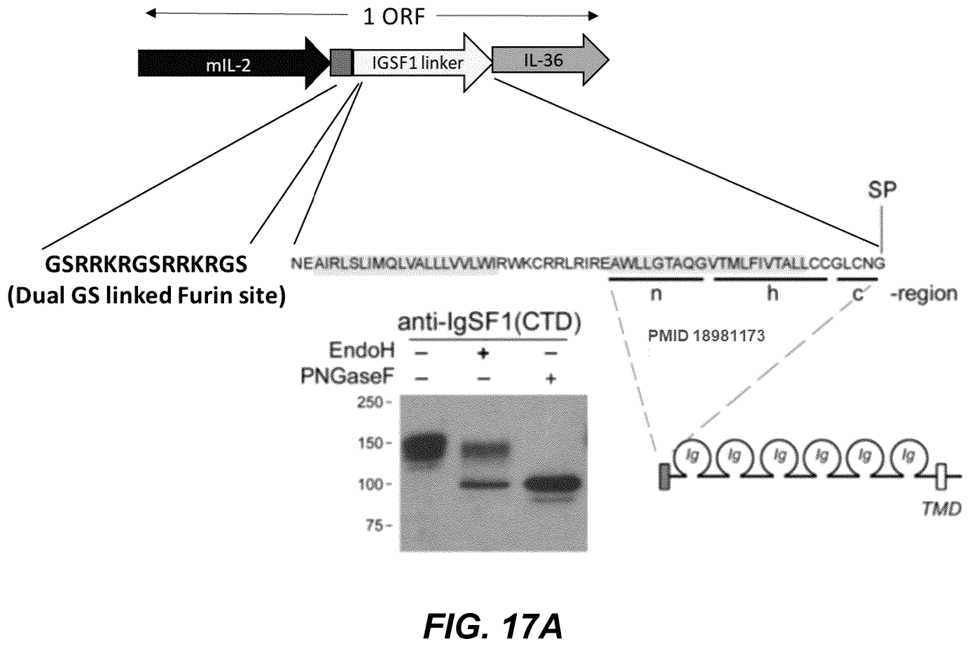

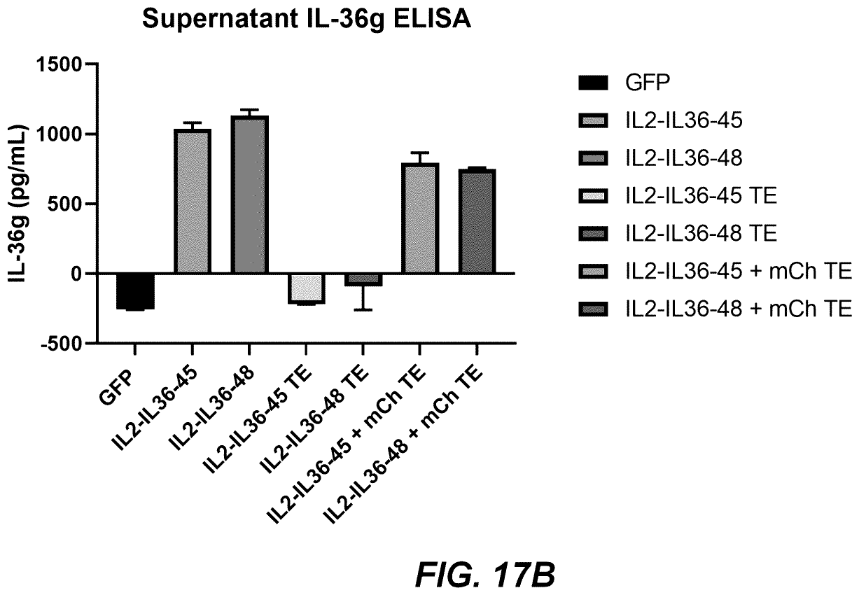

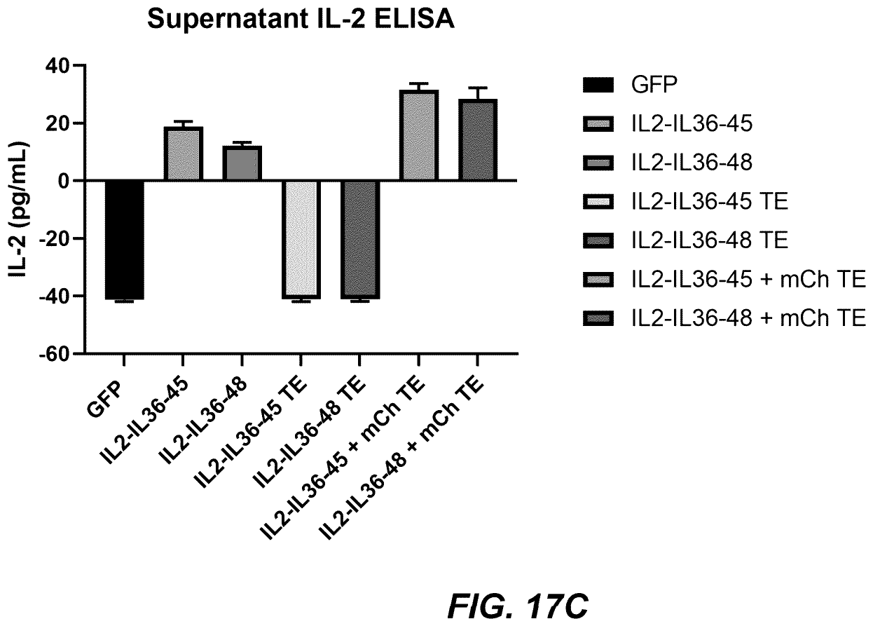

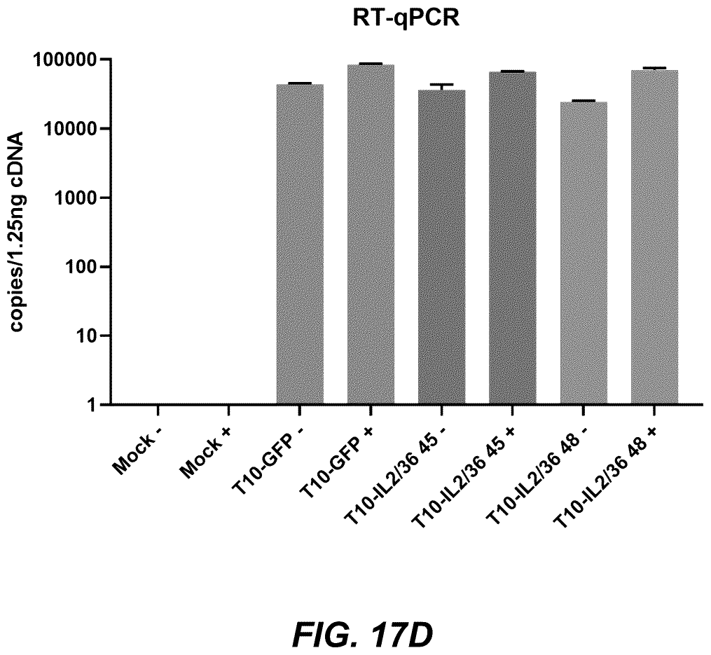

An IGSF1 internal domain linker with an N terminal furin site was tested as the cleavage polypeptide for expression of multiple payloads from a single replicon. A host IGSF1 mediated processing linker was designed to enable expression and secretion of 2 payloads from the same open reading frame (ORF). The human IGSF1 protein contains a transmembrane domain and a tandem signal sequence/signal peptidase site to facilitate secretion of a second secreted payload. On the N-terminus of the IGSF1 linker a 2× furin cleavage sites were included for ER processing of both peptides and to assure release of the N-terminal payload. In this example the N-terminal payload molecule is murine IL-12 and the C-terminal payload molecule is IL-36γamma within the ORF (FIG. 17A and SEQ ID NO: 60). The ORF was inserted into Trunc10 replicon for test.

The replicon template (SEQ ID NO: 59) and SVV-mCherry template were linearized with NotI restriction enzyme and in vitro transcribed (IVT) with the HiScribe T7 RNA Synthesis Kit (NEB). H1299 cells were transfected using Lipofectamine RNAiMax (Invitrogen) with 1 ug of Replicon RNA or 1 ug of Replicon RNA plus 1 ug SVVmCherry. At 48 hours post transfection the supernatant was collected and filtered through a 0.45 um filter and RNA was collected in QIAzol reagent (Qiagen). 100 ul of the filtered supernatant was transferred onto a fresh monolayer of H1299 cells and supernatant was collected at 48 hours post infection. Expression of human IL-36γ and murine IL-2 are detected with hIL-36γ and mIL-2 ELISAs (R&D). Expression of both payloads is detected after transfection and trans-encapsidation (TE) (FIGS. 17B-17C). RNA is isolated and analyzed with a positive and negative strand specific taqman assay (FIG. 17D), which showed that all replicons are competent for positive and negative strand viral RNA synthesis and replicate comparably to a single payload GFP replicon. Overall, the results demonstrated a successful strategy of using IGSF1 mediated processing to cleave between IL2 and IL36 which enables expression of both payloads from a single replicon. Both IL36 and IL2 were expressed after transfection and trans-encapsidation. Positive and negative strand RNA synthesis is equivalent to the T10-eGFP replicon.

### Example 16: HIV-Protease-Mediated Proteolysis Enhances Payload Expression of Two Payloads

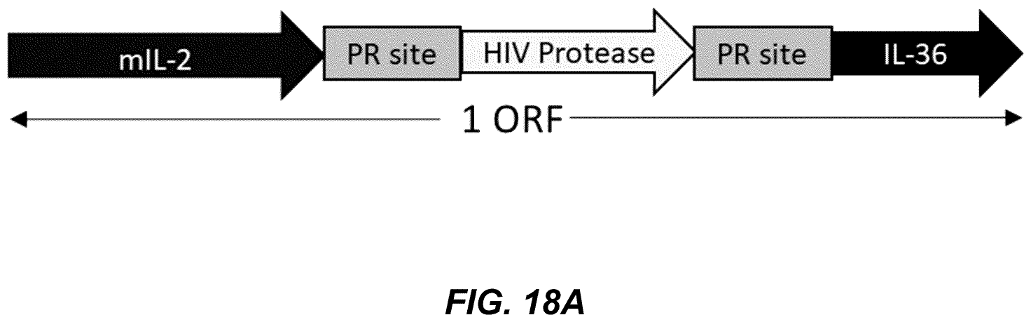

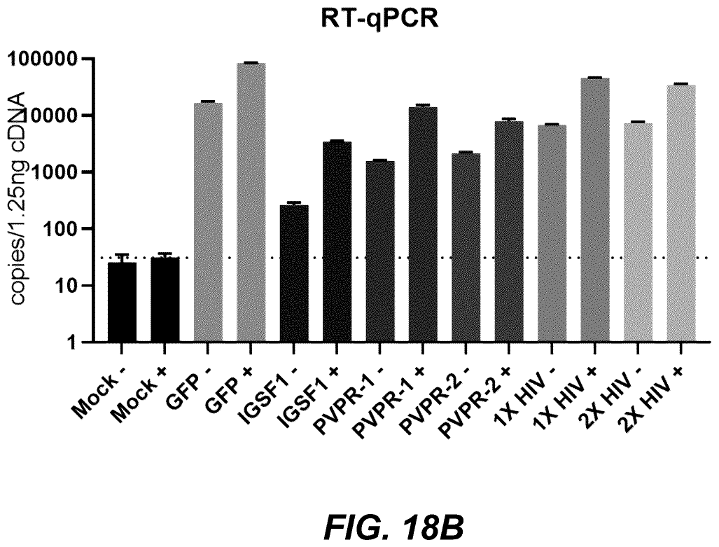

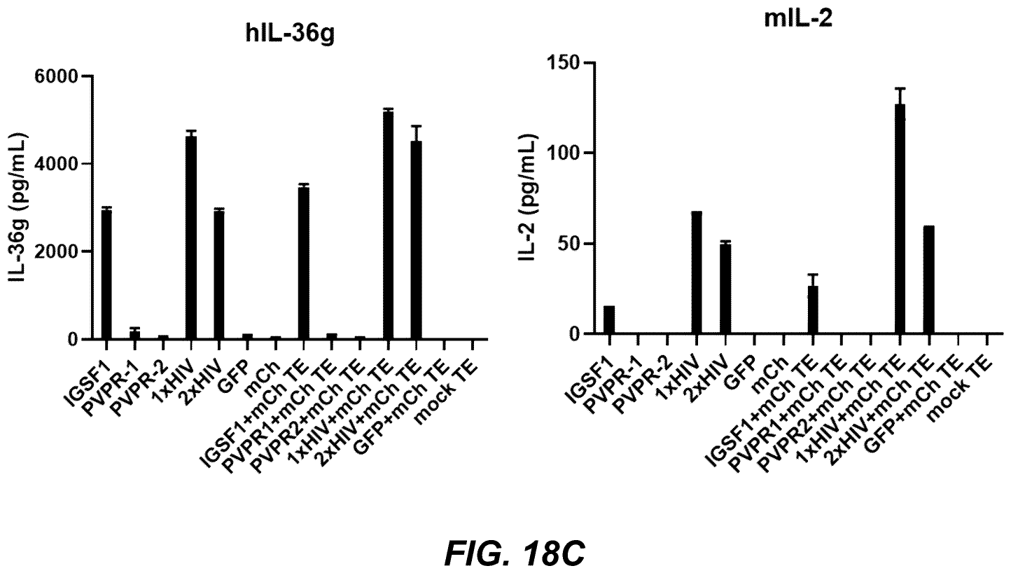

FIG. 18A depicts schematic for HIV-1 protease mediated processing of two secreted payloads in the same open reading frame. Two payloads are separated by either a linked dimer or monomer of HIV-1 protease and flanking HIV protease cleavage (PR) sites. In this example the N-terminal payload molecule is murine IL-12 and the C-terminal payload molecule is IL-36γ within the ORF. The ORF was inserted into Trunc10 replicon for test (SEQ ID NOs: 56, 58). The replicon templates (SEQ ID NO: 55, 57) and SVV-mCherry template were linearized with NotI restriction enzyme and in vitro transcribed (IVT) with the HiScribe T7 RNA Synthesis Kit (NEB). H1299 cells were transfected using Lipofectamine RNAiMax (Invitrogen) with 1 μg of Replicon RNA or 1 ug of Replicon RNA plus 1 μg SVVmCherry. At 48 hours post transfection the supernatant was collected and filtered through a 0.45 um filter and RNA is collected in QIAzol reagent (Qiagen). 100 ul of the filtered supernatant is transferred onto a fresh monolayer of H1299 cells and supernatant is collected at 48 hours post infection. RNA was isolated and analyzed with a positive and negative strand specific taqman assay (FIG. 18B), which showed that all replicons were competent for positive and negative strand viral RNA synthesis and replicate comparably to a single payload GFP replicon. Expression of human IL-36γ and murine IL-2 were detected with hIL-36γ and mIL-2 ELISAs (R&D). Expression of both payloads was detected after transfection and trans-encapsidation (FIG. 18C). Overall, these results demonstrated that a single copy of the HIV-protease enables efficient expression of both payloads from a single replicon, and IL-36γ and IL2 were successfully expressed after transfection and trans-encapsidation. Positive and negative strand RNA synthesis is equivalent to the Trunc10-eGFP replicon. Overall, these results demonstrated that HIV-protease enables of dual payloads from a single ORF.

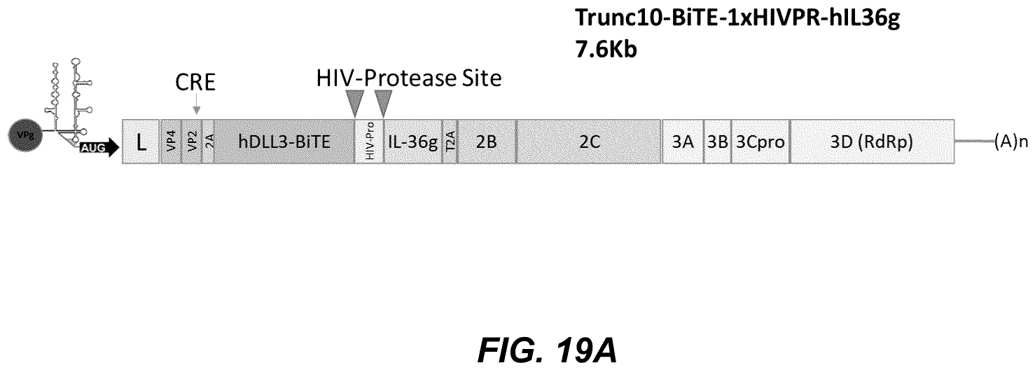

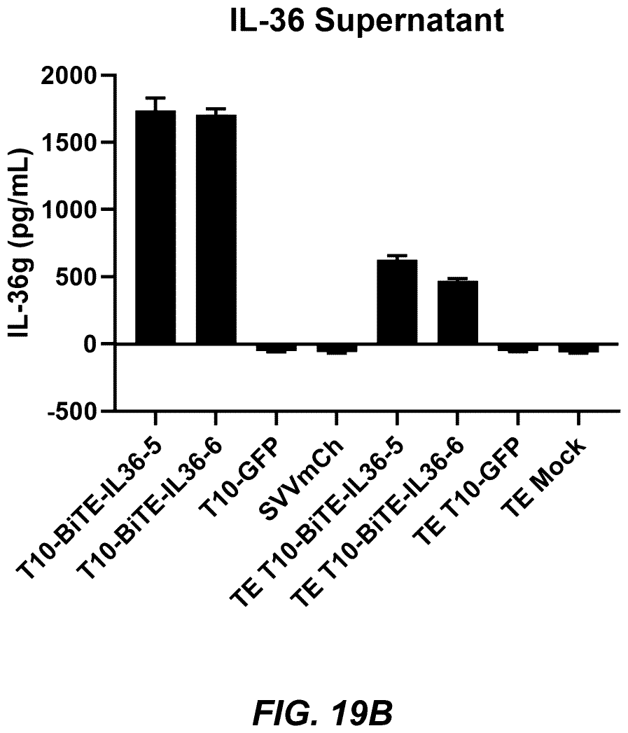

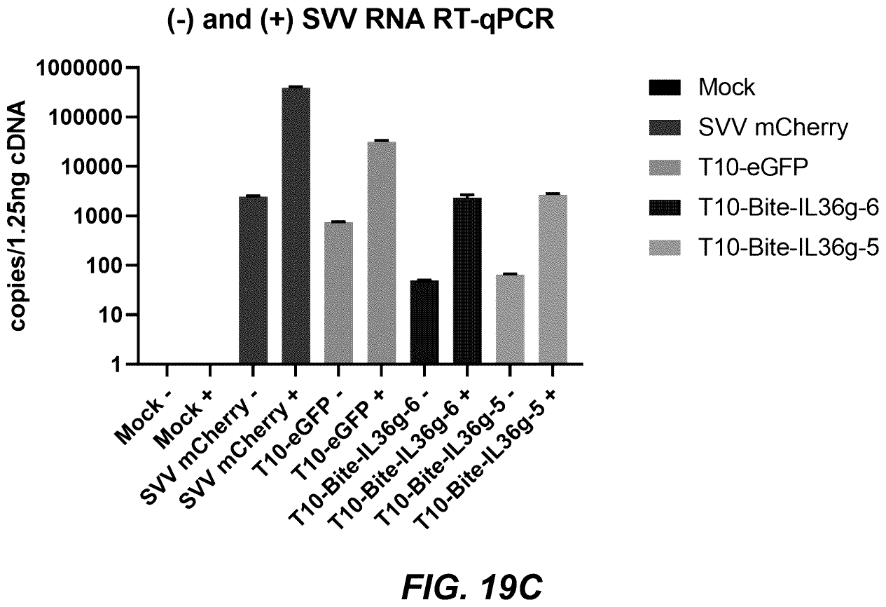

FIG. 19A depicts a dual payload replicon T10-BiTE-hIL-36γ, which comprises a monomeric HIV-1 protease and flanking protease cleavage sites between his-tagged hDLL3-BiTE and human IL-36γ, which was tested for expression of two payloads from a single Trunc10 based replicon. The replicon and SVV-mCherry templates were linearized with NotI restriction enzyme and in vitro transcribed (IVT) with the HiScribe T7 RNA Synthesis Kit (NEB). H1299 cells were transfected using Lipofectamine RNAiMax (Invitrogen) with 1 μg of Replicon RNA or 1 μg of Replicon RNA plus 1μg SVVmCherry. At 48 hours post transfection the supernatant was collected and filtered through a 0.45 μm filter and RNA was collected in QIAzol reagent (Qiagen). 100 ul of the filtered supernatant was transferred onto a fresh monolayer of H1299 cells and supernatant was collected at 48 hours post infection. Expression of human IL-36γ was examined with hIL-36γ ELISA (R&D). Expression of hIL-36γ was detected after both transfection and trans-encapsidation (FIG. 19B). RNA was isolated and analyzed with a positive and negative strand specific taqman assay (FIG. 19C), which showed that all replicons were competent for positive and negative strand viral RNA synthesis. Overall, these results demonstrated that a single copy of the HIV-protease enables expression hIL-36 from a dual payload replicon after transfection and trans-encapsidation. Positive and negative strand RNA synthesis is reduced compared to the T10-eGFP replicon.

### Example 17: HIV-Protease-Mediated Expression of Triple Payloads from a Single Replicon

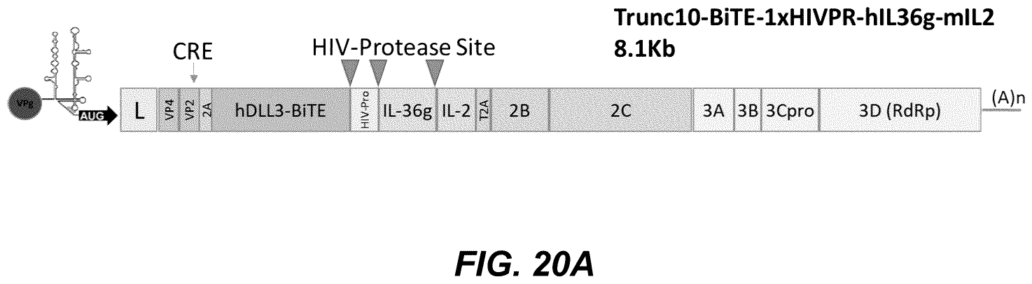

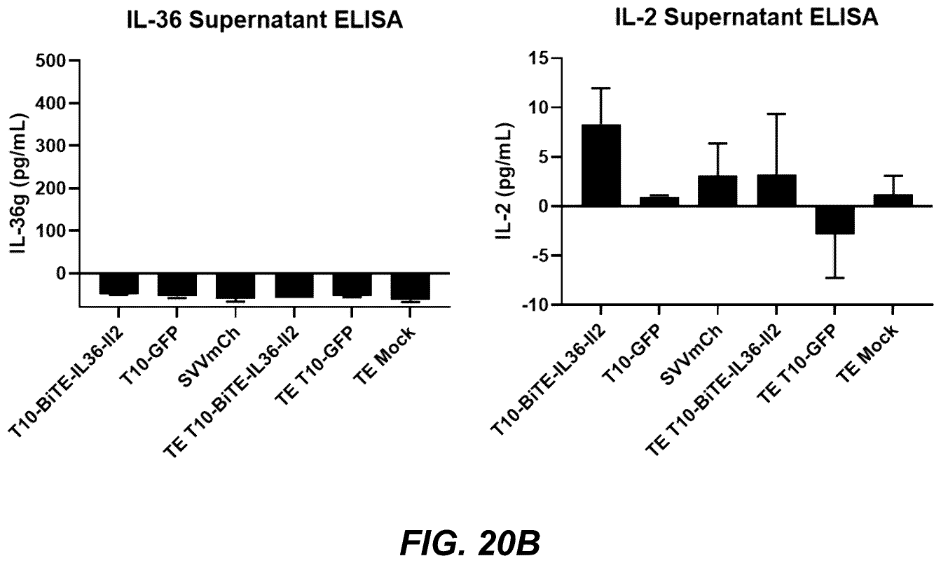

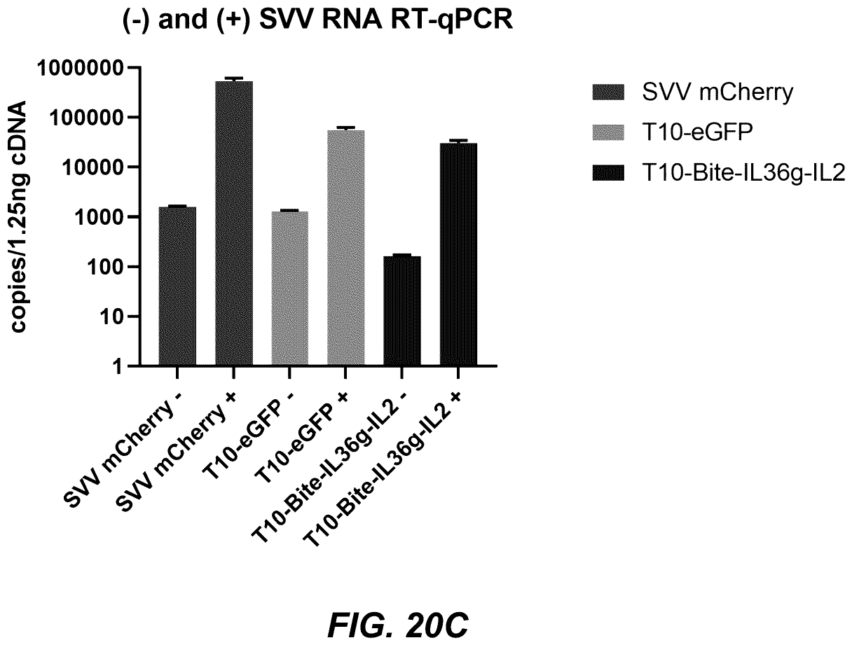

FIG. 20A depicts a triple payload replicon T10-BiTE-IL3g6-IL2, which comprises a monomeric HIV-1 protease and flanking protease cleavage sites between his-tagged hDLL3-BiTE, human IL-36γ, and murine IL-2, which was tested for expression of three payloads from a single replicon Trunc10 (T10). The replicon and SVV-mCherry templates were linearized with NotI restriction enzyme and in vitro transcribed (IVT) with the HiScribe T7 RNA Synthesis Kit (NEB). H1299 cells were transfected using Lipofectamine RNAiMax (Invitrogen) with 1 μg of Replicon RNA or 1 μg of Replicon RNA plus 1 ug SVVmCherry. At 48 hours post transfection the supernatant was collected and filtered through a 0.45 um filter and RNA was collected in QIAzol reagent (Qiagen). 100 μL of the filtered supernatant was transferred onto a fresh monolayer of H1299 cells and supernatant is collected at 48 hours post infection. Expression of human IL-36γ and murine IL-2 were examined with hIL-36γ or mIL-2 specific ELISA (R&D). Expression of hIL-36 was not detected after transfection and trans-encapsidation, and low expression of mIL-2 was detected after transfection and transencapsidation (FIG. 20B). RNA was isolated and analyzed with a positive and negative strand specific taqman assay (FIG. 20C), which showed that all replicons are competent for positive and negative strand viral RNA synthesis, but the ability is reduced compared to the single payload replicon.

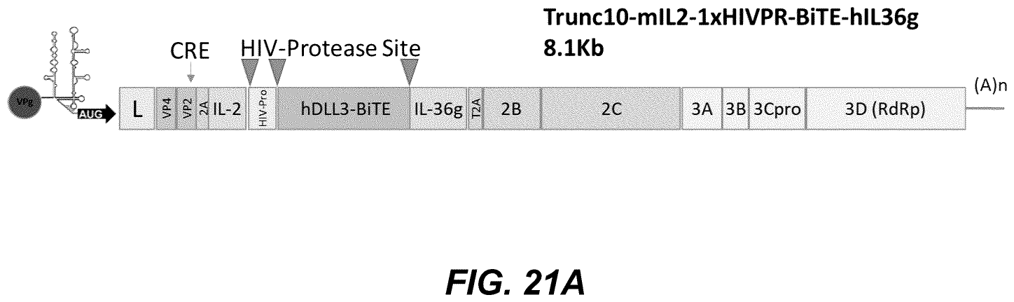

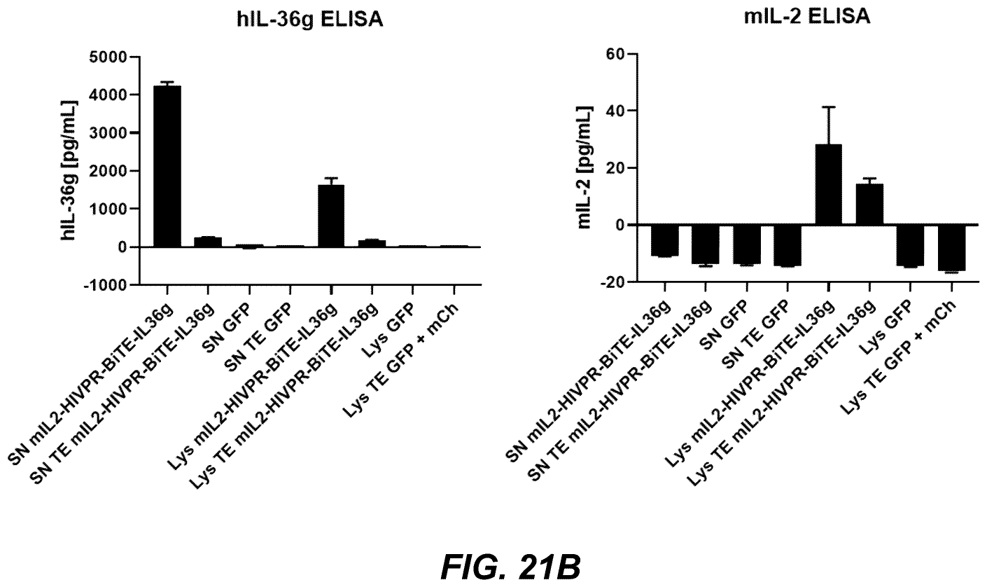

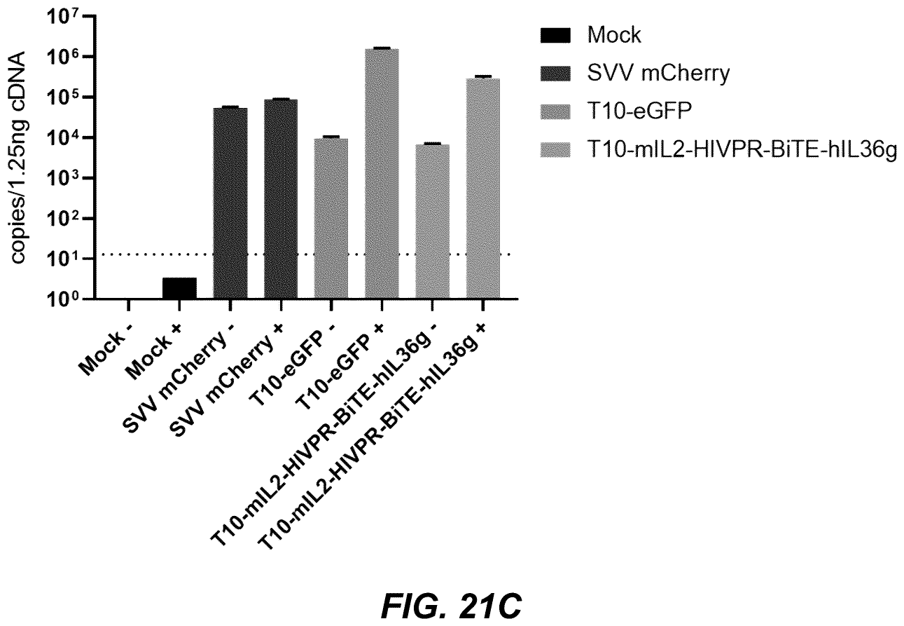

FIG. 21A depicts an alternative design of triple payload replicon T10-mIL2-BiTE-hIL-36γ, which places IL-2 coding region before the other two payload coding regions, and uses a monomeric HIV-1 protease and flanking protease cleavage sites between murine IL-2, his-tagged hDLL3-BiTE, and human IL-36γ. The construct was tested for expression of three payloads from a single replicon following the same protocol as above. Expression of hIL-36 was detected after transfection, but its expression level was reduced after trans-encapsidation (TE). Low expression of mIL-2 was detected after transfection and transencapsidation in the lysate but it was not secreted into the supernatant (FIG. 21B). RNA is isolated and analyzed with a positive and negative strand specific taqman assay (FIG. 21C), which showed that all replicons are competent for positive and negative strand viral RNA synthesis.

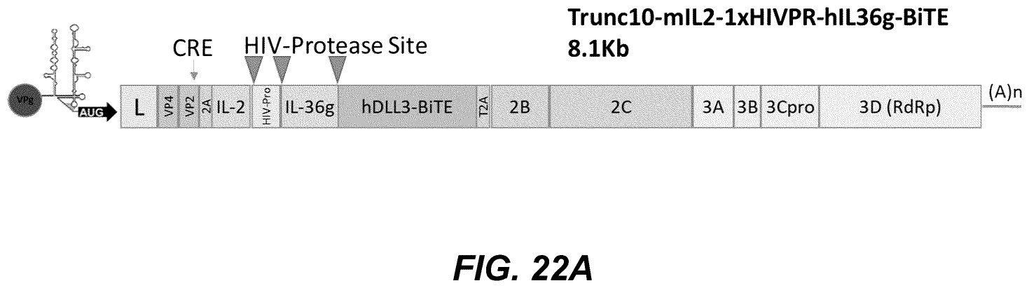

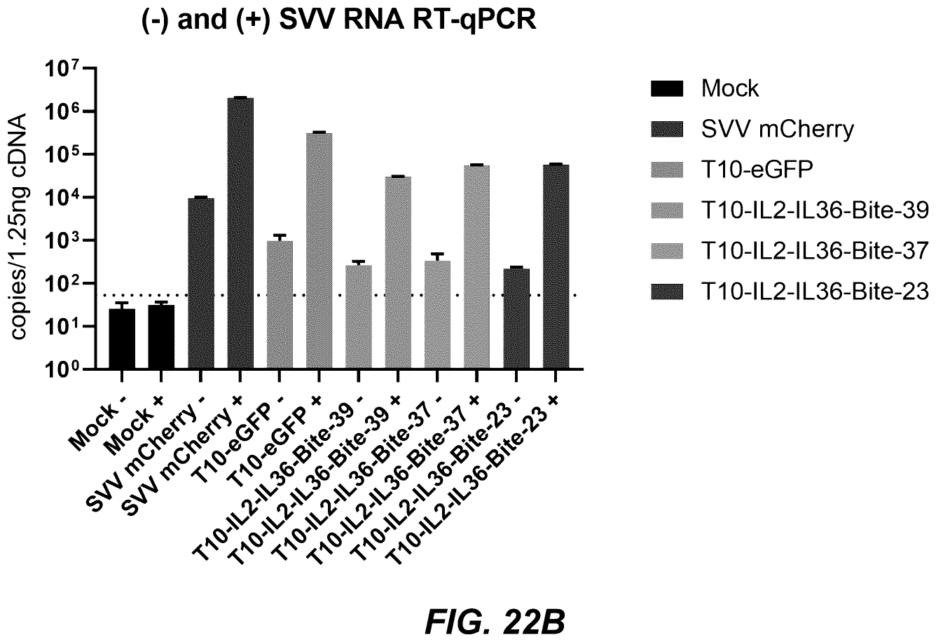

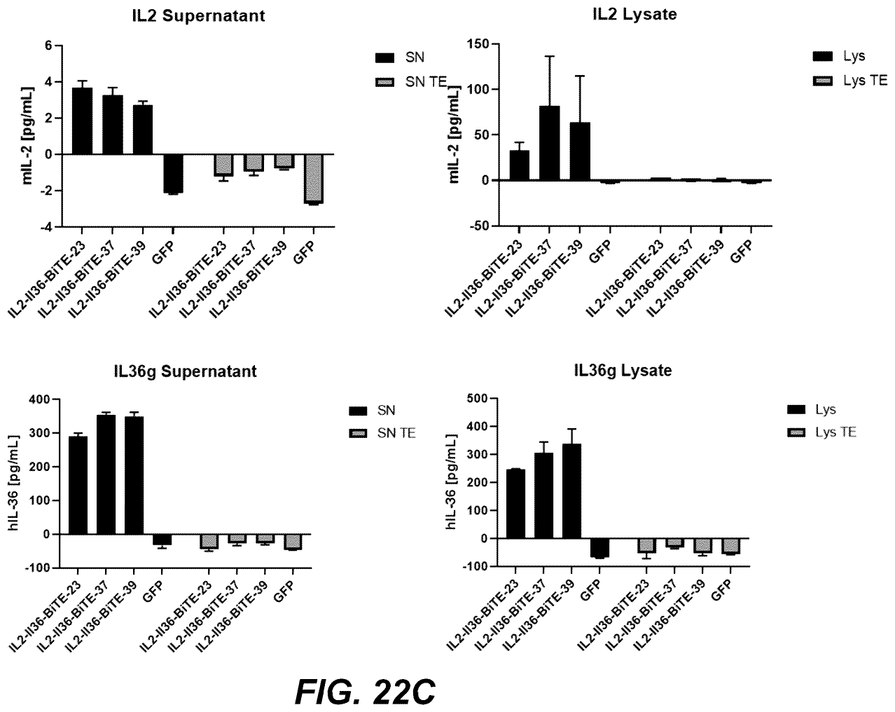

FIG. 22A depicts another design of triple payload replicon T10-mIL2-hIL-36γ-BiTE, which places the hDLL3-BiTE as the last payload, and comprises a monomeric HIV-1 protease and flanking protease cleavage sites between murine IL-2, human IL-36γ, and his-tagged hDLL3-BiTE. The construct was tested for expression of three payloads from a single replicon following the same protocol as above. RNA was isolated and analyzed with a positive and negative strand specific taqman assay (FIG. 22B), which showed that all replicons were competent for positive and negative strand viral RNA synthesis. Expression of hIL-36 and mIL-2 were detected after transfection but not after trans-encapsidation, and higher expression of mIL-2 is detected in the lysate relative to the supernatant (FIG. 22C). Overall, the results showed that from the triple payload replicon T10-mIL2-hIL36-BiTE BiTE, expression of hIL-36 and mIL-2 can be detected in supernatant and lysate at low levels after transfection but not after trans-encapsidation, and positive and negative strand synthesis is slightly lower than T10-eGFP.

### Example 18: In Vivo Studies of Payload Expression Using SVV-Derived RNA Replicon

Payload expression using SVV-derived replicons was tested in animal models.

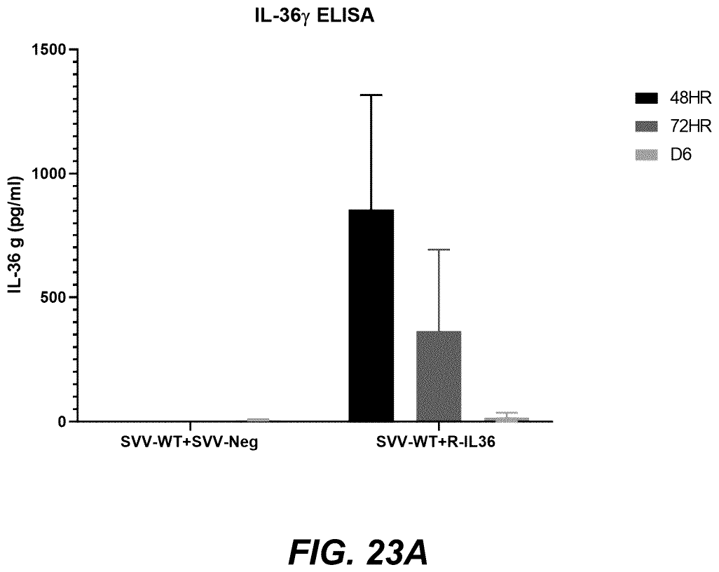

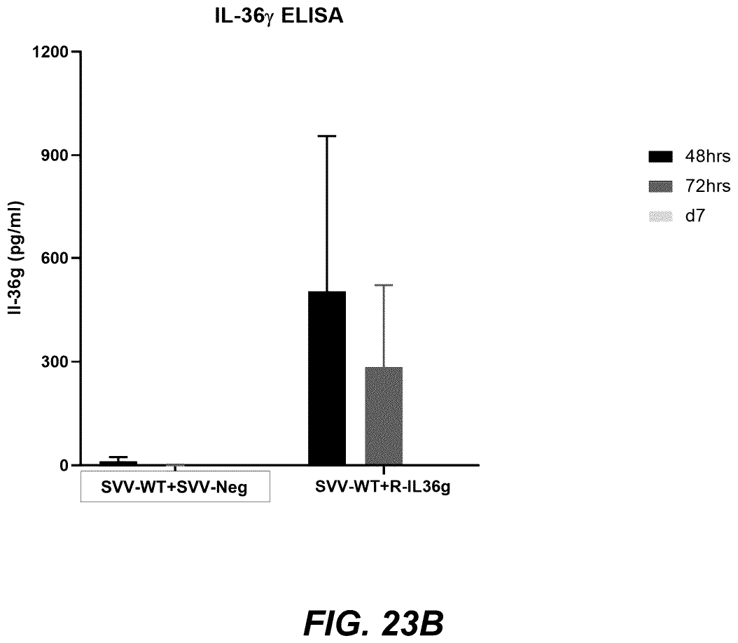

Athymic nude female mice were implanted with NCI-H69 cells (8×10⁶ cells/0.1 mL in a 1:1 mixture of serum-free PBS and Matrigel®) subcutaneously in the right flank. When median tumor size reached approximately 150 mm³ (120-180 mm³ range), mice were cohorted in groups of 3 mice per treatment arm. In one treatment arm, mice were treated with a mixture of lipid nanoparticles (LNPs) that encapsulate either a wildtype SVV RNA viral genome (SVV-WT) or SVV-Trunc10-hIL-36γ RNA replicon (as described in Example 8) via intratumoral administration. In the control arm, mice were treated with a mixture of LNPs that encapsulate the wildtype SVV RNA viral genome and an SVV-negative control RNA (SVV-Neg) via intratumoral administration. Tumor samples were collected after 48 hrs, 72 hrs, and 6 days post dosing, sample tissues were pulverized, and tumor lysate was prepared. IL-36γ expression level was determined by ELISA. The results are shown in FIG. 23A. Expression of human IL-36γ was detected in tumor samples 48 and 72 hrs post dosing.

In another set of experiments, athymic nude female mice were implanted with NCI-H446 cells (5×10⁶ cells/0.1 mL in a 1:1 mixture of serum-free PBS and Matrigel®) subcutaneously in the right flank. When median tumor size reached approximately 150 mm³ (120-180 mm³ range), mice were cohorted in groups of 3 mice per treatment arm. In one treatment arm, mice were treated with a mixture of lipid nanoparticles (LNPs) that encapsulate a wildtype SVV RNA viral genome (SVV-WT) and an SVV-replicon RNA that encodes human IL-36γ (R-IL36g) via intratumoral administration. In the control arm, mice were treated with a mixture of LNPs that encapsulate the wild type SVV RNA viral genome and an SVV-negative control RNA (SVV-Neg) via intratumoral administration. Tumor samples were collected after 48 hrs, 72 hrs, and 7 days post dosing, sample tissues were pulverized, and tumor lysate was prepared. IL-36γ expression level was determined by ELISA. The results are shown in FIG. 23B. Expression of human IL-36γ was detected in tumor samples 48 and 72 hrs post dosing.

### Example 19: Construction of a Coxsackievirus A21 Replicon and Payload Expression from the Same

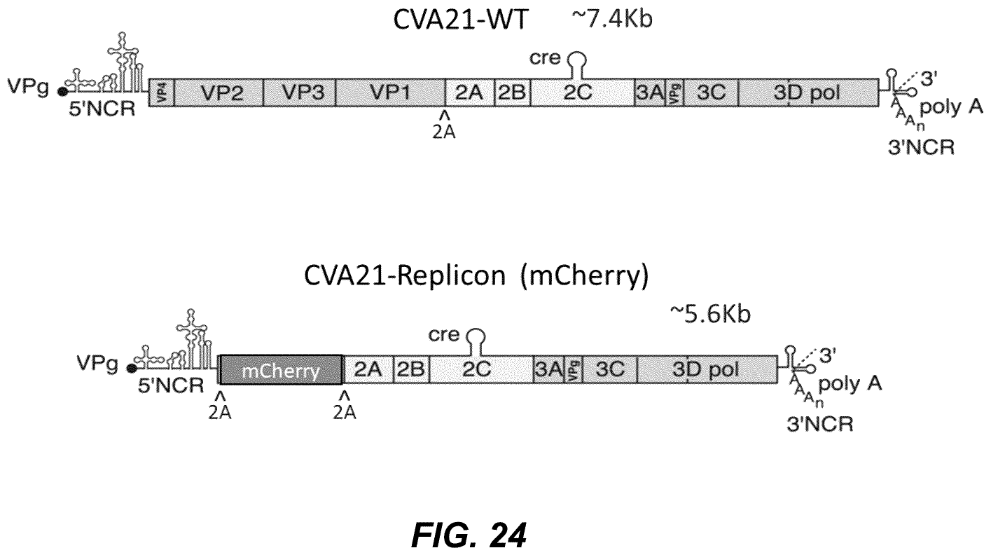

As shown in FIG. 24, a CVA21-Replicon (SEQ ID NO: 62) was created by removing the VP structural proteins (VP1, VP2, VP3) and replacing them with the fluorescent protein mCherry ORF flanked by 2A protease sites. The replicon can be produced using the DNA vector template according to SEQ ID NO: 61.

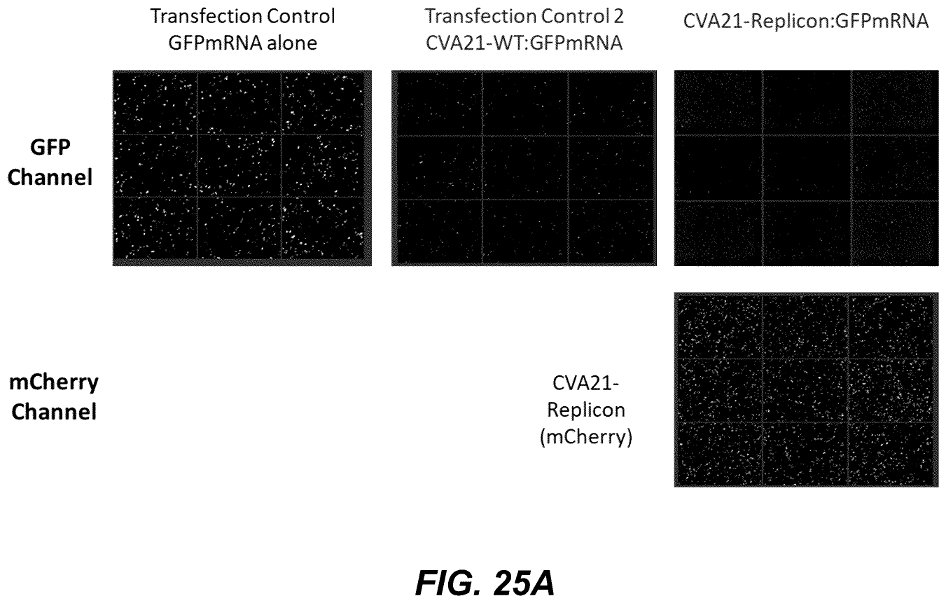

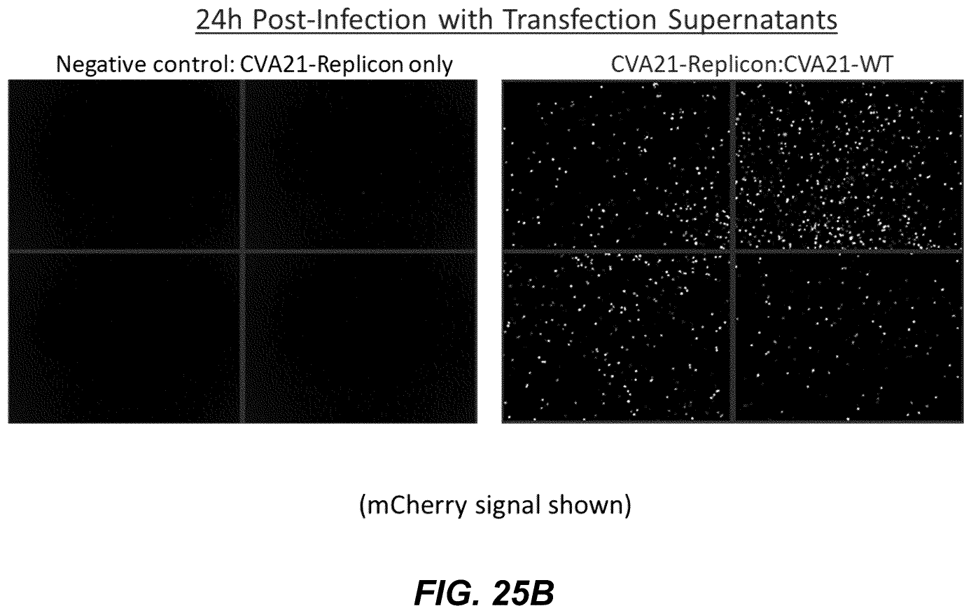

The CVA21-Replicon comprising mCherry payload was tested for expression of payload. 1×10\acute{ }5 NCI-H1299 cells in a six well plate were transfected using RNAiMAx reagent with 500 ng GFP mRNA alone (Transfection control), in equal molar ratio with CVA21-WT RNA (Control 2), or in equal molar ratio with CVA21-Replicon RNA (FIG. 25A). The transfection control showed the maximum transfection efficiency of the H1299 cells. Control 2 showed that the GFP signal was partially inhibited by transfection with CVA21-mRNA. The CVA21-Replicon displays mCherry signal throughout matching that of the transfection control showing very efficient transfection and high expression. Therefore, CVA21 Replicon RNA is capable of expressing payload protein in NCI-H1299 cells.

Next, experiments were performed to determine whether CVA21 mCherry replicons can be trans-encapsidate in the presence of WT CVA21 virus. 1×10\acute{ }5NCI-H1299 cells in a six well plate were transfected using RNAiMAx reagent with 500 ng with CVA21-Replicon RNA alone (Negative control), or with 500 ng CVA21-WT RNA. 48 h post transfection supernatants were collected, spun down, filtered through 45 μM and then 100 μL was used to infect a new 12 well plate well of (1×10\acute{ }5 cells) H1299 cells. Strong infection and mCherry signal was seen in the well infected with the Replicon:WT virus supernatant suggesting successful encapsidation of the mCherry replicon by WT-RNA produced capsids, whereas the negative control which lacks the CVA21-WT shows no mCherry signal (FIG. 25B). Overall, these results demonstrated that CVA21 replicons carrying payload can be successfully trans-encapsidated in the presence of wildtype CVA21 virus.

### Example 20: In Vivo Efficacy of Lipid Nanoparticles Comprising SVV Derived Recombinant RNA Replicons and RNA Molecules Encoding SVV Viral Genome for Lung Cancer Treatment

Various Seneca Valley virus (SVV) derived recombinant RNA replicons are constructed. These recombinant RNA replicons comprise a heterologous polynucleotide encoding one or more immunomodulatory proteins (e.g., anti-DLL3 Bi-specific T-cell engager (BiTE)). Some of these recombinant RNA replicons further comprise coding regions for one or more cytokines (e.g., IL-2, IL-12, IL-36γ) and/or one or more chemokines (e.g., CCL21, CCL4). Some of the SVV derived RNA replicons comprise coding regions of one or more payload molecules according to the following Table 18:

TABLE 18
Payload Molecules for SVV derived Replicon

| SVV derived Replicon | Payload Molecules |
|---|---|
| Replicon Construct#A1: | anti-DLL3 BiTE, IL-2 |
| Replicon Construct#A2: | anti-DLL3 BiTE, IL-12 |
| Replicon Construct#A3: | anti-DLL3 BiTE, IL-36γ |
| Replicon Construct#A4: | anti-DLL3 BiTE, CCL21 |
| Replicon Construct#A5: | anti-DLL3 BiTE, CCL4 |
| Replicon Construct#A6: | anti-DLL3 BiTE, IL-2, IL-12 |
| Replicon Construct#A7: | anti-DLL3 BiTE, IL-2, IL-36γ |
| Replicon Construct#A8: | anti-DLL3 BiTE, IL-2, CCL21 |
| Replicon Construct#A9: | anti-DLL3 BiTE, IL-2, CCL4 |
| Replicon Construct#A10: | anti-DLL3 BiTE, IL-12, IL-36γ |
| Replicon Construct#A11: | anti-DLL3 BiTE, IL-12, CCL21 |
| Replicon Construct#A12: | anti-DLL3 BiTE, IL-12, CCL4 |
| Replicon Construct#A13: | anti-DLL3 BiTE, IL-36γ, CCL21 |
| Replicon Construct#A14: | anti-DLL3 BiTE, IL-36γ, CCL4 |
| Replicon Construct#A15: | anti-DLL3 BiTE, CCL21, CCL4 |

For each of the SVV derived replicon, lipid nanoparticles comprising the SVV derived RNA replicon and RNA molecules encoding SVV viral genome are prepared. Animal experiments are conducted to evaluate the efficacy of these lipid nanoparticles to inhibit lung tumor growth in vivo, which is compared to the efficacy of lipid nanoparticles comprising RNA molecules encoding SVV viral genome but without the RNA replicon.

Briefly, 8-week-old NSG mice are injected with human PBMC on day 1, 2 and 3. On day 10, H1299-DLL3 cells (5×10⁶ cells/0.1 mL in a 1:1 mixture of serum-free PBS and Matrigel®) are implanted subcutaneously in the right flank of PBMC-humanized mice. When median tumor size is approximately 150 mm³ (120-180 mm³ range), mice are cohorted in groups of 8-10 mice per treatment arm. Mice are treated with the LNPs containing RNA molecules encoding SVV viral genome and a particular SVV derived RNA replicon (via intravenous and/or intratumoral administration). In the control group, mice are treated with the LNPs containing RNA molecules encoding SVV viral genome. Tumor volume is measured 2 times a week to assess the efficacy of each treatment arm.

### Example 21: In Vivo Efficacy of Lipid Nanoparticles Comprising CVA21 Derived Recombinant RNA Replicons and RNA Molecules Encoding CVA21viral Genome for Melanoma Treatment

Various Coxsackievirus A21 (CVA21)-derived recombinant RNA replicons are constructed. These recombinant RNA replicons comprise a heterologous polynucleotide encoding one or more immunomodulatory proteins (e.g., anti-DLL3 Bi-specific T-cell engager (BiTE)). Some of these recombinant RNA replicons further comprise coding regions for one or more cytokines (e.g., IL-2, IL-12, IL-36γ) and/or one or more chemokines (e.g., CCL21, CCL4). Some of the CVA21 derived RNA replicons comprise coding regions of one or more payload molecules according to the following Table 19:

TABLE 19
Payload Molecules for CVA21 derived Replicon

| CVA21 derived Replicon | Payload Molecules |
|---|---|
| Replicon Construct#C1: | anti-DLL3 BiTE, IL-2 |
| Replicon Construct#C2: | anti-DLL3 BiTE, IL-12 |
| Replicon Construct#C3: | anti-DLL3 BiTE, IL-36γ |
| Replicon Construct#C4: | anti-DLL3 BiTE, CCL21 |
| Replicon Construct#C5: | anti-DLL3 BiTE, CCL4 |
| Replicon Construct#C6: | anti-DLL3 BiTE, IL-2, IL-12 |
| Replicon Construct#C7: | anti-DLL3 BiTE, IL-2, IL-36γ |
| Replicon Construct#C8: | anti-DLL3 BiTE, IL-2, CCL21 |
| Replicon Construct#C9: | anti-DLL3 BiTE, IL-2, CCL4 |
| Replicon Construct#C10: | anti-DLL3 BiTE, IL-12, IL-36γ |
| Replicon Construct#C11: | anti-DLL3 BiTE, IL-12, CCL21 |
| Replicon Construct#C12: | anti-DLL3 BiTE, IL-12, CCL4 |
| Replicon Construct#C13: | anti-DLL3 BiTE, IL-36γ, CCL21 |
| Replicon Construct#C14: | anti-DLL3 BiTE, IL-36γ, CCL4 |
| Replicon Construct#C15: | anti-DLL3 BiTE, CCL21, CCL4 |

For each of the CVA21 derived replicon, lipid nanoparticles comprising the CVA21 derived RNA replicon and RNA molecules encoding CVA21 viral genome are prepared. Animal experiments are conducted to evaluate the efficacy of these lipid nanoparticles to inhibit melanoma tumor growth in vivo, which is compared to the efficacy of lipid nanoparticles comprising RNA molecules encoding CVA21 viral genome but without the RNA replicon.

Briefly, 8-week-old NSG mice are injected with human PBMC on day 1, 2 and 3. On day 10, SK-MEL-28-EpCAM cells (5×10⁶ cells/0.1 mL in a 1:1 mixture of serum-free PBS and Matrigel®) are implanted subcutaneously in the right flank of PBMC-humanized mice. When median tumor size is approximately 150 mm³ (120-180 mm³ range), mice are cohorted in groups of 8-10 mice per treatment arm. Mice are treated with the LNPs containing RNA molecules encoding CVA21 viral genome and a particular CVA21 derived RNA replicon (via intravenous and/or intratumoral administration). In the control group, mice are treated with the LNPs containing RNA molecules encoding CVA21 viral genome. Tumor volume is measured 2 times a week to assess the efficacy of each treatment arm.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

While preferred embodiments of the present disclosure have been shown and described herein; it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1. A recombinant RNA replicon comprising:
a picornavirus genome, wherein the picornavirus genome comprises a deletion or a truncation in one or more protein coding regions; and
a heterologous polynucleotide.

2. The recombinant RNA replicon of claim 1, wherein the picornavirus genome comprises the deletion or the truncation in one or more VP coding regions.

3. The recombinant RNA replicon of claim 1 or 2, wherein the picornavirus genome comprises the deletion or the truncation in each of the VP1, VP3 and VP2 coding regions.

4. The recombinant RNA replicon of any one of claims 1-3, wherein the picornavirus genome comprises the deletion of the VP1 and VP3 coding regions and the truncation of the VP2 coding region.

5. The recombinant RNA replicon of any one of claims 1-4, wherein the picornavirus is selected from a senecavirus, a cardiovirus, and an enterovirus.

6. The recombinant RNA replicon of any one of claims 1-5, wherein the deletion or the truncation comprises at least 500 bp, at least 1000 bp, at least 1500 bp, at least 2000 bp, at least 2500 bp, or at least 3000 bp.

7. The recombinant RNA replicon of claim 6, wherein the deletion or the truncation comprises at least 2000 bp.

8. The recombinant RNA replicon of any one of claims 1-7, wherein a site of the deletion or a site of the truncation comprises the heterologous polynucleotide

9. The recombinant RNA replicon of any one of claims 1-7, wherein the heterologous polynucleotide is inserted between a 2A coding region and a 2B coding region.

10. The recombinant RNA replicon of any one of claims 1-7, wherein the heterologous polynucleotide is inserted between a 3D coding region and a 3′ untranslated region (UTR).

11. The recombinant RNA replicon of any one of claims 1-10, wherein the heterologous polynucleotide comprises at least 1000 bp, at least 2000 bp, or at least 3000 bp.

12. The recombinant RNA replicon of any one of claims 1-11, wherein the picornavirus is a Seneca Valley Virus (SVV).

13. The recombinant RNA replicon of claim 12, wherein the deletion or the truncation comprises one or more nucleotides between nucleotide 1261 and 3477, inclusive of the endpoints, according to the numbering of SEQ ID NO: 1.

14. The recombinant RNA replicon of claim 12, wherein the deletion or the truncation comprises nucleotide 1261 to 3477, inclusive of the endpoints, according to the numbering of SEQ ID NO: 1.

15. The recombinant RNA replicon of claims 12 or 13, wherein the deletion or the truncation comprises at least 500 bp, at least 1000 bp, at least 1500 bp, or at least 2000 bp.

16. The recombinant RNA replicon of claim 15, wherein the deletion or the truncation comprises at least 2000 bp.

17. The recombinant RNA replicon of any one of claims 12 to 16, wherein the SVV genome comprises a 5′ leader protein coding sequence.

18. The recombinant RNA replicon of any one of claims 12 to 17, wherein the SVV genome comprises a VP4 coding region.

19. The recombinant RNA replicon of any one of claims 12 to 18, wherein the SVV genome comprises a VP2 coding region or a truncation thereof.

20. The recombinant RNA replicon of claim 19, wherein the SVV genome comprises, from 5′ to 3′ direction, the 5′ leader protein coding sequence, the VP4 coding region, and the VP2 coding region or a truncation thereof.

21. The recombinant RNA replicon of claim 20, wherein a portion of the SVV genome comprising the 5′ leader protein coding sequence, the VP4 coding region, and the VP2 coding region or a truncation thereof has at least 90% sequence identity to nucleotide 1 to 1260 of SEQ ID NO: 1.

22. The recombinant RNA replicon of claim 20 or 21, wherein the SVV genome comprises, from 5′ to 3′ direction, the 5′ leader protein coding sequence, the VP4 coding region, the VP2 coding region or a truncation thereof, and the heterologous polynucleotide.

23. The recombinant RNA replicon of any one of claims 1-22, wherein the SVV genome comprises a cis-acting replication element (CRE).

24. The recombinant RNA replicon of claim 23, wherein the CRE comprises between 10-200 bp.

25. The recombinant RNA replicon of claim 23 or 24, wherein the CRE comprises one or more nucleotides within the region corresponding to nucleotide 1000 to nucleotide 1260 according to SEQ ID NO: 1.

26. The recombinant RNA replicon of claim 23 or 24, wherein the CRE comprises one or more nucleotides within the region corresponding to nucleotide 1117 to nucleotide 1260 according to SEQ ID NO: 1.

27. The recombinant RNA replicon of any one of claims 23-26, wherein the CRE comprises a polynucleotide sequence having at least 70%, at least 75%, at least 80%, at least 85%, at least 90%, at least 95%, at least 96%, at least 97%, at least 98%, at least 99%, or 100% identity to SEQ ID NO: 149.

28. The recombinant RNA replicon of any one of claims 12-27, wherein the SVV genome further comprises a 2A coding region.

29. The recombinant RNA replicon of claim 28, wherein the 2A coding region is located between the VP2 coding region or a truncation thereof and the heterologous polynucleotide.

30. The recombinant RNA replicon of any one of claims 12-29, wherein the SVV genome comprises one or more of a 2B coding region, a 2C coding region, a 3A coding region, a 3B coding region, a 3Cpro coding region, and a 3D(RdRp) coding region.

31. The recombinant RNA replicon of any one of claims 12-29, wherein the SVV genome comprises a 2B coding region, a 2C coding region, a 3A coding region, a 3B coding region, a 3Cpro coding region, and a 3D(RdRp) coding region.

32. The recombinant RNA replicon of claim 31, wherein the SVV genome comprises, from 5′ to 3′, the 2B coding region, the 2C coding region, the 3A coding region, the 3B coding region, the 3Cpro coding region, and the 3D(RdRp) coding region.

33. The recombinant RNA replicon of claim 32, wherein a portion of the SVV genome comprising the 2B coding region, the 2C coding region, the 3A coding region, the 3B coding region, the 3Cpro coding region, and the 3D(RdRp) coding region has at least 90% sequence identity to nucleotide 3505 to 7310 according to SEQ ID NO: 1.

34. The recombinant RNA replicon of any one of claims 30-33, wherein the SVV genome comprises, from 5′ to 3′, the heterologous polynucleotide and the 2B coding region.

35. The recombinant RNA replicon of any one of claims 1 to 11, wherein the picornavirus is a coxsackievirus.

36. The recombinant RNA replicon of claim 35, wherein the deletion or the truncation comprises one or more nucleotides between nucleotide 717 to 3332, inclusive of the endpoints, according to the numbering of SEQ ID NO: 3.

37. The recombinant RNA replicon of claim 35, wherein the deletion or the truncation comprises nucleotide 717 to 3332, inclusive of the endpoints, according to the numbering of SEQ ID NO: 3.

38. The recombinant RNA replicon of claim 35 or 36, wherein the deletion or the truncation comprises at least 500 bp, at least 1000 bp, at least 1500 bp, at least 2000 bp, or at least 2600 bp.

39. The recombinant RNA replicon of any one of claims 35 to 38, wherein the coxsackievirus genome comprises a 5′ UTR.

40. The recombinant RNA replicon of any one of claims 35 to 39, wherein a portion of the coxsackievirus genome comprising the 5′ UTR has at least 90% sequence identity to SEQ ID NO: 4.

41. The recombinant RNA replicon of any one of claims 35 to 40, wherein the coxsackievirus genome comprises one or more of a 2A coding region, a 2B coding region, a 2C coding region, a 3A coding region, a 3B coding region, a VPg coding region, a 3C coding region, a 3D pol coding region, and a 3′ UTR.

42. The recombinant RNA replicon of any one of claims 35 to 40, wherein the coxsackievirus genome comprises a 2A coding region, a 2B coding region, a 2C coding region, a 3A coding region, a 3B coding region, a VPg coding region, a 3C coding region, a 3D pol coding region, and a 3′ UTR.

43. The recombinant RNA replicon of claim 42, wherein the coxsackievirus genome comprises, from 5′ to 3′ direction, the 2A coding region, the 2B coding region, the 2C coding region, the 3A coding region, the 3B coding region, the VPg coding region, the 3C coding region, the 3D pol coding region, and the 3′ UTR.

44. The recombinant RNA replicon of claim 42, wherein a portion of the coxsackievirus genome comprising the 2A coding region, the 2B coding region, the 2C coding region, the 3A coding region, the 3B coding region, the VPg coding region, the 3C coding region, the 3D pol coding region, and the 3′ UTR has at least 90% sequence identity to nucleotide 3492 to 7435 in SEQ ID NO: 3.

45. The recombinant RNA replicon of any one of claims 41 to 44, wherein the coxsackievirus genome comprises, from 5′ to 3′, the 5′ UTR, the heterologous polynucleotide, and the 2A coding region.

46. The recombinant RNA replicon of any one of claims 1 to 11, wherein the picornavirus is an encephalomyocarditis virus (EMCV).

47. The recombinant RNA replicon of any one of claims 9 and 11-46, wherein the recombinant RNA replicon comprises an internal ribosome entry site (IRES) inserted between the heterologous polynucleotide and the 2B coding region.

48. The recombinant RNA replicon of any one of claims 1 to 47, wherein the heterologous polynucleotide encodes one or more payload molecules.

49. The recombinant RNA replicon of any one of claims 1 to 47, wherein the heterologous polynucleotide encodes two or more payload molecules.

50. The recombinant RNA replicon of claim 49, wherein the two or more payload molecules are operably linked by one or more cleavage polypeptides.

51. The recombinant RNA replicon of claim 50, wherein the cleavage polypeptide comprises a 2A family self-cleaving peptide, a 3C cleavage site, a furin site, an IGSF1 polypeptide, or a HIV protease site.

52. The recombinant RNA replicon of claim 51, wherein the cleavage polypeptide comprises an IGSF1 polypeptide, and wherein the IGSF1 polypeptide comprises an amino acid sequence having at least 90% identity to SEQ ID NO: 75.

53. The recombinant RNA replicon of claim 51, wherein the cleavage polypeptide comprises an HIV protease site.

54. The recombinant RNA replicon of claim 51, wherein the cleavage polypeptide comprises a 2A family self-cleaving peptide.

55. The recombinant RNA replicon of any one of claims 50 to 54, wherein the cleavage polypeptide comprises a furin site.

56. The recombinant RNA replicon of any one of claims 50 to 55, wherein the heterologous polynucleotide encodes a polypeptide comprising the two or more payload molecules and the cleavage polypeptide comprising, from N-terminus to C-terminus: N′-payload molecule 1-cleavage polypeptide-payload molecule 2-C′.

57. The recombinant RNA replicon of claim 53, wherein the heterologous polynucleotide further comprises a coding region that encodes an HIV protease, and wherein the heterologous polynucleotide comprises a coding region that encodes a polypeptide comprising, from N-terminus to C-terminus: N′-Payload molecule 1-HIV protease site-HIV protease-HIV protease site-Payload molecule 2-C′.

58. The recombinant RNA replicon of claim 57, wherein the heterologous polynucleotide further comprises a coding region that encodes a third payload molecule, and wherein the heterologous polynucleotide comprises a coding region that encodes a polypeptide comprising, from N-terminus to C-terminus:
N′-Payload molecule 1-HIV protease site-HIV protease-HIV protease site-Payload molecule 2-HIV protease site-Payload molecule 3-C′.

59. The recombinant RNA replicon of any one of claims 56 to 58, further comprising a cleavage polypeptide at the C-terminus of the encoded polypeptide.

60. The recombinant RNA replicon of any one of claim 48 to 59, wherein the payload molecules are selected from a fluorescent protein, an enzyme, a cytokine, a chemokine, an antigen, an antigen-binding molecule capable of binding to a cell surface receptor, and a ligand for a cell-surface receptor.

61. The recombinant RNA replicon of any one of claim 48 to 59, wherein the payload molecules are selected from:
a) one or more cytokines comprising IFNγ, GM-CSF, IL-2, IL-12, IL-15, IL-18, IL-23, and IL-36γ;
b) one or more chemokines comprising CXCL10, CCL4, CCL5, and CCL21;
c) one or more antibodies comprising an anti-PD1-VHH-Fc antibody, an anti-CD47-VHH-Fc antibody, and an anti-TGFβ-VHH(or scFv)-Fc antibody;
d) one or more bipartite polypeptides comprising a bipartite polypeptide binding to DLL3 and an effector cell target antigen, a bipartite polypeptide binding to FAP and an effector cell target antigen, and a bipartite polypeptide binding to EpCAM and an effector cell target antigen;
e) one or more tumor-associated antigens comprising survivin, MAGE family proteins, and all antigens according to Table 6;
f) one or more tumor neoantigens;
g) one or more bipartite polypeptides binding to MHC-peptide antigen complex;
h) one or more fusogenic proteins comprising herpes simplex virus (HSV) UL27/glycoprotein B/gB, HSV UL53/glycoprotein K/gK, Respiratory syncytial virus (RSV) F protein, FASTp15, VSV-G, syncitin-1 (from human endogenous retrovirus-W (HERV-W)) or syncitin-2 (from HERVFRDE1), paramyxovirus SV5-F, measles virus-H, measles virus-F, and the glycoprotein from a retrovirus or lentivirus, such as gibbon ape leukemia virus (GALV), murine leukemia virus (MLV), Mason-Pfizer monkey virus (MPMV) and equine infectious anemia virus (EIAV), optionally with the R transmembrane peptide removed (R-versions);
i) one or more other payload molecules comprising IL15R, PGDH, ADA, ADA2, HYAL1, HYAL2, CHIPS, MLKL (or its 4HB domain only), GSDMD (or its L192A mutant, or its amino acids 1-233 fragment, or its amino acids 1-233 fragment with L192A mutation), GSDME (or its amino acid 1-237 fragment), HMGB1 (or its Box B domain only), Melittin (e.g., alpha-Melittin), SMAC/Diablo (or its amino acid 56-239 fragment), Snake LAAO, Snake disintegrin, Leptin, FLT3L, TRAIL, Gasdermin D or a truncation thereof, and Gasdermin E or a truncation thereof;
j) one or more antigens from pathogens comprising Dengue virus, Chikungunya virus, Mycobacterium tuberculosis, Human immunodeficiency viruses, SARS-CoV-2, Coronavirus, Hepatitis B Virus, Togaviridae family virus, Flaviviridae family virus, Influenza A virus, Influenza B virus, and a veterinary virus; or
k) any combination thereof.

62. The recombinant RNA replicon of any one of claims 49 to 59, wherein the two or more payload molecules are selected from the group consisting of a fluorescent protein, an enzyme, a cytokine, a chemokine, an antigen-binding molecule capable of binding to a cell surface receptor, and a ligand for a cell-surface receptor.

63. The recombinant RNA replicon of any one of claims 49 to 59, wherein the heterologous polynucleotide encodes two or more payload molecules comprising:
a. IL-2 and IL-36γ;
b. CXCL10 and an antigen binding molecule binding to FAP and CD3;
c. IL-2 and an antigen binding molecule binding to DLL3 and CD3;
d. IL-36γ and an antigen binding molecule binding to DLL3 and CD3; or
e. IL-2, IL-36γ and an antigen binding molecule binding to DLL3 and CD3.

64. The recombinant RNA replicon of any one of claims 1 to 63, further comprising a microRNA (miRNA) target sequence (miR-TS) cassette comprising one or more miRNA target sequences.

65. The recombinant RNA replicon of claim 64, wherein the one or more miRNAs comprise miR-124, miR-1, miR-143, miR-128, miR-219, miR-219a, miR-122, miR-204, miR-217, miR-137, and miR-126.

66. A recombinant DNA molecule comprising, from 5′ to 3′, a promoter sequence, a 5′ junctional cleavage sequence, a polynucleotide sequence encoding the recombinant RNA replicon of any one of claims 1-65, and a 3′ junctional cleavage sequence.

67. The recombinant DNA molecule of claim 66, wherein the promoter sequence is a T7 promoter sequence.

68. The recombinant DNA molecule of claim 66 or 67, wherein the 5′ junctional cleavage sequence is a ribozyme sequence and the 3′ junctional cleavage sequence is a ribozyme sequence.

69. The recombinant DNA molecule of claim 68, wherein the 5′ ribozyme sequence is a hammerhead ribozyme sequence and wherein the 3′ ribozyme sequence is a hepatitis delta virus ribozyme sequence.

70. The recombinant DNA molecule of claim 66 or 67, wherein the 5′ junctional cleavage sequence is a ribozyme sequence and the 3′ junctional cleavage sequence is a restriction enzyme recognition sequence.

71. The recombinant DNA molecule of claim 70, wherein the 5′ ribozyme sequence is a hammerhead ribozyme sequence, a Pistol ribozyme sequence, or a modified Pistol ribozyme sequence.

72. The recombinant DNA molecule of claim 70 or 71, wherein 3′ restriction enzyme recognition sequence is a Type IIS restriction enzyme recognition sequence.

73. The recombinant DNA molecule of claim 72, wherein the Type IIS recognition sequence is a SapI recognition sequence.

74. The recombinant DNA molecule of claim 66 or 67, wherein the 5′ junctional cleavage sequence is an RNAseH primer binding sequence and the 3′ junctional cleavage sequence is a restriction enzyme recognition sequence.

75. A method of producing the recombinant RNA replicon of any one of claims 1-65, comprising in vitro transcription of the DNA molecule of any one of claims 66-74 and purification of the resulting recombinant RNA replicon.

76. A composition comprising an effective amount of the recombinant RNA replicon of any one of claims 1-65, and a carrier suitable for administration to a mammalian subject.

77. A vector comprising the recombinant RNA replicon of any one of claims 1-65.

78. The vector of claim 77, wherein the vector is a viral vector.

79. The vector of claim 77, wherein the vector is a non-viral vector.

80. A particle comprising the recombinant RNA replicon of any one of claims 1-65.

81. The particle of claim 80, wherein the particle is selected from the group consisting of a nanoparticle, an exosome, a liposome, and a lipoplex.

82. The particle of claim 81, wherein the nanoparticle is a lipid nanoparticle (LNP) comprising a cationic lipid, one or more helper lipids, and a phospholipid-polymer conjugate.

83. The particle of claim 82, wherein the cationic lipid is selected from DLinDMA, DLin-KC2-DMA, DLin-MC3-DMA (MC3), COATSOME® SS-LC (former name: SS-18/4PE-13), COATSOME® SS-EC (former name: SS-33/4PE-15), COATSOME® SS-OC, COATSOME® SS-OP, Di((Z)-non-2-en-1-yl)9-((4-dimethylamino)butanoyl)oxy)heptadecanedioate (L-319), or N-(2,3-dioleoyloxy)propyl)-N,N,N-trimethylammonium chloride (DOTAP).

84. The particle of claim 82 or 83, wherein the helper lipid is selected from 1,2-distearoyl-sn-glycero-3-phosphocholine (DSPC); 1,2-dilauroyl-sn-glycero-3-phosphoethanolamine (DLPE); 1,2-dioleoyl-sn-glycero-3-phosphocholine (DOPC); 1,2-dioleoyl-sn-glycero-3-phosphoethanolamine (DOPE); and cholesterol.

85. The particle of claim 82, wherein the cationic lipid is 1,2-dioleoyl-3-trimethylammonium-propane (DOTAP), and wherein the neutral lipid is 1,2-Dilauroyl-sn-glycero-3-phosphoethanolamine (DLPE) or 1,2-Dioleoyl-sn-glycero-3-phosphoethanolamine (DOPE).

86. The particle of any one of claims 82-85, wherein the PEG-lipid is selected from 1,2-distearoyl-sn-glycero-3-phosphoethanolamine-N-[amino(polyethyleneglycol)] (DSPE-PEG); 1,2-dipalmitoyl-rac-glycerol methoxypolyethylene glycol (DPG-PEG); 1,2-distearoyl-rac-glycero-3-methylpolyoxyethylene (DSG-PEG); 1,2-distearoyl-rac-glycero-3-methylpolyoxyethylene (DSG-PEG); 1,2-dimyristoyl-rac-glycero-3-methylpolyoxyethylene (DMG-PEG); and 1,2-dimyristoyl-rac-glycero-3-methylpolyoxyethylene (DMG-PEG), or 1,2-distearoyl-sn-glycero-3-phosphoethanolamine-N-[amino(polyethylene glycol)] (DSPE-PEG-amine).

87. The particle of any one of claims 82-86, wherein the PEG-lipid is selected from 1,2-distearoyl-sn-glycero-3-phosphoethanolamine-N-[amino(polyethyleneglycol)-5000] (DSPE-PEG5K); 1,2-dipalmitoyl-rac-glycerol methoxypolyethylene glycol-2000 (DPG-PEG2K); 1,2-distearoyl-rac-glycero-3-methylpolyoxyethylene-5000 (DSG-PEG5K); 1,2-distearoyl-rac-glycero-3-methylpolyoxyethylene-2000 (DSG-PEG2K); 1,2-dimyristoyl-rac-glycero-3-methylpolyoxyethylene-5000 (DMG-PEG5K); and 1,2-dimyristoyl-rac-glycero-3-methylpolyoxyethylene-2000 (DMG-PEG2K).

88. The particle of claim 82, wherein the cationic lipid comprises COATSOME® SS-OC, wherein the one or more helper lipids comprise cholesterol (Chol) and DSPC, and wherein the phospholipid-polymer conjugate comprises DPG-PEG2000.

89. The particle of claim 88, wherein the ratio of SS-OC:DSPC:Chol:DPG-PEG2K (as a percentage of total lipid content) is A:B:C:D, wherein:
a. A=40%-60%, B=10%-25%, C=20%-30%, and D=0%-3% and wherein A+B+C+D=100%;
b. A=45%-50%, B=20%-25%, C=25%-30%, and D=0%-1% and wherein A+B+C+D=100%
c. A=40%-60%, B=10%-30%, C=20%-45%, and D=0%-3% and wherein A+B+C+D=100%;
d. A=40%-60%, B=10%-30%, C=25%-45%, and D=0%-3% and wherein A+B+C+D=100%;
e. A=45%-55%, B=10%-20%, C=30%-40%, and D=1%-2% and wherein A+B+C+D=100%;
f. A=45%-50%, B=10%-15%, C=35%-40%, and D=1%-2% and wherein A+B+C+D=100%;
g. A=45%-65%, B=5%-20%, C=20%-45%, and D=0%-3% and wherein A+B+C+D=100%;
h. A=50%-60%, B=5%-15%, C=30%-45%, and D=0%-3% and wherein A+B+C+D=100%;
i. A=55%-60%, B=5%-15%, C=30%-40%, and D=1%-2% and wherein A+B+C+D=100%;
j. A=55%-60%, B=5%-10%, C=30%-35%, and D=1%-2% and wherein A+B+C+D=100%.

90. The particle of claim 88, wherein the ratio of SS-OC:DSPC:Chol:DPG-PEG2K (as a percentage of total lipid content) is:
a. about 49:22:28.5:0.5;
b. about 49:11:38.5:1.5; or
c. about 58:7:33.5:1.5.

91. The particle of claim 88, wherein the ratio of SS-OC:DSPC:Chol:DPG-PEG2K (as a percentage of total lipid content) is about 49:22:28.5:0.5.

92. The particle of claim 82, wherein the cationic lipid is 1,2-dioleoyl-3-trimethylammonium-propane (DOTAP), and wherein the neutral lipid is 1,2-Dilauroyl-sn-glycero-3-phosphoethanolamine (DLPE) or 1,2-Dioleoyl-sn-glycero-3-phosphoethanolamine (DOPE).

93. The particle of claim 82 or 92, further comprising a PEG-lipid, wherein the PEG-lipid is 1, 2-Distearoyl-sn-glycero-3-phosphoethanolamine-Poly(ethylene glycol) (DSPE-PEG) or 1,2-distearoyl-sn-glycero-3-phosphoethanolamine-N-[amino(polyethylene glycol)] (DSPE-PEG-amine).

94. The particle of any one of claims 80-93, further comprising a second recombinant RNA molecule encoding an oncolytic virus.

95. The particle of claim 94, wherein the oncolytic virus is a picornavirus.

96. The particle of claim 95, wherein the picornavirus is selected from a senecavirus, a cardiovirus, and an enterovirus.

97. The particle of claim 95, wherein the picornavirus is a Seneca Valley Virus (SVV).

98. The particle of claim 95, wherein the picornavirus is a Coxsackievirus.

99. The particle of claim 95, wherein the picornavirus is an encephalomyocarditis virus (EMCV).

100. A therapeutic composition comprising a plurality of lipid nanoparticles according to any one of claims 82-99.

101. The therapeutic composition of claim 100 wherein the plurality of LNPs have an average size of about 50 nm to about 120 nm.

102. The therapeutic composition of claim 100 wherein the plurality of LNPs have an average size of about 100 nm.

103. The therapeutic composition of any one of claims 100-102, wherein the plurality of LNPs have an average zeta-potential of between about 20 mV to about −20 mV, about 10 mV to about −10 mV, about 5 mV to about −5 mV, or about 20 mV to about −40 mV, −50 mV to about −20 mV, about −40 mV to about −20 mV, or about −30 mV to about −20 mV.

104. The therapeutic composition of claim 103, wherein the plurality of LNPs have an average zeta-potential of about −30 mV, about −31 mV, about −32 mV, about −33 mV, about −34 mV, about −35 mV, about −36 mV, about −37 mV, about −38 mV, about −39 mV, or about −40 mV.

105. A method of killing a cancerous cell comprising exposing the cancerous cell to the particle of any one of claims 80-97, the vector of any one of claims 77-79, the recombinant RNA replicon of any one of claims 1-65, or compositions thereof.

106. The method of claim 105, wherein the method is performed in vivo, in vitro, or ex vivo.

107. A method of treating a cancer in a subject comprising administering to the subject suffering from the cancer an effective amount of the particle of any one of claims 80-97, the vector of any one of claims 77-79, the recombinant RNA replicon of any one of claims 1-65, or compositions thereof.

108. The method of claim 107, wherein the particle, the recombinant RNA replicon, or composition thereof is administered intravenously, intranasally, as an inhalant, or is injected directly into a tumor.

109. The method of claim 107 or 108, wherein the particle, the recombinant RNA replicon, or composition thereof is administered to the subject repeatedly.

110. The method of any of claims 107-109, wherein the subject is a mouse, a rat, a rabbit, a cat, a dog, a horse, a non-human primate, or a human.

111. The method of any of claims 107-110, wherein the cancer is selected from lung cancer, breast cancer, ovarian cancer, cervical cancer, prostate cancer, testicular cancer, colorectal cancer, colon cancer, pancreatic cancer (e.g., Castration resistant neuroendocrine prostate cancer), liver cancer, gastric cancer, head and neck cancer, thyroid cancer, malignant glioma, glioblastoma, melanoma, B-cell chronic lymphocytic leukemia, diffuse large B-cell lymphoma (DLBCL), sarcoma, a neuroblastoma, a neuroendocrine cancer, a rhabdomyosarcoma, a medulloblastoma, a bladder cancer, marginal zone lymphoma (MZL), Merkel cell carcinoma, and renal cell carcinoma.

112. The method of claim 111, wherein:
a. the lung cancer is small cell lung cancer or non-small cell lung cancer;
b. the liver cancer is hepatocellular carcinoma (HCC); and/or
c. the prostate cancer is treatment-emergent neuroendocrine prostate cancer.

113. The method of claim 111, wherein the cancer is a neuroendocrine cancer.

114. A method of immunizing a subject against a disease, comprising administering to the subject an effective amount of the particle of any one of claims 80-97, the vector of any one of claims 77-79, the recombinant RNA replicon of any one of claims 1-65, or compositions thereof.

115. The method of claim 114, wherein the particle, the recombinant RNA replicon, or composition thereof is administered intravenously, intramuscularly, intradermally, intranasally, or as an inhalant.

116. The method of claim 114 or 115, wherein the particle, the recombinant RNA replicon, or composition thereof is administered to the subject repeatedly.

117. The method of any one of claims 114 to 116, wherein the disease is an infectious disease.

118. The method of claim 117, wherein the infectious disease is caused by one of the pathogens comprising Dengue virus, Chikungunya virus, Mycobacterium tuberculosis, Human immunodeficiency virus, SARS-CoV-2, Coronavirus, Hepatitis B virus, Togaviridae family virus, Flaviviridae family virus, Influenza A virus, Influenza B virus and a veterinary virus.

119. A recombinant RNA replicon comprising a picornavirus genome and a heterologous polynucleotide.

120. The recombinant RNA replicon of claim 119, wherein the heterologous polynucleotide is inserted between a 2A coding region and a 2B coding region.

121. The recombinant RNA replicon of claim 119, wherein the heterologous polynucleotide is inserted between a 5′ UTR and a 2A coding region.

122. The recombinant RNA replicon of claim 119, wherein the heterologous polynucleotide is inserted between a 3D coding region and a 3′ UTR.

123. The recombinant RNA replicon of any one of claims 119-122, wherein the picornavirus is selected from a senecavirus, a cardiovirus, and an enterovirus.
